SEQUENCE LISTING (369 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 97 aa, Homo sapiens
RTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - DNA, 330 bp, synthetic construct
  source 1..330: mol_type = other DNA
cgcacagtgagaacccctaagatacaagtttatagtcggcaccccgcagagaacgggaaaagtaacttccttaattgctatgtctctggcttccatccttctgacatcgaggtggacttgttgaaaaacggggagagaattgaaaaagtggaacacagcgatctgagcttctccaaagattggtctttttatctcctctactacaccgaatttaccccaactgagaaagatgaatatgcctgtcgggttaatcacgtaactttgtctcaaccaaaaattgtcaaatgggacagggacatgtcagggtggagtcaccctcagttcgaaaag

SEQ ID NO: 4 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 5 - protein, 100 aa, synthetic construct
RTVRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 6 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 7 - protein, 97 aa, synthetic construct
RTPKIQVFSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 8 - protein, 100 aa, synthetic construct
RTVRTPKIQVFSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 9 - protein, 97 aa, synthetic construct
RTPKIQVWSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 10 - protein, 100 aa, synthetic construct
RTVRTPKIQVWSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 11 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRFM

SEQ ID NO: 12 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRFM

SEQ ID NO: 13 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRWM

SEQ ID NO: 14 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRWM

SEQ ID NO: 15 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDF

SEQ ID NO: 16 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDF

SEQ ID NO: 17 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDW

SEQ ID NO: 18 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDW

SEQ ID NO: 19 - protein, 96 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRD

SEQ ID NO: 20 - protein, 99 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRD

SEQ ID NO: 21 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDC

SEQ ID NO: 22 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDC

SEQ ID NO: 23 - protein, 97 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCM

SEQ ID NO: 24 - protein, 100 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCM

SEQ ID NO: 25 - protein, 97 aa, synthetic construct
RTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 26 - protein, 100 aa, synthetic construct
RTVRTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 27 - protein, 97 aa, synthetic construct
RTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 28 - protein, 100 aa, synthetic construct
RTVRTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 29 - protein, 97 aa, synthetic construct
RTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 30 - protein, 100 aa, synthetic construct
RTVRTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDM

SEQ ID NO: 31 - protein, 358 aa, Homo sapiens
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 32 - protein, 92 aa, Homo sapiens
EPPKTHVTHHPISDHEATLRCWALGFYPAEITLTWQQDGEGHTQDTELVETRPAGDGTFQKWAAVVVPSGEEQRYTCHVQHEGLPEPVTLRW

SEQ ID NO: 33 - protein, 95 aa, Homo sapiens
LHLEPPKTHVTHHPISDHEATLRCWALGFYPAEITLTWQQDGEGHTQDTELVETRPAGDGTFQKWAAVVVPSGEEQRYTCHVQHEGLPEPVTLRW

SEQ ID NO: 34 - skipped

SEQ ID NO: 35 - protein, 95 aa, synthetic construct
LHLEPPKTHVTHHPISDHEATLRCWALGFYPAEITLTWQQDGEGHTQDTELVETAPAGDGTFQKWAAVVVPSGEEQRYTCHVQHEGLPEPVTLRW

SEQ ID NO: 36 - protein, 95 aa, synthetic construct
LHLEPPKTHVTHHPISDHEATLRCWALGFYPAEITLTWQQDGEGHTQDTELVETLPAGDGTFQKWAAVVVPSGEEQRYTCHVQHEGLPEPVTLRW

SEQ ID NO: 37 - protein, 95 aa, synthetic construct
LHLEPPKTHVTHHPISDHEATLRCWALGFYPAEITLTWQQDGEGHTQDTELVETAPAGDGTFAKWAAVVVPSGEEQRYTCHVQHEGLPEPVTLRW

SEQ ID NO: 38 - protein, 95 aa, synthetic construct
LHLEPPKTHVTHHPISDHEATLRCWALGFYPAEITLTWQQDGEGHTQDTELVETLPAGDGTFLKWAAVVVPSGEEQRYTCHVQHEGLPEPVTLRW

SEQ ID NO: 39 - protein, 95 aa, synthetic construct
LHLEPPKTHVTHHPISDHEATLRCWALGFYPAEITLTWQQDGEGHTQDTELVETAPAGDGTFLKWAAVVVPSGEEQRYTCHVQHEGLPEPVTLRW

SEQ ID NO: 40 - protein, 95 aa, synthetic construct
LHLEPPKTHVTHHPISDHEATLRCWALGFYPAEITLTWQQDGEGHTQDTELVETLPAGDGTFAKWAAVVVPSGEEQRYTCHVQHEGLPEPVTLRW

SEQ ID NO: 41 - protein, 95 aa, synthetic construct
LHLEPPKTHVTHHPISDHEATLACWALGFYPAEITLTWQQDGEGHTQDTELVETRPAGDGTFQKWAAVVVPSGEEQRYTCHVQHEGLPEPVTLRW

SEQ ID NO: 42 - protein, 95 aa, synthetic construct
LHLEPPKTHVTHHPISDHEATLCCWALGFYPAEITLTWQQDGEGHTQDTELVETRPAGDGTFQKWAAVVVPSGEEQRYTCHVQHEGLPEPVTLRW

SEQ ID NO: 43 - protein, 95 aa, synthetic construct
LHLEPPKTHVTHCPISDHEATLRCWALGFYPAEITLTWQQDGEGHTQDTELVETRPAGDGTFQKWAAVVVPSGEEQRYTCHVQHEGLPEPVTLRW

SEQ ID NO: 44 - protein, 95 aa, synthetic construct
LHLEPPKTHVTHHPISDHEATLRCWALGFYPAEITLTWQQDGEGHTQDTELVCTRPAGDGTFQKWAAVVVPSGEEQRYTCHVQHEGLPEPVTLRW

SEQ ID NO: 45 - protein, 95 aa, synthetic construct
LHLEPPKTHVTHHPISDHEATLRCWALGFYPAEITLTWQQDGEGHTQDTELVETCPAGDGTFQKWAAVVVPSGEEQRYTCHVQHEGLPEPVTLRW

SEQ ID NO: 46 - protein, 95 aa, synthetic construct
LHLEPPKTHVTHHPISDHEATLRCWALGFYPAEITLTWQQDGEGHTQDTELVETRPAGCGTFQKWAAVVVPSGEEQRYTCHVQHEGLPEPVTLRW

SEQ ID NO: 47 - protein, 532 aa, Homo sapiens
MAPSSPRPALPALLVLLGALFPGPGNAQTSVSPSKVILPRGGSVLVTCSTSCDQPKLLGIETPLPKKELLLPGNNRKVYELSNVQEDSQPMCYSNCPDGQSTAKTFLTVYWTPERVELAPLPSWQPVGKNLTLRCQVEGGAPRANLTVVLLRGEKELKREPAVGEPAEVTTTVLVRRDHHGANFSCRTELDLRPQGLELFENTSAPYQLQTFVLPATPPQLVSPRVLEVDTQGTVVCSLDGLFPVSEAQVHLALGDQRLNPTVTYGNDSFSAKASVSVTAEDEGTQRLTCAVILGNQSQETLQTVTIYSFPAPNVILTKPEVSEGTEVTVKCEAHPRAKVTLNGVPAQPLGPRAQLLLKATPEDNGRSFSCSATLEVAGQLIHKNQTRELRVLYGPRLDERDCPGNWTWPENSQQTPMCQAWGNPLPELKCLKDGTFPLPIGESVTVTRDLEGTYLCRARSTQGEVTRKVTVNVLSPRYEIVIITVVAAAVIMGTAGLSTYLYNRQRKIKKYRLQQAQKGTPMKPNTQATPP

SEQ ID NO: 48 - protein, 63 aa, Homo sapiens
GGSVLVTCSTSCDQPKLLGIETPLPKKELLLPGNNRKVYELSNVQEDSQPMCYSNCPDGQSTA

SEQ ID NO: 49 - protein, 83 aa, Homo sapiens
QTSVSPSKVILPRGGSVLVTCSTSCDQPKLLGIETPLPKKELLLPGNNRKVYELSNVQEDSQPMCYSNCPDGQSTAKTFLTVY

SEQ ID NO: 50 - skipped

SEQ ID NO: 51 - protein, 83 aa, synthetic construct
QTSVSPSKVILPRGGSVLVTCSTSCDQPKLLGIKTPLPKKELLLPGNNRKVYELSNVQEDSQPMCYSNCPDGQSTAKTFLTVY

SEQ ID NO: 52 - protein, 83 aa, synthetic construct
QVSVSPSKVTLPRGGSVLVTCSASCDQPKLLGIKTPLTKKELLLPGNNRKVYELSNVQEDSQVMCYANCPDGQSTAKAFLTVY

SEQ ID NO: 53 - protein, 84 aa, synthetic construct
QVSVSPSKVTLPCGGSVLVTCSASCDQPKLLGIKTPLTKKELLLPGNNRKVYELSNVQEDSQVMCYANCPDGQSTAKAFLTVYC

SEQ ID NO: 54 - protein, 84 aa, synthetic construct
QVSVSPSKVTLPCGGSVLVTCSASCDQPKLLGIKTPLTKKELLLPGNNEKVYELSNVQEDSQVMCYANCPDGQSTAKAFLTVYC

SEQ ID NO: 55 - protein, 84 aa, synthetic construct
QVSVSPSKVTLPCGGSVLVTCSASCDQPKLLGIKTPLTKKELLLPGNNRKVYRLSNVQEDSQVMCYANCPDGQSTAKAFLTVYC

SEQ ID NO: 56 - protein, 84 aa, synthetic construct
QVSVSPSKVTLPCGGSVFVTCSASCDQPKLLGIKTPLTKKELLLPGNNRKVYELSNVQEDSQVMCYANCPDGQSTAKAFLTVYC

SEQ ID NO: 57 - protein, 84 aa, synthetic construct
QVSVSPSKVTLPCGGSVAVACSASCDQPKLLGIKTPLTKKEALLPGNNRKAYELSNVQEDSQVMCYANCPDGQSTAKAFLTVYC

SEQ ID NO: 58 - protein, 84 aa, synthetic construct
QVSVSPSKVTLPCGGSVWVTCSASCDQPKLLGIKTPLTKKELLLPGNNRKVYELSNVQEDSQVMCYANCPDGQSTAKAFLTVYC

SEQ ID NO: 59 - protein, 6 aa, synthetic construct
RTVGGS

SEQ ID NO: 60 - protein, 9 aa, synthetic construct
RTVGGSRTV

SEQ ID NO: 61 - protein, 4 aa, synthetic construct
ASTK

SEQ ID NO: 62 - protein, 5 aa, synthetic construct
ASTKG

SEQ ID NO: 63 - protein, 6 aa, synthetic construct
ASTKGG

SEQ ID NO: 64 - protein, 7 aa, synthetic construct
ASTKGGS

SEQ ID NO: 65 - protein, 8 aa, synthetic construct
ASTKGGGS

SEQ ID NO: 66 - protein, 9 aa, synthetic construct
ASTKGGGGS

SEQ ID NO: 67 - protein, 10 aa, synthetic construct
ASTKGGGGSG

SEQ ID NO: 68 - protein, 11 aa, synthetic construct
ASTKGGGGSGG

SEQ ID NO: 69 - protein, 12 aa, synthetic construct
ASTKGGGGSGGS

SEQ ID NO: 70 - protein, 13 aa, synthetic construct
ASTKGGGGSGGGS

SEQ ID NO: 71 - protein, 14 aa, synthetic construct
ASTKGGGGSGGGGS

SEQ ID NO: 72 - protein, 4 aa, synthetic construct
RTVA

SEQ ID NO: 73 - protein, 5 aa, synthetic construct
RTVAG

SEQ ID NO: 74 - protein, 6 aa, synthetic construct
RTVAGG

SEQ ID NO: 75 - protein, 7 aa, synthetic construct
RTVAGGS

SEQ ID NO: 76 - protein, 8 aa, synthetic construct
RTVAGGGS

SEQ ID NO: 77 - protein, 9 aa, synthetic construct
RTVAGGGGS

SEQ ID NO: 78 - protein, 10 aa, synthetic construct
RTVAGGGGSG

SEQ ID NO: 79 - protein, 11 aa, synthetic construct
RTVAGGGGSGG

SEQ ID NO: 80 - protein, 12 aa, synthetic construct
RTVAGGGGSGGS

SEQ ID NO: 81 - protein, 13 aa, synthetic construct
RTVAGGGGSGGGS

SEQ ID NO: 82 - protein, 14 aa, synthetic construct
RTVAGGGGSGGGGS

SEQ ID NO: 83 - protein, 10 aa, synthetic construct
GGGGSGGGGS

SEQ ID NO: 84 - protein, 15 aa, synthetic construct
GGGGSGGGGSGGGGS

SEQ ID NO: 85 - protein, 20 aa, synthetic construct
GGGGSGGGGSGGGGSGGGGS

SEQ ID NO: 86 - protein, 25 aa, synthetic construct
GGGGSGGGGSGGGGSGGGGSGGGGS

SEQ ID NO: 87 - protein, 5 aa, synthetic construct
EPKSS

SEQ ID NO: 88 - protein, 5 aa, synthetic construct
EPKSC

SEQ ID NO: 89 - protein, 8 aa, synthetic construct
GGSGECSG

SEQ ID NO: 90 - protein, 9 aa, synthetic construct
GGGSGECSG

SEQ ID NO: 91 - protein, 8 aa, synthetic construct
GGSGESSG

SEQ ID NO: 92 - protein, 9 aa, synthetic construct
GGGSGESSG

SEQ ID NO: 93 - protein, 6 aa, synthetic construct
HHHHHH

SEQ ID NO: 94 - protein, 8 aa, synthetic construct
WSHPQFEK

SEQ ID NO: 95 - protein, 9 aa, synthetic construct
YPYDVPDYA

SEQ ID NO: 96 - protein, 120 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSS

SEQ ID NO: 97 - protein, 111 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIK

SEQ ID NO: 98 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 99 - protein, 107 aa, Homo sapiens [sequence omitted]

SEQ ID NO: 100 - protein, 106 aa, synthetic construct
LHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 101 - protein, 107 aa, synthetic construct
RTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMSGWSHPQFEK

SEQ ID NO: 102 - protein, 107 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 103 - protein, 110 aa, synthetic construct
RTVRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMSGWSHPQFEK

SEQ ID NO: 104 - protein, 110 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 105 - protein, 226 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSLHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 106 - protein, 221 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 107 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 108 - protein, 224 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 109 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCHHHHHH

SEQ ID NO: 110 - protein, 227 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGWSHPQFEK

SEQ ID NO: 111 - protein, 106 aa, synthetic construct
LHLEPPKTHVTHHPISDHEATLRCWALGFYPAEITLTWQQDGEGHTQDTELVETAPAGDGTFQKWAAVVVPSGEEQRYTCHVQHEGLPEPVTLRWEPKSSHHHHHH

SEQ ID NO: 112 - protein, 106 aa, synthetic construct
LHLEPPKTHVTHHPISDHEATLRCWALGFYPAEITLTWQQDGEGHTQDTELVETLPAGDGTFQKWAAVVVPSGEEQRYTCHVQHEGLPEPVTLRWEPKSSHHHHHH

SEQ ID NO: 113 - protein, 106 aa, synthetic construct
LHLEPPKTHVTHHPISDHEATLRCWALGFYPAEITLTWQQDGEGHTQDTELVETAPAGDGTFAKWAAVVVPSGEEQRYTCHVQHEGLPEPVTLRWEPKSSHHHHHH

SEQ ID NO: 114 - protein, 106 aa, synthetic construct
LHLEPPKTHVTHHPISDHEATLRCWALGFYPAEITLTWQQDGEGHTQDTELVETLPAGDGTFLKWAAVVVPSGEEQRYTCHVQHEGLPEPVTLRWEPKSSHHHHHH

SEQ ID NO: 115 - protein, 106 aa, synthetic construct
LHLEPPKTHVTHHPISDHEATLRCWALGFYPAEITLTWQQDGEGHTQDTELVETAPAGDGTFLKWAAVVVPSGEEQRYTCHVQHEGLPEPVTLRWEPKSSHHHHHH

SEQ ID NO: 116 - protein, 106 aa, synthetic construct
LHLEPPKTHVTHHPISDHEATLRCWALGFYPAEITLTWQQDGEGHTQDTELVETLPAGDGTFAKWAAVVVPSGEEQRYTCHVQHEGLPEPVTLRWEPKSSHHHHHH

SEQ ID NO: 117 - protein, 106 aa, synthetic construct
LHLEPPKTHVTHHPISDHEATLACWALGFYPAEITLTWQQDGEGHTQDTELVETRPAGDGTFQKWAAVVVPSGEEQRYTCHVQHEGLPEPVTLRWEPKSSHHHHHH

SEQ ID NO: 118 - protein, 107 aa, synthetic construct
RTPKIQVFSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 119 - protein, 110 aa, synthetic construct
RTVRTPKIQVFSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 120 - protein, 107 aa, synthetic construct
RTPKIQVWSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 121 - protein, 110 aa, synthetic construct
RTVRTPKIQVWSRHPAENGKSNFLNCYVSGFHPSDIEVDLLKNGERIEKVEHSDLSSSKDSSTYLLYYTEFTPTEKDEYACRVNHVTLSQPKIVKWDRDMSGWSHPQFEK

SEQ ID NO: 122 - protein, 107 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRFMSGWSHPQFEK

SEQ ID NO: 123 - protein, 110 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRFMSGWSHPQFEK

SEQ ID NO: 124 - protein, 107 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRWMSGWSHPQFEK

SEQ ID NO: 125 - protein, 110 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRWMSGWSHPQFEK

SEQ ID NO: 126 - protein, 107 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDFSGWSHPQFEK

SEQ ID NO: 127 - protein, 110 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDFSGWSHPQFEK

SEQ ID NO: 128 - protein, 107 aa, synthetic construct
RTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDWSGWSHPQFEK

SEQ ID NO: 129 - protein, 110 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDWSGWSHPQFEK

SEQ ID NO: 130 - protein, 106 aa, synthetic construct
LHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPKSCHHHHHH

SEQ ID NO: 131 - protein, 106 aa, synthetic construct
LHLEPPKTHVTHHPISDHEATLCCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 132 - protein, 106 aa, synthetic construct
LHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 133 - protein, 106 aa, synthetic construct
LHLEPPKTHVTHHPISDHEATLRCWALGFYPAEITLTWQQDGEGHTQDTELVCTRPAGDGTFQKWAAVVVPSGEEQRYTCHVQHEGLPEPVTLRWEPKSSHHHHHH

SEQ ID NO: 134 - protein, 106 aa, synthetic construct
LHLEPPKTHVTHHPISDHEATLRCWALGFYPAEITLTWQQDGEGHTQDTELVETCPAGDGTFQKWAAVVVPSGEEQRYTCHVQHEGLPEPVTLRWEPKSSHHHHHH

SEQ ID NO: 135 - protein, 106 aa, synthetic construct
LHLEPPKTHVTHHPISDHEATLRCWALGFYPAEITLTWQQDGEGHTQDTELVETRPAGCGTFQKWAAVVVPSGEEQRYTCHVQHEGLPEPVTLRWEPKSSHHHHHH

SEQ ID NO: 136 - protein, 116 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDGGSGECSGYPYDVPDYA

SEQ ID NO: 137 - protein, 117 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDGGGSGECSGYPYDVPDYA

SEQ ID NO: 138 - protein, 117 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDCGGSGESSGYPYDVPDYA

SEQ ID NO: 139 - protein, 118 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDCGGGSGESSGYPYDVPDYA

SEQ ID NO: 140 - protein, 117 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGGSGESSGYPYDVPDYA

SEQ ID NO: 141 - protein, 118 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGGGSGESSGYPYDVPDYA

SEQ ID NO: 142 - protein, 117 aa, synthetic construct
RTVRTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 143 - protein, 118 aa, synthetic construct
RTVRTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 144 - protein, 117 aa, synthetic construct
RTVRTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 145 - protein, 118 aa, synthetic construct
RTVRTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 146 - protein, 117 aa, synthetic construct
RTVRTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 147 - protein, 118 aa, synthetic construct
RTVRTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 148 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLRCWALGFYPAEITLTWQQDGEGHTQDTELVETRPAGDGTFQKWAAVVVPSGEEQRYTCHVQHEGLPEPVTLRWEPKSCHHHHHH

SEQ ID NO: 149 - protein, 233 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDGGSGECSGYPYDVPDYA

SEQ ID NO: 150 - protein, 229 aa, synthetic construct; identical to SEQ ID NO: 148

SEQ ID NO: 151 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDGGGSGECSGYPYDVPDYA

SEQ ID NO: 152 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLCCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 153 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDCGGSGESSGYPYDVPDYA

SEQ ID NO: 154 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 155 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGGSGESSGYPYDVPDYA

SEQ ID NO: 156 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLRCWALGFYPAEITLTWQQDGEGHTQDTELVCTRPAGDGTFQKWAAVVVPSGEEQRYTCHVQHEGLPEPVTLRWEPKSSHHHHHH

SEQ ID NO: 157 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 158 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLRCWALGFYPAEITLTWQQDGEGHTQDTELVETCPAGDGTFQKWAAVVVPSGEEQRYTCHVQHEGLPEPVTLRWEPKSSHHHHHH

SEQ ID NO: 159 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 160 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGSLHLEPPKTHVTHHPISDHEATLRCWALGFYPAEITLTWQQDGEGHTQDTELVETRPAGCGTFQKWAAVVVPSGEEQRYTCHVQHEGLPEPVTLRWEPKSSHHHHHH

SEQ ID NO: 161 - protein, 234 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDMGGSGESSGYPYDVPDYA

SEQ ID NO: 162 - protein, 229 aa, synthetic construct; identical to SEQ ID NO: 152

SEQ ID NO: 163 - protein, 235 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRDCGGGSGESSGYPYDVPDYA

SEQ ID NO: 164 - protein, 229 aa, synthetic construct; identical to SEQ ID NO: 154

SEQ ID NO: 165 - protein, 235 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGGGSGESSGYPYDVPDYA

SEQ ID NO: 166 - protein, 229 aa, synthetic construct; identical to SEQ ID NO: 156

SEQ ID NO: 167 - protein, 235 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPCIQVYSRHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 168 - protein, 229 aa, synthetic construct; identical to SEQ ID NO: 158

SEQ ID NO: 169 - protein, 235 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVCSRHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 170 - protein, 229 aa, synthetic construct; identical to SEQ ID NO: 160

SEQ ID NO: 171 - protein, 235 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVGGSRTVRTPKIQVYSCHPAENGKSNFLNCYVSGFHPSDIEVDLLKNGERIEKVEHSDLSSSKDSSTYLLYYTEFTPTEKDEYACRVNHVTLSQPKIVKWDRDMGGGSGESSGYPYDVPDYA

SEQ ID NO: 172 - protein, 229 aa, synthetic construct; identical to SEQ ID NO: 109

SEQ ID NO: 173 - protein, 232 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSYPYDVPDYA

SEQ ID NO: 174 - protein, 109 aa, synthetic construct
ASTLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 175 - protein, 110 aa, synthetic construct
ASTKLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 176 - protein, 111 aa, synthetic construct
ASTKGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 177 - protein, 112 aa, synthetic construct
ASTKGGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 178 - protein, 113 aa, synthetic construct
ASTKGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 179 - protein, 114 aa, synthetic construct
ASTKGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 180 - protein, 115 aa, synthetic construct
ASTKGGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 181 - protein, 116 aa, synthetic construct
ASTKGGGGSGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 182 - protein, 117 aa, synthetic construct
ASTKGGGGSGGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 183 - protein, 118 aa, synthetic construct
ASTKGGGGSGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 184 - protein, 119 aa, synthetic construct
ASTKGGGGSGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 185 - protein, 120 aa, synthetic construct
ASTKGGGGSGGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 186 - protein, 111 aa, synthetic construct
RTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 187 - protein, 112 aa, synthetic construct
RTVARTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 188 - protein, 114 aa, synthetic construct
RTVAGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSGYPYDVPDYA

SEQ ID NO: 189 - protein, 114 aa, synthetic construct
RTVAGGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 190 - protein, 115 aa, synthetic construct
RTVAGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 191 - protein, 116 aa, synthetic construct
RTVAGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 192 - protein, 117 aa, synthetic construct
RTVAGGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 193 - protein, 118 aa, synthetic construct
RTVAGGGGSGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 194 - protein, 119 aa, synthetic construct
RTVAGGGGSGGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 195 - protein, 120 aa, synthetic construct
RTVAGGGGSGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 196 - protein, 121 aa, synthetic construct
RTVAGGGGSGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 197 - protein, 122 aa, synthetic construct
RTVAGGGGSGGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 198 - protein, 229 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 199 - protein, 222 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 200 - protein, 230 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 201 - protein, 223 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVARTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 202 - protein, 231 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 203 - protein, 224 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 204 - protein, 232 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 205 - protein, 225 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 206 - protein, 233 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 207 - protein, 226 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 208 - protein, 234 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 209 - protein, 227 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 210 - protein, 235 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 211 - protein, 228 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 212 - protein, 236 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGGSGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 213 - protein, 229 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGGSGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 214 - protein, 237 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGGSGGLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 215 - protein, 230 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGGSGGRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 216 - protein, 238 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGGSGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 217 - protein, 231 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGGSGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 218 - protein, 239 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSASTKGGGGSGGGSLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSHHHHHH

SEQ ID NO: 219 - protein, 232 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKRTVAGGGGSGGGSRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCMGSYPYDVPDYA

SEQ ID NO: 220 - protein, 239 aa, synthetic construct; identical to SEQ ID NO: 218

SEQ ID NO: 221 - protein, 232 aa, synthetic construct; identical to SEQ ID NO: 219

SEQ ID NO: 222 - protein, 94 aa, synthetic construct
QTSVSPSKVILPRGGSVLVTCSTSCDQPKLLGIETPLPKKELLLPGNNRKVYELSNVQEDSQPMCYSNCPDGQSTAKTFLTVYEPKSSHHHHHH

SEQ ID NO: 223 - protein, 94 aa, synthetic construct
QTSVSPSKVILPRGGSVLVTCSTSCDQPKLLGIKTPLPKKELLLPGNNRKVYELSNVQEDSQPMCYSNCPDGQSTAKTFLTVYEPKSSHHHHHH

SEQ ID NO: 224 - protein, 94 aa, synthetic construct
QVSVSPSKVTLPRGGSVLVTCSASCDQPKLLGIKTPLTKKELLLPGNNRKVYELSNVQEDSQVMCYANCPDGQSTAKAFLTVYEPKSSHHHHHH

SEQ ID NO: 225 - protein, 95 aa, synthetic construct
QVSVSPSKVTLPCGGSVLVTCSASCDQPKLLGIKTPLTKKELLLPGNNRKVYELSNVQEDSQVMCYANCPDGQSTAKAFLTVYCEPKSSHHHHHH

SEQ ID NO: 226 - protein, 95 aa, synthetic construct
QVSVSPSKVTLPCGGSVLVTCSASCDQPKLLGIKTPLTKKELLLPGNNEKVYELSNVQEDSQVMCYANCPDGQSTAKAFLTVYCEPKSSHHHHHH

SEQ ID NO: 227 - protein, 94 aa, synthetic construct
QVSVSPSKVTLPCGGSVLVTCSASCDQPKLLGIKTPLTKKELLLPGNNRKVYRLSNVQEDSQVMCYANCPDGQSTAKAFLTVYCSGWSHPQFEK

SEQ ID NO: 228 - protein, 95 aa, synthetic construct
QVSVSPSKVTLPCGGSVFVTCSASCDQPKLLGIKTPLTKKELLLPGNNRKVYELSNVQEDSQVMCYANCPDGQSTAKAFLTVYCEPKSSHHHHHH

SEQ ID NO: 229 - protein, 94 aa, synthetic construct
QVSVSPSKVTLPCGGSVAVACSASCDQPKLLGIKTPLTKKEALLPGNNRKAYELSNVQEDSQVMCYANCPDGQSTAKAFLTVYCSGWSHPQFEK

SEQ ID NO: 230 - protein, 95 aa, synthetic construct
QVSVSPSKVTLPCGGSVWVTCSASCDQPKLLGIKTPLTKKELLLPGNNRKVYELSNVQEDSQVMCYANCPDGQSTAKAFLTVYCEPKSSHHHHHH

SEQ ID NO: 231 - protein, 225 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCEPKSSHHHHHH

SEQ ID NO: 232 - protein, 215 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCSGWSHPQFEK

SEQ ID NO: 233 - protein, 230 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCEPKSSHHHHHH

SEQ ID NO: 234 - protein, 220 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCSGWSHPQFEK

SEQ ID NO: 235 - protein, 235 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCEPKSSHHHHHH

SEQ ID NO: 236 - protein, 225 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCSGWSHPQFEK

SEQ ID NO: 237 - protein, 240 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCEPKSSHHHHHH

SEQ ID NO: 238 - protein, 230 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSGGGGSQVSVSPSKVTLPCGGSVLVTCSASCDQPKLLGIKTPLTKKELLLPGNNRKVYELSNVQEDSQVMCYANCPDGQSTAKAFLTVYCSGWSHPQFEK

SEQ ID NO: 239 - protein, 216 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSQVSVSPSKVTLPCGGSVLVTCSASCDQPKLLGIKTPLTKKELLLPGNNEKVYELSNVQEDSQVMCYANCPDGQSTAKAFLTVYCEPKSSHHHHHH

SEQ ID NO: 240 - protein, 215 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSQVSVSPSKVTLPCGGSVLVTCSASCDQPKLLGIKTPLTKKELLLPGNNRKVYRLSNVQEDSQVMCYANCPDGQSTAKAFLTVYCSGWSHPQFEK

SEQ ID NO: 241 - protein, 230 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSQVSVSPSKVTLPCGGSVLVTCSASCDQPKLLGIKTPLTKKELLLPGNNEKVYELSNVQEDSQVMCYANCPDGQSTAKAFLTVYCEPKSSHHHHHH

SEQ ID NO: 242 - protein, 220 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSQVSVSPSKVTLPCGGSVLVTCSASCDQPKLLGIKTPLTKKELLLPGNNRKVYRLSNVQEDSQVMCYANCPDGQSTAKAFLTVYCSGWSHPQFEK

SEQ ID NO: 243 - protein, 235 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSQVSVSPSKVTLPCGGSVLVTCSASCDQPKLLGIKTPLTKKELLLPGNNEKVYELSNVQEDSQVMCYANCPDGQSTAKAFLTVYCEPKSSHHHHHH

SEQ ID NO: 244 - protein, 225 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSQVSVSPSKVTLPCGGSVLVTCSASCDQPKLLGIKTPLTKKELLLPGNNRKVYRLSNVQEDSQVMCYANCPDGQSTAKAFLTVYCSGWSHPQFEK

SEQ ID NO: 245 - protein, 240 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSGGGGSQVSVSPSKVTLPCGGSVLVTCSASCDQPKLLGIKTPLTKKELLLPGNNEKVYELSNVQEDSQVMCYANCPDGQSTAKAFLTVYCEPKSSHHHHHH

SEQ ID NO: 246 - protein, 230 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSGGGGSQVSVSPSKVTLPCGGSVLVTCSASCDQPKLLGIKTPLTKKELLLPGNNRKVYRLSNVQEDSQVMCYANCPDGQSTAKAFLTVYCSGWSHPQFEK

SEQ ID NO: 247 - protein, 225 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSQVSVSPSKVTLPCGGSVFVTCSASCDQPKLLGIKTPLTKKELLLPGNNRKVYELSNVQEDSQVMCYANCPDGQSTAKAFLTVYCEPKSSHHHHHH

SEQ ID NO: 248 - protein, 215 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSQVSVSPSKVTLPCGGSVAVACSASCDQPKLLGIKTPLTKKEALLPGNNRKAYELSNVQEDSQVMCYANCPDGQSTAKAFLTVYCSGWSHPQFEK

SEQ ID NO: 249 - protein, 230 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSQVSVSPSKVTLPCGGSVFVTCSASCDQPKLLGIKTPLTKKELLLPGNNRKVYELSNVQEDSQVMCYANCPDGQSTAKAFLTVYCEPKSSHHHHHH

SEQ ID NO: 250 - protein, 220 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSQVSVSPSKVTLPCGGSVAVACSASCDQPKLLGIKTPLTKKEALLPGNNRKAYELSNVQEDSQVMCYANCPDGQSTAKAFLTVYCSGWSHPQFEK

SEQ ID NO: 251 - protein, 235 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSQVSVSPSKVTLPCGGSVFVTCSASCDQPKLLGIKTPLTKKELLLPGNNRKVYELSNVQEDSQVMCYANCPDGQSTAKAFLTVYCEPKSSHHHHHH

SEQ ID NO: 252 - protein, 225 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSQVSVSPSKVTLPCGGSVAVACSASCDQPKLLGIKTPLTKKEALLPGNNRKAYELSNVQEDSQVMCYANCPDGQSTAKAFLTVYCSGWSHPQFEK

SEQ ID NO: 253 - protein, 240 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSGGGGSQVSVSPSKVTLPCGGSVFVTCSASCDQPKLLGIKTPLTKKELLLPGNNRKVYELSNVQEDSQVMCYANCPDGQSTAKAFLTVYCEPKSSHHHHHH

SEQ ID NO: 254 - protein, 230 aa, synthetic construct
DIVMTQSPDSLAVSLGERATINCRASQSVDYNGISYMHWYQQKPGQPPKLLIYAASNPESGVPDRFSGSGSGTDFTLTISSLQAEDVAVYYCQQIIEDPWTFGQGTKVEIKGGGGSGGGGSGGGGSGGGGSGGGGSQVSVSPSKVTLPCGGSVAVACSASCDQPKLLGIKTPLTKKEALLPGNNRKAYELSNVQEDSQVMCYANCPDGQSTAKAFLTVYCSGWSHPQFEK

SEQ ID NO: 255 - protein, 225 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSQVSVSPSKVTLPCGGSVWVTCSASCDQPKLLGIKTPLTKKELLLPGNNRKVYELSNVQEDSQVMCYANCPDGQSTAKAFLTVYCEPKSSHHHHHH

SEQ ID NO: 256 - protein, 215 aa, synthetic construct; identical to SEQ ID NO: 248

SEQ ID NO: 257 - protein, 230 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSQVSVSPSKVTLPCGGSVWVTCSASCDQPKLLGIKTPLTKKELLLPGNNRKVYELSNVQEDSQVMCYANCPDGQSTAKAFLTVYCEPKSSHHHHHH

SEQ ID NO: 258 - protein, 220 aa, synthetic construct; identical to SEQ ID NO: 250

SEQ ID NO: 259 - protein, 235 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSQVSVSPSKVTLPCGGSVWVTCSASCDQPKLLGIKTPLTKKELLLPGNNRKVYELSNVQEDSQVMCYANCPDGQSTAKAFLTVYCEPKSSHHHHHH

SEQ ID NO: 260 - protein, 225 aa, synthetic construct; identical to SEQ ID NO: 252

SEQ ID NO: 261 - protein, 240 aa, synthetic construct
QITLKESGPTLVKPTQTLTLTCTFSGFSLSTSGMGVSWIRQPPGKALEWLAHIYWDDDKRYNPSLKSRLTITKDTSKNQVVLTMTNMDPVDTATYYCARLYGFTYGFAYWGQGTLVTVSSGGGGSGGGGSGGGGSGGGGSGGGGSQVSVSPSKVTLPCGGSVWVTCSASCDQPKLLGIKTPLTKKELLLPGNNRKVYELSNVQEDSQVMCYANCPDGQSTAKAFLTVYCEPKSSHHHHHH

SEQ ID NO: 262 - protein, 230 aa, synthetic construct; identical to SEQ ID NO: 254

SEQ ID NO: 263 - DNA, 360 bp, synthetic construct
  source 1..360: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttct

SEQ ID NO: 264 - DNA, 333 bp, synthetic construct
  source 1..333: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaag

SEQ ID NO: 265 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacaaggcccgccggggatggaacctttcagaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 266 - DNA, 330 bp, synthetic construct; identical to SEQ ID NO: 3
  source 1..330: mol_type = other DNA

SEQ ID NO: 267 - DNA, 330 bp, synthetic construct
  source 1..330: mol_type = other DNA
agaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatatgtcagggtggagtcaccctcagttcgaaaag

SEQ ID NO: 268 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttcagaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 269 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttcagaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 270 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttgctaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 271 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttctgaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 272 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttctgaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 273 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttgccaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 274 - DNA, 318 bp, synthetic construct
  source 1..318: mol_type = other DNA
ctgcatctggaaccccccaaaactcatgtcacccatcatcccatatctgaccacgaagcaaccctggcctgctgggcacttgggttttaccccgcagagatcactctcacttggcaacaagatggggagggtcatacccaggataccgagcttgtcgaaacaaggcccgccggggatggaacctttcagaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 275 - DNA, 318 bp, synthetic construct
  source 1..318: mol_type = other DNA
cttcatcttgaaccacctaagacccacgttactcatcatccaataagcgaccacgaagccaccctgagatgctgggcacttggtttttatccagcagagatcactctgacttggcagcaggacggcgaaggtcacactcaagacacagaactcgtggaaactagaccagccggggatggcactttccaaaaatgggcagccgtcgtagtaccttctggggaagagcagagatacacctgccacgtccagcatgaagggctgcctgagcccgtaaccctgaggtgggaacctaagtcctgccaccaccatcatcatcat

SEQ ID NO: 276 - DNA, 318 bp, synthetic construct
  source 1..318: mol_type = other DNA
ctccatttggaacctcctaaaacacatgtcactcaccatcccatctccgaccacgaagctaccttgtgctgttgggctcttggattctacccagccgaaataactctgacatggcaacaggatggagaaggtcatacccaggatacagagctggtagaaactcggccagccggggatggtacattccagaagtgggcagcagtagttgtgccatccggggaggagcaaagatatacctgtcatgtccaacacgaaggtttgcctgaaccagtgactcttcgttgggagcccaagagtagccaccatcaccaccatcat

SEQ ID NO: 277 - DNA, 318 bp, synthetic construct
  source 1..318: mol_type = other DNA
ttgcatcttgaaccccctaagacacacgttacacattgccctatctccgatcacgaggcaactctccgatgttgggcactgggtttctatcccgcagagataacattgacttggcaacaggatggggaagggcatacacaagatacagaactggtcgaaacaaggcccgccggagacggaacatttcaaaagtgggcagcagttgtagttccctctggggaggaacagcgctacacttgccatgtgcagcacgaaggtctgccagagcctgtcactctcagatgggagcccaagagtagccaccatcaccaccatcat

SEQ ID NO: 278 - DNA, 318 bp, synthetic construct
  source 1..318: mol_type = other DNA
ttgcacctcgaaccacccaaaacccacgtgactcatcatcccatctctgaccacgaggccacacttcggtgttgggctctgggcttctacccagcagagataaccctgacctggcaacaggatggggagggtcacacacaggacacagaattggtctgcactagacctgccggtgacggcacctttcaaaagtgggcagcagtggttgtgccaagcggagaggagcaacgatacacctgccacgtacaacacgaagggctccctgaaccagtaacactgcgctgggagcccaagagtagccaccatcaccaccatcat

SEQ ID NO: 279 - DNA, 318 bp, synthetic construct
  source 1..318: mol_type = other DNA
ttgcatcttgaacctcctaaaactcatgtaactcaccacccaatttcagaccatgaagctactctccgatgctgggctcttggcttttatcctgccgaaatcacattgacatggcaacaggatggagaggggcatacacaggacaccgagttggtggaaacttgtcccgcaggggatggcacatttcaaaaatgggctgctgttgttgtgcccagcggcgaagaacagcggtacacatgtcacgtccaacatgaaggcttgccagagcccgtaactcttcgctgggagcccaagagtagccaccatcaccaccatcat

SEQ ID NO: 280 - DNA, 318 bp, synthetic construct
  source 1..318: mol_type = other DNA
ttgcatctcgagccccccaagacccacgttactcaccaccctataagcgatcacgaggcaacattgaggtgctgggccttgggtttctaccccgctgagatcacactcacttggcagcaagatggagaagggcatacacaggacaccgaactcgtggaaacccgaccagccggttgtggtacattccagaaatgggctgccgtagtagtccctagcggtgaggagcagcgatatacctgtcatgtgcaacacgagggcttgcccgaaccagtcactttgagatgggagcccaagagtagccaccatcaccaccatcat

SEQ ID NO: 281 - DNA, 330 bp, synthetic construct
  source 1..330: mol_type = other DNA
agaaccgttagaactcctaagattcaggtcttctcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatatgtcagggtggagtcaccctcagttcgaaaag

SEQ ID NO: 282 - DNA, 330 bp, synthetic construct
  source 1..330: mol_type = other DNA
agaaccgttagaactcctaagattcaggtctggtcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatatgtcagggtggagtcaccctcagttcgaaaag

SEQ ID NO: 283 - DNA, 330 bp, synthetic construct
  source 1..330: mol_type = other DNA
agaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcggttcatgtcagggtggagtcaccctcagttcgaaaag

SEQ ID NO: 284 - DNA, 330 bp, synthetic construct
  source 1..330: mol_type = other DNA
agaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcggtggatgtcagggtggagtcaccctcagttcgaaaag

SEQ ID NO: 285 - DNA, 330 bp, synthetic construct
  source 1..330: mol_type = other DNA
agaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatttctcagggtggagtcaccctcagttcgaaaag

SEQ ID NO: 286 - DNA, 330 bp, synthetic construct
  source 1..330: mol_type = other DNA
agaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggattggtcagggtggagtcaccctcagttcgaaaag

SEQ ID NO: 287 - DNA, 348 bp, synthetic construct
  source 1..348: mol_type = other DNA
agaactgtacgcacaccaaagatacaagtatattccagacaccccgcagaaaatggtaagagcaatttcttgaactgttatgtatccggttttcacccttctgatatagaggtagatttgctgaaaaatggtgagcggatagagaaagttgagcactccgatcttagctcatctaaggattcaagcacttatctcttgtattacacagagtttactcccaccgagaaagacgagtatgcctgccgtgtcaaccatgtcactttgtcccagcccaagatagtgaaatgggaccgagatggaggcagtggagagtgctctgggtacccttacgatgtccctgattacgca

SEQ ID NO: 288 - DNA, 351 bp, synthetic construct
  source 1..351: mol_type = other DNA
cggacagtgcggactccaaagatacaagtttatagcaggcatcccgctgagaatgggaagtccaactttcttaattgctatgtctccgggttccacccctctgacatcgaggtggacttgctgaagaatggggaaagaatagagaaagttgagcactctgatctcagttcatccaaggattcctcaacatacctcttgtactacactgagttcaccccaaccgaaaaagatgaatatgcttgcagagtcaatcacgtgaccctgtcacagcctaaaatagtcaagtgggatagggacggaggtgggagcggtgagtgctctgggtacccttacgatgtccctgattacgca

SEQ ID NO: 289 - DNA, 351 bp, synthetic construct
  source 1..351: mol_type = other DNA
cggactgttcgcacacccaaaattcaggtttactcccgacatccagctgagaacggtaagtctaatttcctgaactgttacgtgtctggttttcatccttcagatatagaagttgacctcctcaaaaacggcgaaaggatagaaaaggtagagcacagcgacttgagtagcagcaaagattctagcacatatctgctgtattataccgaatttacacccacagagaaggacgagtacgcatgtcgggtaaaccatgtgacactctctcagcctaagatcgtcaagtgggaccgagactgtggaggttctggtgagagttccggctatccttatgatgtgcccgactacgcc

SEQ ID NO: 290 - DNA, 351 bp, synthetic construct
  source 1..351: mol_type = other DNA
cgcacagtacgtacaccaaagatccaggtttactcacgtcaccccgccgagaatggcaagagcaacttcctcaactgctacgtgagtggattccaccctagtgacatcgaggttgacctgctgaaaaatggtgagcgcattgaaaaagttgaacattctgacttgagcagcagtaaagacagttcaacatatctcctctactacaccgagtttacaccaaccgaaaaagacgagtacgcatgtagagttaatcatgtgactctgtcacaacctaaaatcgtaaagtgggaccgatgcatgggaggttctggtgagagttccggctatccttatgatgtgcccgactacgcc

SEQ ID NO: 291 - DNA, 351 bp, synthetic construct
  source 1..351: mol_type = other DNA
cgaaccgtccgaacaccctgcattcaagtttacagccggcatccagccgagaatgggaagtctaattttctgaattgttatgtaagcggattccacccaagcgacattgaggtggaccttttgaagaatggcgaacgcatagagaaagtcgaacatagcgacctttcaagctctaaagattccagtacctacctcctttattatactgagtttacccccactgaaaaggacgaatatgcttgtagggtgaaccacgtcactctcagccagccaaagattgttaaatgggatagggatatgggaggttctggtgagagttccggctatccttatgatgtgcccgactacgcc

SEQ ID NO: 292 - DNA, 351 bp, synthetic construct
  source 1..351: mol_type = other DNA
cgcactgtgagaacacctaagatccaggtctgttctaggcatcccgctgaaaacggcaaatcaaactttttgaactgttatgtgagtggtttccatccctccgatatagaagttgacttgcttaaaaatggagaacgcatcgaaaaagtggaacattctgatctttcttccagcaaggatagctctacatacctcctttactacaccgagtttactccaacagagaaggatgagtacgcttgtagagttaaccatgtcactctcagccaacctaagattgttaaatgggacagagatatgggaggttctggtgagagttccggctatccttatgatgtgcccgactacgcc

SEQ ID NO: 293 - DNA, 351 bp, synthetic construct
  source 1..351: mol_type = other DNA
cgcactgtaaggacccccaaaatacaggtctactcatgtcaccccgccgagaatggaaagtccaatttcctgaactgctacgtaagtgggttccatccttctgatatagaagttgatctgctcaagaacggagagcgtatagaaaaagttgaacatagtgatctttcaagcagcaaagactcttcaacctatctgctttactatactgagttcactccaacagaaaaggacgaatacgcttgccgggtgaaccacgtcactctttctcaacctaagattgtaaaatgggaccgagacatgggaggttctggtgagagttccggctatccttatgatgtgcccgactacgcc

SEQ ID NO: 294 - DNA, 354 bp, synthetic construct
  source 1..354: mol_type = other DNA
cggactgttcgcacacccaaaattcaggtttactcccgacatccagctgagaacggtaagtctaatttcctgaactgttacgtgtctggttttcatccttcagatatagaagttgacctcctcaaaaacggcgaaaggatagaaaaggtagagcacagcgacttgagtagcagcaaagattctagcacatatctgctgtattataccgaatttacacccacagagaaggacgagtacgcatgtcgggtaaaccatgtgacactctctcagcctaagatcgtcaagtgggaccgagactgtggaggaggtagcggagagagcagtggatatccctacgatgtacctgattacgca

SEQ ID NO: 295 - DNA, 354 bp, synthetic construct
  source 1..354: mol_type = other DNA
cgcacagtacgtacaccaaagatccaggtttactcacgtcaccccgccgagaatggcaagagcaacttcctcaactgctacgtgagtggattccaccctagtgacatcgaggttgacctgctgaaaaatggtgagcgcattgaaaaagttgaacattctgacttgagcagcagtaaagacagttcaacatatctcctctactacaccgagtttacaccaaccgaaaaagacgagtacgcatgtagagttaatcatgtgactctgtcacaacctaaaatcgtaaagtgggaccgatgcatgggaggaggtagcggagagagcagtggatatccctacgatgtacctgattacgca

SEQ ID NO: 296 - DNA, 354 bp, synthetic construct
  source 1..354: mol_type = other DNA
cgaaccgtccgaacaccctgcattcaagtttacagccggcatccagccgagaatgggaagtctaattttctgaattgttatgtaagcggattccacccaagcgacattgaggtggaccttttgaagaatggcgaacgcatagagaaagtcgaacatagcgacctttcaagctctaaagattccagtacctacctcctttattatactgagtttacccccactgaaaaggacgaatatgcttgtagggtgaaccacgtcactctcagccagccaaagattgttaaatgggatagggatatgggaggaggtagcggagagagcagtggatatccctacgatgtacctgattacgca

SEQ ID NO: 297 - DNA, 354 bp, synthetic construct
  source 1..354: mol_type = other DNA
cgcactgtgagaacacctaagatccaggtctgttctaggcatcccgctgaaaacggcaaatcaaactttttgaactgttatgtgagtggtttccatccctccgatatagaagttgacttgcttaaaaatggagaacgcatcgaaaaagtggaacattctgatctttcttccagcaaggatagctctacatacctcctttactacaccgagtttactccaacagagaaggatgagtacgcttgtagagttaaccatgtcactctcagccaacctaagattgttaaatgggacagagatatgggaggaggtagcggagagagcagtggatatccctacgatgtacctgattacgca

SEQ ID NO: 298 - DNA, 354 bp, synthetic construct
  source 1..354: mol_type = other DNA
cgcactgtaaggacccccaaaatacaggtctactcatgtcaccccgccgagaatggaaagtccaatttcctgaactgctacgtaagtgggttccatccttctgatatagaagttgatctgctcaagaacggagagcgtatagaaaaagttgaacatagtgatctttcaagcagcaaagactcttcaacctatctgctttactatactgagttcactccaacagaaaaggacgaatacgcttgccgggtgaaccacgtcactctttctcaacctaagattgtaaaatgggaccgagacatgggaggaggtagcggagagagcagtggatatccctacgatgtacctgattacgca

SEQ ID NO: 299 - skipped

SEQ ID NO: 300 - DNA, 18 bp, synthetic construct
  source 1..18: mol_type = other DNA
agaaccgttgggggatca

SEQ ID NO: 301 - DNA, 30 bp, synthetic construct
  source 1..30: mol_type = other DNA
ggcggaggcggctctggtggcggcgggtct

SEQ ID NO: 302 - DNA, 45 bp, synthetic construct
  source 1..45: mol_type = other DNA
gggggcggcggaagtggaggcgggggttcaggtgggggagggagc

SEQ ID NO: 303 - DNA, 60 bp, synthetic construct
  source 1..60: mol_type = other DNA
gggggcggaggatctggcgggggaggttctggaggaggtggaagtggcggcggtggatct

SEQ ID NO: 304 - DNA, 75 bp, synthetic construct
  source 1..75: mol_type = other DNA
ggcggcggggggagcggcggtggcggctccggcggagggggttcaggcggaggtgggtccggcggaggaggttct

SEQ ID NO: 305 - DNA, 318 bp, synthetic construct; identical to SEQ ID NO: 265
  source 1..318: mol_type = other DNA

SEQ ID NO: 306 - DNA, 330 bp, synthetic construct; identical to SEQ ID NO: 267
  source 1..330: mol_type = other DNA

SEQ ID NO: 307 - DNA, 678 bp, synthetic construct
  source 1..678: mol_type = other DNA
cagataaccctcaaggagtcagggccaaccctggtgaagccaactcaaacacttactcttacctgcactttttctggatttagccttagtacaagtgggatgggcgtaagttggatacgccaacctcctggcaaggccctggaatggctggcacacatatactgggacgatgacaagcgctataacccatctttgaagagccggttgactatcactaaggacaccagtaaaaatcaagtagtgcttacaatgactaacatggaccctgtggataccgccacctattactgtgctaggttgtatggctttacctatggcttcgcatactggggacaggggacactcgtaactgtatcctctctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacaaggcccgccggggatggaacctttcagaaatgggctgctgtagtggtccctagcggagaagaacaacggtacacttgtcatgtccagcatgagggtcttccagaaccagtcacccttcggtgggagcccaagagtagccaccatcaccaccatcat

SEQ ID NO: 308 - DNA, 663 bp, synthetic construct
  source 1..663: mol_type = other DNA
gatatcgtgatgacacagagtcctgattccttggccgtctcattgggtgaaagggcaacaattaactgtcgtgccagtcagtctgtcgactacaatggcatctcttacatgcactggtatcaacaaaagccaggacaacctccaaaactcctgatttatgccgcctccaaccccgagagtggcgttcccgatagattctcaggatcagggtctgggactgactttactctcaccattagttccctccaggcagaagatgttgcagtatattattgtcaacaaattatcgaagacccttggactttcggtcagggaacaaaagtagaaatcaaaagaaccgttagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatatgtcagggtggagtcaccctcagttcgaaaag

SEQ ID NO: 309 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttcagaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 310 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttcagaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 311 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttgctaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 312 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttctgaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 313 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacagcccccgccggggatggaacctttctgaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 314 - DNA, 318 bp, synthetic construct
  source 1..318: mol_type = other DNA
ctgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacactgcccgccggggatggaacctttgccaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 315 - DNA, 318 bp, synthetic construct
  source 1..318: mol_type = other DNA
ctgcatctggaaccccccaaaactcatgtcacccatcatcccatatctgaccacgaagcaaccctggcctgctgggcacttgggttttaccccgcagagatcactctcacttggcaacaagatggggagggtcatacccaggataccgagcttgtcgaaacaaggcccgccggggatggaacctttcagaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 316 - DNA, 330 bp, synthetic construct; identical to SEQ ID NO: 281
  source 1..330: mol_type = other DNA

SEQ ID NO: 317 - DNA, 330 bp, synthetic construct; identical to SEQ ID NO: 282
  source 1..330: mol_type = other DNA

SEQ ID NO: 318 - DNA, 330 bp, synthetic construct; identical to SEQ ID NO: 283
  source 1..330: mol_type = other DNA

SEQ ID NO: 319 - DNA, 318 bp, synthetic construct; identical to SEQ ID NO: 312
  source 1..318: mol_type = other DNA

SEQ ID NO: 320 - DNA, 330 bp, synthetic construct; identical to SEQ ID NO: 284
  source 1..330: mol_type = other DNA

SEQ ID NO: 321 - DNA, 330 bp, synthetic construct; identical to SEQ ID NO: 285
  source 1..330: mol_type = other DNA

SEQ ID NO: 322 - DNA, 330 bp, synthetic construct; identical to SEQ ID NO: 286
  source 1..330: mol_type = other DNA

SEQ ID NO: 323 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcactgcatctggaaccccccaaaactcatgtcacccatcatcccatatctgaccacgaagcaaccctgcgttgctgggcacttgggttttaccccgcagagatcactctcacttggcaacaagatggggagggtcatacccaggataccgagcttgtcgaaacaaggcccgccggggatggaacctttcagaaatgggctgctgtagtggtccctagcggagaagaacaacggtacacttgtcatgtccagcatgagggtcttccagaaccagtcacccttcggtgggagcccaagagtagccaccaccatcatcatcat

SEQ ID NO: 324 - DNA, 672 bp, synthetic construct
  source 1..672: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcaagaactcctaagattcaggtctattcccgacatcctgctgagaatggaaagtctaattttctcaactgttatgtatcagggttccatccaagtgatatagaagttgatcttctcaagaacggtgagcgtatcgaaaaagtcgagcattctgacctctccagttccaaggatagttccacatacctgctgtattataccgagtttactccaacagaaaaagatgaatacgcctgtcgagtaaatcacgtcactctttcccagcctaagattgtgaagtgggatcgggatatgtcagggtggagtcaccctcagttcgaaaag

SEQ ID NO: 325 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcactccatttggagccaccaaaaacccatgtaacccaccaccctataagtgaccacgaagctactctgcggtgttgggcactgggcttttacccagcagaaataacactcacttggcaacaagatggagaaggtcatactcaggatacagaactcgtggagacacgtcccgcaggtgatggtacttttcagaagtgggctgctgttgtggtcccctccggggaggaacagaggtacacctgtcatgtccaacacgagggtttgcccgaacccgttactctccgatgggagcccaagtcatgccaccaccatcatcatcat

SEQ ID NO: 326 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcacttcatcttgaaccacctaagacccacgttactcatcatccaataagcgaccacgaagccaccctgagatgctgggcacttggtttttatccagcagagatcactctgacttggcagcaggacggcgaaggtcacactcaagacacagaactcgtggaaactagaccagccggggatggcactttccaaaaatgggcagccgtcgtagtaccttctggggaagagcagagatacacctgccacgtccagcatgaagggctgcctgagcccgtaaccctgaggtgggaacctaagtcctgccaccaccatcatcatcat

SEQ ID NO: 327 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcactccatttggaacctcctaaaacacatgtcactcaccatcccatctccgaccacgaagctaccttgtgctgttgggctcttggattctacccagccgaaataactctgacatggcaacaggatggagaaggtcatacccaggatacagagctggtagaaactcggccagccggggatggtacattccagaagtgggcagcagtagttgtgccatccggggaggagcaaagatatacctgtcatgtccaacacgaaggtttgcctgaaccagtgactcttcgttgggagcccaagagtagccaccaccatcatcatcat

SEQ ID NO: 328 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcattgcatcttgaaccccctaagacacacgttacacattgccctatctccgatcacgaggcaactctccgatgttgggcactgggtttctatcccgcagagataacattgacttggcaacaggatggggaagggcatacacaagatacagaactggtcgaaacaaggcccgccggagacggaacatttcaaaagtgggcagcagttgtagttccctctggggaggaacagcgctacacttgccatgtgcagcacgaaggtctgccagagcctgtcactctcagatgggagcccaagagtagccaccaccatcatcatcat

SEQ ID NO: 329 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcattgcacctcgaaccacccaaaacccacgtgactcatcatcccatctctgaccacgaggccacacttcggtgttgggctctgggcttctacccagcagagataaccctgacctggcaacaggatggggagggtcacacacaggacacagaattggtctgcactagacctgccggtgacggcacctttcaaaagtgggcagcagtggttgtgccaagcggagaggagcaacgatacacctgccacgtacaacacgaagggctccctgaaccagtaacactgcgctgggagcccaagagtagccaccaccatcatcatcat

SEQ ID NO: 330 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcattgcatcttgaacctcctaaaactcatgtaactcaccacccaatttcagaccatgaagctactctccgatgctgggctcttggcttttatcctgccgaaatcacattgacatggcaacaggatggagaggggcatacacaggacaccgagttggtggaaacttgtcccgcaggggatggcacatttcaaaaatgggctgctgttgttgtgcccagcggcgaagaacagcggtacacatgtcacgtccaacatgaaggcttgccagagcccgtaactcttcgctgggagcccaagagtagccaccaccatcatcatcat

SEQ ID NO: 331 - DNA, 687 bp, synthetic construct
  source 1..687: mol_type = other DNA
cagatcaccctgaaagagtctggccccacactggtcaagcccacacagaccctgacactgacctgcaccttctccggcttctccctgtctacctctggcatgggcgtgtcctggatcagacagcctcctggcaaagccctggaatggctggctcacatctactgggacgacgacaagcggtacaaccccagcctgaagtctcggctgaccatcaccaaggacacctccaagaaccaggtggtgctgaccatgacaaacatggaccctgtggacaccgccacctactactgcgctagactgtacggcttcacctacggctttgcctattggggccagggcaccctggttaccgtttcttctgggggatcattgcatctcgagccccccaagacccacgttactcaccaccctataagcgatcacgaggcaacattgaggtgctgggccttgggtttctaccccgctgagatcacactcacttggcagcaagatggagaagggcatacacaggacaccgaactcgtggaaacccgaccagccggttgtggtacattccagaaatgggctgccgtagtagtccctagcggtgaggagcagcgatatacctgtcatgtgcaacacgagggcttgcccgaaccagtcactttgagatgggagcccaagagtagccaccaccatcatcatcat

SEQ ID NO: 332 - DNA, 699 bp, synthetic construct
  source 1..699: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcaagaactgtacgcacaccaaagatacaagtatattccagacaccccgcagaaaatggtaagagcaatttcttgaactgttatgtatccggttttcacccttctgatatagaggtagatttgctgaaaaatggtgagcggatagagaaagttgagcactccgatcttagctcatctaaggattcaagcacttatctcttgtattacacagagtttactcccaccgagaaagacgagtatgcctgccgtgtcaaccatgtcactttgtcccagcccaagatagtgaaatgggaccgagatggaggcagtggagagtgctctgggtacccttacgatgtccctgattacgca

SEQ ID NO: 333 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacggacagtgcggactccaaagatacaagtttatagcaggcatcccgctgagaatgggaagtccaactttcttaattgctatgtctccgggttccacccctctgacatcgaggtggacttgctgaagaatggggaaagaatagagaaagttgagcactctgatctcagttcatccaaggattcctcaacatacctcttgtactacactgagttcaccccaaccgaaaaagatgaatatgcttgcagagtcaatcacgtgaccctgtcacagcctaaaatagtcaagtgggatagggacggaggtgggagcggtgagtgctctgggtacccttacgatgtccctgattacgca

SEQ ID NO: 334 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacggactgttcgcacacccaaaattcaggtttactcccgacatccagctgagaacggtaagtctaatttcctgaactgttacgtgtctggttttcatccttcagatatagaagttgacctcctcaaaaacggcgaaaggatagaaaaggtagagcacagcgacttgagtagcagcaaagattctagcacatatctgctgtattataccgaatttacacccacagagaaggacgagtacgcatgtcgggtaaaccatgtgacactctctcagcctaagatcgtcaagtgggaccgagactgtggaggttctggtgagagttccggctatccttatgatgtgcccgactacgcc

SEQ ID NO: 335 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcacagtacgtacaccaaagatccaggtttactcacgtcaccccgccgagaatggcaagagcaacttcctcaactgctacgtgagtggattccaccctagtgacatcgaggttgacctgctgaaaaatggtgagcgcattgaaaaagttgaacattctgacttgagcagcagtaaagacagttcaacatatctcctctactacaccgagtttacaccaaccgaaaaagacgagtacgcatgtagagttaatcatgtgactctgtcacaacctaaaatcgtaaagtgggaccgatgcatgggaggttctggtgagagttccggctatccttatgatgtgcccgactacgcc

SEQ ID NO: 336 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgaaccgtccgaacaccctgcattcaagtttacagccggcatccagccgagaatgggaagtctaattttctgaattgttatgtaagcggattccacccaagcgacattgaggtggaccttttgaagaatggcgaacgcatagagaaagtcgaacatagcgacctttcaagctctaaagattccagtacctacctcctttattatactgagtttacccccactgaaaaggacgaatatgcttgtagggtgaaccacgtcactctcagccagccaaagattgttaaatgggatagggatatgggaggttctggtgagagttccggctatccttatgatgtgcccgactacgcc

SEQ ID NO: 337 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcactgtgagaacacctaagatccaggtctgttctaggcatcccgctgaaaacggcaaatcaaactttttgaactgttatgtgagtggtttccatccctccgatatagaagttgacttgcttaaaaatggagaacgcatcgaaaaagtggaacattctgatctttcttccagcaaggatagctctacatacctcctttactacaccgagtttactccaacagagaaggatgagtacgcttgtagagttaaccatgtcactctcagccaacctaagattgttaaatgggacagagatatgggaggttctggtgagagttccggctatccttatgatgtgcccgactacgcc

SEQ ID NO: 338 - DNA, 702 bp, synthetic construct
  source 1..702: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcactgtaaggacccccaaaatacaggtctactcatgtcaccccgccgagaatggaaagtccaatttcctgaactgctacgtaagtgggttccatccttctgatatagaagttgatctgctcaagaacggagagcgtatagaaaaagttgaacatagtgatctttcaagcagcaaagactcttcaacctatctgctttactatactgagttcactccaacagaaaaggacgaatacgcttgccgggtgaaccacgtcactctttctcaacctaagattgtaaaatgggaccgagacatgggaggttctggtgagagttccggctatccttatgatgtgcccgactacgcc

SEQ ID NO: 339 - DNA, 705 bp, synthetic construct
  source 1..705: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacggactgttcgcacacccaaaattcaggtttactcccgacatccagctgagaacggtaagtctaatttcctgaactgttacgtgtctggttttcatccttcagatatagaagttgacctcctcaaaaacggcgaaaggatagaaaaggtagagcacagcgacttgagtagcagcaaagattctagcacatatctgctgtattataccgaatttacacccacagagaaggacgagtacgcatgtcgggtaaaccatgtgacactctctcagcctaagatcgtcaagtgggaccgagactgtggaggaggtagcggagagagcagtggatatccctacgatgtacctgattacgca

SEQ ID NO: 340 - DNA, 705 bp, synthetic construct
  source 1..705: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcacagtacgtacaccaaagatccaggtttactcacgtcaccccgccgagaatggcaagagcaacttcctcaactgctacgtgagtggattccaccctagtgacatcgaggttgacctgctgaaaaatggtgagcgcattgaaaaagttgaacattctgacttgagcagcagtaaagacagttcaacatatctcctctactacaccgagtttacaccaaccgaaaaagacgagtacgcatgtagagttaatcatgtgactctgtcacaacctaaaatcgtaaagtgggaccgatgcatgggaggaggtagcggagagagcagtggatatccctacgatgtacctgattacgca

SEQ ID NO: 341 - DNA, 705 bp, synthetic construct
  source 1..705: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgaaccgtccgaacaccctgcattcaagtttacagccggcatccagccgagaatgggaagtctaattttctgaattgttatgtaagcggattccacccaagcgacattgaggtggaccttttgaagaatggcgaacgcatagagaaagtcgaacatagcgacctttcaagctctaaagattccagtacctacctcctttattatactgagtttacccccactgaaaaggacgaatatgcttgtagggtgaaccacgtcactctcagccagccaaagattgttaaatgggatagggatatgggaggaggtagcggagagagcagtggatatccctacgatgtacctgattacgca

SEQ ID NO: 342 - DNA, 705 bp, synthetic construct
  source 1..705: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcactgtgagaacacctaagatccaggtctgttctaggcatcccgctgaaaacggcaaatcaaactttttgaactgttatgtgagtggtttccatccctccgatatagaagttgacttgcttaaaaatggagaacgcatcgaaaaagtggaacattctgatctttcttccagcaaggatagctctacatacctcctttactacaccgagtttactccaacagagaaggatgagtacgcttgtagagttaaccatgtcactctcagccaacctaagattgttaaatgggacagagatatgggaggaggtagcggagagagcagtggatatccctacgatgtacctgattacgca

SEQ ID NO: 343 - DNA, 705 bp, synthetic construct
  source 1..705: mol_type = other DNA
gacatcgtgatgacccagtctccagacagcctggctgtgtctctgggcgagagagccaccatcaactgcagagcctctcagtccgtggactacaacggcatctcctacatgcactggtatcagcagaagcccggccagcctcctaagctgctgatctacgccgcctccaatcctgagtctggcgtgcccgatagattctccggctctggctctggcaccgactttaccctgacaatcagctccctgcaggccgaggatgtggccgtgtactactgccagcagatcatcgaggacccctggacctttggccagggcaccaaggtggaaatcaagagaaccgttgggggatcacgcactgtaaggacccccaaaatacaggtctactcatgtcaccccgccgagaatggaaagtccaatttcctgaactgctacgtaagtgggttccatccttctgatatagaagttgatctgctcaagaacggagagcgtatagaaaaagttgaacatagtgatctttcaagcagcaaagactcttcaacctatctgctttactatactgagttcactccaacagaaaaggacgaatacgcttgccgggtgaaccacgtcactctttctcaacctaagattgtaaaatgggaccgagacatgggaggaggtagcggagagagcagtggatatccctacgatgtacctgattacgca

SEQ ID NO: 344 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 345 - protein, 207 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCM

SEQ ID NO: 346 - protein, 449 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 347 - protein, 220 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 348 - protein, 450 aa, synthetic construct; identical to SEQ ID NO: 344

SEQ ID NO: 349 - protein, 207 aa, synthetic construct; identical to SEQ ID NO: 345

SEQ ID NO: 350 - protein, 452 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 351 - protein, 214 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 352 - protein, 452 aa, synthetic construct
EVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSSASTLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 353 - protein, 207 aa, synthetic construct
DIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCM

SEQ ID NO: 354 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 355 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 449 aa, synthetic construct
EVQLVESGGGLVQPGGSLRLSCAASGYTFTSYWLHWVRQAPGKGLEWVGMIDPSNSDTRFNPNFKDRFTISADTSKNTAYLQMNSLRAEDTAVYYCATYRSYVTPLDYWGQGTLVTVSSASTLHLEPPKTHVTHCPISDHEATLRCWALGFYPAEITLTWQQDGEGHTQDTELVETRPAGDGTFQKWAAVVVPSGEEQRYTCHVQHEGLPEPVTLRWEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 213 aa, synthetic construct
DIQMTQSPSSLSASVGDRVTITCKSSQSLLYTSSQKNYLAWYQQKPGKAPKLLIYWASTRESGVPSRFSGSGSGTDFTLTISSLQPEDFATYYCQQYYAYPWTFGQGTKVEIKRTVRTPKIQVYSRHPAENGKSNFLNCYVSGFHPSDIEVDLLKNGERIEKVEHSDLSSSKDSSTYLLYYTEFTPTEKDEYACRVNHVTLSQPKIVKWDRCM

SEQ ID NO: 358 - protein, 450 aa, synthetic construct; identical to SEQ ID NO: 354

SEQ ID NO: 359 - protein, 214 aa, synthetic construct; identical to SEQ ID NO: 355

SEQ ID NO: 360 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 361 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRFTQKSLSLSPGK

SEQ ID NO: 362 - protein, 107 aa, synthetic construct [sequence omitted]

SEQ ID NO: 363 - protein, 120 aa, synthetic construct [sequence omitted]

SEQ ID NO: 364 - protein, 113 aa, synthetic construct [sequence omitted]

SEQ ID NO: 365 - protein, 119 aa, synthetic construct [sequence omitted]

SEQ ID NO: 366 - protein, 107 aa, synthetic construct [sequence omitted]

SEQ ID NO: 367 - protein, 122 aa, synthetic construct [sequence omitted]

SEQ ID NO: 368 - protein, 106 aa, Homo sapiens
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 369 - protein, 97 aa, Homo sapiens
LHLEPPKTHVTHHPISDHEATLRCWALGFYPAEITLTWQQDGEGHTQDTELVETRPAGDGTFQKWAAVVVPSGEEQRYTCHVQHEGLPEPVTLRWKP

(19) United States
(12) Patent Application Publication
(10) Pub. No.: US 2026/0258158 A1
(43) Pub. Date: Sep. 3, 2026

(54) Material and Methods for Improved Bioengineered Pairing of Antigen-Binding Variable Regions

(71) Applicant: Janssen Biotech, Inc., Horsham, PA (US)

(72) Inventors: Elisabeth G. Prinslow, Spring House, PA (US); Linxiao Chen, Spring House, PA (US); Adam Zwolak, Spring House, PA (US); Neeraj Kohli, Spring House, PA (US); Jinquan Luo, Spring House, PA (US); Partha S. Chowdhury, Spring House, PA (US)

(21) Appl. No.: 18/994,507

(22) PCT Filed: Jul. 17, 2023
(86) PCT No.: PCT/IB2023/057291

§ 371 Date: Jan. 14, 2025

Related U.S. Application Data
(60) Provisional application No. 63/389,814, filed on Jul. 15, 2022.

(51) Int. Cl. C07K 16/46; C07K 14/705; C07K 19/00; C12N 15/63

(52) U.S. Cl. CPC C07K 16/46; C07K 14/70525; C07K 14/70539; C07K 19/00; C12N 15/63; C07K 2317/56; C07K 2317/62; C07K 2319/735

(57) ABSTRACT

Antigen-binding molecules that are engineered to replace the light chain constant domain (CL) and heavy chain constant domain 1 (CHi) with HLA class I histocompatibility antigen alpha chain-E alpha-3 (HLA-E)/Beta-2 microglobulin (B2M) or intercellular adhesion molecule 1 domain 1 (ICAM-1 D1)/ICAM-1 D1 dimerization domains are described.

### 1. CROSS-REFERENCE TO RELATED APPLICATIONS

This application claims priority to U.S. Provisional Patent Application No. 63/389,814, filed on Jul. 15, 2022, the disclosures of which is herein incorporated by reference in its entirety.

### 2. SEQUENCE LISTING

The instant application contains a Sequence Listing which has been submitted electronically in XML file format and is hereby incorporated by reference in its entirety. Said XML copy, created on Jul. 12, 2023, is named 253505_000341_SL and is 391,390 bytes in size.

### 3. FIELD

Provided herein are antigen-binding molecules that comprise one or more antigen-binding polypeptides, which form a variable region that binds a target antigen, and, in lieu of an antibody light chain constant domain (CL) and antibody heavy chain constant domain 1 (CH1), have dimerization domains as described herein. Replacement of the dimerization interface between a light chain constant domain and heavy chain constant domain 1 provides a means for selective assembly of cognate chains in multiparatopic antigen-binding molecules.

### 4. BACKGROUND

Production of bispecific heterodimeric antibodies with modified heavy chain IgG constant regions to promote efficient formation of heavy chain heterodimer pairs and arm specific pairing of heavy and light chains, have been described (WO 2018/237192 A1).

### 5. SUMMARY

In one aspect, provided herein are antigen-binding molecules comprising one or more antigen-binding polypeptides each comprising a variable domain that binds a target antigen, and, in lieu of a light chain constant domain (CL) or heavy chain constant domain 1 (CH1), a dimerization domain as described herein.

In specific embodiments, the antigen-binding molecule binds to one or more target antigens. In specific embodiments, an antigen-binding molecule as described herein comprises two or more polypeptides each comprising a variable domain, wherein the variable domains form paratopes that bind to one or more target antigens.

In specific embodiments, an antigen-binding molecule as described herein comprises two, three, four, five, six, seven, or eight polypeptides, optionally wherein each of the two, three, four, five, six, seven, or eight polypeptides comprises a dimerization domain and a variable domain that (in conjunction with a cognate variable domain) binds to a target antigen as described herein. In specific embodiments, a dimerization domain binds to another dimerization domain to form a dimer (in particular, heterodimers, e.g., six polypeptides can form up to three dimers with each other).

In specific embodiments, a polypeptide of an antigen-binding molecule as described herein comprises a light chain polypeptide comprising a light chain variable domain and a light chain dimerization domain, and a heavy chain polypeptide comprising a heavy chain variable domain and a heavy chain dimerization domain. In specific embodiments, the light chain variable domain and the heavy chain variable domain compose a variable region that binds a target antigen.

In specific embodiments of an antigen-binding molecule as described herein, a light chain polypeptide comprises an immunoglobulin or antibody light chain or one or more fragments thereof, optionally, the light chain polypeptide comprises a kappa (κ) chain, lambda (λ) chain, sigma (σ) chain, iota (ι) chain, or one or more fragments thereof. In specific embodiments of an antigen-binding molecule as described herein, a heavy chain polypeptide comprises an immunoglobulin or antibody heavy chain or one or more fragments thereof, optionally the heavy chain polypeptide comprises a gamma (γ) chain, delta (δ) chain, alpha (α) chain, mu (μ) chain, epsilon (ε) chain, or one or more fragments thereof.

In one aspect, an antigen-binding molecule as described herein comprises a first light chain polypeptide comprising, in amino-terminus to carboxy-terminus order: VL1-LD1, wherein VL1 is a first light chain variable domain and LD1 is a first light chain dimerization domain; and a first heavy chain polypeptide comprising, in amino-terminus to carboxy-terminus order: VH1-HD1, wherein VH1 is a first heavy chain variable domain and HD1 is a first heavy chain dimerization domain, wherein a) i) LD1 comprises a beta-2 microglobulin (B2M) domain and HD1 comprises a HLA-E A3 (EA3) domain, or LD1 comprises an EA3 domain and HD1 comprises a B2M domain, wherein the B2M domain and the EA3 domain bind to each other to form a dimer; or ii) LD1 comprises a first ICAM-1 D1 domain and HD1 comprises a second ICAM-1 D1 domain, wherein the first and second ICAM-1 D1 domains bind to each other to form a dimer, b) i) at least one of the B2M domain or EA3 domain differs from the wild-type B2M domain (SEQ ID NO:2) or wild-type EA3 domain (SEQ ID NO:33), respectively; and ii) the first ICAM-1 D1 domain differs from the second ICAM-1 D1 domain, and VL1 and VH1 form a first paratope.

In one aspect, an antigen-binding molecule as described herein comprises a first light chain polypeptide comprising, in amino-terminus to carboxy-terminus order, a first light chain variable domain; a first light chain elbow region comprising 1) from one to eight contiguous amino acids selected from amino acid positions 108-115 of human immunoglobulin kappa constant domain according to EU or Kabat numbering, or 2) an amino acid sequence that is at least three amino acids in length; and a first light chain dimerization domain selected from an HLA-E A3 (EA3) domain; a beta 2 microglobulin (B2M) domain; or a first ICAM-1 D1 domain, and a first heavy chain polypeptide comprising, in amino-terminus to carboxy-terminus order, a first heavy chain variable domain; a first heavy chain elbow region comprising 1) from one to eight contiguous amino acids selected from amino acid positions 118-125 of human IgG1 according to EU numbering, or amino acid positions 114-121 of human IgG1 according to Kabat numbering, or 2) an amino acid sequence that is at least three amino acids in length; and a first heavy chain dimerization domain, wherein i) the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer; or ii) the first light chain dimerization domain comprises the first ICAM-1 D1 domain and the first heavy chain dimerization comprises a second ICAM-1 D1 domain, wherein the first and second ICAM-1 D1 domains bind to each other to form a dimer; i) at least one of the B2M domain or EA3 domain differs from the wild-type B2M domain (SEQ ID NO:2) or wild-type EA3 domain (SEQ ID NO:33), respectively, and ii) the first ICAM-1 D1 domain differs from the second ICAM-1 D1 domain; and the first light chain variable domain and first heavy chain variable domain form a first paratope.

In one aspect, an antigen-binding molecule as described herein comprises a dimer of a first polypeptide and a second polypeptide, wherein the first polypeptide comprises, in amino-terminus to carboxy-terminus order, (i) a first immunoglobulin fragment that does not comprise a dimerization sequence of a CH1 or CL domain, and (ii) a first dimerization domain comprising an HLA-E A3 (EA3) domain, a beta-2 microglobulin (B2M) domain, or a first ICAM-1 D1 domain, and the second polypeptide comprises, in amino-terminus to carboxy-terminus order, (i) a second immunoglobulin fragment that does not comprise a dimerization sequence of a CH1 or CL domain, and (ii) a second dimerization domain comprising an EA3 domain, a B2M domain, or a second ICAM-1 D1 domain, wherein i) the first dimerization domain comprises the B2M domain and the second dimerization domain comprises the EA3 domain, or the first dimerization domain comprises the EA3 domain and second dimerization domain comprises the B2M domain, wherein the B2M domain and the EA3 domain bind to each other to form a dimer; or ii) the first dimerization domain comprises the first ICAM-1 D1 domain and the second dimerization domain comprises the second ICAM-1 D1 domain, wherein the first and second ICAM-1 D1 domains bind to each other to form a dimer; i) at least one of the B2M domain or EA3 domain differs from the wild-type B2M domain (SEQ ID NO:2) or wild-type EA3 domain (SEQ ID NO:33), respectively, and ii) the first ICAM-1 D1 domain differs from the second ICAM-1 D1 domain; and the first immunoglobulin fragment comprises a first immunoglobulin variable domain and the second immunoglobulin fragment comprises a second immunoglobulin variable domain, wherein the first and second immunoglobulin variable domains form a first paratope.

In one aspect, provided herein is a composition comprising a plurality of species of polypeptides, wherein at least one first species of the polypeptide comprises a first polypeptide comprising, in amino-terminus to carboxy-terminus order, a first immunoglobulin fragment that does not comprise the dimerization sequence of CH1 or CL domain, and a first dimerization domain comprising an HLA-E A3 (EA3) domain, beta-2 microglobulin (B2M) domain, or first ICAM-1 D1 domain; and at least one second species of the polypeptide comprises a second polypeptide comprising, in amino-terminus to carboxy-terminus order, a second immunoglobulin fragment that does not comprise a dimerization sequence of a CH1 or CL domain, and a second dimerization domain comprising an HLA-E A3 domain, a B2M domain, or a second ICAM-1 D1 domain, wherein (a) the first dimerization domain comprises the B2M domain and the second dimerization domain comprises the EA3 domain, or the first dimerization domain comprises the EA3 domain and second dimerization domain comprises the B2M domain, wherein the B2M domain and the EA3 domain bind to each other to form a dimer; or (b) the first dimerization domain comprises a first ICAM-1 D1 domain and the second dimerization domain comprises a second ICAM-1 D1 domain, wherein the first and second ICAM-1 D1 domains bind to each other to form a dimer; (a) at least one of the B2M domain or EA3 domain differs from the wild type B2M domain or EA3 domain, respectively, and (b) the first ICAM-1 D1 domain differs from the second ICAM-1 D1 domain; the first immunoglobulin fragment comprises a first immunoglobulin variable domain and the second immunoglobulin fragment comprises a second immunoglobulin variable domain, wherein the first and second immunoglobulin variable domains form a first paratope.

In one aspect, provided herein is a composition comprising a plurality of species of polypeptides, wherein each species of the polypeptide comprises a means for binding to a first target antigen; and a means for dimerization, wherein in at least one species of the polypeptides the means for binding comprises a first means for binding to a first target antigen, and a first means of dimerization comprising an HLA-E A3 (EA3) domain that binds to a beta-2 microglobulin (B2M) domain to form a dimer; a B2M domain that binds to an EA3 domain to form a dimer; or a first ICAM-1 D1 domain that binds to a second ICAM-1 D1 domain to form a dimer, and in another at least one species of the polypeptides the means for binding comprises a second means for binding to a first target antigen, and a second means for dimerization comprising a B2M domain that binds to the EA3 domain to form a dimer; an EA3 domain that binds to the B2M domain to form a dimer; or a second ICAM-1 D1 domain that binds to the first ICAM-1 D1 domain to form a dimer, and wherein the first and second means for dimerization bind to each other to form a dimer, at least one of the EA3 domain or B2M domain differs from the wild-type B2M domain (SEQ ID NO:2) or wild-type EA3 domain (SEQ ID NO:33), and the first ICAM-1 D1 domain differs from the second ICAM-1 D1 domain, and the first and second means for binding to a first target antigen form a first paratope.

In one aspect, provided herein is a method of producing a multiparatopic antibody in a single host cell, the method comprising providing one or more polynucleotides encoding a first light chain polypeptide comprising a first light chain variable domain; and a first light chain dimerization domain that is an HLA-E A3 (EA3) domain, a beta-2 microglobulin (B2M) domain, or a first ICAM-1 D1 domain; a first heavy chain comprising a first heavy chain variable domain; a first heavy chain dimerization domain that is an EA3 domain, a B2M domain, or a second ICAM-1 D1 domain, wherein (i) the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer; or (ii) the first light chain dimerization domain comprises a first ICAM-1 D1 domain and the first heavy chain dimerization comprises a second ICAM-1 D1 domain, wherein the first and second ICAM-1 D1 domains bind to each other to form a dimer; (i) at least one of the B2M domain or EA3 domain differs from the wild-type B2M domain (SEQ ID NO:2) or wild-type EA3 domain (SEQ ID NO:33), respectively, and (ii) the first ICAM-1 D1 domain differs from the second ICAM-1 D1 domain; and the first light chain variable domain and first heavy chain variable domain form a first paratope, and a connection to a second antibody heavy chain; a second antibody heavy chain comprising a second heavy chain variable domain; a second heavy chain dimerization domain that is not an EA3 domain, a B2M domain, or an ICAM-1 domain, or is a heavy chain first constant (CH1) domain, and wherein the second heavy chain dimerization domain dimerizes to a second light chain dimerization domain; and a connection to the first antibody heavy chain, and a second antibody light chain comprising a second light chain variable domain; and a second light chain dimerization domain that is not an EA3 domain, a B2M domain, or an ICAM-1 domain, or is a light chain constant (CL) domain, and wherein the second light chain dimerization domain dimerizes to a second heavy chain dimerization domain, delivering the one or more polynucleotide sequences to a host cell, culturing the host cell under conditions allowing for expression of the multiparatopic antibody, thereby producing said multiparatopic antibody, and wherein the first light chain variable domain and first heavy chain variable domain form a first paratope specific for a first target, the first light chain dimerization domain and first heavy chain dimerization domain dimerize together, the second light chain dimerization domain and the second heavy chain dimerization domain dimerize together, the first and second heavy chain are connected together, and i) at least one of the B2M domain or EA3 domain differs from the wild-type B2M domain (SEQ ID NO:2) or wild-type EA3 domain (SEQ ID NO:33), respectively, and ii) the first ICAM-1 D1 domain differs from the second ICAM-1 D1 domain.

In specific embodiments, a first light chain dimerization domain comprises a beta 2 microglobulin (B2M) domain, and a first heavy chain dimerization domain comprises an HLA-E A3 (EA3) domain. In specific embodiments, a first light chain dimerization domain comprises an EA3 domain and a first heavy chain dimerization domain comprises a B2M domain. In specific embodiments, the B2M domain and the EA3 domain bind to each other to form a dimer. In specific embodiments, at least one of the B2M domain or EA3 domain differs from the wild-type B2M domain (SEQ ID NO:2) or wild-type EA3 domain (SEQ ID NO:33), respectively. In specific embodiments, the B2M differs from the wild-type B2M domain (SEQ ID NO:2). In specific embodiments, the EA3 domain differs from the wild-type EA3 domain (SEQ ID NO:33).

In specific embodiments, a first light chain dimerization domain comprises a first ICAM-1 D1 domain and a first heavy chain dimerization domain comprises a second ICAM-1 D1 domain. In specific embodiments, the first ICAM-1 D1 domain and second ICAM-1 D1 domain bind to each other to form a dimer. In specific embodiments, the first ICAM-1 D1 domain differs from the second ICAM-1 D1 domain.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of an amino acid sequence having at least 91% sequence identity to SEQ ID NO:2.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T.

In specific embodiments of the antigen-binding molecule as described herein, the first light chain dimerization domain comprises or consists of an amino acid sequence of a human B2M sequence as set forth in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of an amino acid sequence of a human B2M sequence as set forth in SEQ ID NO:2 but comprising a set of substitutions selected from the following, with positions numbered according to the B2M amino acid numbering in TABLE 1: F56S, W60S, F62T, and K6C; F56S, W60S, F62T, and any one of Y10C, Y10F, and Y10W; F56S, W60S, F62T, and R12C; F56S, W60S, F62T, and any one of D98C; D98F, and D98W; F56S, W60S, F62T, and any one of M99C, M99F, and M99W.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and the B2M domain comprises or consists of the amino acid sequence selected of any one of SEQ ID NOS:2-30.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of the amino acid sequence of

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of an amino acid sequence having at least 85% sequence identity to SEQ ID NO:33.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of an amino acid sequence having one, two, three, four, five, or six single amino acid substitutions to the sequence of SEQ ID NO:33 at one or more amino acid positions 13, 23, 53, 55, 59, and 63 of SEQ ID NO:33.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, and L.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of an amino acid sequence of a human EA3 sequence as set forth in SEQ ID NO:33 but comprising a substitution or set of substitutions selected from the following, with positions numbered according to the EA3 amino acid numbering in TABLE 2: H192C; R202A or R202C; E232C; R234A, R234L, or R234C; D238C; Q242A or Q242L; R234A and Q242A; R234A and Q242L; R234A and Q242A; or R234L and Q242L.

In specific embodiments of the antigen-binding molecule as described herein, the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and wherein the EA3 domain comprises or consists of the amino acid sequence of any one of SEQ ID NOS:32-46.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of the amino acid sequence of

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of an amino acid sequence having at least 81% sequence identity to SEQ ID NO:49.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to the ICAM-1 D1 amino acid numbering in TABLE 3.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3: E34K; T2V, I10T, T23A, E34K, P38T, P63V, S67A, and T78A; T2V, I10T, R13C, T23A, E34K, P38T, R49E, P63V, S67A, T78A, and 84C; T2V, I10T, R13C, T23A, E34K, P38T, E53R, P63V, S67A, T78A, and 84C; T2V, I10T, R13C, L18F, T23A, E34K, P38T, P63V, S67A, T78A, and 84C; T2V, I10T, R13C, L18A, T20A, T23A, E34K, P38T, L42A, V51A, P63V, S67A, T78A, and 84C; or T2V, I10T, R13C, L18W, T23A, E34K, P38T, P63V, S67A, T78A, and 84C.

In specific embodiments of the antigen-binding molecule as described herein, the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain or second ICAM-1 D1 domain comprises or consists of the amino acid sequence of any one of SEQ ID NOS:48-58.

In specific embodiments of the antigen-binding molecule as described herein, a light chain polypeptide (e.g., a first light chain polypeptide) comprises an elbow region between its light chain variable domain and its light chain dimerization domain. In specific embodiments, a heavy chain polypeptide (e.g., a first heavy chain polypeptide) comprises an elbow region between its heavy chain variable domain and its heavy chain dimerization domain. In specific embodiments, the elbow region comprises an amino acid sequence of at least 3 amino acids in length. In specific embodiments, the elbow region comprises or consists of an amino acid sequence of from 3-25 amino acids in length.

In specific embodiments of the antigen-binding molecule as described herein, the elbow region comprises or consists of an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO: 63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO: 68); ASTKGGGGSGGS (SEQ ID NO:69); ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); RTVAGGGGSGGGGS (SEQ ID NO: 82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

In specific embodiments of the antigen-binding molecule as described herein, a polypeptide of the antigen-binding molecule comprises a spacer region fused to the C-terminus of its dimerization domain. In specific embodiments, a light chain polypeptide (e.g., a first light chain polypeptide) comprises a spacer region fused to the C-terminus of its dimerization domain. In specific embodiments, a heavy chain polypeptide (e.g., a first heavy chain polypeptide) comprises a spacer region fused to the C-terminus of its dimerization domain. In specific embodiments, the spacer region comprises an amino acid sequence of at least 2 amino acids in length. In specific embodiments, the spacer region comprises or consists of an amino acid sequence of from 2-9 amino acids in length.

In specific embodiments of the antigen-binding molecule as described herein, the spacer region comprises or consists of an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

In specific embodiments, the spacer region further comprises a hinge region.

In specific embodiments of the antigen-binding molecule as described herein, a polypeptide of the antigen-binding molecule further comprises a C-terminal tag, optionally wherein the C-terminal tag is a 6× His tag (SEQ ID NO: 93), a streptavidin tag (e.g., a Strep-tag II tag), or a human influenza hemagglutinin tag.

In specific embodiments of the antigen-binding molecule as described herein, the antigen-binding molecule is an immunoglobulin, a Fab, a Fab′, a F(ab′)₂, an antibody, a biparatopic antibody, a bispecific antibody, a triparatopic antibody, a trispecific antibody, a tetraparatopic antibody, tetraspecific antibody, a multiparatopic antibody, a multispecific antibody, or any fragment of the antigen-binding molecule that binds to a target antigen.

In specific embodiments, the antigen-binding molecule as described herein further comprises a second light chain polypeptide (LC2) comprising a second light chain variable domain (VL2) and a second heavy chain polypeptide (HC2) comprising a second heavy chain variable domain (VH2), wherein VL2 and VH2 form a second paratope. In specific embodiments, the first and second paratopes bind to different antigens.

In specific embodiments, the antigen-binding molecule as described herein further comprises a third light chain polypeptide (LC3) comprising a third light chain variable domain (VL3) and a third heavy chain polypeptide (HC3) comprising a third heavy chain variable domain (VH3), wherein VL3 and VH3 form a third paratope. In specific embodiments, the first, second, and third paratopes bind to different antigens.

In specific embodiments, the antigen-binding molecule as described herein further comprises a fourth light chain polypeptide (LC4) comprising a fourth light chain variable domain (VL4) and a fourth heavy chain polypeptide (HC4) comprising a fourth heavy chain variable domain (VH4), wherein VL4 and VH4 form a fourth paratope. In specific embodiments, the first, second, third, and fourth paratopes bind to different antigens.

In specific embodiments of the antigen-binding molecule as described herein, the first paratope specifically binds to a first Tumor Associated Antigen (TAA1), the second paratope specifically binds to a second Tumor Associated Antigen (TAA2), the third paratope specifically binds to a third Tumor Associated Antigen (TAA3), and/or the fourth paratope specifically binds to a fourth Tumor Associated Antigen (TAA4).

In another aspect, provided herein is an isolated polynucleotide encoding the antigen-binding molecule as described herein.

In another aspect, provided herein is a vector comprising an isolated polynucleotide encoding the antigen-binding molecule as described herein.

In another aspect, provided herein is a host cell containing a vector comprising an isolated polynucleotide encoding the antigen-binding molecule as described herein.

In another aspect, provided herein is a pharmaceutical composition comprising the antigen-binding molecule as described herein, the isolated polynucleotide as described herein, or the host cell as described herein, and a pharmaceutically acceptable excipient.

In another aspect, provided herein is a method of producing an antigen-binding molecule as described herein, comprising: culturing a host cell as described herein under suitable conditions such that the antigen-binding molecule is expressed by the host cell; and isolating the antigen-binding molecule.

In another aspect, provided herein is a method for treating a disease or disorder in a subject comprising administering to the subject the antigen-binding molecule or pharmaceutical composition as described herein.

In another aspect, provided herein is a method for treating a cancer in a subject in a subject comprising administering to the subject the antigen-binding molecule or pharmaceutical composition as described herein.

### 6. BRIEF DESCRIPTION OF THE DRAWINGS

FIGS. 1A-1C depict CH1-CL replacement strategies as described herein. FIG. 1A shows a generalized bispecific antibody (bsAb) featuring two unique variable domains (hashed lines and gray dots), each with its unique VH and VL situated at the N-terminus of a CH or CL domain which is native in both light chain and heavy chain constant regions. FIG. 1B shows replacement of the CH1 and CL domains of one arm of a native bsAb in a “pseudo Fab” (pFab) design, in which the CH1 and CL domains are replaced by alternate Ig domains (horizontal lines). FIG. 1C shows an extension of the CH1 and CL replacement strategy to a multi-specific antibody, wherein two CH1 and CL domains are replaced with different pFab regions (horizontal lines for one pFab and vertical lines for another pFab). Standard antibody constant domains are shown as white ovals.

FIGS. 2A-2C depict a comparison of the structure of the HLA-E A3 (EA3) domain/beta-2 microglobulin (B2M) domain heterodimer to the structure of a native CH1-CL heterodimer. FIG. 2A shows the Fab structure from RCSB PDB ID:5TZ2. The heavy chain is shown in dark gray while the light chain is shown in light gray. The distance from the heavy chain VH residue S112 (Kabat) to CH1 residue S119 (EU) was 8.1 Å, and the distance from VL I106 (Kabat) to CL K111 (EU) was 13.3 Å, respectively. FIG. 2B shows a EA3/B2M domain heterodimer overlayed on the Fab structure. Distances between the variable domain residues indicated above and N-terminus of the aligned EA3 subunit (residue L201) and B2M (residue R23) were 13.9 Å and 14.0 Å, respectively. FIG. 2C shows ICAM-1 D1 domains overlayed on the Fab structure. Distances between the variable domain residues indicated above and N-terminus of the aligned ICAM-1 D1 homodimer (residue Q1) were 8.0 Å and 21.9 Å, respectively.

FIGS. 3A-3B depict sequence alignments and percent identity of HLA-E A3 (EA3) and beta-2 microglobulin (B2M) wild-type sequences with a human IgG1 G1m CH1 domain, a human IgG1 kappa light chain constant domain, and a human IgG1 lambda light chain constant domain. FIG. 3A shows a sequence alignment of the CH1 domain, and kappa and lambda constant domains with EA3 and B2M. FIG. 3B shows that the EA3 displays higher identity to the CH1 domain than to CL domains while B2M displays higher identity to CL domains. In specific “pseudo-Fab” domains, EA3 replaced CH1 while B2M replaced CL. Figure discloses SEQ ID NOS 98-99, 368-369, and 2, respectively, in order of appearance.

FIGS. 4A-4B depict the design of the B2M (SST) variant as described herein. FIG. 4B shows the structure of HLA-E A3 (EA3) in which the hydrophobic interactions between B2M and the alpha1/alpha2 of EA3 domains are indicated. FIG. 4B shows the sequence alignment of human B2M and B2M (SST). Figure discloses SEQ ID NOS 2 and 4, respectively, in order of appearance.

FIG. 5 depicts in vitro analysis of the abilities of HLA-E A3(EA3)/beta-2 microglobulin (B2M) pseudo-Fabs (pFabs) to bind to RSV-F glycoprotein. In the absence of engineered salt bridges or disulfide bonds, EA3/B2M pseudo-Fabs HLPPB117 and HLPPB270 show weaker binding to RSV-F glycoprotein than the B23B173 native Fab control. A wild-type EA3/B2M dimer lacking anti-RSV-F variable domains served as a negative control.

FIGS. 6A-6B depict intact LC-MS molecular weight analysis of HLA-E A3 (EA3)/beta-2 microglobulin (B2M) pseudo-Fab HLPPB17 and native Fab control (B23B173). FIG. 6A shows the presence of individual light chain and heavy chain arms of the EA3/B2M pFab. FIG. 6B shows the presence of intact B23B173.

FIGS. 7A-7D depict comparison of the expression and solubility levels of engineered pseudo-Fabs (pFabs) that comprise electrostatic mutations present in HLA-E A3 (EA3)/beta-2 microglobulin (B2M) domains by reduced SDS-PAGE. FIG. 7A shows results for pFabs HLPPW10, HLPPW13, HLPPW14, HLPPW15, HLPPW16, HLPPW17, and HLPPW18. FIG. 7B shows results for pFabs HLPPW19, HLPPW20, HLPPW21, HLPPW22, HLPPW23, HLPPW24, HLPPW25, HLPPW26. FIG. 7C shows results for pFabs HLPPW27, HLPPW28, HLPPW29, HLPPW30, HLPPW32, HLPPW33, HLPPW34. FIG. 7D shows results for pFabs HLPPW35, HLPPW36, HLPPW37, HLPPW38, HLPPW40, and HLPPW41. In FIGS. 7A-7D, lane S is the neat CHO supernatant, and lane P is the resuspended CHO pellet fraction.

FIGS. 8A-8D depict comparison of the expression levels of engineered HLA-E A3 (EA3)/beta-2 microglobulin (B2M) pseudo-Fab (pFabs) HLPPW42, HLPPW43, HLPPW54, HLPPW55, HLPPW56, HLPPW57, HLPPW48, HLPPW49, HLPPW50, HLPPW51, HLPPW52, HLPPW53, HLPPW42, HLPPB17, HLPPB270 to a normal Fab HLPPW58, and analysis of disulfide bond integrity by SDS-PAGE. FIG. 8A shows expression leveled detected by luminescence using anti-HIS capture and anti-HA/anti-StrepII detection. FIGS. 8B-8D show reduced (R) and non-reduced (N) SDS-PAGE results. In FIGS. 8B-8D, lane R is the resuspended CHO pellet fraction.

FIGS. 9A-9B depict ELISA analysis of the abilities of engineered HLA-E A3 (EA3)/beta-2 microglobulin (B2M) pseudo-Fab (pFabs) from neat CHO supernatant to bind to RSV-F glycoprotein. FIG. 9A shows luminescence signals for HLPPW42, HLPPW43, HLPPW54, HLPPW55, HLPPW56, HLPPW57, HLPPW48, HLPPW49, HLPPW50, HLPPW51, HLPPW52, HLPPW53, HLPPW42, HLPPB17, HLPPB270, in comparison to a normal Fab HLPPW58. FIG. 9B shows calculated EC50 and 95% confidence interval values.

FIGS. 10A-10B depict ELISA binding of purified disulfide engineered HLA-E A3 (EA3)/beta-2 microglobulin (B2M) pseudo-Fab (pFabs) to RSV glycoprotein. FIG. 10A shows luminescence signals from HLPPW42, HLPPW43, HLPPW54, HLPPW55, HLPPW56, HLPPW57, HLPPW48, HLPPW49, HLPPW50, HLPPW51, HLPPW52, and HLPPW53 in comparison to a normal Fab HLPPW58. FIG. 10B shows calculated EC50 and max signal values.

FIGS. 11A-11D depict size exclusion chromatography (SEC) profiles of HLA-E A3 (EA3)/beta-2 microglobulin (B2M) disulfide variants. Results are shown for HLPPB17 (FIG. 11A), HLPPW57 (FIG. 11B), HLPPW49 (FIG. 11C), and HLPPW53 (FIG. 11D), which show as monodisperse species. Gel filtration standard is overlayed in dashed line. Signals are presented as relative to maximum signal of each sample.

FIG. 12 depicts mass spectrometry analysis of HLA-E A3 (EA3)/beta-2 microglobulin (B2M) disulfide variants. Results are shown for HLPPB17 (FIG. 12A), HLPPW57 (FIG. 12B), HLPPW49 (FIG. 12C), and HLPPW53 (FIG. 12D).

FIGS. 13A-13B depict stability analysis by thermal melting using nanoscale differential scanning fluorimetry (NanoDSF) measurements. FIG. 13A shows melting curves and the first derivative. FIG. 13B shows temperature of melting onset (Ton), melting temperature 1 (Tm1), melting temperature 2 (Tm2), melting temperature 3 (Tm3), and onset temperature of aggregation (Tagg). Two unique disulfide ‘pin’ pairs were identified to have comparable stability to Fab control.

FIG. 14 depicts non-reduced PAGE of HLPPW57-derived HLA-E A3 (EA3)/beta-2 microglobulin (B2M) pseudo-Fab variants HLPPW59, HLPPW60, HLPPW61, HLPPW62, HLPPW63, HLPPW64, HLPPW65, HLPPW66, HLPPW67, HLPPW68, HLPPW69, and HLPPW70.

FIGS. 15A-15C depict size exclusion chromatography (SEC) profiles of select HLA-E A3 (EA3)/beta-2 microglobulin (B2M) elbow variants. FIGS. 15A-15C show results for HLPPW59, HLPPW60, and HLPPW63, which show as monodisperse species. Gel filtration standard overlayed in light grey. Signals are presented as relative to maximum signal of each sample.

FIG. 16 depicts ELISA binding of purified disulfide engineered HLA-E A3 (EA3)/beta-2 microglobulin (B2M) pseudo Fabs to RSV glycoprotein. Luminescence signals are shown for HLPPW59, HLPPW60, HLPPW61, HLPPW62, HLPPW63, HLPPW64, HLPPW65, HLPPW66, HLPPW67, HLPPW68, HLPPW69, and HLPPW70 in comparison with HLPPB271 and HLPPW58.

FIGS. 17A-17D depict stability analysis by thermal melting using nanoscale differential scanning fluorimetry (NanoDSF) measurements to evaluate impact on stability of identify two unique disulfide ‘pin’ pairs of interest, with comparable stability to Fab control. FIG. 17A shows the melting curves and FIG. 17B shows the first derivatives for HLPPW60, HLPPW61, HLPPW62, HLPPW63, HLPPW64, HLPPW65, HLPPW66, HLPPW67, HLPPW68, HLPPW69, and HLPPW70. FIG. 17C shows the melting curves and FIG. 17D shows the first derivatives for HLPPB271 and HLPPW58.

FIGS. 18A-18B depict comparison of purified ICAM-1 D1/ICAM-1 D1 dimers to determine presence of both chains of the heterodimer. Western blot was performed on purified ICAM-1 D1/ICAM-1 D1 dimers HLPPB5, HLPPB6, HLPPB9, and HLPPB10. HLPPB17 and B23B173 serve as an HLA-E A3 (EA3)/beta-2 microglobulin (B2M) heterodimer and native Fab control, respectively.

FIGS. 19A-19D depict intact LC-MS molecular weight analysis of ICAM-1 D1/ICAM-1 D1 pseudo-Fab variants HLPPB5, HLPPB6, HLPPB9, and HLPPB10.

FIG. 20 depicts ELISA analysis of the abilities of engineered ICAM-1 D1/ICAM-1 D1 pseudo-Fabs to bind RSV-F glycoprotein from neat CHO supernatants. Shown are results for HLPPB6 and HLPPB10 in comparison with B23B173 native Fab control.

FIGS. 21A-21B depict stability analysis by thermal melting using nanoscale differential scanning fluorimetry (NanoDSF) measurements for bispecific antibodies HLPPB421, HLPPB423, HLPPB425, and HLPPB426. FIG. 21A shows melting curves and the first derivative. FIG. 21B shows temperature of melting onset (Ton), melting temperature 1 (Tm1), melting temperature 2 (Tm2), melting temperature 3 (Tm3), and onset temperature of aggregation (Tagg).

FIG. 22 depicts nonreduced (NR) and reduced (R) PAGE of purified bispecific antibodies HLPPB423, HLPPB425, HLPPB421, and HLPPB426 where one arm consists of HLPPW59-derived pseudo-Fab and the other arm consists of standard IgG Fab. All samples were normalized by total protein A280 and equal amounts were loaded. Ladder is represented by (L) and molecular weights given in kD.

FIG. 23 depicts size exclusion chromatography (SEC) profiles purified bispecific antibodies HLPPB423, HLPPB425, HLPPB421, and HLPPB426 where one arm consists of HLPPW59-derived pseudo-Fab and the other arm consists of standard IgG Fab. Gel filtration standard is overlayed in light grey. Signals are presented as relative to maximum signal of each sample.

FIGS. 24A-24C depict biolayer interferometry (BLI) binding profiles of purified bispecific antibodies. FIG. 24A shows BLI binding profiles of human epidermal growth factor receptor 2 (HER2)-targeting HLPPB423, HLPPB425, HLPPB421, and HLPPB426 to HER2. FIG. 24B shows BLI binding profiles of mesenchymal epithelial transition (MET) targeting antibodies HLPPB423 and HLPPB425 to MET. FIG. 24C shows BLI binding profiles of cluster of differentiation 3 (CD3) targeting antibodies HLPPB421 and HLPPB426 to CD3.

### 7. DETAILED DESCRIPTION

Provided herein are antigen-binding molecules that comprise one or more polypeptides (e.g., antigen-binding polypeptides) that have an antibody variable domain that binds a target antigen and, in lieu of a light chain constant domain (CL) or heavy chain constant domain 1 (CH1), have a dimerization domain as described herein. In specific embodiments, an antigen-binding molecule binds to one or more target antigens. In specific embodiments, an antigen-binding molecule comprises two or more polypeptides, wherein the polypeptides form paratopes that bind to one or more target antigens. In specific embodiments, a pair of polypeptide each comprise a variable domain and a dimerization domain, wherein said variable domains form a paratope that binds to a target antigen.

### 7.1 Definition

The disclosed methods may be understood more readily by reference to the following detailed description taken in connection with the accompanying Figures, which form a part of this disclosure. It is to be understood that the disclosed methods are not limited to the specific methods described and/or shown herein, and that the terminology used herein is for the purpose of describing particular embodiments by way of example only and is not intended to be limiting.

All patents, published patent applications and publications cited herein are incorporated by reference as if set forth fully herein.

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C”, “A or B,” “A or C,” “B or C,” or “A, B, or C.”

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“Antibody-dependent cellular cytotoxicity,” “antibody-dependent cell-mediated cytotoxicity” or “ADCC” refers to the mechanism of inducing cell death that depends upon the interaction of antibody-coated target cells with effector cells possessing lytic activity, such as natural killer cells (NK), monocytes, macrophages and neutrophils via Fc gamma receptors (FcγR) expressed on effector cells.

“Antibody-dependent cellular phagocytosis” or “ADCP” refers to the mechanism of elimination of antibody-coated target cells by internalization by phagocytic cells, such as macrophages or dendritic cells.

“Antigen” refers to any molecule (e.g., protein, peptide, polysaccharide, glycoprotein, glycolipid, nucleic acid, portions thereof, or combinations thereof) that is capable of mediating an immune response. Exemplary immune responses include antibody production and activation of immune cells, such as T cells, B cells or NK cells.

“Antigen binding fragment” or “antigen binding domain” refers to a portion of a protein that binds the antigen. Antigen binding fragments may be synthetic, enzymatically obtainable or genetically engineered polypeptides and include portions of an immunoglobulin that bind an antigen, such as a VH, a VL, the VH and the VL, Fab, Fab′, F(ab′)₂, Fd and Fv fragments, domain antibodies (dAb) consisting of one VH domain or one VL domain, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as the VH and the VL) may be linked together via a synthetic linker to form various types of single antibody designs in which the VH/VL domains may pair intramolecularly, or intermolecularly in those cases when the VH and the VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibodies” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific, etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g., IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Bispecific” refers to a molecule (such as an antibody) that specifically binds two distinct antigens or two distinct epitopes within the same antigen. The bispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between two or more distinct antigens.

“Complement-dependent cytotoxicity” or “CDC”, refers to the mechanism of inducing cell death in which the Fc effector domain of a target-bound protein binds and activates complement component C1q which in turn activates the complement cascade leading to target cell death. Activation of complement may also result in deposition of complement components on the target cell surface that facilitate CDC by binding complement receptors (e.g., CR3) on leukocytes.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al., (1970) J Exp Med 132: 211-250; Kabat et al., Sequences of Proteins of Immunological Interest, 5th Ed. Public Health Service, National Institutes of Health, Bethesda, Md., 1991), Chothia (Chothia et al., (1987) J Mol Biol 196: 901-17), IMGT (Lefranc et al., (2003) Dev Comp Immunol 27: 55-77) and AbM (Martin and Thornton (1996) J Bmol Biol 263: 800-815). The correspondence between the various delineations and variable region numbering is described (see e.g., Lefranc et al. (2003) Dev Comp Immunol 27: 55-77; Honegger and Pluckthun, J Mol Biol (2001) 309:657-670; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Decrease,” “lower” or “reduce,” refers generally to the ability of a test molecule to mediate a reduced response (i.e., downstream effect) when compared to the response mediated by a control or a vehicle. Exemplary responses include binding of a protein to its antigen or receptor, enhanced binding to FcγR or enhanced Fc effector functions such as enhanced ADCC, CDC and/or ADCP. Decrease may be a statistically significant difference in the measured response between the test molecule and the control (or the vehicle), or a decrease in the measured response, such as a decrease of about 1.1, 1.2, 1.5, 2, 3, 4, 5, 6, 7, 8, 9, 10, 15, 20 or 30 fold or more, such as 500, 600, 700, 800, 900 or 1000 fold or more.

“Enhance,” “promote” or “increase,” refers generally to the ability of the test molecule to mediate a greater response (i.e., downstream effect) when compared to the response mediated by a control or a vehicle. Exemplary responses are binding of a protein to its antigen or receptor, enhanced binding to FcγR or enhanced Fc effector functions such as enhanced ADCC, CDC and/or ADCP. Enhance may be a statistically significant difference in the measured response between the test molecule and control (or vehicle), or an increase in the measured response, such as an increase of about 1.1, 1.2, 1.5, 2, 3, 4, 5, 6, 7, 8, 9, 10, 15, 20 or 30 fold or more, such as 500, 600, 700, 800, 900 or 1000 fold or more.

“Expression vector” refers to a vector that can be utilized in a biological system or in a reconstituted biological system to direct the translation of a polypeptide encoded by a polynucleotide sequence present in the expression vector.

“Heterologous” refers to a polypeptide or a polynucleotide that comprises two or more polypeptides or two or more polynucleotides which are not found in the same relationship to each other in nature.

“Heterologous polynucleotide” refers to a polynucleotide that comprises two or more polynucleotides which are not found in the same relationship to each other in nature.

“Heterologous polypeptide” refers to a polypeptide that comprises two or more polypeptides which are not found in the same relationship to each other in nature.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-396, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Modulate” refers to either enhanced or decreased ability of a test molecule to mediate an enhanced or a reduced response (i.e., downstream effect) when compared to the response mediated by a control or a vehicle.

“Monoclonal antibody” refers to an antibody obtained from a substantially homogenous population of antibody molecules, i.e., the individual antibodies comprising the population are identical except for possible well-known alterations such as removal of C-terminal lysine from the antibody heavy chain or post-translational modifications such as amino acid isomerization or deamidation, methionine oxidation or asparagine or glutamine deamidation. Monoclonal antibodies typically bind one antigenic epitope. A bispecific monoclonal antibody binds two distinct antigenic epitopes. Monoclonal antibodies may have heterogeneous glycosylation within the antibody population. Monoclonal antibody may be monospecific or multispecific such as bispecific, monovalent, bivalent or multivalent.

“Multispecific” refers to a molecule that binds two or more distinct antigens or two or more distinct epitopes within the same antigen. Multispecific molecule may have cross-reactivity to other related antigens, for example to the same antigen from other species (homologs), such as human or monkey, for example Macaca fascicularis (cynomolgus, cyno) or Pan troglodytes, or may bind an epitope that is shared between two or more distinct antigens.

“Polynucleotide” refers to a molecule comprising a chain of nucleotides covalently linked by a sugar-phosphate backbone or other equivalent covalent chemistry. cDNA is a typical example of a polynucleotide.

“Protein” or “polypeptide” are used interchangeably herein are refers to a molecule that comprises one or more polypeptides each comprised of at least two amino acid residues linked by a peptide bond. Protein may be a monomer, or may be a protein complex of two or more subunits, the subunits being identical or distinct. Small polypeptides of less than 50 amino acids may be referred to as “peptides”. Protein may be a heterologous fusion protein, a glycoprotein, or a protein modified by post-translational modifications such as phosphorylation, acetylation, myristoylation, palmitoylation, glycosylation, oxidation, formylation, amidation, citrullination, polyglutamylation, ADP-ribosylation, pegylation or biotinylation.

“Recombinant” refers to polynucleotides, polypeptides, vectors, viruses and other macromolecules that are prepared, expressed, created or isolated by recombinant means.

“Specifically binds,” “specific binding,” “specifically binding” or “binds” refers to a protein binding to an antigen or an epitope within the antigen with greater affinity than for other antigens. Typically, the protein, such as the an antigen-binding protein described herein, binds to the antigen or the epitope within the antigen with an equilibrium dissociation constant (K_{D}) of about 1×10⁻⁶ M or less, about 1×10⁻⁷ M or less, about 5×10⁻⁸ M or less, about 1×10⁻⁸ M or less, about 1×10⁻⁹ M or less, about 1×10⁻¹⁰ M or less, about 1×10⁻¹¹ M or less, or about 1×10⁻¹² M or less, typically with the K_{D} that is at least one hundred fold less than its K_{D} for binding to a non-specific antigen (e.g., BSA, casein).

“Subject” includes any human or nonhuman animal. “Nonhuman animal” includes all vertebrates, e.g., mammals and non-mammals, such as nonhuman primates, sheep, dogs, cats, horses, cows, chickens, amphibians, reptiles, etc. The terms “subject” and “patient” can be used interchangeably herein.

“Therapeutically effective amount” refers to an amount effective, at dosages and for periods of time necessary, to achieve a desired therapeutic result. A therapeutically effective amount may vary according to factors such as the disease state, age, sex, and weight of the individual, and the ability of a therapeutic or a combination of therapeutics to elicit a desired response in the individual.

“Treat,” “treating” or “treatment” of a disease or disorder refers to accomplishing one or more of the following: reducing the severity and/or duration of the disorder, inhibiting worsening of symptoms characteristic of the disorder being treated, limiting or preventing recurrence of the disorder in subjects that have previously had the disorder, or limiting or preventing recurrence of symptoms in subjects that were previously symptomatic for the disorder.

“Trispecific” refers to a molecule (such as an antibody) that specifically binds three distinct antigens or three distinct epitopes within the same antigen. The trispecific molecule may have cross-reactivity to other related antigens, for example to the same antigen from other species (homologs), such as human or monkey, for example Macaca cynomolgus (cynomolgus, cyno) or Pan troglodytes, or may bind an epitope that is shared between three or more distinct antigens.

“Variant,” “mutant” or “altered” refers to a polypeptide or a polynucleotide that differs from a reference polypeptide or a reference polynucleotide by one or more modifications, for example one or more substitutions, insertions or deletions.

The numbering of amino acid residues of the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

### 7.2 Antigen-Binding Polypeptides

In certain aspects, an antigen-binding molecule comprises polypeptides that form paratopes that bind to one or more target antigens. In specific embodiments, the polypeptide of an antigen-binding molecule is an antigen-binding polypeptide. In specific embodiments, a polypeptide of an antigen-binding molecule comprises a variable domain that binds to a target antigen. In specific embodiments, a polypeptide of an antigen-binding molecule comprises at least one dimerization domain as described herein. In specific embodiments, the at least one dimerization domain binds to another dimerization domain of an antigen-binding polypeptide as described herein to form a dimer. In a preferred embodiment, a polypeptide of an antigen-binding molecule comprises a variable domain and at least one dimerization domain.

In specific embodiments, an antigen-binding molecule comprises two, three, four, five, six, seven, or eight polypeptides. In specific embodiments, each of the two, three, four, five, six, seven, or eight polypeptides comprises a variable domain that binds to a target antigen. In specific embodiments, each of the two, three, four, five, six, seven, or eight polypeptides comprises at least one dimerization domain. In specific embodiments, each of the two, three, four, five, six, seven, or eight polypeptides comprises a variable domain that binds to a target antigen and at least one dimerization domain. In specific embodiments, a dimerization domain of an antigen-binding polypeptide binds to another dimerization domain of another antigen-binding polypeptide to form a dimer (in particular, heterodimers, e.g., six polypeptides can form up to three dimers with each other). In a preferred embodiment, an antigen-binding molecule comprises two polypeptides, wherein each polypeptide comprises a variable domain and at least one dimerization domain, wherein the variable domains form a variable region that binds to a target antigen and the dimerization domains bind to each other to form dimers, preferably heterodimers. In a preferred embodiment, an antigen-binding molecule comprises four polypeptides, wherein each polypeptide comprises a variable domain and at least one dimerization domain, wherein the variable domains form two variable regions that each bind to a target antigen, and the dimerization domains bind to each other to form two dimers. In a preferred embodiment, an antigen-binding molecule comprises six polypeptides, wherein each polypeptide comprises a variable domain and at least one dimerization domain, wherein the variable domains form three variable regions that each bind to a target antigen, and the dimerization domains bind to each other to form three dimers.

In specific embodiments, a polypeptide of an antigen-binding molecule comprises a light chain polypeptide comprising a variable domain, and a heavy chain polypeptide comprising a variable domain. In specific embodiments, the variable domains of the light and heavy chains form a variable region comprising a paratope that binds to a target antigen. In specific embodiments, a light chain polypeptide is a first light chain polypeptide (LC1), second light chain polypeptide (LC2), third light chain polypeptide (LC3), fourth light chain polypeptide (LC4), or further light chain polypeptide of an antigen-binding molecule (e.g., LC5+). In specific embodiments, a heavy chain polypeptide is the first heavy chain polypeptide (HC1), second heavy chain polypeptide (HC2), third heavy chain polypeptide (HC3), fourth heavy chain polypeptide (HC4), or further heavy chain polypeptide of an antigen-binding molecule (e.g., HC5+).

In specific embodiments, a light chain polypeptide comprises an immunoglobulin or antibody light chain or one or more fragments thereof. In specific embodiments, a light chain polypeptide comprises an immunoglobulin or antibody kappa (κ) chain, lambda (λ) chain, sigma (σ) chain, iota (ι) chain, or one or more fragments thereof. In specific embodiments, a light chain polypeptide is an intact immunoglobulin light chain comprising a light chain variable domain (VL) and light chain constant domain (CL). In a preferred embodiment, the light chain constant domain is replaced fully or partially with a dimerization domain as described herein. In specific embodiments, a light chain polypeptide can but not need contain an immunoglobulin light chain constant region or a fragment thereof. In specific embodiments, a light chain polypeptide is not an intact immunoglobulin light chain. In specific embodiments, a light chain polypeptide does not comprise a constant domain (CL), or a fragment thereof, of an immunoglobulin light chain. In specific embodiments, a light chain polypeptide does not comprise a dimerization sequence of a light chain constant domain. In specific embodiments, the dimerization sequence of a light chain constant domain binds to a heavy chain constant domain to form a dimer. In specific embodiments, the dimerization sequence of a light chain constant domain mediates dimerization between the light chain and the heavy chain. In specific embodiments, a light chain polypeptide comprises a fragment of a constant light chain domain (CL). In specific embodiments, a light chain polypeptide comprises of from one to eight contiguous amino acids selected from amino acid positions 108-115 of human immunoglobulin kappa constant domain according to EU numbering. In specific embodiments, a light chain polypeptide comprises of from one to eight contiguous amino acids selected from amino acid positions 108-115 of human immunoglobulin kappa constant domain according to Kabat numbering.

In specific embodiments, a heavy chain polypeptide comprises an immunoglobulin or antibody heavy chain, or one or more fragments thereof. In specific embodiments, a heavy chain polypeptide comprises an immunoglobulin or antibody gamma (γ) chain, delta (δ) chain, alpha (α) chain, mu (μ) chain, epsilon (ε) chain, or one or more fragments thereof. In specific embodiments, a heavy chain polypeptide is an intact immunoglobulin heavy chain comprising a heavy chain variable domain (VH), a heavy chain constant domain 1 (CH1), a hinge region, a heavy chain constant domain 2 (CH2), and a heavy chain constant domain 3 (CH3). In a preferred embodiment, the heavy chain constant domain 1 (CH1) is replaced fully or partially with a dimerization domain as described herein. In specific embodiments, a heavy chain polypeptide can but not need contain an immunoglobulin heavy chain constant domain 1 (CH1) or a fragment thereof. In specific embodiments, a heavy chain polypeptide can but not need contain an immunoglobulin heavy chain constant domain 2 (CH2) or a fragment thereof. In specific embodiments, a heavy chain polypeptide can but not need contain an immunoglobulin heavy chain constant domain 3 (CH3) or a fragment thereof. In specific embodiments, a heavy chain polypeptide can but not need contain an immunoglobulin heavy chain constant domain 2 (CH2) or a fragment thereof and an immunoglobulin heavy chain constant domain 3 (CH3). In specific embodiments, a heavy chain polypeptide is not an intact immunoglobulin heavy chain. In specific embodiments, a heavy chain polypeptide does not comprise an immunoglobulin heavy chain constant domain 1 (CH1) or a fragment thereof. In specific embodiments, a heavy chain polypeptide does not comprise a dimerization sequence of a heavy chain constant domain 1 (CH1). In specific embodiments, the dimerization sequence of a heavy chain constant domain 1 (CH1) binds to a light chain constant domain (CL) to form a dimer. In specific embodiments, the dimerization sequence of a heavy chain constant domain mediates dimerization between the heavy chain and the light chain. In specific embodiments, a heavy chain polypeptide comprises a fragment of a heavy chain constant domain (CH1). In specific embodiments, a heavy chain polypeptide comprises of from one to eight contiguous amino acids selected from amino acid positions 118-125 of human IgG1 according to EU numbering. In specific embodiments, a light chain polypeptide comprises of from one to eight contiguous amino acids selected from amino acid positions 114-121 of human IgG1 according to Kabat numbering.

In specific embodiments, a light chain polypeptide is a first light chain polypeptide comprising, in amino-terminus to carboxy-terminus order: VL1-LD1, wherein VL1 is a first light chain variable domain and LD1 is a first light chain dimerization domain. In specific embodiments, a heavy chain polypeptide is a first heavy chain polypeptide comprising, in amino-terminus to carboxy-terminus order: VH1-HD1, wherein VH1 is a first heavy chain variable domain and HD1 is a first heavy chain dimerization domain. In specific embodiments, the VL1 and VH1 form a first paratope that binds to a first target antigen. In specific embodiments, the VL1 and VH1 form a first variable region that comprises a first paratope that binds to a first target antigen. In specific embodiments LD1 comprises a beta-2 microglobulin (B2M) domain and HD1 comprises an HLA-E A3 (EA3) domain. In specific embodiments, LD1 comprises an HLA-E A3 (EA3) domain and HD1 comprises a beta-2 microglobulin (B2M) domain. In specific embodiments, the beta-2 microglobulin (B2M) domain and the HLA-E A3 (EA3) domain bind to each other to form a dimer. In specific embodiments, at least one of the B2M domain or EA3 domain differs from the wild-type B2M domain (SEQ ID NO:2) or wild-type EA3 domain (SEQ ID NO:33), respectively. In specific embodiments, the B2M differs from the wild-type B2M domain (SEQ ID NO:2). In specific embodiments, the EA3 domain differs from the wild-type EA3 domain (SEQ ID NO:33). In specific embodiments, at least one of the B2M or EA3 domain differs from the wild-type B2M domain (SEQ ID NO:2) and wild-type EA3 domain (SEQ ID NO:33), respectively. In specific embodiments, LD1 comprises a first ICAM-1 D1 domain and HD1 comprises a second ICAM-1 D1 domain. In specific embodiments, the first and second ICAM-1 D1 domains bind to each other to form a dimer. In specific embodiments, the first ICAM-1 D1 domain differs from the second ICAM-1 D1 domain.

In specific embodiments, the VH1 and/or VL1 are derived from an antibody of any class of immunoglobulins, including IgM, IgG, IgD, IgA and IgE, and any isotype, including IgG1, IgG2, IgG3 and IgG4, and IgA1, and IgA2. In specific embodiments, the VH1 and/or VL1 are derived from an IgG antibody, such as an IgG1 antibody, IgG2 antibody or IgG4 antibody (e.g., IgG4 nullbody and variants of IgG4 antibodies).

In specific embodiments, the VH1 is derived from an immunoglobulin or antibody gamma (γ) chain, delta (δ) chain, alpha (α) chain, mu (μ) chain, or epsilon (ε) heavy chain. In specific embodiments, the VL1 is derived from an immunoglobulin or antibody kappa (κ) chain, lambda (λ) chain, sigma (σ) chain, or iota (ι) light chain.

In specific embodiments, the VH1 and/or VL1 are derived from a human antibody, a humanized antibody, or an antibody from a nonhuman animal such as a mouse antibody, a rat antibody, a camel antibody, a llama antibody, or a chimeric antibody thereof.

In specific embodiments, the first light chain polypeptide comprises an elbow region between VL1 and LD1. In specific embodiments, a first light chain polypeptide comprises, in amino-terminus to carboxy-terminus order: VL1-LE1-LD1, wherein VL1 is a first light chain variable domain, LE1 is a first light chain elbow region, and LD1 is a first light chain dimerization domain.

In specific embodiments, the first light chain polypeptide comprises a first light chain spacer region fused to the C-terminus of LD1. In specific embodiments, the first light chain polypeptide comprises, in amino-terminus to carboxy-terminus order: VL1-LD1-LS1, wherein VL1 is a first light chain variable domain, LD1 is a first light chain dimerization domain, and LS1 is a first light chain spacer region.

In specific embodiments, the first light chain polypeptide comprises, in amino-terminus to carboxy-terminus order: VL1-LE1-LD1-LS1, wherein VL1 is a first light chain variable domain, LE1 is a first light chain elbow region, LD1 is a first light chain dimerization domain, and LS1 is a first light chain spacer region.

In specific embodiments, the first heavy chain polypeptide comprises an elbow region between VH1 and HD1. In specific embodiments, a first heavy chain polypeptide comprises, in amino-terminus to carboxy-terminus order: VH1-HE1-HD1, wherein VH1 is a first heavy chain variable domain, HE1 is a first heavy chain elbow region, and HD1 is a first heavy chain dimerization domain.

In specific embodiments, the first heavy chain polypeptide comprises a first heavy chain spacer region fused to the C-terminus of HD1. In specific embodiments, the first heavy chain polypeptide comprises, in amino-terminus to carboxy-terminus order: VH1-HD1-HS1, wherein VH1 is a first heavy chain variable domain, HD1 is a first heavy chain dimerization domain, and HS1 is a first heavy chain spacer region.

In specific embodiments, the first heavy chain polypeptide comprises, in amino-terminus to carboxy-terminus order: VH1-HE1-HD1-HS1, wherein VH1 is a first heavy chain variable domain, HE1 is a first heavy chain elbow region, HD1 is a first heavy chain dimerization domain, and HS1 is a first heavy chain spacer region.

A light chain polypeptide as described herein may be a first, second, third, or fourth light chain polypeptide of an antigen-binding molecule, which are each contemplated to be embodied as described herein for a first light chain polypeptide but renumbered accordingly (e.g., VLn, LEn, LDn, and LSn, wherein n is 1 for a first light chain polypeptide, n is 2 for a second light chain polypeptide, n is 3 for a third light chain polypeptide, or n is 4 for a fourth light chain polypeptide). A heavy chain polypeptide as described herein may be a first, second, third, or fourth heavy chain polypeptide of an antigen-binding molecule, which are each contemplated to be embodied as described herein for a first heavy chain polypeptide but renumbered accordingly (e.g., VHn, HEn, HDn, and HSn, wherein n is 1 for a first heavy chain polypeptide, n is 2 for a second heavy chain polypeptide, n is 3 for a third heavy chain polypeptide, or n is 4 for a fourth heavy chain polypeptide).

In specific embodiments, the VHn and/or VLn (wherein n is 1 for a first light chain polypeptide, n is 2 for a second light chain polypeptide, n is 3 for a third light chain polypeptide, or n is 4 for a fourth light chain polypeptide) are derived from an antibody of any class of immunoglobulins, including IgM, IgG, IgD, IgA and IgE, and any isotype, including IgG1, IgG2, IgG3 and lgG4, and IgAQ1, and IgA2. In specific embodiments, the VHn and/or VLn are derived from an IgG antibody, such as an IgG1 antibody, IgG2 antibody or IgG4 antibody (e.g., IgG4 nullbody and variants of IgG4 antibodies).

In specific embodiments, the VHn (wherein n is 1 for a first light chain polypeptide, n is 2 for a second light chain polypeptide, n is 3 for a third light chain polypeptide, or n is 4 for a fourth light chain polypeptide) is derived from an immunoglobulin or antibody gamma (γ) chain, delta (δ) chain, alpha (α) chain, mu (μ) chain, or epsilon (ε) heavy chain. In specific embodiments, the VLn (wherein n is 1 for a first light chain polypeptide, n is 2 for a second light chain polypeptide, n is 3 for a third light chain polypeptide, or n is 4 for a fourth light chain polypeptide) is derived from an immunoglobulin or antibody kappa (κ) chain, lambda (λ) chain, sigma (σ) chain, or iota (ι) light chain.

In specific embodiments, the VHn and/or VLn (wherein n is 1 for a first light chain polypeptide, n is 2 for a second light chain polypeptide, n is 3 for a third light chain polypeptide, or n is 4 for a fourth light chain polypeptide) are derived from a human antibody, a humanized antibody, or an antibody from a nonhuman animal such as a mouse antibody, a rat antibody, a camel antibody, a llama antibody, or a chimeric antibody thereof.

In specific embodiments, the first heavy chain polypeptide further comprises an immunoglobulin constant domain. In specific embodiments, the first heavy chain polypeptide further comprises a heavy chain constant domain 2 (CH2) of an antibody or a fragment thereof, optionally wherein the CH2 domain is fused to the C-terminus of the first heavy chain dimerization domain or first heavy chain spacer region. In specific embodiments, the first heavy chain polypeptide further comprises a heavy chain constant domain 3 (CH3) of an antibody or a fragment thereof, optionally wherein the CH3 domain is fused to the C-terminus of the first heavy chain dimerization domain, C-terminus of the first heavy chain spacer region, or C-terminus of the CH2 domain when present. In specific embodiments, the first heavy chain polypeptide further comprises a heavy chain fourth constant (CH4) domain of an antibody or a fragment thereof, optionally wherein the CH4 domain is fused to the C-terminus of the first heavy chain dimerization domain, C-terminus of the first heavy chain spacer region, C-terminus of the CH2 domain when present, or C-terminus of the CH3 domain when present.

In specific embodiments, the CH2 and/or CH3 are derived from an antibody of any class of immunoglobulins, including IgM, IgG, IgD, IgA and IgE, and any isotype, including IgG1, IgG2, IgG3 and lgG4, and IgAQ1, and IgA2. In specific embodiments, the CH2 and/or CH3 are derived from an IgG antibody, such as an IgG1 antibody, IgG2 antibody or IgG4 antibody (e.g., IgG4 nullbody and variants of IgG4 antibodies).

In specific embodiments, the CH2 and/or CH3 are derived from an immunoglobulin or antibody gamma (γ) chain, delta (δ) chain, alpha (α) chain, mu (μ) chain, or epsilon (ε) heavy chain.

In specific embodiments, the CH2 and/or CH3 are derived from a human antibody, a humanized antibody, or an antibody from a nonhuman animal such as a mouse antibody, a rat antibody, a camel antibody, a llama antibody, or a chimeric antibody thereof.

### 7.3 Dimerization Domains

In certain aspects, a dimerization domain of a polypeptide of an antigen-binding molecule forms a dimer with another dimerization domain of another polypeptide of the antigen-binding molecule. In specific embodiments, a polypeptide of an antigen-binding molecule as described herein comprises a means for dimerization with another polypeptide of the antigen-binding molecule. In specific embodiments, the means for dimerization is a dimerization domain as described herein.

In specific embodiments, a dimerization domain binds to another dimerization domain to form a dimer. In specific embodiments, a light chain dimerization domain replaces fully or partially a light chain constant domain (CL). In specific embodiments, a light chain dimerization domain replaces fully or partially the dimerization sequence of a light chain constant domain (CL). In specific embodiments, a heavy chain dimerization domain replaces fully or partially a heavy chain constant domain 1 (CH1). In specific embodiments, a heavy chain dimerization domain replaces fully or partially the dimerization sequence of a heavy chain constant domain 1 (CH1).

### 7.3.1 Beta-2 Microglobulin (B2M) Domains

In specific embodiments, a dimerization domain as described herein comprises a beta-2 microglobulin (B2M) domain. In specific embodiments, a B2M domain is a human B2M domain. In specific embodiments, a B2M domain is a wild-type human B2M domain, or a fragment thereof. In specific embodiments, a B2M domain comprises or consists of the amino acid sequence of B2M as set forth in UniProt Accession Number P61769, or a fragment thereof (e.g., the mature B2M lacking the signal sequence). The amino acid sequence of B2M as set forth in UniProt Accession Number P61769 is: MSRSVALAVLALLSLSGLEAIQRTPKIQVYSRHPAENGKSNFLNCYVSGFHPSDIEVD LLKNGERIEKVEHSDLSFSKDWSFYLLYYTEFTPTEKDEYACRVNHVTLSQPKIVKW DRDM (SEQ ID NO:1). In specific embodiments, the B2M domain comprises or consists of the amino acid sequence of:

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM.

In specific embodiments, the B2M domain is a variant B2M domain comprising an amino acid sequence that differs from the wild-type B2M domain of SEQ ID NO:2. In specific embodiments, a variant B2M domain comprises at least one amino acid substitution, deletion, or insertion relative to the wild-type B2M domain at least one position numbered according to the B2M amino acid numbering in TABLE 1, below. In specific embodiments, a variant B2M domain comprises at least one amino acid substitution, deletion, or insertion relative to the wild-type B2M domain at least one position numbered according to the amino acid sequence of SEQ ID NO:2. In specific embodiments, a variant B2M domain differs from the wild-type B2M domain of SEQ ID NO:1 or SEQ ID NO:2 in substitutions, deletions, or insertions of one, two, three, four, five, six, seven, or eight amino acids.

TABLE 1
B2M Amino Acid Numbering

| | |
|---|---|
| B2M Amino Acid Numbering | 0--------1---------2---------3---------4---------5 |
| (residues 3-50) | 12345678901234567890123456789012345678901234567890 |
| B2M WT Sequence | RTPKIQVYSRHPAENGKSNFLNCYVSGFHPSDIEVDLLKNGERIEKVE |
| (positions K6, Y10, and R12 are | (continues below) |
| bolded and underlined) | |
| B2M Amino Acid Numbering | 5--------6---------7---------8---------9--------- |
| (residues 51-99) | 1234567890123456789012345678901234567890123456789 |
| B2M WT Sequence | HSDLSFSKDWSFYLLYYTEFTPTEKDEYACRVNHVTLSQPKIVKWDRDM |
| (positions F56, W60, F62, D98, and | (SEQ ID NO: 2) |
| M99 are bolded and underlined) | |

In specific embodiments, the B2M domain comprises or consists of an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:2. In preferred embodiments, the B2M domain comprises an amino acid sequence having at least 91% sequence identity to SEQ ID NO:2.

In specific embodiments, the B2M domain comprises or consists of an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2. In specific embodiments, the B2M domain comprises or consists of an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T.

In specific embodiments, the B2M domain comprises or consists of an amino acid sequence of a human B2M sequence as set forth in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1. In specific embodiments, the B2M domain comprises or consists of an amino acid sequence of a human B2M sequence as set forth in SEQ ID NO:2 but comprising a set of substitutions selected from the following, with positions numbered according to the B2M amino acid numbering in TABLE 1: i) F56S, W60S, F62T, and K6C; ii) F56S, W60S, F62T, and any one of Y10C, Y10F, or Y10W; iii) F56S, W60S, F62T, and R12C; iv) F56S, W60S, F62T, and any one of D98C; D98F, or D98W; or v) F56S, W60S, F62T, and any one of M99C, M99F, or M99W.

In specific embodiments, the B2M domain comprises or consists of the amino acid sequence of any one of SEQ ID NOS:2-30. In specific embodiments, the B2M domain comprises or consists of the amino acid sequence of SEQ ID NO:2. In specific embodiments, the B2M domain comprises or consists of the amino acid sequence of SEQ ID NO:4. In specific embodiments, the B2M domain comprises or consists of the amino acid sequence of SEQ ID NO:5. In specific embodiments, the B2M domain comprises or consists of the amino acid sequence of SEQ ID NO:6. In specific embodiments, the B2M domain comprises or consists of the amino acid sequence of SEQ ID NO:7. In specific embodiments, the B2M domain comprises or consists of the amino acid sequence of SEQ ID NO:8. In specific embodiments, the B2M domain comprises or consists of the amino acid sequence of SEQ ID NO:9. In specific embodiments, the B2M domain comprises or consists of the amino acid sequence of SEQ ID NO:10. In specific embodiments, the B2M domain comprises or consists of the amino acid sequence of SEQ ID NO:11. In specific embodiments, the B2M domain comprises or consists of the amino acid sequence of SEQ ID NO:12. In specific embodiments, the B2M domain comprises or consists of the amino acid sequence of SEQ ID NO:13. In specific embodiments, the B2M domain comprises or consists of the amino acid sequence of SEQ ID NO:14. In specific embodiments, the B2M domain comprises or consists of the amino acid sequence of SEQ ID NO:15. In specific embodiments, the B2M domain comprises or consists of the amino acid sequence of SEQ ID NO:16. In specific embodiments, the B2M domain comprises or consists of the amino acid sequence of SEQ ID NO:17. In specific embodiments, the B2M domain comprises or consists of the amino acid sequence of SEQ ID NO:18. In specific embodiments, the B2M domain comprises or consists of the amino acid sequence of SEQ ID NO:19. In specific embodiments, the B2M domain comprises or consists of the amino acid sequence of SEQ ID NO:20. In specific embodiments, the B2M domain comprises or consists of the amino acid sequence of SEQ ID NO:21. In specific embodiments, the B2M domain comprises or consists of the amino acid sequence of SEQ ID NO:22. In specific embodiments, the B2M domain comprises or consists of the amino acid sequence of SEQ ID NO:23. In specific embodiments, the B2M domain comprises or consists of the amino acid sequence of SEQ ID NO:24. In specific embodiments, the B2M domain comprises or consists of the amino acid sequence of SEQ ID NO:25. In specific embodiments, the B2M domain comprises or consists of the amino acid sequence of SEQ ID NO:26. In specific embodiments, the B2M domain comprises or consists of the amino acid sequence of SEQ ID NO:27. In specific embodiments, the B2M domain comprises or consists of the amino acid sequence of SEQ ID NO:28. In specific embodiments, the B2M domain comprises or consists of the amino acid sequence of SEQ ID NO:29. In specific embodiments, the B2M domain comprises or consists of the amino acid sequence of SEQ ID NO:30.

### 7.3.2 HLA-E A3 (EA3) Domains

In specific embodiments, a dimerization domain as described herein comprises an HLA class I histocompatibility antigen alpha chain-E alpha-3 domain (referred to herein as HLA-E A3 or EA3). In specific embodiments, an EA3 domain is a human EA3 domain. In specific embodiments, an EA3 domain is a human wild-type EA3 domain, or a fragment thereof. In specific embodiments, an EA3 domain comprises or consists of the amino acid sequence of EA3 as set forth in UniProt Accession Number P13747, or a fragment thereof. The amino acid sequence of the entire HLA class I histocompatibility antigen alpha chain-E (HLA-E) as set forth in UniProt Accession Number P13747, which comprises an EA3 domain, is: MVDGTLLLLLSEALALTQTWAGSHSLKYFHTSVSRPGRGEPRFISVGYVDDTQFVRF DNDAASPRMVPRAPWMEQEGSEYWDRETRSARDTAQIFRVNLRTLRGYYNQSEAG SHTLQWMHGCELGPDGRFLRGYEQFAYDGKDYLTLNEDLRSWTAVDTAAQISEQK SNDASEAEHQRAYLEDTCVEWLHKYLEKGKETLLHLEPPKTHVTHHPISDHEATLRC WALGFYPAEITLTWQQDGEGHTQDTELVETRPAGDGTFQKWAAVVVPSGEEQRYT CHVQHiEGLPEPVTLRWKPASQPTIPIVGIIAGLVLLGSVVSGAVVAAVIWRKKSSGGK GGSYSKAEWSDSAQGSESHSL (SEQ ID NO:31). The amino acid sequence of EA3 as set forth in UniProt Accession Number P13747 (e.g., positions 204-295 of SEQ ID NO:31) is: EPPKTHVTHHPISDHEATLRCWALGFYPAEITLTWQQDGEGHTQDTELVETRPAGDG TFQKWAAVVVPSGEEQRYTCHVQHEGLPEPVTLRW (SEQ ID NO:32). In specific embodiments, the EA3 domain comprises or consists of the amino acid sequence of:

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW.

In specific embodiments, the EA3 domain is a variant EA3 domain comprising an amino acid sequence that differs from the wild-type EA3 domain of SEQ ID NO:33. In specific embodiments, a variant EA3 domain comprises at least one amino acid substitution, deletion, or insertion relative to the wild-type EA3 domain at least one position numbered according to the EA3 amino acid numbering in TABLE 2, below. In specific embodiments, a variant EA3 domain comprises at least one amino acid substitution, deletion, or insertion relative to the wild-type EA3 domain at least one position numbered according to the amino acid sequence of SEQ ID NO:33. In specific embodiments, a variant EA3 domain differs from the wild-type EA3 domain of SEQ ID NO:31, SEQ ID NO:32 or SEQ ID NO:33 in substitutions, deletions, or insertions of one, two, three, four, five, or six amino acids.

TABLE 2
EA3 Amino Acid Numbering

| | |
|---|---|
| EA3 Amino Acid Numbering | 18--------19--------20--------21--------22--------23 |
| (residues 180-230) | 012345678901234567890123456789012345678901234567890 |
| EA3 WT Sequence | LHLEPPKTHVTHHPISDHEATLRCWALGFYPAEITLTWQQDGEGHTQDTEL |
| (positions H192 and R202 are | (continues below) |
| bolded and-underlined) | |
| EA3 Amino Acid Numbering | 23-------24--------25--------26--------27--------- |
| (residues 231-274) | 1234567890123456789012345678901234567890123456789 |
| EA3 WT Sequence | VETRPAGDGTFQKWAAVVVPSGEEQRYTCHVQHEGLPEPVTLRW (SEQ ID NO: 33) |
| (positions E232, R234, D238, | |
| and Q242 are bolded and | |
| underlined) | |

In specific embodiments, the EA3 domain comprises or consists of an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:33. In preferred embodiments, the EA3 domain comprises an amino acid sequence having at least 93% sequence identity to SEQ ID NO:33.

In specific embodiments, the EA3 domain comprises or consists of an amino acid sequence having one, two, three, four, five, or six single amino acid substitutions to the sequence of SEQ ID NO:33 at one or more amino acid positions 13, 23, 53, 55, 59, and 63 of SEQ ID NO:33. In specific embodiments, the EA3 domain comprises or consists of an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, and L.

In specific embodiments, the EA3 domain comprises or consists of an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2. In specific embodiments, the EA3 domain comprises or consists of an amino acid sequence of a human EA3 sequence as set forth in SEQ ID NO:33 but comprising a set of substitutions selected from the following, with positions numbered according to the EA3 amino acid numbering in TABLE 2, i) H192C; ii) R202A or R202C; iii) E232C; iv) R234A, R234L, or R234C; v) D238C; vi) Q242A or Q242L; vii) R234A and Q242A; viii) R234A and Q242L; ix) R234A and Q242A; x) R234L and Q242L.

In specific embodiments, the EA3 domain comprises or consists of the amino acid sequence of any one of SEQ ID NOS:32-33 and 35-46. In specific embodiments, the EA3 domain comprises or consists of the amino acid sequence of SEQ ID NO:32. In specific embodiments, the EA3 domain comprises or consists of the amino acid sequence of SEQ ID NO:33. In specific embodiments, the EA3 domain comprises or consists of the amino acid sequence of SEQ ID NO:35. In specific embodiments, the EA3 domain comprises or consists of the amino acid sequence of SEQ ID NO:36. In specific embodiments, the EA3 domain comprises or consists of the amino acid sequence of SEQ ID NO:37. In specific embodiments, the EA3 domain comprises or consists of the amino acid sequence of SEQ ID NO:38. In specific embodiments, the EA3 domain comprises or consists of the amino acid sequence of SEQ ID NO:39. In specific embodiments, the EA3 domain comprises or consists of the amino acid sequence of SEQ ID NO:40. In specific embodiments, the EA3 domain comprises or consists of the amino acid sequence of SEQ ID NO:41. In specific embodiments, the EA3 domain comprises or consists of the amino acid sequence of SEQ ID NO:42. In specific embodiments, the EA3 domain comprises or consists of the amino acid sequence of SEQ ID NO:43. In specific embodiments, the EA3 domain comprises or consists of the amino acid sequence of SEQ ID NO:44. In specific embodiments, the EA3 domain comprises or consists of the amino acid sequence of SEQ ID NO:45. In specific embodiments, the EA3 domain comprises or consists of the amino acid sequence of SEQ ID NO:46.

### 7.3.3 ICAM-1 D1 Domains

In specific embodiments, a dimerization domain as described herein comprises or consists of an intercellular adhesion molecule 1 domain 1 (ICAM-1 D1) domain. In specific embodiments, an ICAM-1 D1 domain is a human ICAM-1 D1 domain. In specific embodiments, an ICAM-1 D1 domain is a wild-type human ICAM-1 D1 domain, or a fragment thereof. In specific embodiments, an ICAM-1 D1 domain comprises or consists of the amino acid sequence of SEQ ID NO:49.

In specific embodiments, an ICAM-1 D1 domain comprises or consists of the amino acid sequence of ICAM-1 D1 as set forth in UniProt Accession Number P05362, or a fragment thereof. The amino acid sequence of the intercellular adhesion molecule 1 (ICAM-1) as set forth in UniProt Accession Number P05362, which comprises an ICAM-1 D1 domain, is: MAPSSPRPALPALLVLLGALFPGPGNAQTSVSPSKVILPRGGSVLVTCSTSCDQPKLL GIETPLPKKELLLPGNNRKVYELSNVQEDSQPMCYSNCPDGQSTAKTFLTVYWTPER VELAPLPSWQPVGKNLTLRCQVEGGAPRANLTVVLLRGEKELKREPAVGEPAEVTT TVLVRRDHHGANFSCRTELDLRPQGLELFENTSAPYQLQTFVLPATPPQLVSPRVLEV DTQGTVVCSLDGLFPVSEAQVHLALGDQRLNPTVTYGNDSFSAKASVSVTAEDEGT QRLTCAVILGNQSQETLQTVTIYSFPAPNVILTKPEVSEGTEVTVKCEAHPRAKVTLN GVPAQPLGPRAQLLLKATPEDNGRSFSCSATLEVAGQLIIIKNQTRELRVLYGPRLDE RDCPGNWTWPENSQQTPMCQAWGNPLPELKCLKDGTFPLPIGESVTVTRDLEGTYL CRARSTQGEVTRKVTVNVLSPRYEIVIITVVAAAVIMGTAGLSTYLYNRQRKIKKYR LQQAQKGTPMKPNTQATPP (SEQ ID NO:47). The amino acid sequence of ICAM-1 D1 as set forth in UniProt Accession Number P05362 (e.g., positions 41-103 of SEQ ID NO:47) is: GGSVLVTCSTSCDQPKLLGIETPLPKKELLLPGNNRKVYELSNVQEDSQPMCYSNCP DGQSTA (SEQ ID NO:48). In specific embodiments, the ICAM-1 D1 domain comprises or consists of the amino acid sequence of:

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY.

In specific embodiments, the ICAM-1 D1 domain is a variant ICAM-1 D1 domain comprising an amino acid sequence that differs from the wild-type ICAM-1 D1 domain of SEQ ID NO:49. In specific embodiments, a variant ICAM-1 D1 domain comprises at least one amino acid substitution, deletion, or insertion relative to the wild-type EA3 domain at at least one position numbered according to the ICAM-1 D1 amino acid numbering in TABLE 3, below. In specific embodiments, a variant ICAM-1 D1 domain comprises at least one amino acid substitution, deletion, or insertion relative to the wild-type ICAM-1 D1 domain at at least one position numbered according to the amino acid sequence of SEQ ID NO:49. In specific embodiments, a variant ICAM-1 D1 domain differs from the wild-type ICAM-1 D1 domain of SEQ ID NO:47, SEQ ID NO:48, or SEQ ID NO:49 in substitutions, deletions, or insertions of 1, 2, 3, 4, 5, 6, 7, 8, 9, 10, 11, 12, 13, 14, or 15 amino acids.

TABLE 3
ICAM-1 D1 Amino Acid Numbering

| | |
|---|---|
| ICAM-1 D1 Amino Acid Numbering | |
| (residues 1-50) | 0---------1---------2---------3---------4---------5 |
| ICAM-1 D1 WT Sequence | 012345678901234567890123456789012345678901234567890 |
| (positions T2, 110, R13, L18, T20, | QTSVSPSKVILPRGGSVLVTCSTSCDQPKLLGIETPLPKKELLLPGNNRK |
| T23, E34, P38, L42, and R49 are | (continues below) |
| bolded and underlined) | |
| ICAM-1 D1 Amino Acid Numbering | 5--------6---------7---------8---- |
| (residues 51-83) | 1234567890123456789012345678901234 |
| ICAM-1 D1 WT Sequence | VYELSNVQEDSQPMCYSNCPDGQSTAKTFLTVY (SEQ ID NO: 49) |
| (positions V51, E53, P63, S67, and | |
| T78 are bolded and underlined) | |

In specific embodiments, the ICAM-1 D1 domain comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to SEQ ID NO:49. In preferred embodiments, the ICAM-1 D1 domain comprises an amino acid sequence having at least 81% sequence identity to SEQ ID NO:49.

In specific embodiments, the ICAM-1 D1 domain comprises or consists of an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition. In specific embodiments, the ICAM-1 D1 domain comprises or consists of an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition.

In specific embodiments, the ICAM-1 D1 domain comprises or consists of an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered to the ICAM-1 D1 amino acid numbering in TABLE 3. In specific embodiments, ICAM-1 D1 domain comprises or consists of an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3, i) E34K; ii) T2V, I10T, T23A, E34K, P38T, P63V, S67A, and T78A; iii) T2V, I10T, R13C, T23A, E34K, P38T, R49E, P63V, S67A, T78A, and 84C; iv) T2V, I10T, R13C, T23A, E34K, P38T, E53R, P63V, S67A, T78A, 84C; R234A, R234L, and R234C; v) T2V, I10T, R13C, L18F, T23A, E34K, P38T, P63V, S67A, T78A, and 84C; vi) T2V, I10T, R13C, L18A, T20A, T23A, E34K, P38T, L42A, V51A, P63V, S67A, T78A, and 84C; or vii) T2V, I10T, R13C, L18W, T23A, E34K, P38T, P63V, S67A, T78A, and 84C.

In specific embodiments, the ICAM-1 D1 domain comprises or consists of the amino acid sequence of any one of SEQ ID NOS:48-49 and 51-58. In specific embodiments, the ICAM-1 D1 domain comprises or consists of the amino acid sequence of SEQ ID NO:48. In specific embodiments, the ICAM-1 D1 domain comprises or consists of the amino acid sequence of SEQ ID NO:49. In specific embodiments, the ICAM-1 D1 domain comprises or consists of the amino acid sequence of SEQ ID NO:51. In specific embodiments, the ICAM-1 D1 domain comprises or consists of the amino acid sequence of SEQ ID NO:52. In specific embodiments, the ICAM-1 D1 domain comprises or consists of the amino acid sequence of SEQ ID NO:53. In specific embodiments, the ICAM-1 D1 domain comprises or consists of the amino acid sequence of SEQ ID NO:54. In specific embodiments, the ICAM-1 D1 domain comprises or consists of the amino acid sequence of SEQ ID NO:55. In specific embodiments, the ICAM-1 D1 domain comprises or consists of the amino acid sequence of SEQ ID NO:56. In specific embodiments, the ICAM-1 D1 domain comprises or consists of the amino acid sequence of SEQ ID NO:57. In specific embodiments, the ICAM-1 D1 domain comprises or consists of the amino acid sequence of SEQ ID NO:58.

### 7.4 Elbow Regions

In certain aspects, an elbow region of a polypeptide of an antigen-binding molecule connects its variable domain to its dimerization domain. In specific embodiments, a light chain polypeptide or heavy chain polypeptide of an antigen-binding molecule comprises an elbow region. In specific embodiments, an elbow region is between a variable domain and a dimerization domain of a light chain, or between a variable domain and a dimerization domain of a heavy chain polypeptide. In specific embodiments, a light chain polypeptide (e.g., a first, second, third, fourth, or further light chain polypeptide) comprises a light chain elbow region (e.g., a first, second, third, fourth, or further light chain elbow region, respectively). In specific embodiments, a heavy chain polypeptide (e.g., a first, second, third, fourth, or further heavy chain polypeptide) comprises a heavy chain elbow region (e.g., a first, second, third, fourth, or further heavy chain elbow region, respectively).

In specific embodiments, a light chain elbow region comprises or consists of an amino acid sequence of from 3-25 amino acids in length. In some embodiments, an elbow region is at least three amino acids in length. In specific embodiments, a light chain elbow region comprises or consists of an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69); ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); RTVAGGGGSGGGGS (SEQ ID NO:82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

In specific embodiments, the light chain dimerization domain is the B2M domain and the light chain elbow region comprises or consists of an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); and RTVAGGGGSGGGGS (SEQ ID NO:82).

In specific embodiments, the heavy chain dimerization domain is the EA3 domain and the heavy chain elbow region comprises or consists of an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); or ASTKGGGGSGGGGS (SEQ ID NO:71).

In specific embodiments, the light chain dimerization domain is the first ICAM-1 D1 domain and the light chain elbow region comprises or consists of an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86). In specific embodiments, the heavy chain dimerization domain is the second ICAM-1 D1 domain and the heavy chain elbow region comprises or consists of an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

### 7.5 Spacers and Tags

In certain aspects, a spacer region of a polypeptide of an antigen-binding molecule is fused to the C-terminus of its dimerization domain. In specific embodiments, a light chain spacer region is fused to the C-terminus of a light chain dimerization domain of an antigen-binding molecule as described herein. In specific embodiments, a heavy chain spacer region is fused to the C-terminus of a heavy chain dimerization domain of an antigen-binding molecule as described herein.

In specific embodiments, a light chain spacer region comprises a hinge region. In specific embodiments, a light chain spacer region connects to a hinge region. In specific embodiments, the heavy chain spacer region comprises a hinge region. In specific embodiments, a heavy chain spacer region connects to a hinge region. In specific embodiments, the hinge region is an antibody hinge region (e.g., an IgG1, IgG2, IgG3, or IgG4 hinge region).

In specific embodiments, the light chain spacer region or heavy chain spacer region comprises or consists of an amino acid sequence of from 2-9 amino acids in length. In specific embodiments, the light chain spacer region or heavy chain spacer region comprises or consists of an amino acid sequence selected from the group consisting of EPKSS (SEQ ID NO:87); G, SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

In specific embodiments, the spacer region of a polypeptide of an antigen-binding molecule connects to another moiety (e.g., another protein). In specific embodiments, the spacer region of a polypeptide of an antigen-binding molecule connects to a C-terminal tag, optionally wherein the C-terminal tag is an affinity tag or purification tag. In specific embodiments, the C-terminal tag is a 6× His tag (SEQ ID NO: 93), a streptavidin tag (e.g., a Strep-tag II tag), or a human influenza hemagglutinin tag. In specific embodiments, a 6× His tag is a 6× poly-histidine (i.e., His-His-His-His-His-His (SEQ ID NO:93). In specific embodiments, the C-terminal tag comprises or consists of the amino acid sequence of HHHHHH (SEQ ID NO:93). In specific embodiments, the C-terminal tag comprises or consists of the amino acid sequence of WSHPQFEK (SEQ ID NO:94). In specific embodiments, the C-terminal tag comprises or consists of the amino acid sequence of YPYDVPDYA (SEQ ID NO:95).

### 7.6 Target Antigens

In specific embodiments, a paratope of an antigen-binding molecule as described herein binds to an epitope of a target antigen. In specific embodiments, the paratope is formed by the variable region, preferably composed of a light chain variable domain associated with a heavy chain variable domain, of an antigen-binding molecule as described herein. In specific embodiments, the paratope is formed by two variable domains (e.g., a light chain variable domain and a heavy chain variable domain) of an antigen-binding molecule as described herein.

In specific embodiments, a target antigen is an antigen associated with a disease or disorder in a subject. In specific embodiments, a target antigen is an antigen associated with a cancer in a subject. In specific embodiments, a target antigen is an antigen specific for a cancer in a subject. In specific embodiments, a target antigen is an antigen of respiratory syncytial virus (RSV).

In specific embodiments, a target antigen is a tumor associated antigen (TAA), which is an antigen that is overexpressed in tumor cells (preferably of a cancer) relative to non-tumor cells in the subject. In specific embodiments, a first paratope of an antigen-binding molecule as described herein binds a first tumor associated antigen (TAA1), a second paratope of the antigen-binding molecule, if present, binds a second tumor associated antigen (TAA2), a third paratope of the antigen-binding molecule, if present, binds a third tumor associated antigen (TAA3), and a fourth paratope of the antigen-binding molecule, if present, binds a fourth tumor associated antigen (TAA4).

In specific embodiments, a target antigen is a tumor specific antigen (TSA), which is unique to tumor cells, or expressed only on tumor cells in the subject. In specific embodiments, a first paratope of an antigen-binding molecule as described herein binds a first tumor specific antigen (TSA1), a second paratope of the antigen-binding molecule, if present, binds a second tumor specific antigen (TSA2), a third paratope of the antigen-binding molecule, if present, binds a third tumor specific antigen (TSA3), and a fourth paratope of the antigen-binding molecule, if present, binds a fourth tumor specific antigen (TAA4).

### 7.7 Variable Regions, Variable Domains, and Paratopes

In one aspect, the antigen-binding molecules of the present invention bind to a target antigen (i.e., a paratope of the antigen binding molecule binds an epitope of the target antigen). In specific embodiments, an antigen-binding molecule as described herein comprises one or more variable regions that form one or more paratopes that bind to one or more target antigens. In specific embodiments, a variable region comprises two variable domains (e.g., two variable domains from two antigen-binding polypeptides as described herein that bind a target antigen). In specific embodiments, a variable region comprises a light chain variable domain and a heavy chain variable domain of an antibody that binds to a target antigen. In specific embodiments, a variable region comprises a light chain variable domain and a heavy chain variable domain of one arm of an antibody that binds to a target antigen.

In specific embodiments, a polypeptide of an antigen-binding molecule as described herein comprises a means for binding with a target antigen. In a specific embodiment, the means for binding is a variable domain (e.g., a light chain variable domain or a heavy chain variable domain). In specific embodiments, the means for binding of two polypeptide chains of an antigen-binding molecule as described herein form a paratope.

In specific embodiments, the variable region of an antigen-binding molecule as described herein forms a paratope that binds to a target antigen (e.g., an epitope of the target antigen). In specific embodiments, the variable domains of two polypeptides chains of an antigen-binding molecule as described herein form a paratope that binds to a target antigen (e.g., an epitope of the target antigen). In specific embodiments, the light chain complementary determining region 1 (LCDR1), chain complementary determining region 2 (LCDR2), light chain complementary determining region 3 (LCDR3), heavy chain complementary determining region 1 (HCDR1), heavy chain complementary determining region 2 (HCDR2), and heavy chain complementary determining region 3 (HCDR3) form a paratope that binds to a target antigen (e.g., an epitope of the target antigen).

In specific embodiments, a paratope as described herein binds to a target antigen. In specific embodiments, a paratope as described herein binds to an epitope of a target antigen. In specific embodiments, different paratopes of the same antigen-binding molecule (e.g., a first paratope, a second paratope, a third paratope, or a fourth paratope) respectively bind to different target antigens.

In specific embodiments, an antigen-binding molecule comprises two variable regions that bind to two epitopes. In specific embodiments, an antigen-binding molecule comprises two variable regions that bind to two target antigens. In specific embodiments, an antigen-binding molecule comprises two variable regions that bind to two different epitopes, optionally wherein the two different epitopes are from different target antigens.

In specific embodiments, an antigen-binding molecule comprises three variable regions that bind to three epitopes. In specific embodiments, an antigen-binding molecule comprises three variable regions that bind to three target antigens. In specific embodiments, an antigen-binding molecule comprises three variable regions that bind to three different epitopes, optionally wherein each of the three different epitopes are from different target antigens.

In specific embodiments, an antigen-binding molecule comprises four variable regions that bind to four epitopes. In specific embodiments, an antigen-binding molecule comprises four variable regions that bind to four target antigens. In specific embodiments, an antigen-binding molecule comprises four variable regions that bind to four different epitopes, optionally wherein each of the four different epitopes are from different target antigens.

In specific embodiments, a variable domain of a polypeptide as described herein comprises an antibody light chain variable domain or a fragment thereof. In specific embodiments, a variable domain or a fragment thereof comprises the amino acid sequence of an antibody light chain framework region 1 (LFR1), light chain complementary determining region 1 (LCDR1), light chain framework region 2 (LFR2), light chain complementary determining region 2 (LCDR2), light chain framework region 3 (LFR3), light chain complementary determining region 3 (LCDR3), light chain framework region 4 (LFR4), or any combination thereof that binds to a target antigen or forms a paratope that binds to a target antigen.

In specific embodiments, a variable domain of a polypeptide as described herein comprises an antibody heavy chain variable domain or a fragment thereof. In specific embodiments, a variable domain or a fragment thereof comprises the amino acid sequence of an antibody heavy chain framework region 1 (HFR1), heavy chain complementary determining region 1 (HCDR1), heavy chain framework region 2 (HFR2), heavy chain complementary determining region 2 (HCDR2), heavy chain framework region 3 (HFR3), heavy chain complementary determining region 3 (HCDR3), heavy chain framework region 4 (HFR4), or any combination thereof that binds to a target antigen or forms a paratope that binds to a target antigen.

In specific embodiments, an antigen-binding molecule as described herein binds to respiratory syncytial virus (RSV). In specific embodiments, a polypeptide of an antigen-binding molecule as described herein comprises a light chain variable domain (VL) comprising or consisting of the amino acid sequence of SEQ ID NO:97. In specific embodiments, a polypeptide of an antigen-binding molecule as described herein comprises a heavy chain variable domain (VH) comprising or consisting of the amino acid sequence of SEQ ID NO:96. In specific embodiments, an antigen-binding molecule as described herein comprises a light chain variable domain (VL) comprising or consisting of the amino acid sequence of SEQ ID NO:97 and a heavy chain variable domain (VH) comprising or consisting of the amino acid sequence of SEQ ID NO:96.

In specific embodiments, an antigen-binding molecule as described herein binds to human epidermal growth factor receptor 2 (HER2). In specific embodiments, a polypeptide of an antigen-binding molecule as described herein comprises a light chain variable domain (VL) comprising or consisting of the amino acid sequence of DIQMTQSPSSLSASVGDRVTITCRASQDVNTAVAWYQQKPGKAPKLLIYSASFLYSG VPSRFSGSRSGTDFTLTISSLQPEDFATYYCQQHYTTPPTFGQGTKVEIK (SEQ ID NO: 362). In specific embodiments, a polypeptide of an antigen-binding molecule as described herein comprises a heavy chain variable domain (VH) comprising or consisting of the amino acid sequence of EVQLVESGGGLVQPGGSLRLSCAASGFNIKDTYIHWVRQAPGKGLEWVARIYPTNG YTRYADSVKGRFTISADTSKNTAYLQMNSLRAEDTAVYYCSRWGGDGFYAMDYW GQGTLVTVSS (SEQ ID NO: 363). In specific embodiments, an antigen-binding molecule as described herein comprises a light chain variable domain (VL) comprising or consisting of the amino acid sequence of SEQ ID NO:362 and a heavy chain variable domain (VH) comprising or consisting of the amino acid sequence of SEQ ID NO:363.

In specific embodiments, an antigen-binding molecule as described herein binds to mesenchymal epithelial transition (MET). In specific embodiments, a polypeptide of an antigen-binding molecule as described herein comprises a light chain variable domain (VL) comprising or consisting of the amino acid sequence of DIQMTQSPSSLSASVGDRVTITCKSSQSLLYTSSQKNYLAWYQQKPGKAPKLLIYWA STRESGVPSRFSGSGSGTDFTLTISSLQPEDFATYYCQQYYAYPWTFGQGTKVEIK (SEQ ID NO: 364). In specific embodiments, a polypeptide of an antigen-binding molecule as described herein comprises a heavy chain variable domain (VH) comprising or consisting of the amino acid sequence of EVQLVESGGGLVQPGGSLRL SCAASGYTFTSYWLHWVRQAPGKGLEWVGMIDPSN SDTRFNPNFKDRFTISADTSKNTAYLQMNSLRAEDTAVYYCATYRSYVTPLDYWGQ GTLVTVSS (SEQ ID NO: 365). In specific embodiments, an antigen-binding molecule as described herein comprises a light chain variable domain (VL) comprising or consisting of the amino acid sequence of SEQ ID NO:364 and a heavy chain variable domain (VH) comprising or consisting of the amino acid sequence of SEQ ID NO:365.

In specific embodiments, an antigen-binding molecule as described herein binds to cluster of differentiation 3 (CD3). In specific embodiments, a polypeptide of an antigen-binding molecule as described herein comprises a light chain variable domain (VL) comprising or consisting of the amino acid sequence of DIQMTQSPSSLSASVGDRVTITCRASQDIRNYLNWYQQKPGKAPKLLIYYTSRLESGV PSRFSGSGSGTDYTLTISSLQPEDFATYYCQQGNTLPWTFGQGTKVEIK (SEQ ID NO:366). In specific embodiments, a polypeptide of an antigen-binding molecule as described herein comprises a heavy chain variable domain (VH) comprising or consisting of the amino acid sequence of EVQLVESGGGLVQPGGSLRLSCAASGYSFTGYTMNWVRQAPGKGLEWVALINPYK GVSTYNQKFKDRFTISVDKSKNTAYLQMNSLRAEDTAVYYCARSGYYGDSDWYFD VWGQGTLVTVSS (SEQ ID NO: 367). In specific embodiments, an antigen-binding molecule as described herein comprises a light chain variable domain (VL) comprising or consisting of the amino acid sequence of SEQ ID NO:366 and a heavy chain variable domain (VH) comprising or consisting of the amino acid sequence of SEQ ID NO:367.

In specific embodiments, an antigen-binding molecule as described herein binds to both HER2 and MET. In specific embodiments, a polypeptide of an antigen-binding molecule as described herein comprises a light chain variable domain (VL1) comprising or consisting of the amino acid sequence of SEQ ID NO:362. In specific embodiments, a polypeptide of an antigen-binding molecule as described herein comprises a first heavy chain variable domain (VH1) comprising or consisting of the amino acid sequence of SEQ ID NO:363. In specific embodiments, an antigen-binding molecule as described herein comprises a first light chain variable domain (VL) comprising or consisting of the amino acid sequence of SEQ ID NO:362 and a first heavy chain variable domain (VH) comprising or consisting of the amino acid sequence of SEQ ID NO:363. In specific embodiments, a polypeptide of an antigen-binding molecule as described herein comprises a second light chain variable domain (VL2) comprising or consisting of the amino acid sequence of SEQ ID NO:364. In specific embodiments, a polypeptide of an antigen-binding molecule as described herein comprises a second heavy chain variable domain (VH2) comprising or consisting of the amino acid sequence of SEQ ID NO:365. In specific embodiments, an antigen-binding molecule as described herein comprises a second light chain variable domain (VL2) comprising or consisting of the amino acid sequence of SEQ ID NO:364 and a second heavy chain variable domain (VH2) comprising or consisting of the amino acid sequence of SEQ ID NO:365.

In specific embodiments, an antigen-binding molecule as described herein binds to both HER2 and CD3. In specific embodiments, a polypeptide of an antigen-binding molecule as described herein comprises a light chain variable domain (VL) comprising or consisting of the amino acid sequence of SEQ ID NO:362. In specific embodiments, a polypeptide of an antigen-binding molecule as described herein comprises a heavy chain variable domain (VH) comprising or consisting of the amino acid sequence of SEQ ID NO:363. In specific embodiments, an antigen-binding molecule as described herein comprises a light chain variable domain (VL) comprising or consisting of the amino acid sequence of SEQ ID NO:362 and a heavy chain variable domain (VH) comprising or consisting of the amino acid sequence of SEQ ID NO:363. In specific embodiments, a polypeptide of an antigen-binding molecule as described herein comprises a second light chain variable domain (VL2) comprising or consisting of the amino acid sequence of SEQ ID NO:366. In specific embodiments, a polypeptide of an antigen-binding molecule as described herein comprises a second heavy chain variable domain (VH2) comprising or consisting of the amino acid sequence of SEQ ID NO:367. In specific embodiments, an antigen-binding molecule as described herein comprises a second light chain variable domain (VL2) comprising or consisting of the amino acid sequence of SEQ ID NO:366 and a second heavy chain variable domain (VH2) comprising or consisting of the amino acid sequence of SEQ ID NO:367.

### 7.8 Antigen-Binding Molecules

In one aspect, the antigen-binding molecules of the present invention comprise dimers of two polypeptides, each comprising a variable domain and a dimerization domain as described herein, wherein said variable domains form a paratope, and said dimerization domains bind to each other to form a dimer. Full or partial replacement of the dimerization interface between a light chain constant domain (CL) and heavy chain constant domain 1 (CH1) provides a means for selective assembly of cognate antibody chains. For example, a biparatopic antibody has one arm (e.g., a light chain polypeptide and a heavy chain polypeptide) modified with either the B2M/EA3 or ICAM-1 D1/ICAM-1 D1 dimerization domains as described herein, and its other arm is not modified. In another example, a triparatopic antibody has one arm (e.g., a light chain polypeptide and a heavy chain polypeptide) modified with the B2M/EA3 dimerization domains as described herein, one arm (e.g., a light chain polypeptide and a heavy chain polypeptide) modified with the ICAM-1 D1/ICAM-1 D1 dimerization domains as described herein, and its other arm is not modified.

In specific embodiments, an antigen-binding molecule is an immunoglobulin or antigen-binding fragment thereof. In specific embodiments, an antigen-binding molecule is an antibody or antigen-binding fragment thereof. In specific embodiments, an antigen-binding molecule is an immunoglobulin fragment or antibody fragment comprising a variable region and at least one constant domain and that binds a target antigen. In specific embodiments, the immunoglobulin or antibody fragment is a Fab, Fab′, F(ab′)₂, or bispecific Fab. In specific embodiments the antibody is a biparatopic antibody, a bispecific antibody, a triparatopic antibody, a trispecific antibody, a tetraparatopic antibody, tetraspecific antibody, a multiparatopic antibody, or a multispecific antibody. In specific embodiments, an antigen-binding molecule is an intact immunoglobulin or antibody. In specific embodiments, an antigen-binding molecule is not an intact immunoglobulin or antibody. In specific embodiments, an antigen-binding molecule is any fragment of an antigen-binding molecule as described herein that binds to a target antigen.

### 7.8.1 Fab-Based Antigen-Binding Molecules

In specific embodiments, an antigen-binding molecule is a Fab. In specific embodiments, the Fab comprises an antibody fragment having a variable region that binds to a target antigen and comprises a light chain and a fragment of a heavy chain bridged by a disulfide bond. In specific embodiments, an antigen-binding molecule is a pseudo-Fab (pFab). In specific embodiments, the pFab comprises a variable region that binds to a target antigen and two dimerization domains as described herein. In specific embodiments, an antigen-binding molecule is a Fab′. In specific embodiments, the Fab′ comprises an antibody fragment having a single variable region that binds to a target antigen, comprising an Fab and an additional portion of the heavy chain through the hinge region. In specific embodiments, an antigen-binding molecule is a pseudo-Fab′ (pFab′). In specific embodiments, the pFab′ comprises a variable region that binds to a target antigen, two dimerization domains as described herein, and a heavy chain hinge region. In specific embodiments, an antigen-binding molecule is a F(ab′)₂. In specific embodiments, the F(ab′)₂ comprises two Fab′ molecules joined by interchain disulfide bonds in the hinge regions of the heavy chains. In specific embodiments, the Fab′ molecules of the F(ab′)₂ may be directed toward the same or different epitopes. In specific embodiments, an antigen-binding molecule is a pseudo-F(ab′)₂ (pF(ab′)₂). In specific embodiments, the pF(ab′)₂ comprises two variable regions that each bind to a target antigen, two antigen-binding domains, two or more dimerization domains as described herein, and a heavy chain hinge region. In specific embodiments, an antigen-binding molecule is a bispecific Fab. In specific embodiments, the bispecific Fab comprises a Fab molecule having two variable regions that each bind to a target antigen, each of which may be directed to a different epitope.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab-like antibody fragments can be expressed in and secreted from E. coli or yeast cells, thus allowing the facile production of large amounts of these fragments. Antibody fragments can be isolated from the antibody phage libraries discussed above. According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. The antibody fragment may also be a “linear antibody,” for example, as described in the references cited above. Such linear antibodies may be monospecific or multi-specific, such as bispecific.

Antibodies provided herein include, but are not limited to, immunoglobulin molecules and immunologically active portions of immunoglobulin molecules, for example, molecules that contain an antigen-binding site that bind to a target antigen. The immunoglobulin molecules provided herein can be of any class (e.g., IgG, IgE, IgM, IgD, and IgA) or any subclass (e.g., IgG1, IgG2, IgG3, IgG4, IgAQ1, and IgA2) of immunoglobulin molecule that contains an antigen-binding site that binds to a target antigen. In a specific embodiment, an antibody provided herein is an IgG antibody, such as an IgG1 antibody, IgG2 antibody or IgG4 antibody (e.g., IgG4 nullbody and variants of IgG4 antibodies). In a preferred embodiment, the IgG antibody is an IgG1 antibody.

In specific embodiments, an antigen-binding molecule is a Fab, Fab′, F(ab′)₂, or bispecific Fab that comprises a variable region derived from an antibody specific for one or more target antigens. In specific embodiments, an antigen-binding molecule comprises a variable region that is derived from an antibody specific for one or more target antigens, and a dimerization domain as described herein.

### 7.8.2 Antibody-Based Antigen-Binding Molecules

An antigen-binding molecule of the present disclosure may be derived from an antibody by full or partial replacement of the dimerization interface between a light chain constant domain and a heavy chain constant domain 1 (CH1) of an antibody.

### 7.8.2.1 Monoclonal Antibodies

In specific embodiments, the antigen-binding molecule provided herein comprises a monoclonal antibody or a fragment thereof. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized with a target antigen as described above to elicit lymphocytes that produce or are capable of producing antibodies that will specifically bind to an epitope of the target antigen used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium, which, in specific embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., 1987, Monoclonal Antibody Production Techniques and Applications 51-63).

Culture medium in which hybridoma cells are growing is assayed for production of monoclonal antibodies directed against the target antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxyapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In a further embodiment, monoclonal antibodies or antibody fragments can be isolated from antibody phage libraries generated using the techniques described in, for example, Antibody Phage Display: Methods and Protocols (O'Brien and Aitken eds., 2002). In phage display methods, functional antibody domains are displayed on the surface of phage particles which carry the polynucleotide sequences encoding them. Examples of phage display methods that can be used to make the antibodies described herein include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT Application No. PCT/GB91/01134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

In principle, synthetic antibody clones are selected by screening phage libraries containing phages that display various fragments of antibody variable region (Fv) fused to phage coat protein. Such phage libraries are screened against the desired target antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self-antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, a target antigen can be used to coat the wells of adsorption plates, expressed on host cells affixed to adsorption plates or used in cell sorting, conjugated to biotin for capture with Strep-tag II-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Antibodies can be obtained by designing a suitable antigen screening procedure to select for the phage clone of interest followed by construction of a full length antibody clone using VH and/or VL sequences (e.g., the Fv sequences), or various CDR sequences from VH and VL sequences, from the phage clone of interest and suitable constant region (e.g., Fc) sequences described in Kabat et al., supra.

Antibodies described herein can also, for example, include chimeric antibodies. A chimeric antibody is a molecule in which different portions of the antibody are derived from different immunoglobulin molecules. For example, a chimeric antibody can contain a variable region of a mouse or rat monoclonal antibody fused to a constant region of a human antibody. Methods for producing chimeric antibodies are known in the art. See, e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415.

Antibodies or antigen-binding fragments produced using techniques such as those described herein can be isolated using standard, well known techniques. For example, antibodies or antigen-binding fragments can be suitably separated from, e.g., culture medium, ascites fluid, serum, cell lysate, synthesis reaction material or the like by conventional immunoglobulin purification procedures such as, for example, protein A-Sepharose, hydroxyapatite chromatography, gel electrophoresis, dialysis, or affinity chromatography. In specific embodiments, an isolated or purified antibody is substantially free of cellular material or other proteins from the cell or tissue source from which the antibody is derived, or substantially free of chemical precursors or other chemicals when chemically synthesized.

### 7.8.2.2 Humanized Antibodies

In specific embodiments, the antigen-binding molecule provided herein comprises a humanized antibody (e.g., deimmunized or composite human antibody) or a fragment thereof. In specific embodiments, a humanized antibody comprises human framework region and/or human constant region sequences. In specific embodiments, a humanized antibody can be selected from any class of immunoglobulins, including IgM, IgG, IgD, IgA and IgE, and any isotype, including IgG1, IgG2, IgG3 and IgG4 (e.g., variants of IgG4 and IgG4 nullbody). In specific embodiments, a humanized antibody comprises kappa or lambda light chain constant sequences.

Humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and other techniques disclosed in, e.g., U.S. Pat. Nos. 6,407,213, 5,766,886, WO 93/17105, Tan et al., J. Immunol. 169:1119 25 (2002), Caldas et al., Protein Eng. 13(5):353-60 (2000), Morea et al., Methods 20(3):267 79 (2000), Baca et al., J. Biol. Chem. 272(16):10678-84 (1997), Roguska et al., Protein Eng. 9(10):895 904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), each of which is incorporated by reference herein in its entirety.

Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In specific embodiments, humanized antibodies are constructed by CDR grafting, wherein the amino acid sequences of the six CDRs of a parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In specific cases, the framework is derived from the consensus sequences of the most abundant human subclasses, VL6 subgroup I (VL6I) and VH subgroup III (VHIII). In another method, human germline genes are used as the source of the framework regions.

In an alternative paradigm based on comparison of CDRs, called superhumanization, FR homology is irrelevant. The method comprises comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its target antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen-binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable region with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

A composite human antibody can be generated using, for example, Composite Human Antibody™ technology (Antitope Ltd., Cambridge, United Kingdom). To generate composite human antibodies, variable region sequences are designed from fragments of multiple human antibody variable region sequences in a manner that avoids T cell epitopes, thereby minimizing the immunogenicity of the resulting antibody. Such antibodies can comprise human constant region sequences, e.g., human light chain and/or heavy chain constant regions.

In specific embodiments, an antigen-binding molecule comprises a deimmunized antibody whose T-cell epitopes have been removed. Methods for making deimmunized antibodies have been described. (See, e.g., Jones et al., Methods Mol Biol. 2009; 525:405-23, xiv, and De Groot et al., Cell. Immunol. 244:148-153(2006)). Deimmunized antibodies comprise T-cell epitope-depleted variable regions and human constant regions. Briefly, VH and VL of an antibody are cloned and T-cell epitopes are subsequently identified by testing overlapping peptides derived from the VH and VL of the antibody in a T cell proliferation assay. T cell epitopes are identified via in silico methods to identify peptide binding to human MHC class II. Mutations are introduced in the VH and VL to abrogate binding to human MHC class II. Mutated VH and VL are then utilized to generate the deimmunized antibody.

### 7.8.2.3 Human Antibodies

In specific embodiments, the antigen-binding molecule provided herein comprises a fully human anti-human antibody or a fragment thereof. Fully human antibodies may be produced by any method known in the art. Human antibodies provided herein can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Bruggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen-binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In specific instances, however, VH CDR3 and VL CDR3, as well as VH CDR2, VL CDR2, and VL CDR1 of the non-human antibody may be retained.

### 7.8.2.4 Multispecific Antibodies

Multispecific antibodies such as bispecific antibodies are monoclonal antibodies that have binding specificities for at least two different antigens. In specific embodiments, the multispecific antibodies provided herein are bispecific antibodies. In specific embodiments, bispecific antibodies are mouse, chimeric, human or humanized antibodies. In specific embodiments, one of the binding specificities is for one target and/or target antigen and the other is for another target and/or target antigen. In specific embodiments, bispecific antibodies may bind to two different epitopes of the same target and/or target antigen. Bispecific antibodies can be prepared as full-length antibodies or antibody fragments (e.g., F(ab′)₂ bispecific antibody).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

### 7.8.2.5 Fc Engineering

It may be desirable to modify an antibody provided herein by Fc engineering. In specific embodiments, the modification to the Fc region of the antibody results in the decrease or elimination of an effector function of the antibody. In specific embodiments, the effector function is ADCC, ADCP, and/or CDC. In specific embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody.

In specific embodiments, the modification to the Fc region of the antibody results in the enhancement of an effector function of the antibody. In specific embodiments, the effector function is ADCC, ADCP, and/or CDC. In specific embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody.

In specific embodiments, Knob-in-Hole (KiH) technology can be used to engineer the antibody. The “Knob-in-Hole” technology may include one or more mutations selected from Y349C, T366S, L368A and Y407V in the CH3 domain of the Fc region in the Hole arm; and mutations S354C and/or T366W in the CH3 domain of the Fc region of the Knob arm. The mutations can promote heteromultimer formation. The Knob-into-Hole technology has been described in U.S. Pat. Nos. 5,731,168 and 8,216,805, which are herein incorporated by reference in their entireties.

In specific embodiments, one or both Fc regions of the antibody can be engineered to comprise the RF mutation. The “RF mutation” generally refers to the mutation of the amino acids HY into RF in the CH3 domain of Fc regions, such as the mutation H435R and Y436F in CH3 domain as described by Jendeberg, L. et al. (1997, J. Immunological Meth., 201: 25-34). The RF mutation is described as advantageous for purification purposes as it abolishes binding to protein A. In some embodiments, one Fc region of the antibody comprises the RF mutation and the other Fc region does not comprise the RF mutation.

In specific embodiments, an antigen-binding molecule described herein comprises a Knob arm comprising a T366W mutation in the CH3 domain of the Fc region. In one embodiment, the Knob arm comprises or consists of the following amino acid sequence

(SEQ ID NO: 360)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLWC
LVKGFYPSDIAVEWESNGQPENNYKTTPPVLDSDGSFFLYSKLTVDKSR
WQQGNVFSCSVMHEALHNHYTQKSLSLSPGK

In some embodiments, an antigen-binding molecule described herein comprises a Hole arm comprising mutations T366S, L368A and Y407V in the CH3 domain of the Fc region. The Hole arm may further comprise the RF mutation. In one embodiment, the Hole arm comprises or consists of the following amino acid sequence

(SEQ ID NO: 361)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSREEMTKNQVSLSC
AVKGFYPSDIAVEWESNGQPENNYKTTPPVLDSDGSFFLVSKLTVDKSR
WQQGNVFSCSVMHEALHNRFTQKSLSLSPGK

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### 7.8.2.6 Antibody Variants

In specific embodiments, amino acid sequence modification(s) of the antibodies or antigen-binding fragments provided herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the antibodies described herein, it is contemplated that antibody variants can be prepared. For example, antibody variants can be prepared by introducing appropriate nucleotide changes into the encoding DNA, and/or by synthesis of the desired antibody or polypeptide. Those skilled in the art would appreciate that amino acid changes may alter post-translational processes of the antibody, such as changing the number or position of glycosylation sites or altering the membrane anchoring characteristics.

In specific embodiments, antibodies provided herein are chemically modified, for example, by the covalent attachment of any type of molecule to the antibody. The antibody derivatives may include antibodies that have been chemically modified, for example, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formulation, metabolic synthesis of tunicamycin, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Insertions or deletions may optionally be in the range of about 1 to 5 amino acids. In specific embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or antigen-binding fragment thereof that binds to a target epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for examples, the antibodies described in Section 7 below. In another embodiment, an antibody or antigen-binding fragment thereof that binds to a target antigen comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an antibody described herein, for examples, the antibodies described in Section 7 below.

The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the antigen-binding molecule variant DNA.

Any cysteine residue not involved in maintaining the proper conformation of the antibody provided herein also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In specific embodiments, an antibody molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T-cell epitopes of the antibody molecule. In a first step, the immunogenicity of the antibody molecule can be determined by several methods, for example, by in vitro determination of T-cell epitopes or in silico prediction of such epitopes, as known in the art. Once the critical residues for T-cell epitope function have been identified, mutations can be made to remove immunogenicity and retain antibody activity. For review, see, for example, Jones et al., 2009, Methods in Molecular Biology 525:405-23.

### 7.8.2.7 In Vitro Affinity Maturation

In specific embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as Strep-tag II conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step. In specific embodiments, mammalian display systems may be used.

Diversity may also be introduced into the CDRs of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, the antibodies can be immobilized onto solid supports, columns, pins, or cellulose/poly(vinylidene fluoride) membranes/other filters, expressed on host cells affixed to adsorption plates or used in cell sorting, or conjugated to biotin for capture with Strep-tag II-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 7.8.2.8 Antibody Modifications

Covalent modifications of the antibodies binding to a target antigen are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody provided herein included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody or a fragment thereof of the present disclosure may also be modified to form chimeric molecules comprising the antibody or a fragment thereof fused to another, heterologous polypeptide or amino acid sequence, for example, an epitope tag (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antigen-binding molecule provided herein that binds to a target antigen, and a heterologous polypeptide.

Also provided herein are panels of antigen-binding molecules that bind to one or more target antigens. In specific embodiments, the panels of antigen-binding domains have different association rates, different dissociation rates, different affinities for a target antigen, and/or different specificities for a target antigen. In specific embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antigen-binding domains can be used, for example, in 96-well or 384-well plates, for assays such as ELISAs.

### 7.8.2.9 Immunoconjugates

The present disclosure also provides conjugates comprising any one of the antibodies or antigen-binding fragments thereof of the present disclosure covalently bound by a synthetic linker to one or more non-antibody agents.

In specific embodiments, antibodies provided herein are conjugated or recombinantly fused, e.g., to a therapeutic agent (e.g., a cytotoxic agent) or a diagnostic or detectable molecule. The conjugated or recombinantly fused antibodies can be useful, for example, for treating or preventing a disease or disorder. The conjugated or recombinantly fused antibodies can be useful, for example, for monitoring or prognosing the onset, development, progression, and/or severity of a disease or disorder.

Such diagnosis and detection can be accomplished, for example, by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, Strep-tag II/biotin or avidin/biotin; fluorescent materials, such as, but not limited to, umbelliferone, fluorescein, fluorescein isothiocyanate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Also provided herein are antibodies that are recombinantly fused or chemically conjugated (covalent or non-covalent conjugations) to a heterologous protein or polypeptide (or a fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising an antigen-binding fragment of an antibody provided herein (e.g., CDR1, CDR2, and/or CDR3) and a heterologous protein, polypeptide, or peptide. In one embodiment, the heterologous protein, polypeptide, or peptide that the antibody is fused to is useful for targeting the antibody to a particular cell type.

Moreover, antibodies provided herein can be fused to marker or “tag” sequences, such as a peptide, to facilitate purification. In specific embodiments, the marker or tag amino acid sequence is a hexa-histidine peptide (SEQ ID NO: 93), such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine (SEQ ID NO: 93) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for fusing or conjugating moieties (including polypeptides) to antibodies are known (see, e.g., Arnon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodies for Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of the antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

An antibody provided herein can also be conjugated to a second antibody to form an antibody heteroconjugate as described, for example, in U.S. Pat. No. 4,676,980.

Antibodies as provided herein may also be attached to solid supports, which are particularly useful for immunoassays or purification of the target antigen. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see, e.g., Chari et al., 1992, Cancer Res. 52:127-31; and U.S. Pat. No. 5,208,020), thioether linkers, or hydrophilic linkers designed to evade multidrug transporter-mediated resistance (see, e.g., Kovtun et al., 2010, Cancer Res. 70:2528-37).

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the F-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen-binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 7.9 Polynucleotides

In specific embodiments, the disclosure encompasses polynucleotides (interchangeably referred to herein as nucleic acids) that encode the antigen-binding molecules or fragments thereof as described herein. The term “polynucleotides that encode a polypeptide” encompasses a polynucleotide that includes only coding sequences for the polypeptide as well as a polynucleotide which includes additional coding and/or non-coding sequences. The polynucleotides of the disclosure can be in the form of RNA or in the form of DNA. DNA includes cDNA, genomic DNA, and synthetic DNA; and can be double-stranded or single-stranded, and if single stranded can be the coding strand or non-coding (anti-sense) strand.

In specific embodiments, a polynucleotide comprises the coding sequence for a polypeptide fused in the same reading frame to a polynucleotide which aids, for example, in expression and secretion of a polypeptide from a host cell (e.g., a leader sequence which functions as a secretory sequence for controlling transport of a polypeptide). The polypeptide can have the leader sequence cleaved by the host cell to form a “mature” form of the polypeptide.

In specific embodiments, a polynucleotide comprises the coding sequence for a polypeptide fused in the same reading frame to a marker or tag sequence. For example, in specific embodiments, a marker sequence is a hexa-histidine tag (SEQ ID NO: 93) supplied by a vector that allows efficient purification of the polypeptide fused to the marker in the case of a bacterial host. In specific embodiments, a marker is used in conjunction with other affinity tags.

The present disclosure further relates to variants of the polynucleotides described herein, wherein the variant encodes, for example, fragments, analogs, and/or derivatives of a polypeptide. In specific embodiments, the present disclosure provides a polynucleotide comprising a polynucleotide having a nucleotide sequence at least about 80% identical, at least about 85% identical, at least about 90% identical, at least about 95% identical, and in specific embodiments, at least about 96%, 97%, 98% or 99% identical to a polynucleotide encoding a polypeptide comprising an antibody or antigen-binding fragment thereof described herein.

As used herein, the phrase “a polynucleotide having a nucleotide sequence at least, for example, 95% “identical” to a reference nucleotide sequence” is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In specific embodiments, a polynucleotide variant contains alterations that produce silent substitutions, additions, or deletions, but does not alter the properties or activities of the encoded polypeptide. In specific embodiments, a polynucleotide variant comprises silent substitutions that results in no change to the amino acid sequence of the polypeptide (due to the degeneracy of the genetic code). Polynucleotide variants can be produced for a variety of reasons, for example, to optimize codon expression for a particular host (i.e., change codons in the human mRNA to those preferred by a bacterial host such as E. coli). In specific embodiments, a polynucleotide variant comprises at least one silent mutation in a non-coding or a coding region of the sequence.

In specific embodiments, a polynucleotide variant is produced to modulate or alter expression (or expression levels) of the encoded polypeptide. In specific embodiments, a polynucleotide variant is produced to increase expression of the encoded polypeptide. In specific embodiments, a polynucleotide variant is produced to decrease expression of the encoded polypeptide. In specific embodiments, a polynucleotide variant has increased expression of the encoded polypeptide as compared to a parental polynucleotide sequence. In specific embodiments, a polynucleotide variant has decreased expression of the encoded polypeptide as compared to a parental polynucleotide sequence.

In specific embodiments, the present disclosure provides a polynucleotide comprising a nucleotide sequence at least about 80% identical, at least about 85% identical, at least about 90% identical, at least about 95% identical, and in specific embodiments, at least about 96%, 97%, 98% or 99% identical to a polynucleotide listed in the Sequence Listing (e.g., SEQ ID NOS: 3 and 263-343) provided herein.

In specific embodiments, the present disclosure provides a polynucleotide comprising a nucleotide sequence at least about 80% identical, at least about 85% identical, at least about 90% identical, at least about 95% identical, and in specific embodiments, at least about 96%, 97%, 98% or 99% identical to a polynucleotide selected from the polynucleotides (e.g., SEQ ID NOS: 3 and 263-343) provided herein.

In specific embodiments, a polynucleotide is isolated. In specific embodiments, a polynucleotide is substantially pure.

Vectors and cells comprising the polynucleotides described herein are also provided. In specific embodiments, an expression vector comprises a polynucleotide molecule. In specific embodiments, a host cell comprises an expression vector comprising the polynucleotide molecule. In specific embodiments, a host cell comprises one or more expression vectors comprising polynucleotide molecules. In specific embodiments, a host cell comprises a polynucleotide molecule as described herein. In specific embodiments, a host cell comprises one or more polynucleotide molecules as described herein.

### 7.10 Methods or Processes of Making the Antigen-Binding Molecules

In yet another aspect, provided herein are methods or processes for making the antigen-binding molecule as described herein. While described hereinbelow for an antigen-binding molecule that is an antibody (e.g., a biparatopic antibody comprising the dimerization domains as described herein), the following will be understood as also applicable to other antigen-binding molecules of the invention, unless such would be understood as inapplicable from the context.

Recombinant expression of an antigen-binding molecule provided herein can be achieved by construction of an expression vector containing one or more polynucleotides that encodes the antigen-binding molecule. Once a polynucleotide encoding an antigen-binding molecule, heavy or light chain of an antigen-binding molecule, or a fragment thereof (such as, but not necessarily, containing the heavy and/or light chain variable domain) provided herein has been obtained, the vector for the production of the antigen-binding molecule may be produced by recombinant DNA technology using techniques well-known in the art. Thus, methods for preparing a protein by expressing a polynucleotide containing an antigen-binding molecule encoding nucleotide sequence are described herein. Methods which are well known to those skilled in the art can be used to construct expression vectors containing antigen-binding molecule coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antigen-binding molecule provided herein, a heavy or light chain of an antigen-binding molecule, a heavy or light chain variable domain of an antigen-binding molecule or a fragment thereof, or a heavy or light chain CDR, operably linked to a promoter. Such vectors may include the nucleotide sequence encoding the constant region of the antigen-binding molecule (see, e.g., International Publication Nos. WO 86/05807 and WO 89/01036; and U.S. Pat. No. 5,122,464) and the variable domain of the antibody may be cloned into such a vector for expression of the entire heavy, the entire light chain, or both the entire heavy and light chains. In specific embodiments, the antigen-binding molecules comprise one or more antigen-binding polypeptides that have an antibody variable domain and, in lieu of a light chain constant domain (CL) or heavy chain constant domain 1 (CH1), have a dimerization domain as described herein.

The expression vector is transferred to a host cell by conventional techniques and the transfected cells are then cultured by conventional techniques to produce an antigen-binding molecule provided herein. Thus, also provided herein are host cells containing a polynucleotide encoding an antigen-binding molecule provided herein or fragments thereof, or a heavy or light chain thereof, or a fragment thereof, operably linked to a heterologous promoter. A host cell can be any type of cell, e.g., a primary cell, a cell in culture, or a cell from a cell line. In specific embodiments, a host cell is a cell transfected with a nucleic acid molecule (e.g., vector) provided herein. In specific embodiments, a host cell is a progeny or potential progeny of a cell transfected with a nucleic acid molecule (e.g., vector) provided herein. In specific embodiments for the expression of double-chained antigen-binding molecules (e.g., monoparatopic antigen-binding molecules), vectors encoding both the heavy and light chains may be co-expressed in the host cell for expression of the entire immunoglobulin molecule. In specific embodiments for the expression of quadruple-chained antigen-binding molecules (e.g., biparatopic antigen-binding molecules), vectors encoding each of the heavy and light chains may be co-expressed in the host cell for expression of the entire immunoglobulin molecule. In specific embodiments for the expression of sextuple-chained antigen-binding molecules (e.g., triparatopic antigen-binding molecules), vectors encoding each the heavy and light chains may be co-expressed in the host cell for expression of the entire immunoglobulin molecule, as detailed below. In specific embodiments for the expression of octuple-chained antigen-binding molecules (e.g., tetraparatopic antigen-binding molecules), vectors encoding each the heavy and light chains may be co-expressed in the host cell for expression of the entire immunoglobulin molecule, as detailed below. In specific embodiments, a multiparatopic antigen-binding molecule as described herein is produced in a single host cell.

A variety of host-expression vector systems may be utilized to express the antigen-binding molecules provided herein (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antigen-binding molecule provided herein in situ. These include but are not limited to microorganisms such as bacteria (e.g., E. coli and B. subtilis) transformed with recombinant bacteriophage DNA, plasmid DNA or cosmid DNA expression vectors containing antigen-binding molecule coding sequences; yeast (e.g., Saccharomyces Pichia) transformed with recombinant yeast expression vectors containing antigen-binding molecule coding sequences; insect cell systems infected with recombinant virus expression vectors (e.g., baculovirus) containing antigen-binding molecule coding sequences; plant cell systems infected with recombinant virus expression vectors (e.g., cauliflower mosaic virus, CaMV, tobacco mosaic virus, TMV) or transformed with recombinant plasmid expression vectors (e.g., Ti plasmid) containing antigen-binding molecule coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Bacterial cells such as Escherichia coli, or, eukaryotic cells, especially for the expression of whole recombinant antigen-binding molecules, can be used for the expression of a recombinant antigen-binding molecules. For example, mammalian cells such as Chinese hamster ovary cells (CHO), in conjunction with a vector such as the major intermediate early gene promoter element from human cytomegalovirus is an effective expression system for antigen-binding molecules (Foecking et al., 1986, Gene 45:101; and Cockett et al., 1990, Bio/Technology 8:2). In specific embodiments, antigen-binding molecules provided herein are produced in CHO cells. In a specific embodiment, the expression of nucleotide sequences encoding antigen-binding molecules provided herein which immunospecifically bind to a target antigen is regulated by a constitutive promoter, inducible promoter or tissue specific promoter. In a specific embodiment, the expression of nucleotide sequences encoding antigen-binding molecules provided herein which immunospecifically bind to a target antigen is regulated by a constitutive promoter, inducible promoter or tissue specific promoter.

In bacterial systems, a number of expression vectors may be advantageously selected depending upon the use intended for the antigen-binding molecule being expressed. For example, when a large quantity of such an antigen-binding molecule is to be produced, for the generation of pharmaceutical compositions of an antigen-binding molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antigen-binding molecule coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antigen-binding molecule coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antigen-binding molecule coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antigen-binding molecule coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells that possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7030 and HsS78Bst cells. In specific embodiments, fully human antigen-binding molecules provided herein are produced in mammalian cells, such as CHO cells.

For long-term, high-yield production of recombinant proteins, stable expression can be utilized. For example, cell lines which stably express the antigen-binding molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method may advantageously be used to engineer cell lines that express the antigen-binding molecule. Such engineered cell lines may be particularly useful in screening and evaluation of compositions that interact directly or indirectly with the antigen-binding molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds.), Current Protocols in Human Genetics, John Wiley & Sons, NY (1994); Colberre-Garapin et al., 1981, J. Mol. Biol. 150:1, which are incorporated by reference herein in their entireties.

The expression levels of an antigen-binding molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing the antigen-binding molecule is amplifiable, increase in the level of inhibitor present in culture of host cell will increase the number of copies of the marker gene. Since the amplified region is associated with the antigen-binding molecule gene, production of the antigen-binding molecule will also increase (Crouse et al., 1983, Mol. Cell. Biol. 3:257).

The host cell may be co-transfected with two or more expression vectors provided herein. The two or more vectors may contain identical selectable markers which enable equal expression of, e.g., heavy and light chain polypeptides of an antigen-binding molecule. Alternatively, a single vector may be used which encodes, and is capable of expressing different component polypeptides of the present antigen-binding molecule, e.g., both heavy and light chain polypeptides of an antigen-binding molecule. The coding sequences may comprise cDNA or genomic DNA.

Once an antigen-binding molecule provided herein has been produced by recombinant expression, it may be purified or isolated by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antigen-binding molecule provided herein can be fused to heterologous amino acid sequences described herein or otherwise known in the art to facilitate purification.

### 7.11 Pharmaceutical Compositions

In one aspect, the present disclosure further provides pharmaceutical compositions comprising at least one antigen-binding molecule of the present disclosure. In specific embodiments, a pharmaceutical composition comprises therapeutically effective amount of an antigen-binding molecule provided herein and a pharmaceutically acceptable excipient. In a specific embodiment, the antigen-binding molecule is isolated. In a specific embodiment, the antigen-binding molecule is purified. Any of the antigen-binding molecules provided herein are contemplated in the pharmaceutical compositions.

Pharmaceutical compositions comprising an antigen-binding molecule or a fragment thereof are prepared for storage by mixing the protein having the desired degree of purity with optional physiologically acceptable excipients (see, e.g., Remington, Remington's Pharmaceutical Sciences (18th ed. 1980)) in the form of aqueous solutions or lyophilized or other dried forms.

The antigen-binding molecule of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An antigen-binding molecule provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antigen-binding molecule as described herein, including, but not limited to, encapsulation in liposomes, microparticles, microcapsules, recombinant cells capable of expressing the antigen-binding molecule, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody or antigen-binding fragment thereof as described herein) or a composition provided herein (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibody or antigen-binding fragment thereof as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 7.12 Methods of Use

In another aspect, provided herein are methods for using and uses of the antigen-binding molecules provided herein. Such methods and uses include therapeutic methods and uses, for example, involving administration of the antigen-binding molecules, or compositions containing the same, to a subject having a disease or disorder. In specific embodiments, the subject is in need of treatment. In specific embodiments, the composition is administered in an effective amount to effect treatment of the disease or disorder in the subject. Uses include uses of the compositions in such methods and treatments, and in the preparation of a medicament in order to carry out such therapeutic methods. In specific embodiments, the methods are carried out by administering the compositions to the subject having or suspected of having the disease or condition. In specific embodiments, the methods thereby treat the disease or disorder in the subject.

In specific embodiments, the treatment provided herein cause complete or partial amelioration or reduction of a disease or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms include, but do not imply, complete curing of a disease or complete elimination of any symptom or effect(s) on all symptoms or outcomes.

As used herein, in specific embodiments, the treatment provided herein delay development of a disease or disorder, e.g., defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease or disorder. For example, a late stage cancer, such as development of metastasis, may be delayed. In other embodiments, the method or the use provided herein prevents a disease or disorder.

In yet another aspect, provided herein is a method of enriching, isolating, separating, purifying, sorting, selecting, capturing, detecting or depleting cells expressing one or more target antigens, comprising providing a sample comprising the cells expressing the one or more target antigens; contacting the sample with an antigen-binding molecule; and enriching, isolating, separating, purifying, sorting, selecting, capturing, detecting or depleting the cells expressing the target antigen and bound to the antigen-binding molecule, wherein the antigen-binding molecule comprises a first paratope capable of binding to a first target antigen, and optionally a second paratope capable of binding to a second target antigen, further optionally a third paratope capable of binding to a third target antigen, further optionally a fourth paratope capable of binding to a fourth target antigen. In specific embodiments, the sample is a blood sample. In other embodiments, the sample is a tissue sample.

In another aspect, provided herein is a method of inhibiting or depleting cancer cells or T cells, comprising contacting the cancer cells or T cells with an effective amount of an antigen-binding molecule that binds to at least one tumor associated antigen or tumor specific antigen.

In another aspect, provided herein is a method of inhibiting or depleting cancer cells or T cells in a subject having cancer, comprising administering to the subject an effective amount of an antigen-binding molecule that binds to at least one tumor associated antigen or tumor specific antigen.

In another aspect, provided herein is a method of treating a disease or disorder in a subject comprising administering to the subject an effective amount of an antigen-binding molecule that binds to at least one target antigen.

In another aspect, provided herein is a method of treating cancer in a subject, comprising administering to the subject an effective amount of an antigen-binding molecule that binds to at least one tumor associated antigen or tumor specific antigen.

In another aspect, provided herein is a method of treatment of a disease or disorder, wherein the subject is administered one or more therapeutic agents in combination with an effect amount of an antigen-binding molecule that binds to at least one target antigen.

In another aspect, provided herein is the use of an antigen-binding molecule provided herein in the manufacture of a medicament for treating a disease or disorder in a subject.

In another aspect, provided herein is the use of a pharmaceutical composition provided herein in the manufacture of a medicament for treating a disease or disorder in a subject.

In a specific embodiment, provided herein is a composition for use in the prevention and/or treatment of a disease or condition comprising an antigen-binding molecule provided herein. In specific embodiments, the subject is a subject in need thereof. In specific embodiments, the subject has the disease or condition. In other embodiments, the subject is at risk of having the disease or condition. In specific embodiments, the administration results in the prevention, management, treatment or amelioration of the disease or condition.

In another embodiment, provided herein is a method of preventing and/or treating a symptom of a disease or condition in a subject, comprising administering an effective amount of an antigen-binding molecule provided herein. In one embodiment, provided herein is a method of preventing a symptom of a disease or condition in a subject, comprising administering an effective amount of an antigen-binding molecule provided herein. In specific embodiments, the subject is a subject in need thereof. In specific embodiments, the subject has the disease or condition. In other embodiments, the subject is at risk of having the disease or condition. In specific embodiments, the administration results in the prevention or treatment of the symptom of the disease or condition.

In specific embodiments, the disease is cancer and the antigen-binding molecule is multiparatopic or multispecific. In specific embodiments, the disease is cancer and the antigen-binding molecule is biparatopic and monospecific for one target antigen that is a tumor associated antigen (TAA) or tumor specific antigen (TSA) (e.g., both paratopes bind to a single target antigen). In specific embodiments, the disease is cancer and the antigen-binding molecule is biparatopic and bispecific for two target antigens selected from a tumor associated antigen (TAA), tumor specific antigen (TSA), or combination thereof (e.g., each paratope binds to a different target antigen). In specific embodiments, the disease is cancer and the antigen-binding molecule is triparatopic and monospecific for one target antigen that is a tumor associated antigen (TAA) or tumor specific antigen (TSA) (e.g., each paratope binds to a single target antigen). In specific embodiments, the disease is cancer and the antigen-binding molecule is triparatopic and bispecific for two target antigens selected from a tumor associated antigen (TAA), tumor specific antigen (TSA), or combination thereof (e.g., two paratopes binds to a single target antigen, and one paratope binds to a different target antigen). In specific embodiments, the disease is cancer and the antigen-binding molecule is triparatopic and trispecific for three target antigens selected from a tumor associated antigen (TAA), tumor specific antigen (TSA), or combination thereof (e.g., each paratope binds to a different target antigen). In specific embodiments, the disease is cancer and the antigen-binding molecule is tetraparatopic and bispecific for two target antigens selected from a tumor associated antigen (TAA), tumor specific antigen (TSA), or combination thereof (e.g., two paratopes binds to a target antigen, and two paratopes bind to a different target antigen). In specific embodiments, the disease is cancer and the antigen-binding molecule is tetraparatopic and tetraspecific for four target antigens selected from a tumor associated antigen (TAA), tumor specific antigen (TSA), or combination thereof (e.g., each paratopes binds to a different target antigen). In specific embodiments, the disease is cancer and the antigen-binding molecule is as shown in TABLE 4, below. In specific embodiments, the disease is cancer and the antigen-binding molecule is as shown in TABLE 4 below but wherein each of the target antigens TAA1, TAA2, TAA3, or TAA4 is independently and optionally substituted with a tumor specific antigen (e.g., TSA1, TSA2, TSA3, or TSA4). In specific embodiments, a paratope or at least the first paratope of an antigen-binding molecule as described herein binds to: i) a target antigen associated with the cancer, or ii) a target antigen specific to the cancer.

TABLE 4
Multi-Paratopic and Multi-Specific Antibody Designs

| Molecule | Specificity |
|---|---|
| Tumor Associated Antigen 1 (TAA1) × | Biparatopic; Monospecific |
| Tumor Associated Antigen 1 (TAA1) | Anti-TAA1 and anti-TAA1 |
| Tumor Associated Antigen 1 (TAA1) × | Biparatopic; Bispecific |
| Tumor Associated Antigen 2 (TAA2) | Anti-TAA1 and anti-TAA2 |
| Tumor Associated Antigen 1 (TAA1) × | Triparatopic; Trispecific |
| Tumor Associated Antigen 2 (TAA2) × | Anti-TAA1, anti-TAA2, |
| Tumor Associated Antigen 3 (TAA3) | and anti-TAA3 |
| Tumor Associated Antigen 1 (TAA1) × | Tetraparatopic; Tetraspecific |
| Tumor Associated Antigen 2 (TAA2) × | Anti-TAA1, anti-TAA2, |
| Tumor Associated Antigen 3 (TAA3) × | anti-TAA3, anti-TAA4, etc. |
| Tumor Associated Antigen 4 (TAA4) | |

In specific embodiments, the tumor associated antigen (TAA) or tumor specific antigen (TSA) is present on the surface of a cancer cell.

In specific embodiments, the cancer cell is a cell of an adrenal cancer, anal cancer, appendix cancer, bile duct cancer, bladder cancer, bone cancer, brain cancer, breast cancer, cervical cancer, colorectal cancer, esophageal cancer, gallbladder cancer, gestational trophoblastic, head and neck cancer, Hodgkin lymphoma, intestinal cancer, kidney cancer, leukemia, liver cancer, lung cancer, melanoma, mesothelioma, multiple myeloma, neuroendocrine tumor, non-Hodgkin lymphoma, oral cancer, ovarian cancer, pancreatic cancer, prostate cancer, sinus cancer, skin cancer, soft tissue sarcoma spinal cancer, stomach cancer, testicular cancer, throat cancer, thyroid cancer, uterine cancer endometrial cancer, vaginal cancer, or vulvar cancer.

In specific embodiments, the tumor associated antigen (TAA) or tumor specific antigen (TSA) is an angiopoietin, BCMA, CD19, CD20, CD22, CD25 (IL2-R), CD30, CD33, CD37, CD38, CD52, CD56, CD123 (IL-3R), cMET, DLL/Notch, EGFR, EpCAM, FGF, FGF-R, GD2, HER2, Mesothelin, Nectin-4, PAP, PDGFRα, PSA, PSA3, PSMA, RANKL, SLAMF7, STEAPI, TARP, TROP2, VEGF, or VEGF-R antigen. In some embodiments, the tumor associated antigen (TAA) or tumor specific antigen (TSA) is a CEA, immature laminin receptor, TAG-72, HPV E6, HPV E7, BING-4, calcium-activated chloride channel 2, cyclin-B1, 9D7, EpCAM, EphA3, Her2/neu, telomerase, mesothelin, SAP-1, surviving, a BAGE family antigen, CAGE family antigen, GAGE family antigen, MAGE family antigen, SAGE family antigen, XAGE family antigen, NY-ESO-1/LAGE-1, PRAME, SSX-2, Melan-A, MART-1, Gp100, pmel17, tyrosinase, TRP-1, TRP-2, P. polypeptide, MC1R, prostate-specific antigen, β-catenin, or BRCA1 antigen.

In specific embodiments, the tumor associated antigen (TAA) or tumor specific antigen (TSA) is HER2 or MET.

Provided herein are methods of preventing and/or treating a disease or condition by administrating to a subject of an effective amount of an antigen-binding molecule provided herein, or pharmaceutical composition comprising an antigen-binding molecule provided herein. In one aspect, the antigen-binding molecule is substantially purified (i.e., substantially free from substances that limit its effect or produce undesired side-effects). In specific embodiments, a subject is any animal, preferably a mammal (e.g., any mammal), most preferably a human. In specific embodiments, a mammal is a non-primate or a primate. In specific embodiments, a mammal is selected from the group consisting of a cow, horse, sheep, pig, cat, dog, mice, rat, rabbit, guinea pig, monkey, and human. In a preferred embodiment, the subject is a human. In a specific embodiment, the subject is a human with a disease or condition. In a specific embodiment, the subject is a human with a cancer.

Various delivery systems are known and can be used to administer a prophylactic or therapeutic agent (e.g., an antigen-binding molecule provided herein), including, but not limited to, encapsulation in liposomes, microparticles, microcapsules, recombinant cells capable of expressing the antibody or antigen-binding fragment thereof, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent (e.g., an a antigen-binding molecule herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., an antigen-binding molecule provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as silastic membranes, or fibers. In specific embodiments, when administering an antigen-binding molecule provided herein, care must be taken to use materials to which the antigen-binding molecule does not absorb.

In another embodiment, a prophylactic or therapeutic agent, or a composition provided herein can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., an antibody provided herein) or a composition provided herein (see e.g., Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see, e.g., Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more antigen-binding molecules provided herein. See, e.g., U.S. Pat. No. 4,526,938, PCT publication WO 91/05548, PCT publication WO 96/20698, Ning et al., 1996, “Intratumoral Radioimmunotherapy of a Human Colon Cancer Xenograft Using a Sustained-Release Gel,” Radiotherapy & Oncology 39:179-189, Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment, where the composition provided herein is a polynucleotide encoding a prophylactic or therapeutic agent (e.g., an antigen-binding molecule provided herein), the polynucleotide can be administered in vivo to promote expression of its encoded prophylactic or therapeutic agent, by constructing it as part of an appropriate polynucleotide expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see, e.g., Joliot et al., 1991, Proc. Natl. Acad. Sci. USA 88:1864-1868), etc. Alternatively, a polynucleotide can be introduced intracellularly and incorporated within host cell DNA for expression by homologous recombination.

In a specific embodiment, a composition provided herein comprises one, two or more antigen-binding molecules provided herein. In another embodiment, a composition provided herein comprises one, two or more antigen-binding molecules provided herein and a prophylactic or therapeutic agent other than an antigen-binding molecule provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., an antigen-binding molecule provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, PA. Such compositions will contain a prophylactically or therapeutically effective amount of the antigen-binding molecule provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

An antigen-binding molecule provided herein can be packaged in a hermetically sealed container such as an ampoule or sachette indicating the quantity of the antigen-binding molecule. In one embodiment, the antigen-binding molecule is supplied as a dry sterilized lyophilized powder or water free concentrate in a hermetically sealed container and can be reconstituted, e.g., with water or saline to the appropriate concentration for administration to a subject. The lyophilized antigen-binding molecule can be stored at between 2 and 8° C. in its original container and the antibody or antigen-binding fragment thereof can be administered within 12 hours, such as within 6 hours, within 5 hours, within 3 hours, or within 1 hour after being reconstituted. In an alternative embodiment, an antigen-binding molecule provided herein is supplied in liquid form in a hermetically sealed container indicating the quantity and concentration of the antigen-binding molecule.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., an antigen-binding molecule provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In specific embodiments, the route of administration for a dose of an antigen-binding molecule provided herein to a patient is intranasal, intramuscular, intravenous, subcutaneous, or a combination thereof, but other routes described herein are also acceptable. Each dose may or may not be administered by an identical route of administration. In specific embodiments, an antigen-binding molecule provided herein may be administered via multiple routes of administration simultaneously or subsequently to other doses of the same or a different antigen-binding molecule provided herein.

In specific embodiments, the antigen-binding molecule provided herein are administered prophylactically or therapeutically to a subject. The antigen-binding molecule provided herein can be prophylactically or therapeutically administered to a subject so as to prevent, lessen or ameliorate a disease or symptom thereof.

### 7.13 Gene Therapy

In a specific embodiment, polynucleotides comprising sequences encoding antigen-binding molecule or functional derivatives thereof, are administered to a subject for use in a method provided herein, for example, to prevent, manage, treat and/or ameliorate a disease, disorder or condition, by way of gene therapy. Such therapy encompasses that performed by the administration to a subject of an expressed or expressible polynucleotide. In an embodiment, the polynucleotides produce their encoded antigen-binding molecule, and the antigen-binding molecule mediates a prophylactic or therapeutic effect. Any of the methods for recombinant gene expression (or gene therapy) available in the art can be used.

For general review of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a specific embodiment, a composition comprises polynucleotides encoding an antigen-binding molecule provided herein, the polynucleotides being part of an expression vector that expresses the antigen-binding molecule or heavy or light chain polypeptides thereof in a suitable host. In particular, such polynucleotides have promoters, such as heterologous promoters, operably linked to the antigen-binding molecule coding region, the promoter being inducible or constitutive, and, optionally, tissue-specific. In another particular embodiment, polynucleotide molecules are used in which the antigen-binding molecule coding sequences and any other desired sequences are flanked by regions that promote homologous recombination at a desired site in the genome, thus providing for intrachromosomal expression of the antibody-encoding polynucleotides (Koller and Smithies, 1989, Proc. Natl. Acad. Sci. USA 86:8932-8935; Zijlstra et al., 1989, Nature 342:435-438).

Delivery of the polynucleotides (e.g., nucleic acids) into a subject can be either direct, in which case the subject is directly exposed to the polynucleotide or polynucleotide-carrying vectors, or indirect, in which case, cells are first transformed with the polynucleotides in vitro, then transplanted into the subject. These two approaches are known, respectively, as in vivo or ex vivo gene therapy.

In a specific embodiment, the polynucleotide sequences are directly administered in vivo, where the sequences are expressed to produce the encoded product. This can be accomplished by any of numerous methods known in the art, e.g., by constructing them as part of an appropriate nucleic acid expression vector and administering the vector so that the sequences become intracellular, e.g., by infection using defective or attenuated retroviral or other viral vectors (see U.S. Pat. No. 4,980,286), or by direct injection of naked DNA, or by use of microparticle bombardment (e.g., a gene gun; Biolistic, Dupont), or coating with lipids or cell surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635; WO 92/20316; WO93/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains polynucleotide sequences encoding an antigen-binding molecule are used. For example, a retroviral vector can be used (see Miller et al., 1993, Meth. Enzymol. 217:581-599). These retroviral vectors contain the components necessary for the correct packaging of the viral genome and integration into the host cell DNA. The polynucleotide sequences encoding the antigen-binding molecule to be used in gene therapy can be cloned into one or more vectors, which facilitates delivery of the gene into a subject. More detail about retroviral vectors can be found in Boesen et al., 1994, Biotherapy 6:291-302, which describes the use of a retroviral vector to deliver the MDR1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in the recombinant production of antigen-binding molecules. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication WO94/12649; and Wang et al., 1995, Gene Therapy 2:775-783. In a specific embodiment, adenovirus vectors are used.

Adeno-associated virus (AAV) can also be utilized (Walsh et al., 1993, Proc. Soc. Exp. Biol. Med. 204:289-300; and U.S. Pat. No. 5,436,146). In a specific embodiment, AAV vectors are used to express an antigen-binding molecule as provided herein. In specific embodiments, the AAV comprises a polynucleotide encoding one or more polypeptides of an antigen-binding molecule. In specific embodiments, the AAV comprises a polynucleotide encoding one or more light chain polypeptides of an antigen-binding molecule. In specific embodiments, the AAV comprises a polynucleotide encoding one or more heavy chain polypeptides of an antigen-binding molecule.

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In a particular embodiment, the polynucleotide (e.g., nucleic acid) is introduced into a cell prior to administration in vivo of the resulting recombinant cell. Such introduction can be carried out by any method known in the art, including but not limited to transfection, electroporation, microinjection, infection with a viral or bacteriophage vector containing the polynucleotide sequences, cell fusion, chromosome-mediated gene transfer, microcell mediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and can be used in accordance with the methods provided herein, provided that the necessary developmental and physiological functions of the recipient cells are not disrupted. The technique should provide for the stable transfer of the nucleic a endospeccid to the cell, so that the polynucleotide is expressible by the cell, such as heritable and expressible by its cell progeny.

The resulting recombinant cells can be delivered to a subject by various methods known in the art. Recombinant blood cells (e.g., hematopoietic stem or progenitor cells) can be administered intravenously. The amount of cells envisioned for use depends on the desired effect, patient state, etc., and can be determined by one skilled in the art.

Cells into which a polynucleotide can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a specific embodiment, the cell used for gene therapy is autologous to the subject.

In an embodiment in which recombinant cells are used in gene therapy, polynucleotide sequences encoding an antigen-binding molecule are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the methods provided herein (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the polynucleotide to be introduced for purposes of gene therapy comprises an inducible promoter operably linked to the coding region, such that expression of the polynucleotide is controllable by controlling the presence or absence of the appropriate inducer of transcription.

### 7.14 Diagnostic Assays and Methods

Labeled antigen-binding molecules and derivatives and analogs thereof, which immunospecifically bind to a target antigen can be used for diagnostic purposes to detect, diagnose, or monitor a disease or disorder.

Antigen-binding molecules provided herein can be used to assay an antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antigen-binding molecule-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (125I, 121I), carbon (14C), sulfur (35S), tritium (3H), indium (121In), and technetium (99Tc); luminescent labels, such as luminol; and fluorescent labels, such as fluorescein and rhodamine, and biotin. One aspect provided herein is the detection and diagnosis of a disease or disorder in a human.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of 99Tc. The labeled antigen-binding molecule will then accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982).

Depending on several variables, including the type of label used and the mode of administration, the time interval following the administration for permitting the labeled antibody to concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a disease or disorder is carried out by repeating the method for diagnosing the a disease or disorder, for example, one month after initial diagnosis, six months after initial diagnosis, one year after initial diagnosis, etc.

Presence of the labeled antigen-binding molecule can be detected in the subject using methods known in the art for in vivo scanning. These methods depend upon the type of label used. Skilled artisans will be able to determine the appropriate method for detecting a particular label. Methods and devices that may be used in the diagnostic methods provided herein include, but are not limited to, computed tomography (CT), whole body scan such as position emission tomography (PET), magnetic resonance imaging (MRI), and sonography.

In a specific embodiment, the antigen-binding molecule is labeled with a radioisotope and is detected in the patient using a radiation responsive surgical instrument (Thurston et al., U.S. Pat. No. 5,441,050). In another embodiment, the antigen-binding molecule is labeled with a fluorescent compound and is detected in the patient using a fluorescence responsive scanning instrument. In another embodiment, the antigen-binding molecule is labeled with a positron emitting metal and is detected in the patient using positron emission-tomography. In yet another embodiment, the antigen-binding molecule is labeled with a paramagnetic label and is detected in a patient using magnetic resonance imaging (MRI).

### 7.15 Kits

Also provided herein are kits comprising an antigen-binding molecule provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain polynucleotides encoding the antibodies provided herein. The cells in the kit can be maintained under appropriate storage conditions until ready to use.

Also provided herein are panels of antigen-binding molecules that immunospecifically bind to a target antigen. In specific embodiments, provided herein are panels of antigen-binding molecule having different association rate constants, different dissociation rate constants, different affinities for an antigen, and/or different specificities for a target antigen. In specific embodiments, provided herein are panels of about 10, preferably about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antigen-binding molecules can be used, for example, in 96 well or 384 well plates, such as for assays such as ELISAs.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

For the sake of conciseness, certain abbreviations are used herein. One example is the single letter abbreviation to represent amino acid residues. The amino acids and their corresponding three letter and single letter abbreviations are shown in TABLE 5 below:

TABLE 5
Amino Acid Abbreviations

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

As used herein, the term “percent identity” in the context of two or more polypeptide or nucleotide sequences (e.g., an antigen-binding molecule or a fragment thereof as described herein, and polynucleotides that encode them), refer to two or more sequences or subsequences that have a specified percentage of amino acid residues or nucleotides that are the same. Percent identity can be determined by alignment of two relevant sequences for maximum correspondence using a sequence comparison algorithm known in the art or by visual inspection.

Optimal alignment of sequences for comparison can be conducted by a sequence comparison algorithm, which can be: the local homology algorithm of Smith & Waterman, Adv. Appl. Math. 2:482 (1981); the homology alignment algorithm of Needleman & Wunsch, J. Mol. Biol. 48:443 (1970), by the search for similarity method of Pearson & Lipman, Proc. Nat'l. Acad. Sci. USA 85:2444 (1988); computerized implementations of these algorithms (GAP, BESTFIT, FASTA, and TFASTA in the Wisconsin Genetics Software Package, Genetics Computer Group, 575 Science Dr., Madison, WI), or can be conducted by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Comparison algorithms that are also suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an25 expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a polynucleotide (e.g., nucleic acid) is considered similar to a reference sequence if the smallest sum probability in a comparison of the test polynucleotide to the reference polynucleotide is less than about 0.1, more preferably less than about 0.01, and most preferably less than about 0.001.

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 8. Embodiments

This invention provides the following non-limiting embodiments. As contemplated herein, any of embodiments A1-A39, B1-B33, C1-C39, D1-D31, and E1-E16 may be combined and/or applied to the claims described herein.

In one set of embodiments (embodiment set A), provided are:

A1. An antigen-binding molecule, comprising
a first light chain polypeptide comprising, in amino-terminus to carboxy-terminus order: VL1-LD1, wherein VL1 is a first light chain variable domain and LD1 is a first light chain dimerization domain; and
a first heavy chain polypeptide comprising, in amino-terminus to carboxy-terminus order: VH1-HD1, wherein VH1 is a first heavy chain variable domain and HD1 is a first heavy chain dimerization domain,
wherein
a) i) LD1 comprises a beta-2 microglobulin (B2M) domain and HD1 comprises a HLA-E A3 (EA3) domain, or LD1 comprises an EA3 domain and HD1 comprises a B2M domain, wherein the B2M domain and the EA3 domain bind to each other to form a dimer; or
ii) LD1 comprises a first ICAM-1 D1 domain and HD1 comprises a second ICAM-1 D1 domain, wherein the first and second ICAM-1 D1 domains bind to each other to form a dimer,
b) i) at least one of the B2M domain or EA3 domain differs from the wild-type B2M domain (SEQ ID NO:2) or wild-type EA3 domain (SEQ ID NO:33), respectively; and
ii) the first ICAM-1 D1 domain differs from the second ICAM-1 D1 domain, and
c) VL1 and VH1 form a first paratope.

A2. The antigen-binding molecule of embodiment A1,
wherein LD1 comprises the B2M domain and HD1 comprises the EA3 domain, or LD1 comprises the EA3 domain and HD1 comprises the B2M domain, wherein the B2M domain and the EA3 domain bind to each other to form a dimer, and
wherein the B2M domain comprises
a) the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM;

b) an amino acid sequence having at least 91% sequence identity to SEQ ID NO:2;
c) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set forth in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1;
f) an amino acid sequence of a human B2M sequence as set forth in SEQ ID NO:2 but comprising a set of substitutions selected from the following, with positions numbered according to the B2M amino acid numbering in TABLE 1,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or
the amino acid sequence of any one of SEQ ID NO:4-30.

A3. The antigen-binding molecule of embodiment A1 or A2, wherein LD1 comprises the B2M domain and HD1 comprises the EA3 domain, or LD1 comprises the EA3 domain and HD1 comprises the B2M domain, wherein the B2M domain and the EA3 domain bind to each other to form a dimer, and
wherein the EA3 domain comprises
a) the amino acid sequence of

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW;

b) an amino acid sequence having at least 93% sequence identity to SEQ ID NO:33;
c) an amino acid sequence having one, two, three, four, five, or six single amino acid substitutions to the sequence of SEQ ID NO:33 at one or more amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, L; or
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2.
f) an amino acid sequence of a human EA3 sequence as set forth in SEQ ID NO:33 but comprising a substitution or set of substitutions selected from the following, with positions numbered according to the EA3 amino acid numbering in TABLE 2,
i) H192C;
ii) R202A or R202C;
iii) E232C;
iv) R234A, R234L, or R234C;
v) D238C;
vi) Q242A or Q242L;
vii) R234A and Q242A;
viii) R234A and Q242L;
ix) R234A and Q242A; or
x) R234L and Q242L; or
g) the amino acid sequence of any one of SEQ ID NOS:35-46.

A4. The antigen-binding molecule of embodiment A1, wherein LD1 comprises the first ICAM-1 D1 domain and HD1 comprises the second ICAM-1 D1 domain, wherein the first and second ICAM-1 D1 domains bind to each other to form a dimer,
wherein the first ICAM-1 D1 domain and the second ICAM-1 D1 domain each comprise a different amino acid sequence selected from the group consisting of
a) the amino acid sequence of

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY;

b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to the ICAM-1 D1 amino acid numbering in TABLE 3;
f) an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3,
i) E34K;
ii) T2V, I10T, T23A, E34K, P38T, P63V, S67A, and T78A;
iii) T2V, I10T, R13C, T23A, E34K, P38T, R49E, P63V, S67A, T78A, and 84C;
iv) T2V, I10T, R13C, T23A, E34K, P38T, E53R, P63V, S67A, T78A, and 84C;
v) T2V, I10T, R13C, L18F, T23A, E34K, P38T, P63V, S67A, T78A, and 84C;
vi) T2V, I10T, R13C, L18A, T20A, T23A, E34K, P38T, L42A, V51A, P63V, S67A, T78A, and 84C; or
vii) T2V, I10T, R13C, L18W, T23A, E34K, P38T, P63V, S67A, T78A, and 84C; or
g) the amino acid sequence of any one of SEQ ID NOS:51-58.

A5. The antigen-binding molecule of any one of embodiments A1-A4, comprising a first light chain elbow region between VL1 and LD1.

A6. The antigen-binding molecule of any one of embodiments A1-A5, comprising a first heavy chain elbow region between VH1 and HD1.

A7. The antigen-binding molecule of any one of embodiments A1-A6, comprising
a first light chain polypeptide comprising, in amino-terminus to carboxy-terminus order: VL1-LE1-LD1, wherein LE1 is a first light chain elbow region; and
a first heavy chain polypeptide comprising, in amino-terminus to carboxy-terminus order: VH1-HE1-HD1, wherein HE1 is a first heavy chain elbow region,
wherein LE1 connects the carboxy-terminus of VL1 to the amino-terminus of LD1 and HE1 connects the carboxy-terminus of VH1 to the amino-terminus of HD1.

A8. The antigen-binding molecule of embodiment A7, wherein LE1 or HE1 is an amino acid sequence of from 3-25 amino acids in length.

A9. The antigen-binding molecule of embodiment A8, wherein LE1 or HE1 is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69) ASTKGGGGSGGGS (SEQ ID NO:70); ASTKGGGGSGGGGS (SEQ ID NO:71); RTVA (SEQ ID NO: 72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); RTVAGGGGSGGGGS (SEQ ID NO:82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

A10. The antigen-binding molecule of any one of embodiments A7-A9, wherein
a) LD1 is the B2M domain, and LE1 is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO: 74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); and RTVAGGGGSGGGGS (SEQ ID NO: 82);
b) HD1 is the EA3 domain, and HE1 is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); or ASTKGGGGSGGGGS (SEQ ID NO:71); or
c) LD1 or HD1 is the first or second ICAM-1 D1 domain, and LE1 or HE1 is an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

A11. The antigen-binding molecule of any one of embodiments A1-A10, comprising a first light chain spacer region fused to the C-terminus of LD1.

A12. The antigen-binding molecule of any one of embodiments A1-A11, comprising a first heavy chain spacer region fused to the C-terminus of HD1.

A13. The antigen-binding molecule of any one of embodiments A1-A12, comprising
a first light chain polypeptide comprising, in amino-terminus to carboxy-terminus order: VL1-LD1-LS1, wherein LS1 is a first light chain spacer region, and
a first heavy chain polypeptide comprising, in amino-terminus to carboxy-terminus order: VH1-HD1-HS1, wherein HS1 is a first heavy chain spacer region.

A14. The antigen-binding molecule of embodiment A13, wherein HS1 or LS1 is an amino acid sequence of from 2-9 amino acids in length.

A15. The antigen-binding molecule of embodiment A13 or A14, wherein HS1 or LS1 is an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

A16. The antigen-binding molecule of any one of embodiment A12-A15, wherein the heavy chain spacer region further comprises a hinge region.

A17. The antigen-binding molecule of any one of embodiment A1-A16, wherein the first light chain polypeptide or first heavy chain polypeptide further comprises a C-terminal tag, optionally wherein the C-terminal tag is a 6× His tag (SEQ ID NO: 93), a Strep-tag II tag, or a human influenza hemagglutinin tag.

A18. The antigen-binding molecule of any one of embodiment A1-A17, wherein the first heavy chain polypeptide further comprises a heavy chain constant domain 2 (CH2).

A19. The antigen-binding molecule of any one of embodiment A1-A18, wherein the first heavy chain polypeptide further comprises a heavy chain constant domain 3 (CH3).

A20. The antigen-binding molecule of any one of embodiment A1-A19, which does not comprise a dimerization sequence of a heavy chain constant domain 1 (CH1) or light chain constant domain (CL).

A21. The antigen-binding molecule of any one of embodiment A1-A20, which is an immunoglobulin, a Fab, a Fab′, a F(ab′)₂, an antibody, a biparatopic antibody, a bispecific antibody, a triparatopic antibody, a trispecific antibody, a tetraparatopic antibody, tetraspecific antibody, a multiparatopic antibody, a multispecific antibody, or any fragment of the antigen-binding molecule that binds to a target antigen.

A22. The antigen-binding molecule of any one of embodiment A1-A21, which further comprises a second light chain polypeptide comprising a second light chain variable domain (VL2) and a second heavy chain polypeptide comprising a second heavy chain variable domain (VH2), wherein VL2 and VH2 form a second paratope.

A23. The antigen-binding molecule of embodiment A22, wherein the first and second paratopes bind to different antigens.

A24. The antigen-binding molecule of embodiment A22 or A23, which further comprises a third light chain polypeptide comprising a third light chain variable domain (VL3) and a third heavy chain polypeptide comprising a third heavy chain variable domain (VH3), wherein VL3 and VH3 form a third antigen-binding domain.

A25. The antigen-binding molecule of embodiment A24, wherein the first, second, and third paratopes bind to different antigens.

A26. The antigen-binding molecule of embodiment A24 or A25, which further comprises a fourth light chain polypeptide comprising a fourth light chain variable domain (VL4) and a fourth heavy chain polypeptide comprising a fourth heavy chain variable domain (VH4), wherein VL4 and VH4 form a fourth paratope.

A27. The antigen-binding molecule of embodiment A26, wherein the first, second, third, and fourth paratopes bind to different antigens.

A28. The antigen-binding molecule of any one of embodiments A1-A27, wherein the first paratope specifically binds to a first Tumor Associated Antigen (TAA1).

A29. The antigen-binding molecule of embodiment A22 or A23, wherein the first paratope specifically binds to a first Tumor Associated Antigen (TAA1), and the second paratope specifically binds to a second Tumor Associated Antigen (TAA2).

A30. The antigen-binding molecule of embodiment A24 or A25, wherein the first paratope specifically binds to a first Tumor Associated Antigen (TAA1), the second paratope specifically binds to a second Tumor Associated Antigen (TAA2), and the third paratope specifically binds to a third Tumor Associated Antigen (TAA3).

A31. The antigen-binding molecule of embodiment A26 or A27, wherein the first paratope specifically binds to a first Tumor Associated Antigen (TAA1), the second paratope specifically binds to a second Tumor Associated Antigen (TAA2), the third paratope specifically binds to a third Tumor Associated Antigen (TAA3), and the fourth paratope specifically binds to a fourth Tumor Associated Antigen (TAA4).

A32. An isolated polynucleotide encoding the antigen-binding molecule of any one of embodiments A1-A31 or a fragment thereof, optionally wherein the isolated polynucleotide sequence comprises a nucleotide sequence as shown in TABLE 29 (e.g., SEQ ID NOS: 3 and 263-343), or a nucleotide sequence having at least 85% sequence identity thereto, or a fragment thereof.

A33. A vector comprising the isolated polynucleotide of embodiment A32.

A34. A host cell containing the vector of embodiment A33.

A35. A pharmaceutical composition, comprising the antigen-binding molecule of any one of embodiments A1-A31, the isolated polynucleotide of embodiment A32, the vector of embodiment A33, or the host cell of embodiment A34, and a pharmaceutically acceptable excipient.

A36. A method for treating a disease or disorder in a subject comprising administering to the subject the pharmaceutical composition of embodiment A35, wherein the paratope or at least the first paratope binds to a target antigen associated with the disease or disorder.

A37. A method for treating a cancer in a subject comprising administering to the subject the pharmaceutical composition of embodiment A35, wherein the paratope or at least the first paratope binds to a target antigen associated with the cancer.

A38. A method of producing an antigen-binding molecule, comprising:
i) culturing the host cell of embodiment A34 under suitable conditions such that the antigen-binding molecule is expressed by the host cell; and
ii) isolating the antigen-binding molecule.

In one set of embodiments (embodiment set B), provided are:

B1. An antigen-binding molecule, comprising,
a first light chain polypeptide comprising, in amino-terminus to carboxy-terminus order,
a) a first light chain variable domain;
b) a first light chain elbow region comprising of 1) from one to eight contiguous amino acids selected from amino acid positions 108-115 of human immunoglobulin kappa constant domain according to EU or Kabat numbering, or 2) an amino acid sequence that is at least three amino acids in length;
c) and a first light chain dimerization domain selected from
i) an HLA-E A3 (EA3) domain;
ii) a beta-2 microglobulin (B2M) domain; or
iii) a first ICAM-1 D1 domain, and
a first heavy chain polypeptide comprising, in amino-terminus to carboxy-terminus order,
d) a first heavy chain variable domain;
e) a first heavy chain elbow region comprising 1) from one to eight contiguous amino acids selected from amino acid positions 118-125 of human IgG1 according to EU numbering, or amino acid positions 114-121 of human IgG1 according to Kabat numbering, or 2) an amino acid sequence that is at least three amino acids in length; and
f) a first heavy chain dimerization domain,
wherein
(1) i) the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer; or
ii) the first light chain dimerization domain comprises the first ICAM-1 D1 domain and the first heavy chain dimerization comprises a second ICAM-1 D1 domain, wherein the first and second ICAM-1 D1 domains bind to each other to form a dimer;
(2) i) at least one of the B2M domain or EA3 domain differs from the wild-type B2M domain (SEQ ID NO:2) or wild-type EA3 domain (SEQ ID NO:33), respectively, and
ii) the first ICAM-1 D1 domain differs from the second ICAM-1 D1 domain; and
(3) the first light chain variable region and first heavy chain variable region form a first paratope.

B2. The antigen-binding molecule of embodiment B1, wherein the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and
wherein the B2M domain comprises
a) the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM;

b) an amino acid sequence having at least 91% sequence identity to SEQ ID NO:2;
c) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set forth in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1;
f) an amino acid sequence of a human B2M sequence as set forth in SEQ ID NO:2 but comprising a set of substitutions selected from the following, with positions numbered according to the B2M amino acid numbering in TABLE 1,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or
g) the amino acid sequence selected of any one of SEQ ID NOS:4-30.

B3. The antigen-binding molecule of embodiment B1 or B2, wherein the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer, and
wherein the EA3 domain comprises
a) the amino acid sequence of

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW;

b) an amino acid sequence having at least 93% sequence identity to SEQ ID NO:33;
c) an amino acid sequence having one, two, three, four, five, or six single amino acid substitutions to the sequence of SEQ ID NO:33 at one or more amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, and L;
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2;
f) an amino acid sequence of a human EA3 sequence as set forth in SEQ ID NO:33 but comprising a substitution or set of substitutions selected from the following, with positions numbered according to the EA3 amino acid numbering in TABLE 2,
i) H192C;
ii) R202A or R202C
iii) E232C;
iv) R234A, R234L, or R234C;
v) D238C;
vi) Q242A or Q242L;
vii) R234A and Q242A;
viii) R234A and Q242L;
ix) R234A and Q242A; or
x) R234L and Q242L; or
g) the amino acid sequence of any one of SEQ ID NO:35-46.

B4. The antigen-binding molecule of embodiment B1, wherein the first light chain dimerization domain comprises the first ICAM-1 D1 domain and the first heavy chain dimerization domain comprises the second ICAM-1 D1 domain, wherein the first and second ICAM-1 D1 domains bind to each other to form a dimer, and wherein the first ICAM-1 D1 domain and second ICAM-1 D1 domain each comprise a different amino acid sequence selected from the group consisting of
a) the amino acid sequence of

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY;

b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:5 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to the ICAM-1 D1 amino acid numbering in TABLE 3.
f) an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3,
i) E34K;
ii) T2V, I10T, T23A, E34K, P38T, P63V, S67A, and T78A;
iii) T2V, I10T, R13C, T23A, E34K, P38T, R49E, P63V, S67A, T78A, and 84C;
iv) T2V, I10T, R13C, T23A, E34K, P38T, E53R, P63V, S67A, T78A, and 84C;
v) T2V, I10T, R13C, L18F, T23A, E34K, P38T, P63V, S67A, T78A, and 84C;
vi) T2V, I10T, R13C, L18A, T20A, T23A, E34K, P38T, L42A, V51A, P63V, S67A, T78A, and 84C;
vii) T2V, I10T, R13C, L18W, T23A, E34K, P38T, P63V, S67A, T78A, and 84C; or
g) the amino acid sequence of any one of SEQ ID NOS:51-58.

B5. The antigen-binding molecule of any one of embodiments B1-B4, wherein the first light chain elbow region or first heavy chain elbow region is an amino acid sequence of from 3-25 amino acids in length.

B6. The antigen-binding molecule of embodiment B5, wherein the first light chain elbow region or first heavy chain elbow region is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO: 63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO: 68); ASTKGGGGSGGS (SEQ ID NO:69)l ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); RTVAGGGGSGGGGS (SEQ ID NO: 82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

B7. The antigen-binding molecule of any one of embodiments B1-B6, wherein
a) the first light chain dimerization domain is the B2M domain, and the first light chain elbow region is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); and RTVAGGGGSGGGGS (SEQ ID NO:82);
b) the first heavy chain dimerization domain is the EA3 domain, and the first heavy chain elbow region is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO: 68); ASTKGGGGSGGS (SEQ ID NO:69)l ASTKGGGGSGGGS (SEQ ID NO:70); and ASTKGGGGSGGGGS (SEQ ID NO:71); or
c) the first light chain dimerization domain or first heavy chain dimerization domain is the first or second ICAM-1 D1 domain, and the first light chain elbow region or first heavy chain elbow region is an amino acid sequence selected from the group consisting of

(SEQ ID NO: 83)
GGGGSGGGGS;
(SEQ ID NO: 84)
GGGGSGGGGSGGGGS;
(SEQ ID NO: 85)
GGGGSGGGGSGGGGSGGGGS;
and
(SEQ ID NO: 86)
GGGGSGGGGSGGGGSGGGGSGGGGS.

B8. The antigen-binding molecule of any one of embodiments B1-B7, comprising a first light chain spacer region fused to the C-terminus of the first light chain dimerization domain.

B9. The antigen-binding molecule of any one of embodiments B1-B8, comprising a first heavy chain spacer region fused to the C-terminus of the first heavy chain dimerization domain.

B10. The antigen-binding molecule of embodiment B8 or B9, wherein the first light chain spacer region or first heavy chain spacer region is an amino acid sequence of from 2-9 amino acids in length.

B11. The antigen-binding molecule of any one of embodiments B8-B10, wherein the first light chain spacer region or first heavy chain spacer region is an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

B12. The antigen-binding molecule of any one of embodiments B1-B13, wherein the first light chain or first heavy chain further comprise a C-terminal tag, optionally wherein the C-terminal tag is a 6× His tag (SEQ ID NO: 93), a Strep-tag II tag, or a human influenza hemagglutinin tag.

B13. The antigen-binding molecule of any one of embodiments B1-B12, wherein first heavy chain further comprises a heavy chain constant domain 2 (CH2).

B14. The antigen-binding molecule of any one of embodiments B1-B13, wherein first heavy chain further comprises a heavy chain constant domain 3 (CH3).

B15. The antigen-binding molecule of any one of embodiments B1-B14, which does not comprise a dimerization sequence of a heavy chain constant domain 1 (CH1) or light chain constant domain (CL).

B16. The antigen-binding molecule of any one of embodiments B1-B15, which is an immunoglobulin, a Fab, a Fab′, a F(ab′)₂, an antibody, a biparatopic antibody, a bispecific antibody, a triparatopic antibody, a trispecific antibody, a tetraparatopic antibody, a tetraspecific antibody, a multiparatopic antibody, a multispecific antibody, or any fragment of the antigen-binding molecule that binds to a target antigen.

B17. The antigen-binding molecule of any one of embodiments B1-B16, which further comprises a second light chain polypeptide comprising a second light chain variable domain and a second heavy chain polypeptide comprising a second heavy chain variable domain, wherein the second light chain variable domain and second heavy chain variable domain form a second paratope.

B18. The antigen-binding molecule of embodiment B17, wherein the first and second paratopes bind to different antigens.

B19. The antigen-binding molecule of embodiment B17 or B18, which further comprises a third light chain comprising a third light chain variable domain and a third heavy chain comprising a third heavy chain variable domain, wherein the third light chain variable domain and third heavy chain variable domain form a third paratope.

B20. The antigen-binding molecule of embodiment B19, wherein the first, second, and third paratopes bind to different antigens.

B21. The antigen-binding molecule of embodiment B19 or B20, which further comprises a fourth light chain comprising a fourth light chain variable domain and a fourth heavy chain comprising a fourth heavy chain variable domain, wherein the fourth light chain variable domain and fourth heavy chain variable domain form a fourth paratope.

B22. The antigen-binding molecule of embodiment B21, wherein the first, second, third, and fourth paratopes bind to different antigens.

B23. The antigen-binding molecule of any one of embodiments B1-B22, wherein the first paratope specifically binds to a first Tumor Associated Antigen (TAA1).

B24. The antigen-binding molecule of embodiment B17 or B18, wherein the first paratope specifically binds to a first Tumor Associated Antigen (TAA1), and the second paratope specifically binds to a second Tumor Associated Antigen (TAA2).

B25. The antigen-binding molecule of embodiment B19 or B20, wherein the first paratope specifically binds to a first Tumor Associated Antigen (TAA1), the second paratope specifically binds to a second Tumor Associated Antigen (TAA2), and the third paratope specifically binds to a third Tumor Associated Antigen (TAA3).

B26. The antigen-binding molecule of embodiment B21 or B22, wherein the first paratope specifically binds to a first Tumor Associated Antigen (TAA1), the second paratope specifically binds to a second Tumor Associated Antigen (TAA2), the third paratope specifically binds to a third Tumor Associated Antigen (TAA3), and the fourth paratope specifically binds to a fourth Tumor Associated Antigen (TAA4).

B27. An isolated polynucleotide encoding the antigen-binding molecule of any one of embodiments B1-B26 or a fragment thereof, optionally wherein the isolated polynucleotide sequence comprises a nucleotide sequence as shown in TABLE 29, or a fragment thereof.

B28. A vector comprising the polynucleotide of embodiment B27.

B29. A host cell containing the vector of embodiment B28.

B30. A pharmaceutical composition, comprising the antigen-binding molecule of any one of embodiments B1-B26, the isolated polynucleotide of embodiment B27, the vector of embodiment B28, or the host cell of embodiment B29, and a pharmaceutically acceptable excipient.

B31. A method for treating a disease or disorder in a subject comprising administering to the subject the pharmaceutical composition of embodiment B30, wherein the paratope or at least the first paratope binds to a target antigen associated with the disease or disorder.

B32. A method for treating a cancer in a subject comprising administering to the subject the pharmaceutical composition of embodiment B30, wherein the paratope or at least the first paratope binds to a target antigen associated with the cancer.

B33. A method of producing an antigen-binding molecule, comprising:
i) culturing the host cell of embodiment B29 under suitable conditions such that the antigen-binding molecule is expressed by the host cell; and
ii) isolating the antigen-binding molecule.

In one set of embodiments (embodiment set C), provided are:

C1. An antigen-binding molecule, comprising a dimer of a first polypeptide and a second polypeptide, wherein
a) the first polypeptide comprises, in amino-terminus to carboxy-terminus order, (i) a first immunoglobulin fragment that does not comprise a dimerization sequence of a CH1 or CL domain, and (ii) a first dimerization domain comprising an HLA-E A3 (EA3) domain, a beta-2 microglobulin (B2M) domain, or a first ICAM-1 D1 domain, and
b) the second polypeptide comprises, in amino-terminus to carboxy-terminus order, (i) a second immunoglobulin fragment that does not comprise a dimerization sequence of a CH1 or CL domain, and (ii) a second dimerization domain comprising an EA3 domain, a B2M domain, or a second ICAM-1 D1 domain,
wherein
(1) i) the first dimerization domain comprises the B2M domain and the second dimerization domain comprises the EA3 domain, or the first dimerization domain comprises the EA3 domain and second dimerization domain comprises the B2M domain, wherein the B2M domain and the EA3 domain bind to each other to form a dimer; or
ii) the first dimerization domain comprises the first ICAM-1 D1 domain and the second dimerization domain comprises the second ICAM-1 D1 domain, wherein the first and second ICAM-1 D1 domains bind to each other to form a dimer;
(2) i) at least one of the B2M domain or EA3 domain differs from the wild-type B2M domain (SEQ ID NO:2) or wild-type EA3 domain (SEQ ID NO:33), respectively, and
ii) the first ICAM-1 D1 domain differs from the second ICAM-1 D1 domain; and
(3) the first immunoglobulin fragment comprises a first immunoglobulin light chain variable domain and the second immunoglobulin fragment comprises a second immunoglobulin heavy chain variable domain, wherein the immunoglobulin light chain variable domain and the immunoglobulin heavy chain variable domain form a first paratope.

C2. A composition comprising a plurality of species of polypeptides, wherein
a) at least one first species of the polypeptides comprises a first polypeptide comprising, in amino-terminus to carboxy-terminus order: (i) a first immunoglobulin fragment that does not comprise the dimerization sequence of CH1 or CL domain, and (ii) a first dimerization domain comprising an HLA-E A3 (EA3) domain, beta 2 microglobulin (B2M) domain, or first ICAM-1 D1 domain; and
b) at least one second species of the polypeptides comprises a second polypeptide comprising, in amino-terminus to carboxy-terminus order: (i) a second immunoglobulin fragment that does not comprise a dimerization sequence of a CH1 or CL domain, and (ii) a second dimerization domain comprising an HLA-E A3 domain, a B2M domain, or a second ICAM-1 D1 domain,
wherein
(1) (A) the first dimerization domain comprises the B2M domain and the second dimerization domain comprises the EA3 domain, or the first dimerization domain comprises the EA3 domain and second dimerization domain comprises the B2M domain, wherein the B2M domain and the EA3 domain bind to each other to form a dimer; or
(B) the first dimerization domain comprises a first ICAM-1 D1 domain and the second dimerization domain comprises a second ICAM-1 D1 domain, wherein the first and second ICAM-1 D1 domains bind to each other to form a dimer;
(2) (A) at least one of the B2M domain or EA3 domain differs from the wild type B2M domain or EA3 domain, respectively, or (B) the first ICAM-1 D1 domain differs from the second ICAM-1 D1 domain;
(3) the first immunoglobulin fragment comprises a first immunoglobulin variable region and the second immunoglobulin fragment comprises a second immunoglobulin variable region, wherein the first and second immunoglobulin variable regions form a first paratope.

C3. The antigen-binding molecule or composition of embodiment C1 or C2,
wherein the first dimerization domain comprises the B2M domain and the second dimerization domain comprises the EA3 domain, or the first dimerization domain comprises the EA3 domain and the second dimerization domain comprises the B2M domain, wherein the B2M domain and the EA3 domain bind to each other to form a dimer, and
wherein the B2M domain comprises
a) the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM;

b) an amino acid sequence having at least 91% sequence identity to SEQ ID NO:2;
c) an amino acid sequence having one, two, three, four, five, six, seven, or eight single amino acid substitutions to the sequence of SEQ ID NO:2 at one or more of amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more of amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set form in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1;
f) an amino acid sequence of a human B2M sequence as set form in SEQ ID NO:2 but comprising a set of substitutions selected from the following, with positions numbered according to the B2M amino acid numbering in TABLE 1,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or
g) the amino acid sequence of any one of SEQ ID NOS:4-30.

C4. The antigen-binding molecule or composition of any one of embodiments C1-C3, wherein the first dimerization domain comprises the B2M domain and the second dimerization domain comprises the EA3 domain, or the first dimerization domain comprises the EA3 domain and the second dimerization domain comprises the B2M domain, wherein the B2M domain and the EA3 domain bind to each other to form a dimer, and
wherein the EA3 domain comprises
a) the amino acid sequence of

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW;

b) an amino acid sequence having at least 93% sequence identity to SEQ ID NO:33;
c) an amino acid sequence having one, two, three, four, five, or six single amino acid substitutions to the sequence of SEQ ID NO:33 at one or more of amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more of amino acid positions 13, 23, 53, 55, 59, and 63 of SEQ ID NO:33, wherein each of the single amino acid substitutions is independently selected from the group consisting of A, C, and L;
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2;
f) an amino acid sequence of a human EA3 sequence as set forth in SEQ ID NO:33 but comprising a substitution or set of substitutions selected from the following, with positions numbered according to the EA3 amino acid numbering in TABLE 2,
i) H192C;
ii) R202A or R202C
iii) E232C;
iv) R234A, R234L, or R234C;
v) D238C;
vi) Q242A or Q242L;
vii) R234A and Q242A;
viii) R234A and Q242L;
ix) R234A and Q242A; or
x) R234L and Q242L; or
g) the amino acid sequence of any one of SEQ ID NO:35-46.

C5. The antigen-binding molecule or composition of any one of embodiments C1-C4, wherein the first dimerization domain comprises a first ICAM-1 D1 domain and the second dimerization domain comprises a second ICAM-1 D1 domain, wherein the first and second ICAM-1 D1 domains bind to each other to form a dimer,
wherein the first ICAM-1 D1 domain and the second ICAM-1 D1 domain each comprise a different amino acid sequence selected from the group consisting of
a) the amino acid sequence of

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY;

b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:49 at one or more of amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more of amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to according to the ICAM-1 D1 amino acid numbering in TABLE 3.
f) an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3
i) E34K;
ii) T2V, I10T, T23A, E34K, P38T, P63V, S67A, and T78A;
iii) T2V, I10T, R13C, T23A, E34K, P38T, R49E, P63V, S67A, T78A, and 84C;
iv) T2V, I10T, R13C, T23A, E34K, P38T, E53R, P63V, S67A, T78A, and 84C;
v) T2V, I10T, R13C, L18F, T23A, E34K, P38T, P63V, S67A, T78A, and 84C;
vi) T2V, I10T, R13C, L18A, T20A, T23A, E34K, P38T, L42A, V51A, P63V, S67A, T78A, and 84C; or
vii) T2V, I10T, R13C, L18W, T23A, E34K, P38T, P63V, S67A, T78A, and 84C; or
g) the amino acid sequence of any one of SEQ ID NOS:51-58.

C6. The antigen-binding molecule or composition of any one of embodiments C1-C5, wherein the first polypeptide comprises a first elbow region between the first immunoglobulin fragment and first dimerization domain.

C7. The antigen-binding molecule or composition of any one of embodiments C1-C6, wherein the second polypeptide comprises a second elbow region between the second immunoglobulin fragment and second dimerization domain.

C8. The antigen-binding molecule or composition of embodiment C6 or C7, wherein the first or second elbow region is an amino acid sequence of from 3-25 amino acids in length.

C9. The antigen-binding molecule or composition of embodiment C8, wherein the first or second elbow region is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); RTVAGGGGSGGGGS (SEQ ID NO:82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

C10. The antigen-binding molecule or composition of any one of embodiments C6-C9, wherein
a) the first dimerization domain is the B2M domain, and the first elbow region is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); and RTVAGGGGSGGGGS (SEQ ID NO:82);
b) the second dimerization domain is the EA3 domain, and the second elbow region is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); and ASTKGGGGSGGGGS (SEQ ID NO:71); or
c) the first and second dimerization domains are the first and second ICAM-1 D1 domains, respectively, and the first and second elbow regions are the same and are each an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

C11. The antigen-binding molecule or composition of any one of embodiments C1-C10, wherein the first polypeptide comprises a first spacer region fused to the C-terminus of the first dimerization domain.

C12. The antigen-binding molecule or composition of any one of embodiments C1-C11, wherein the second polypeptide comprises a second spacer region fused to the C-terminus of the second dimerization domain.

C13. The antigen-binding molecule or composition of embodiment C12, wherein the first spacer region, second spacer region, or both the first and second spacer regions is an amino acid sequence of from 2-9 amino acids in length.

C14. The antigen-binding molecule or composition of any one of embodiments C11-C13, wherein the first spacer region, second spacer region, or both the first and second spacer regions independently is an amino acid sequence selected from the group consisting of

(SEQ ID NO: 87)
EPKSS;
(SEQ ID NO: 88)
SG; SPKSC;
(SEQ ID NO: 89)
GGSGECSG;
(SEQ ID NO: 90)
GGGSGECSG;
(SEQ ID NO: 91)
GGSGESSG;
and
(SEQ ID NO: 92)
GGGSGESSG.

C15. The antigen-binding molecule or composition of any one of embodiments C11-C14, wherein the first spacer region, the second spacer region, or both the first and second spacer regions further comprises a hinge region.

C16. The antigen-binding molecule or composition of any one of embodiments C1-C15, wherein the first polypeptide or second polypeptide further comprises a C-terminal tag, optionally wherein the C-terminal tag is a 6× His tag (SEQ ID NO: 93), a Strep-tag II tag, or a human influenza hemagglutinin tag.

C17. The antigen-binding molecule or composition of any one of embodiments C1-C16, wherein first polypeptide further comprises a heavy chain constant domain 2 (CH2).

C18. The antigen-binding molecule or composition of any one of embodiments C1-C17, wherein first polypeptide further comprises a heavy chain constant domain 3 (CH3).

C19. The antigen-binding molecule or composition of any one of embodiments C1-C18, wherein second polypeptide further comprises a heavy chain constant domain 2 (CH2).

C20. The antigen-binding molecule or composition of any one of embodiments C1-C19, wherein second polypeptide further comprises a heavy chain constant domain 3 (CH3).

C21. The antigen-binding molecule or composition of any one of embodiments C1-C20, wherein the antigen-binding molecule does not comprise a dimerization sequence of a heavy chain constant domain 1 (CH1) or light chain constant domain (CL).

C22. The antigen-binding molecule or composition of any one of embodiments C1-C21, wherein the antigen-binding molecule or plurality of species of polypeptides is an immunoglobulin, a Fab, a Fab′, a F(ab′)₂, an antibody, a biparatopic antibody, a bispecific antibody, a triparatopic antibody, a trispecific antibody, a tetraparatopic antibody, a tetraspecific antibody, a multiparatopic antibody, a multispecific antibody, or any fragment of the antigen-binding molecule that binds to a target antigen.

C23. The antigen-binding molecule or composition of any one of embodiments C1-C22, wherein the antigen-binding molecule or plurality of species of polypeptides further comprises a third polypeptide comprising a second immunoglobulin light chain variable domain, and a fourth polypeptide comprising a second immunoglobulin heavy chain variable domain, wherein the second immunoglobulin light chain variable domain and the second immunoglobulin heavy chain variable domain form a second paratope.

C24. The antigen-binding molecule or composition of embodiment C23, wherein the first and second paratopes bind to different antigens.

C25. The antigen-binding molecule or composition of embodiment C23 or C24, wherein the antigen-binding molecule or plurality of species of polypeptides further comprises a fifth polypeptide comprising a third immunoglobulin light chain variable domain, and a sixth polypeptide comprising a third immunoglobulin heavy chain variable domain, wherein the third immunoglobulin light chain variable domain and third immunoglobulin heavy chain variable domain form a third paratope.

C26. The antigen-binding molecule or composition of embodiment C25, wherein the first, second, and third paratopes bind to different antigens.

C27. The antigen-binding molecule or composition of embodiment C25 or C26, wherein the antigen-binding molecule or plurality of species of polypeptides further comprises a seventh polypeptide comprising a fourth immunoglobulin light chain variable domain, and an eighth polypeptide comprising a fourth immunoglobulin heavy chain variable domain, wherein the fourth immunoglobulin light chain variable domain and fourth immunoglobulin heavy chain variable domain form a fourth paratope.

C28. The antigen-binding molecule or composition of embodiment C27, wherein the first, second, third, and fourth paratopes bind to different antigens.

C29. The antigen-binding molecule or composition of any one of embodiments C1-C28, wherein the first paratope specifically binds to a first Tumor Associated Antigen (TAA1).

C30. The antigen-binding molecule or composition of embodiment C23 or C24, wherein the first paratope specifically binds to a first Tumor Associated Antigen (TAA1), and the second paratope specifically binds to a second Tumor Associated Antigen (TAA2).

C31. The antigen-binding molecule or composition of embodiment C25 or C26, wherein the first paratope specifically binds to a first Tumor Associated Antigen (TAA1), the second paratope specifically binds to a second Tumor Associated Antigen (TAA2), and the third paratope specifically binds to a third Tumor Associated Antigen (TAA3).

C32. The antigen-binding molecule or composition of embodiment C27 or C28, wherein the first paratope specifically binds to a first Tumor Associated Antigen (TAA1), the second paratope specifically binds to a second Tumor Associated Antigen (TAA2), the third paratope specifically binds to a third Tumor Associated Antigen (TAA3), and the fourth paratope specifically binds to a fourth Tumor Associated Antigen (TAA4).

C33. An isolated polynucleotide encoding the antigen-binding molecule or plurality of species of polypeptides of any one of embodiments C1-C32 or a fragment thereof, optionally wherein the isolated polynucleotide sequence comprises a nucleotide sequence as shown in TABLE 29, or a fragment thereof.

C34. A vector comprising the polynucleotide of embodiment C33.

C35. A host cell containing the vector of embodiment C34.

C36. A pharmaceutical composition, comprising the antigen-binding molecule or composition of any one of embodiments C1-C32, the isolated polynucleotide of embodiment C33, the vector of embodiment C34, or the host cell of embodiment C35, and a pharmaceutically acceptable excipient.

C37. A method for treating a disease or disorder in a subject comprising administering to the subject the pharmaceutical composition of embodiment C36, wherein the paratope or at least the first paratope binds to a target antigen associated with the disease or disorder.

C38. A method for treating a cancer in a subject comprising administering to the subject the pharmaceutical composition of embodiment C36, wherein the paratope or at least the first paratope binds to a target antigen associated with the cancer.

C39. A method of producing an antigen-binding molecule or plurality of species of polypeptides, comprising:
i) culturing the host cell of embodiment C35 under suitable conditions such that the antigen-binding molecule is expressed by the host cell; and
ii) isolating the antigen-binding molecule.

In one set of embodiments (embodiment set D), provided are:

D1. A composition comprising a plurality of species of polypeptides, wherein each species of the polypeptides comprises
a) a means for binding to a first target antigen; and
b) a means for dimerization,
wherein in a first at least one species of the polypeptides its means for binding comprises a first means for binding to a first target antigen, and its means for dimerization comprises a first means of dimerization comprising
i) an HLA-E A3 (EA3) domain that binds to a beta-2 microglobulin (B2M) domain to form a dimer;
ii) a B2M domain that binds to an EA3 domain to form a dimer; or
iii) a first ICAM-1 D1 domain that binds to a second ICAM-1 D1 domain to form a dimer, and
wherein in a second at least one species of the polypeptides its means for binding comprises a second means for binding to a first target antigen, and its means for dimerization comprises a second means for dimerization comprising
(1) a B2M domain that binds to the EA3 domain to form a dimer;
(2) an EA3 domain that binds to the B2M domain to form a dimer; or
(3) a second ICAM-1 D1 domain that binds to the first ICAM-1 D1 domain to form a dimer, and
wherein
the first and second means for dimerization differ from each other and bind to each other to form a dimer,
at least one of the EA3 domain or B2M domain differs from the wild-type B2M domain (SEQ ID NO:2) or wild-type EA3 domain (SEQ ID NO:33), and the first ICAM-1 D1 domain differs from the second ICAM-1 D1 domain, and
wherein the first means for dimerization comprises an B2M domain and the second means for dimerization comprises an EA3 domain, or the first means for dimerization comprises an EA3 domain and the second means for dimerization comprises a B2M domain, or the first means for dimerization comprises the first ICAM-1 D1 domain and the second means for dimerization comprises the second ICAM-1 D1 domain; and
the first and second means for binding to a first target antigen form a first paratope.

D2. The composition of embodiment D1, wherein heterodimeric interaction between the first at least one species and the second at least one species of polypeptides is stronger than each of the homodimeric interactions between the same species of the polypeptides.

D3. The composition of embodiment D1 or D2,
wherein the first means for dimerization comprises the B2M domain and the second means for dimerization comprises the EA3 domain, or the first means for dimerization comprises the EA3 domain and the second means for dimerization comprises the B2M domain, wherein the B2M domain and the EA3 domain bind to each other to form a dimer, and
wherein the B2M domain comprises
a) the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM;

b) an amino acid sequence having at least 91% sequence identity to SEQ ID NO:2;
c) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set forth in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1;
f) an amino acid sequence of a human B2M sequence as set forth in SEQ ID NO:2 but comprising a set of substitutions selected from the following, with positions numbered according to the B2M amino acid numbering in TABLE 1,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or
g) the amino acid sequence of any one of SEQ ID NO:4-30.

D4. The composition of any one of embodiments D1-D3, wherein the first means for dimerization comprises the B2M domain and the second means for dimerization comprises the EA3 domain, or the first means for dimerization comprises the EA3 domain and the second means for dimerization comprises the B2M domain, wherein the B2M domain and the EA3 domain bind to each other to form a dimer, and
wherein the EA3 domain comprises
a) the amino acid sequence of

(SEQ ID NO: 33)
LHLEPPKTHVTHHPISDHEATLRCWALGFYPAEITLTWQQDGEGHTQDT
ELVETRPAGDGTFQKWAAVVVPSGEEQRYTCHVQHEGLPEPVTLRW;

b) an amino acid sequence having at least 93% sequence identity to SEQ ID NO:33;
c) an amino acid sequence having one, two, three, four, five, or six single amino acid substitutions to the sequence of SEQ ID NO:33 at one or more amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, and L; or
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2.
f) an amino acid sequence of a human EA3 sequence as set forth in SEQ ID NO:33 but comprising a substitution or set of substitutions selected from the following, with positions numbered according to the EA3 amino acid numbering in TABLE 2,
i) H192C;
ii) R202A or R202C
iii) E232C;
iv) R234A, R234L, or R234C;
v) D238C;
vi) Q242A or Q242L;
vii) R234A and Q242A;
viii) R234A and Q242L;
ix) R234A and Q242A; or
x) R234L and Q242L; or
g) the amino acid sequence of any one of SEQ ID NOS:35-46.

D5. The composition of embodiment D1, wherein the first means for dimerization comprises the first ICAM-1 D1 domain and second means for dimerization comprises the second ICAM-1 D1 domain, wherein the first and second ICAM-1 D1 domains bind to each other to form a dimer, and
wherein the first ICAM-1 D1 domain and the second ICAM-1 D1 domain each comprise a different amino acid sequence selected from the group consisting of
a) the amino acid sequence of

(SEQ ID NO: 49)
QTSVSPSKVILPRGGSVLVTCSTSCDQPKLLGIETPLPKKELLLPGNNR
KVYELSNVQEDSQPMCYSNCPDGQSTAKTFLTVY;

b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to the ICAM-1 D1 amino acid numbering in TABLE 3.
f) an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3,
i) E34K;
ii) T2V, I10T, T23A, E34K, P38T, P63V, S67A, and T78A;
iii) T2V, I10T, R13C, T23A, E34K, P38T, R49E, P63V, S67A, T78A, and 84C;
iv) T2V, I10T, R13C, T23A, E34K, P38T, E53R, P63V, S67A, T78A, and 84C;
v) T2V, I10T, R13C, L18F, T23A, E34K, P38T, P63V, S67A, T78A, and 84C;
vi) T2V, I10T, R13C, L18A, T20A, T23A, E34K, P38T, L42A, V51A, P63V, S67A,
T78A, and 84C; or
vii) T2V, I10T, R13C, L18W, T23A, E34K, P38T, P63V, S67A, T78A, and 84C; or
g) the amino acid sequence of any one of SEQ ID NOS:51-58.

D6. The composition of any one of embodiments D1-D5, wherein the first at least one and second at least one species of the polypeptides each comprises an elbow region between the means for binding to a first antigen, and the means for dimerization.

D7. The composition of embodiment D6, wherein the elbow region of a species of the polypeptides is an amino acid sequence of from 3-25 amino acids in length.

D8 The composition of embodiment D6 or D7, wherein the elbow region of a species of the polypeptides is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); RTVAGGGGSGGGGS (SEQ ID NO:82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

D9. The composition of any one of embodiments D6-D8, wherein
a) the first means for dimerization is the B2M domain, and the elbow region between the first means for binding and the first means for dimerization is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO: 73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); RTVAGGGGSGGGGS (SEQ ID NO:82);
b) the second means for dimerization is an EA3 domain, and elbow region between the second means for binding and the second means for dimerization is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); or ASTKGGGGSGGGGS (SEQ ID NO:71); or
c) the first and second means for dimerization are the first and second ICAM-1 D1 domains, respectively, and the elbow region between the first means for binding and the first means for dimerization and the elbow region between the second means for binding and the second means for dimerization are the same and are each an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

D10. The composition of any one of embodiments D1-D9, wherein one or more species of the polypeptides comprises a spacer region fused to the C-terminus of the means for dimerization.

D11. The composition of embodiment D10, wherein the spacer region is an amino acid sequence of from 2-9 amino acids in length.

D12. The composition of embodiment D10 or D11, wherein the spacer region is an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

D13. The composition of any one of embodiments D10-D12, wherein the spacer region further comprises a hinge region.

D14. The composition of any one of embodiments D1-D13, wherein one or more species of the polypeptides further comprises a C-terminal tag, optionally wherein the C-terminal tag is a 6× His tag (SEQ ID NO: 93), a Strep-tag II tag, or a human influenza hemagglutinin tag

D15. The antigen-binding molecule of any one of embodiments D1-D14, wherein one or more of the species of molecules further comprises a heavy chain constant domain 2 (CH2).

D16. The antigen-binding molecule of any one of embodiments D1-D15, wherein one or more of the species of molecules further comprises a heavy chain constant domain 3 (CH3).

D17. The antigen-binding molecule of any one of embodiments D1-D16, which does not comprise a dimerization sequence of a heavy chain constant domain 1 (CH1) or light chain constant domain (CL).

D18. The antigen-binding molecule of any one of embodiments D1-D17, which is an immunoglobulin, a Fab, a Fab′, a F(ab′)₂, an antibody, a biparatopic antibody, a bispecific antibody, a triparatopic antibody, a trispecific antibody, a tetraparatopic antibody, a tetraspecific antibody, a multiparatopic antibody, a multispecific antibody, or any fragment of the antigen-binding molecule that binds to a target antigen.

D19. The composition of any one of embodiment D1-D18, which further comprises
a) a third at least one species of polypeptide comprising a third means for binding to a second target antigen, and a third means for dimerization; and
b) a fourth at least one species of polypeptide comprising a fourth means for binding to a second target antigen, and a fourth means for dimerization and
wherein the third and fourth means for binding to a second target antigen form a second paratope.

D20. The composition of embodiment D19, wherein the first and second paratopes bind to different target antigens.

D21. The composition of embodiment D19 or D20, which further comprises
a) a fifth at least one species of polypeptide comprising a fifth means for binding to a third antigen, and a fifth means for dimerization; and
b) a sixth at least one species of polypeptide comprising a sixth means for binding to a third target antigen and a sixth means for dimerization, and
wherein the fifth and sixth means for binding to a third target antigen form a third paratope.

D22. The composition of embodiment D21, wherein the first, second, and third paratopes bind to different antigens.

D23. The composition of embodiment D21 or D22, which further comprises
a) a seventh species of polypeptide comprising a seventh means for binding to a fourth target antigen and a seventh means for dimerization, and
b) an eighth species of polypeptide comprising an eighth means for binding to a fourth target antigen and an eighth means for dimerization, and
wherein the seventh and eighth means for binding to a fourth target antigen form a fourth paratope.

D24. The composition of embodiment D23, wherein the first, second, third, and fourth paratopes bind to different antigens.

D25. The composition of any one of embodiments D1-D24, wherein the first paratope specifically binds to a first Tumor Associated Antigen (TAA1).

D26. The composition of any one of embodiment D19 or D20, wherein the first paratope specifically binds to a first Tumor Associated Antigen (TAA1), and the second paratope specifically binds to a second Tumor Associated Antigen (TAA2).

D27. The composition of any one of embodiments D21-D22, wherein the first paratope specifically binds to a first Tumor Associated Antigen (TAA1), the second paratope specifically binds to a second Tumor Associated Antigen (TAA2), and the third paratope specifically binds to a third Tumor Associated Antigen (TAA3).

D28. The composition of any one of embodiments D23-D24, wherein the first paratope specifically binds to a first Tumor Associated Antigen (TAA1), the second paratope specifically binds to a second Tumor Associated Antigen (TAA2), the third paratope specifically binds to a third Tumor Associated Antigen (TAA3), and the fourth paratope specifically binds to a fourth Tumor Associated Antigen (TAA4).

D29. A pharmaceutical composition, comprising the composition of any one of embodiments D1-D28, and a pharmaceutically acceptable excipient.

D30. A method for treating a disease or disorder in a subject comprising administering to the subject the pharmaceutical composition of embodiment D29, wherein the paratope or at least the first paratope binds to a target antigen associated with the disease or disorder.

D31. A method for treating a cancer in a subject comprising administering to the subject the pharmaceutical composition of embodiment D29, wherein the paratope or at least the first paratope binds to a target antigen associated with or specific for the cancer.

In one set of embodiments (embodiment set E), provided are

E1. A method of producing a multiparatopic antibody in a single host cell, the method comprising
culturing a host cell containing one or more polynucleotides encoding
a) a first light chain polypeptide comprising
i) a first light chain variable domain; and
ii) a first light chain dimerization domain that is an HLA-E A3 (EA3) domain, a beta-2 microglobulin (B2M) domain, or a first ICAM-1 D1 domain;
b) a first heavy chain polypeptide comprising
i) a first heavy chain variable domain;
ii) a first heavy chain dimerization domain that is an EA3 domain, a B2M domain, or a second ICAM-1 D1 domain; and
iii) a connection to a second heavy chain polypeptide,
wherein
(1) (i) the first light chain dimerization domain comprises the B2M domain and the first heavy chain dimerization domain comprises the EA3 domain, or the first light chain dimerization domain comprises the EA3 domain and the first heavy chain dimerization domain comprises the B2M domain, wherein the B2M domain and the EA3 domain bind to each other to form a dimer; or
(ii) the first light chain dimerization domain comprises a first ICAM-1 D1 domain and the first heavy chain dimerization comprises a second ICAM-1 D1 domain, wherein the first and second ICAM-1 D1 domains bind to each other to form a dimer;
(2) (i) at least one of the B2M domain or EA3 domain differs from the wild-type B2M domain (SEQ ID NO:2) or wild-type EA3 domain (SEQ ID NO:33), respectively, and (ii) the first ICAM-1 D1 domain differs from the second ICAM-1 D1 domain; and
(3) the first light chain variable domain and first heavy chain variable domain form a first paratope, and
the second heavy chain polypeptide comprises
i) a second heavy chain variable domain;
ii) a second heavy chain dimerization domain that is a heavy chain constant domain 1 (CH1), and wherein the second heavy chain dimerization domain dimerizes to a second light chain dimerization domain of a second light chain polypeptide; and
iii) a connection to the first heavy chain polypeptide, and the second light chain polypeptide comprises
iv) a second light chain variable domain; and
v) a second light chain dimerization domain that is a light chain constant (CL) domain, and wherein the second light chain dimerization domain dimerizes to the second heavy chain dimerization domain,
wherein said culturing is under conditions allowing for expression of the multiparatopic antibody, and
wherein the first light chain variable region and first heavy chain variable region form a first variable region specific for a first target,
the first light chain dimerization domain and first heavy chain dimerization domain dimerize together,
the second light chain dimerization domain and the second heavy chain dimerization domain dimerize together,
the first and second heavy chain are connected together, and
i) at least one of the B2M domain or EA3 domain differs from the wild-type B2M domain (SEQ ID NO:2) or wild-type EA3 domain (SEQ ID NO:33), respectively, and ii) the first ICAM-1 D1 domain differs from the second ICAM-1 D1 domain.

E2. The method of any embodiment E1,
wherein the first dimerization domain comprises the B2M domain and the second dimerization domain comprises the EA3 domain, or the first dimerization domain comprises the EA3 domain and the second dimerization domain comprises the B2M domain, wherein the B2M domain and the EA3 domain bind to each other to form a dimer, and
wherein the B2M domain comprises
a) the amino acid sequence of

(SEQ ID NO: 2)
RTPKIQVYSRHPAENGKSNFLNCYVSGFHPSDIEVDLLKNGERIEKVEH
SDLSFSKDWSFYLLYYTEFTPTEKDEYACRVNHVTLSQPKIVKWDRDM;

b) an amino acid sequence having at least 91% sequence identity to SEQ ID NO:2;
c) an amino acid sequence having one, two, three, four, five, six, seven, or eight single amino acid substitutions to the sequence of SEQ ID NO:2 at one or more of amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more of amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set form in SEQ ID NO:2 but comprising a set of substitutions of F56S, W60S, and F62T with positions numbered according to the B2M amino acid numbering in TABLE 1, optionally further comprising one, two, three, four, or five single amino acid substitutions at positions K6, Y10, R12, D98, or M99 with positions numbered according to the B2M amino acid numbering in TABLE 1;
f) an amino acid sequence of a human B2M sequence as set form in SEQ ID NO:2 but comprising a set of substitutions selected from the following, with positions numbered according to the B2M amino acid numbering in TABLE 1,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or
g) the amino acid sequence of any one of SEQ ID NO:4-30.

E3. The method of embodiment E1, wherein the first dimerization domain comprises the B2M domain and the second dimerization domain comprises the EA3 domain, or the first dimerization domain comprises the EA3 domain and the second dimerization domain comprises the B2M domain, wherein the B2M domain and the EA3 domain bind to each other to form a dimer, and
wherein the EA3 domain comprises
a) the amino acid sequence of

(SEQ ID NO: 33)
LHLEPPKTHVTHHPISDHEATLRCWALGFYPAEITLTWQQDGEG
HTQDTELVETRPAGDGTFQKWAAVVVPSGEEQRYTCHVQHEGLP
EPVTLRW;

b) an amino acid sequence having at least 93% sequence identity to SEQ ID NO:33;
c) an amino acid sequence having one, two, three, four, five, or six single amino acid substitutions to the sequence of SEQ ID NO:33 at one or more of amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more of amino acid positions 13, 23, 53, 55, 59, and 63 of SEQ ID NO:33, wherein each of the single amino acid substitutions is independently selected from the group consisting of A, C, and L;
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in TABLE 2;
f) an amino acid sequence of a human EA3 sequence as set forth in SEQ ID NO:33 but comprising a substitution or set of substitutions selected from the following, with positions numbered according to the EA3 amino acid numbering in TABLE 2,
i) H192C;
ii) R202A or R202C;
iii) E232C;
iv) R234A, R234L, or R234C;
v) D238C;
vi) Q242A or Q242L;
vii) R234A and Q242A;
viii) R234A and Q242L;
ix) R234A and Q242A; or
x) R234L and Q242L; or
g) the amino acid sequence of any one of SEQ ID NO:35-46.

E4. The method of embodiments E1, wherein the first dimerization domain comprises a first ICAM-1 D1 domain and the second dimerization domain comprises a second ICAM-1 D1 domain, wherein the first and second ICAM-1 D1 domains bind to each other to form a dimer,
wherein the first ICAM-1 D1 domain and the second ICAM-1 D1 domain each comprise a different amino acid sequence selected from the group consisting of
a) the amino acid sequence of

(SEQ ID NO: 49)
QTSVSPSKVILPRGGSVLVTCSTSCDQPKLLGIETPLPKKELLL
PGNNRKVYELSNVQEDSQPMCYSNCPDGQSTAKTFLTVY;

b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:49 at one or more of amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more of amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in TABLE 3, optionally further comprising a C-terminal cysteine amino acid addition (84C) with positions numbered according to the ICAM-1 D1 amino acid numbering in TABLE 3.
f) an amino acid sequence of a human ICAM-1 D1 sequence as set forth in SEQ ID NO:49 but comprising a set of substitutions selected from the following, with positions numbered according to the ICAM-1 D1 amino acid numbering in TABLE 3
i) E34K,
ii) T2V, I10T, T23A, E34K, P38T, P63V, S67A, and T78A,
iii) T2V, I10T, R13C, T23A, E34K, P38T, R49E, P63V, S67A, T78A, and 84C,
iv) T2V, I10T, R13C, T23A, E34K, P38T, E53R, P63V, S67A, T78A, and 84C,
v) T2V, I10T, R13C, L18F, T23A, E34K, P38T, P63V, S67A, T78A, and 84C,
vi) T2V, I10T, R13C, L18A, T20A, T23A, E34K, P38T, L42A, V51A, P63V, S67A, T78A, and 84C,
vii) T2V, I10T, R13C, L18W, T23A, E34K, P38T, P63V, S67A, T78A, and 84C; or
g) the amino acid sequence of any one of SEQ ID NOS:51-58.

E5. The method of any one of embodiments E1-E4, wherein the first light chain polypeptide comprises a first light chain elbow region between the first light chain variable domain and first light chain dimerization domain.

E6. The method of any one of embodiments E1-E5, wherein the first heavy chain polypeptide comprises a first heavy chain elbow region between the first heavy chain variable domain and the first heavy chain dimerization domain.

E7. The method of embodiment E5 or E6, wherein the first light chain elbow region or first heavy chain elbow region each are an amino acid sequence of from 3-25 amino acids in length.

E8. The method of embodiment E6 or E7, wherein the first light chain elbow region or first heavy chain elbow region is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); RTVAGGGGSGGGGS (SEQ ID NO:82); GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO: 86).

E9. The method of any one of embodiments E5-E8, wherein
a) the first dimerization domain is the B2M domain, and the first elbow region is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); and RTVAGGGGSGGGGS (SEQ ID NO:82);
b) the second dimerization domain is the EA3 domain, and the second elbow region is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); and ASTKGGGGSGGGGS (SEQ ID NO:71); or
c) the first and second dimerization domains are the first and second ICAM-1 D1 domains, respectively, and the first and second elbow regions are the same and are each an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

E10. The method of any one of embodiments E1-E9, wherein the first light chain polypeptide comprises a first light chain spacer region fused to the C-terminus of the first light chain dimerization domain.

E11. The method of any one of embodiments E1-E10, wherein the first heavy chain polypeptide comprises a first heavy chain spacer region fused to the C-terminus of the second dimerization domain.

E12 The method of embodiment E11, wherein the first light chain spacer region, first heavy chain spacer region, or both the first light chain spacer region and first heavy chain spacer region is an amino acid sequence of from 2-9 amino acids in length.

E13. The method of any one of embodiments E10-E12, wherein the first light chain spacer region, first heavy chain spacer region, or both the first light chain spacer region and first heavy chain spacer region independently is an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

E14. The method of any one of embodiments E10-E13, wherein the first spacer region or the second spacer region or both the first and second spacer regions further comprises a hinge region.

E15. The method of any one of embodiments E1-E14, wherein the first polypeptide or second polypeptide further comprises a C-terminal tag, optionally wherein the C-terminal tag is a 6× His tag (SEQ ID NO: 93), a Strep-tag II tag, or a human influenza hemagglutinin tag.

E16. The method of any one of embodiments E1-E15, wherein the method further comprises isolating the multiparatopic antibody from the culture medium or cells.

### 9. EXAMPLES

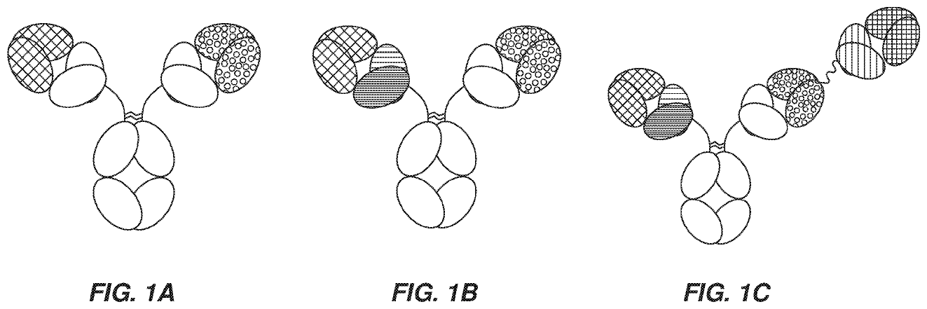

Described herein is a platform in which the CH1 and CL domains of a Fab region were substituted with different immunoglobulin superfamily domains in a “pseudo-Fab” design (See e.g., FIG. 1). This platform is useful for directing appropriate heavy chain-light chain pairing in multiparatopic or multispecific (e.g., bispecific (bsAb)) antibodies, since the CH1 and CL domains of the native Fab preferentially dimerize with each other, but not with the substituted Ig domains of the “pseudo-Fab”.

Direct pairing of VH and cognate VL domains via replacement of the CH1 and CL domains of one Fab of a bsAb with different Ig domains are described in two examples. First, the CH1 and CL domains of a Fab were replaced with the α-3 subunit of human HLA-E and 3-2m, respectively. Second, the CH1 and CL domains of a Fab were replaced with D1 domains of ICAM-1, each of which was engineered to heterodimerize preferentially over homodimerizing. This platform provides a mechanism for generating Fabs having alternative CH1 and CL domains for preparing Fab-based multispecific antibodies that can potentially support therapeutic development.

Described in the examples is a method to direct pairing of VH and cognate VL domains via replacement of the CH1 and CL domains of one Fab of a bsAb with different Ig domains (See, e.g., FIG. 1).

### 9.1 Example 1: MHC-Based CH1-CL Replacement

### 9.1.1 Introduction

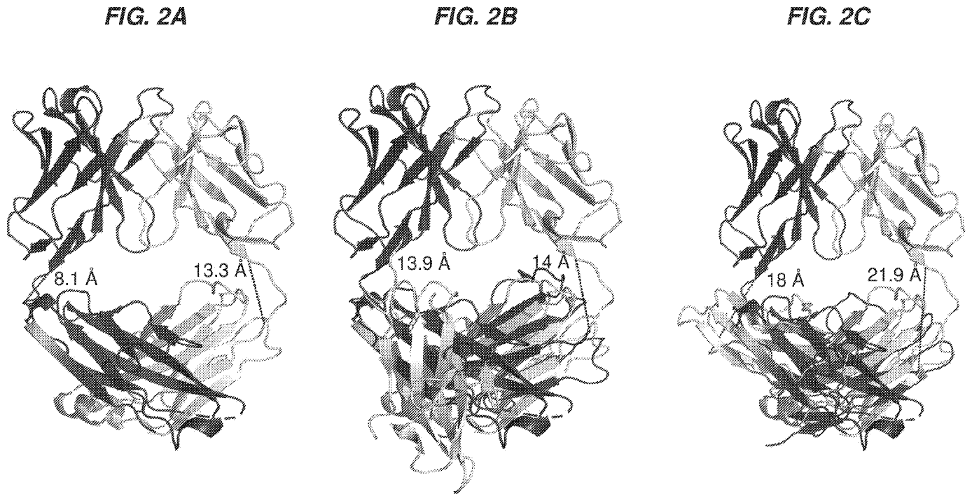

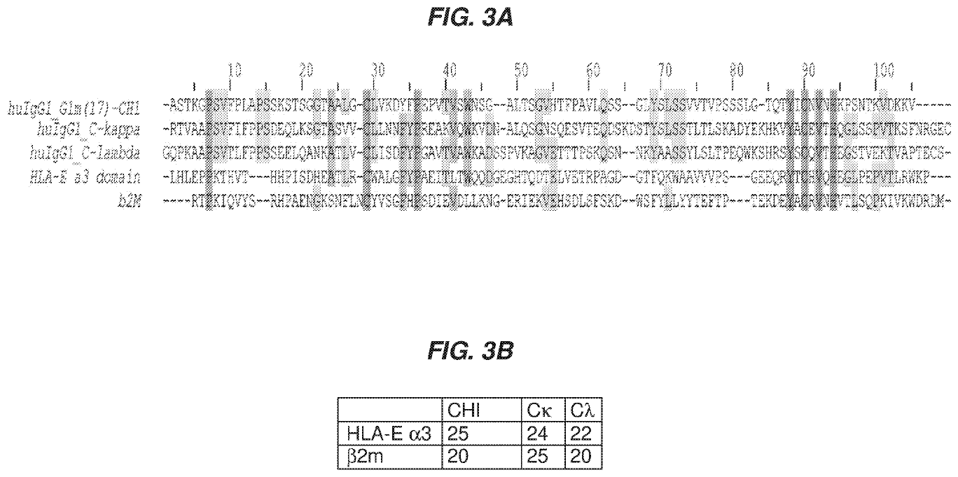

MHC molecules can form heterodimers similar to CH1-CL heterodimer. MHC class I molecules are heterodimers comprised of an a subunit that binds β2m via their α3 domain. These heterodimers are oriented similarly to the CH1-CL heterodimer (See, e.g., FIG. 2A).

HLA-E was chosen as an exemplary dimerization domain (UniProt ID P13747) since it has ~25% sequence identity with the CH1 domain and binds Ig-like transcripts 2 and 4 (ILT2/4) more weakly than HLA-G. MHC Class I ligands, particularly HLA-G, bind the immune-inhibitory receptors ILT2/4, and this interaction contributes to immune cell inactivation. Since designed pseudo-Fabs containing MHC Class I-derived α3-β 2m heterodimers could potentially maintain these interactions, HLA-E α3 (“Eα3”) was chosen since these interactions were weaker.

The HLA-E α3/β2m heterodimer structurally aligned with the CH1-CL heterodimer with r.m.s.d. of ~6.6 Å (FIG. 2B). The combined distance between the N-terminus of the CH1-CL heterodimer and the C-terminus of the VH-VL heterodimer was ~22 Å, whereas the analogous distance when the CH1-CL dimer replaced by the α3/β2m heterodimer was ~28 Å. This suggested that HLA-E α3/βm heterodimers could be used for CH1 replacement. However, the “elbow” sequences linking the variable domains to the HLA-E α3 or βm domains may be modified as described below, to accommodate appropriate VH-VL pairing.

### 9.1.2 Materials and Methods

### 9.1.2.1 Production of Pseudo-Fab Molecules

Expression constructs encoding for pseudo-Fab molecules were transfected into Expi293 cells (ThermoFisher) according to manufacturer's instructions. Cell supernatants were harvested 7 days after transfection by centrifugation at 400×g for 20 min. The clarified supernatant was then passed through a 0.45 μm filter. Purification was performed by immobilized metal affinity chromatography. Supernatants were mixed overnight with Ni Sepharose 6 Fast Flow resin (Cytiva) and then transferred to a gravity flow column. Proteins were eluted with 250 mM imidazole in PBS. Eluates were then either dialyzed against PBS pH 7.2 overnight, or buffer exchanged against PBS. Quantification of dialyzed protein was determined by absorbance at 280 nm using the NanoDrop2000 (ThermoFisher).

### 9.1.2.2 Gel Electrophoresis and Western Blot Analysis of Molecules

Gel electrophoresis of clarified supernatants and proteins was performed to determine expression and purity, respectively. Samples were loaded into 4-20% Mini-PROTEAN TGX Stain-Free polyacrylamide gels (BioRad) and run at 150 V for 40 min. Gels were imaged using the ChemiDoc Imaging System (BioRad). For western blotting, proteins were transferred from the PAGE gels onto PVDF membranes (BioRad) and then blocked with 3% milk in dPBS-T (dPBS with 0.05% Tween-20) for 1 hour at room temperature. Membranes were washed and then probed with the appropriate horseradish-peroxidase conjugated antibody diluted in dPBS-T.

### 9.1.2.3 Size Exclusion Chromatography (SEC)

Purified pseudo-Fab samples were analyzed by size exclusion chromatography on an Agilent 1200 infinity system equipped with a TOSOH TSKgel Bioassist G3SWxl column. The column was equilibrated with dPBS and 20 μL of each sample was injected at a flow rate of 1 mL/min. Based on the gel filtration molecular weight standard (BioRad), the expected retention time of the pseudo-Fab dimers is at ~9 min. Data analysis was performed in OpenLab Chemstation to determine % purity of the samples.

### 9.1.2.4 Intact Molecular Weight Analysis

Formation of desired heterodimer products was assessed using LC-MS. Samples were analyzed using Agilent Infinity System coupled to a Sciex X500B. Samples were loaded onto a Waters Acuity BEH C4 2.1 mm×50 mm, 300 A, 1.7 μm column (Waters). A gradient from 10-50% of acetonitrile and 0.1% formic acid was applied. Mass spectra was acquired in the 500-3000 m/z range in positive ion mode. Data were acquired with SciexOS V1.7 software and processed with SciexOSV2.0 and ProteinMetrics v4.1.

### 9.1.2.5 ELISA of Neat CHO Supernatant of Molecules

Binding samples were analyzed for binding to recombinant RSV-F fusion protein (Sino Biological). ELISAs were carried out according to standard protocols and plates were washed three time with TBS containing 0.05% Tween 20 (TBS-T) between each incubation step. To assess antigen-binding, ninety-six-well Maxisorp plates (Nunc) were coated with dilutions of recombinant RSV-F in 1× dPBS for 18 hours at 4° C. Plates were blocked with 3% BSA in dPBS-T (1× dPBS with 0.05% tween 20) for 1 hour at room temperature. After blocking, plates were incubated with neat, clarified supernatant culture containing molecule of interest for 1 h at RT. After washing, plates were then incubated with either Strep-tag II-HRP (R&D Systems) or anti-HA-HRP (Abcam) in 1× dPBS-T for 1 h at RT. After washing, BM Chemiluminesence ELISA Substrate (POD) was added (Millipore) and plates were immediately read on an Envision plate reader (Perkin Elmer) using ultrasensitive illuminessence. Raw data were exported to GraphPad Prism, where curves were generated and analyzed with a nonlinear regression curve fit. To assess expression levels, the above protocol was implemented as described, with the following modifications. Maxisorp plates (Nunc) were coated with anti-HIS antibody (R&D Systems) at 1 μg/mL in 1× dPBS for 18 hours at 4° C. After blocking and addition of neat supernatants, plates were incubated with either anti-HA-HRP (Abcam) in 1× dPBS-T or anti-Strep tag II-HRP (BioRad, Clone Strep-tagII) in 1× dPBS-T for secondary detection for 1 h at RT.

### 9.1.2.6 ELISA of Purified Pseudo-Fabs

Purified pseudo-fabs were analyzed for binding to cognate antigen, recombinant RSV-F fusion protein (Sino Biological). ELISAs were carried out according to standard protocols and plates were washed three time with TBS containing 0.05% Tween 20 (TBS-T) between each incubation step. To assess antigen-binding, ninety-six-well Maxisorp plates (Nunc) were coated with recombinant RSV-F in 1× dPBS (1 μg/mL) for 18 hours at 4° C. Plates were blocked with 3% milk in PBS-T for 1 hour at room temperature. Plates were then incubated with purified pseudo-Fabs starting at 100 nM and titrated in two-fold dilutions in dPBS-T for 1 hour at room temperature. After washing, plates were then incubated with either anti-StrepTag (BioRad, Clone Strep-tagII) or anti-HA (Abcam) antibodies conjugated to horseradish peroxidase diluted in dPBS-T. After washing, BM Chemiluminesence ELISA Substrate (POD) was added (Millipore) and plates were immediately read on an Envision plate reader (Perkin Elmer) using ultrasensitive illuminessence. Raw data was exported to GraphPad Prism, where curves were generated and analyzed with a nonlinear regression curve fit.

### 9.1.2.7 Differential Scanning Fluorimetry (DSF) of HLA-E Variants

Conformational stability of HLA-containing proteins with B21M binding arms was measured using differential scanning fluorimetry (nanoDSF), by monitoring the intrinsic fluorescence of tryptophan upon thermal unfolding. The unfolding was measured by loading each sample into 24 well capillary (NanoTemper, Cat #PR-AC002) from a 384 well sample plate (ThermoNunc, Cat #264573), with a heating ramp of 1° C./minute between 20~95° C. using the Prometheus NT.48 instrument (NanoTemper Technologies GmbH). Each sample was measured at 0.5 mg/mL in phosphate buffer saline (PBS) in duplicate. The intrinsic fluorescence of each sample at 330 and 350 nm was used to monitor unfolding during temperature ramp and recorded as changes in fluorescence intensity over time. Data were collected and processed using the PR.StabilityAnalysis v1.0.2 software. The processed data contains integrated thermal melting profiles, first derivatives for fluorescence at 330 nm, 350 nm, ratio 330/350, and light scattering data for all the samples. Thermal melting mid-point (Tm) values as well as onset of aggregation (Tagg) are identified and reported.

### 9.1.3 Experimental Development (HLA-E)

Co-expression of the HLA-E α3 domain (EA3) with β2m (B2M) was tested for production of heterodimeric protein with titers sufficient for therapeutic development.

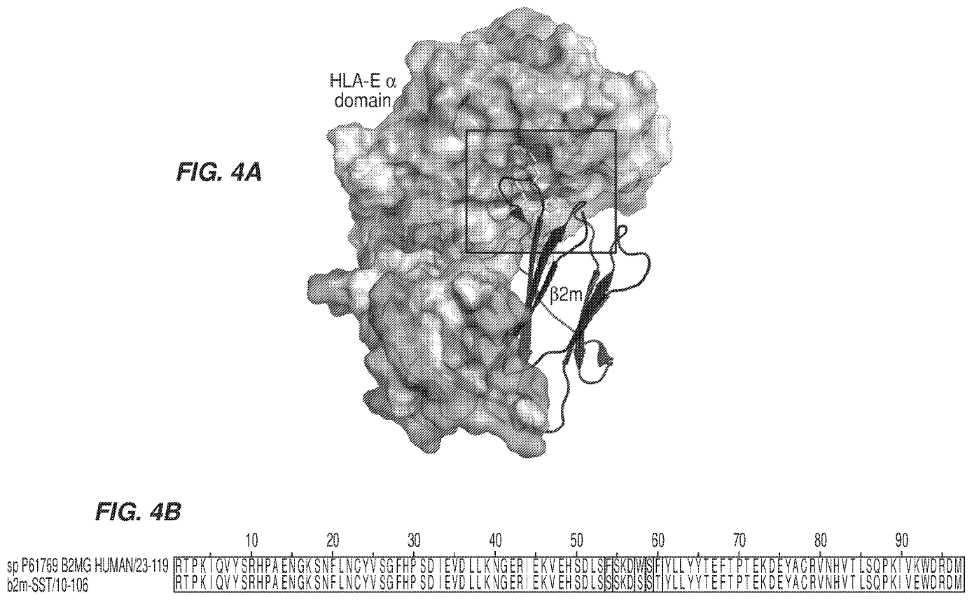

The β2m subunit contains hydrophobic residues in the loop regions that mediate interactions with the α1/α2 domains of MHC Class I ligands (FIG. 4A). To eliminate binding to endogenous HLA/FcRn and to increase the hydrophilicity of the β2m molecule, the mutations F56S, W60S, and F62T were introduced into wild-type human β2m sequence (“B2M(SST)”). An alignment of wild-type human β2m (“B2M”) and B2M (SST) is shown in FIG. 4B. HLA-E α3 and β2m domain insert sequences are shown in TABLE 6. The three amino acids Arg-Thr-Val were optionally appended to the N-terminus of the β2m domain in insert sequences “RTV-[B2M]” and “RTV-[B2M (SST)]”, and thus form part of the elbow sequence in those insert sequences. HLA-E α3 and β2m domain insert-containing polypeptides were prepared having C-terminal affinity tags (6× His (SEQ ID NO: 93) or Strep-tag II) along with spacer sequences (See TABLE 7) amino-terminal to the tags.

TABLE 6
HLA-E α3 and β2m Domain Insert Sequences

| | Insert Sequence |
|---|---|
| Name | (Mutations relative to WT are bolded and underlined) |
| EA3 (wild type) | LHLEPPKTHVTHHPISDHEATLRCWALGFYPAEITLTWQQDGEGHTQDTELVETRPAGDGTFQK |
| | WAAVVVPSGEEQRYTCHVQHEGLPEPVTLRW (SEQ ID NO: 33) |
| B2M (wild type) | RTPKIQVYSRHPAENGKSNFLNCYVSGFHPSDIEVDLLKNGERIEKVEHSDLSFSKDWSFY |
| | LLYYTEFTPTEKDEYACRVNHVTLSQPKIVKWDRDM (SEQ ID NO: 2) |
| B2M (SST) | RTPKIQVYSRHPAENGKSNFLNCYVSGFHPSDIEVDLLKNGERIEKVEHSDLSSSKDSSTY |
| | LLYYTEFTPTEKDEYACRVNHVTLSQPKIVKWDRDM (SEQ ID NO: 4) |
| RTV-[B2M] | RTVRTPKIQVYSRHPAENGKSNFLNCYVSGFHPSDIEVDLLKNGERIEKVEHSDLSFSKDWSFY |
| | LLYYTEFTPTEKDEYACRVNHVTLSQPKIVKWDRDM (SEQ ID NO: 5) |
| RTV-[B2M (SST)] | RTVRTPKIQVYSRHPAENGKSNFLNCYVSGFHPSDIEVDLLKNGERIEKVEHSDLSSSKDSSTY |
| | LLYYTEFTPTEKDEYACRVNHVTLSQPKIVKWDRDM (SEQ ID NO: 6) |

TABLE 7
HLA-E α3 and β2m Domain Polypeptides

| | Polypeptide Sequence | | | | |
|---|---|---|---|---|---|
| | (Mutations relative to | | | | |
| | WT are bolded and | | | C-term | |
| Name | underlined) | Elbow | Insert | Spacer | Tag |
| [EA3]-EPKSS- | LHLEPPKTHVTHHPISDHEATLRCWALGFYPAEI | None | SEQ | EPKSS | HHHHHH |
| His6 | TLTWQQDGEGHTQDTELVETRPAGDGTFQKWAAV | | ID | (SEQ ID | (SEQ ID |
| | VVPSGEEQRYTCHVQHEGLPEPVTLRWEPKSSHH | | NO: | NO: | NO: |
| | HHHH (SEQ ID NO: 100) | | 33 | 87) | 93) |
| [B2M]-SG- | RTPKIQVYSRHPAENGKSNFLNCYVSGFHPS | None | SEQ | SG | WSHPQFE |
| Strep | DIEVDLLKNGERIEKVEHSDLSFSKDWSFYLLYY | | ID | | K |
| | TEFTPTEKDEYACRVNHVTLSQPKIVKWDRDMSG | | NO: | | (SEQ ID |
| | WSHPQFEK (SEQ ID NO: 101) | | 2 | | NO: |
| | | | | | 94) |
| [B2M (SST)]- | RTPKIQVYSRHPAENGKSNFLNCYVSGFHPS | None | SEQ | SG | WSHPQFE |
| SG-Strep | DIEVDLLKNGERIEKVEHSDLSSSKDSSTYLLYY | | ID | | K |
| | TEFTPTEKDEYACRVNHVTLSQPKIVKWDRDMSG | | NO: | | (SEQ ID |
| | WSHPQFEK (SEQ ID NO: 102) | | 4 | | NO: |
| | | | | | 94) |
| RTV-[B2M]- | RTVRTPKIQVYSRHPAENGKSNFLNCYVSGFHPS | RTV | SEQ | SG | WSHPQFE |
| SG-Strep | DIEVDLLKNGERIEKVEHSDLSFSKDWSFYLLYY | | ID | | K |
| | TEFTPTEKDEYACRVNHVTLSQPKIVKWDRDMSG | | NO: | | (SEQ ID |
| | WSHPQFEK (SEQ ID NO: 103) | | 5 | | NO: |
| | | | | | 94) |
| RTV- | RTVRTPKIQVYSRHPAENGKSNFLNCYVSGFHPS | RTV | SEQ | SG | WSHPQFE |
| [B2M (SST)]- | DIEVDLLKNGERIEKVEHSDLSSSKDSSTYLLYY | | ID | | (SEQ ID |
| SG-Strep | TEFTPTEKDEYACRVNHVTLSQPKIVKWDRDMSG | | NO: | | NO: |
| | WSHPQFEK (SEQ ID NO: 104) | | 6 | | 94) |

Anti-RSV F-glycoprotein binding molecules were designed and constructed having a VH domain (“VH”) fused to the N-terminus of HLA-E α3 to form a heavy chain fragment (“HC frag”) cognate VL domain (“VL”) fused to the N-terminus of β2m to form a light chain fragment (“LC frag”), in a pseudo-Fab (“pFab”) design. The elbow sequence for connecting the V to HLA-E α3 was either GGS or absent, and the elbow sequence for connecting the VL to β2m was either V or RTVGGS (SEQ ID NO:59).

The anti-RSV F-glycoprotein variable domain of antibody clone B21M was selected for pseudo-Fab construction, since it requires both VH and VL for binding to its target antigen. See TABLE 7 for the VH and VL sequences. Pseudo-Fab TD Nos. HLPPB17, HLPPB270, and B23B173 were constructed with the sequences shown in TABLE 9.

TABLE 8
VH and VL Sequences of Antibody Clone B21M

| Identifier | Sequence |
|---|---|
| VH (anti-RSV) | QITLKESGPTLVKPTQTLTLTCTFSGFSLSTSG |
| | MGVSWIRQPPGKALEWLAHIYWDDDKRYNPSLK |
| | SRLTITKDTSKNQVVLTMTNMDPVDTATYYCAR |
| | LYGFTYGFAYWGQGTLVTVSS |
| | (SEQ ID NO: 96) |
| VL (anti-RSV) | DIVMTQSPDSLAVSLGERATINCRASQSVDYNG |
| | ISYMHWYQQKPGQPPKLLIYAASNPESGVPDRF |
| | SGSGSGTDFTLTISSLQAEDVAVYYCQQIIEDP |
| | WTFGQGTKVEIK (SEQ ID NO: 97) |

TABLE 9
Pseudo-Fabs of HLA-E α3 and β2m Dimerization Domains

| | | | Polypeptide | | | | | |
|---|---|---|---|---|---|---|---|---|
| | | | (Mutations relative | | | | | |
| | | | to WT are bold | Variable | | | | |
| pFab ID | Chain | Chain ID | and underlined) | Domain | Elbow | Insert | Spacer | Tag |
| HLPPB17 | HC | [VH]- | QITLKESGPTLVKPTQTLTLTCT | SEQ ID | none | SEQ ID | EPKSS | HHHHHH |
| | frag | [EA3]- | FSGFSLSTSGMGVSWIRQPPGKA | NO: 96 | | NO: 33 | (SEQ ID | (SEQ ID |
| | | EPKSS- | LEWLAHIYWDDDKRYNPSLKSRL | | | | NO: 87) | NO: 93) |
| | | His6 | TITKDTSKNQVVLTMTNMDPVDT | | | | | |
| | | | ATYYCARLYGFTYGFAYWGQGTL | | | | | |
| | | | VTVSSLHLEPPKTHVTHHPISDH | | | | | |
| | | | EATLRCWALGFYPAEITLTWQQD | | | | | |
| | | | GEGHTQDTELVETRPAGDGTFQK | | | | | |
| | | | WAAVVVPSGEEQRYTCHVQHEGL | | | | | |
| | | | PEPVTLRWEPKSSHHHHHH | | | | | |
| | | | (SEQ ID NO: 105) | | | | | |
| | LC | [VL]-RTV- | DIVMTQSPDSLAVSLGERATINC | SEQ ID | RTV | SEQ ID | SG | WSHPQF |
| | frag | [B2M | RASQSVDYNGISYMHWYQQKPGQ | NO: 97 | | NO: 4 | | EK |
| | | (SST)]-SG- | PPKLLIYAASNPESGVPDRESGS | | | | | (SEQ ID |
| | | Strep | GSGTDFTLTISSLQAEDVAVYYC | | | | | NO: 94) |
| | | | QQIIEDPWTFGQGTKVEIKRTVR | | | | | |
| | | | TPKIQVYSRHPAENGKSNFLNCY | | | | | |
| | | | VSGFHPSDIEVDLLKNGERIEKV | | | | | |
| | | | EHSDLSSSKDSSTYLLYYTEFTP | | | | | |
| | | | TEKDEYACRVNHVTLSQPKIVKW | | | | | |
| | | | DRDMSGWSHPQFEK | | | | | |
| | | | (SEQ ID NO: 106) | | | | | |
| HLPPB270 | HC | [VH]-GGS- | QITLKESGPTLVKPTQTLTLTCT | SEQ ID | GGS | SEQ ID | EPKSS | HHHHHH |
| | frag | [EA3]- | FSGFSLSTSGMGVSWIRQPPGKA | NO: 96 | | NO: 33 | (SEQ ID | (SEQ ID |
| | | EPKSS- | LEWLAHIYWDDDKRYNPSLKSRL | | | | NO: 87) | NO: 93) |
| | | His6 | TITKDTSKNQVVLTMTNMDPVDT | | | | | |
| | | | ATYYCARLYGFTYGFAYWGQGTL | | | | | |
| | | | VTVSSGGSLHLEPPKTHVTHHPI | | | | | |
| | | | SDHEATLRCWALGFYPAEITLTW | | | | | |
| | | | QQDGEGHTQDTELVETRPAGDGT | | | | | |
| | | | FQKWAAVVVPSGEEQRYTCHVQH | | | | | |
| | | | EGLPEPVTLRWEPKSSHHHHHH | | | | | |
| | | | (SEQ ID NO: 107) | | | | | |
| | LC | [VL]- | DIVMTQSPDSLAVSLGERATINC | SEQ ID | RTVG | SEQ ID | SG | WSHPQF |
| | frag | RTVGGS- | RASQSVDYNGISYMHWYQQKPGQ | NO: 97 | GS | NO: 4 | | EK |
| | | [B2M | PPKLLIYAASNPESGVPDRFSGS | | (SEQ | | | (SEQ ID |
| | | (SST)]-SG- | GSGTDFTLTISSLQAEDVAVYYC | | ID NO: | | | NO: 94) |
| | | Strep | QQIIEDPWTFGQGTKVEIKRTVG | | 59) | | | |
| | | | GSRTPKIQVYSRHPAENGKSNFL | | | | | |
| | | | NCYVSGFHPSDIEVDLLKNGERI | | | | | |
| | | | EKVEHSDLSSSKDSSTYLLYYTE | | | | | |
| | | | FTPTEKDEYACRVNHVTLSQPKI | | | | | |
| | | | VKWDRDMSGWSHPQFEK | | | | | |
| | | | (SEQ ID NO: 108) | | | | | |
| B23B173 | HC | [VH]- | QITLKESGPTLVKPTQTLTLTCT | SEQ ID | none | ASTKGPSVE | EPKSC | HHHHHH |
| | frag | [hIgGIHC] | FSGFSLSTSGMGVSWIRQPPGKA | NO: 96 | | PLAPSSKST | (SEQ ID | (SEQ ID |
| | | -EPKSC- | LEWLAHIYWDDDKRYNPSLKSRL | | | SGGTAALGC | NO: 88) | NO: 93) |
| | | His6 | TITKDTSKNQVVLTMTNMDPVDT | | | LVKDYFPEP | | |
| | | | ATYYCARLYGFTYGFAYWGQGTL | | | VTVSWNSGA | | |
| | | | VTVSSASTKGPSVFPLAPSSKST | | | LTSGVHTFP | | |
| | | | SGGTAALGCLVKDYFPEPVTVSW | | | AVLQSSGLY | | |
| | | | NSGALTSGVHTFPAVLQSSGLYS | | | SLSSVVTVP | | |
| | | | LSSVVTVPSSSLGTQTYICNVNH | | | SSSLGTQTY | | |
| | | | KPSNTKVDKKVEPKSCHHHHHH | | | ICNVNHKPS | | |
| | | | (SEQ ID NO: 109) | | | NTKVDKKV | | |
| | | | | | | (SEQ ID | | |
| | | | | | | NO: 98) | | |
| | LC | [VL]-[hIg | DIVMTQSPDSLAVSLGERATINC | SEQ ID | none | RTVAAPSVE | G | WSHPQF |
| | frag | Kappa]-G- | RASQSVDYNGISYMHWYQQKPGQ | NO: 97 | | IFPPSDEQL | | EK |
| | | Strep | PPKLLIYAASNPESGVPDRESGS | | | KSGTASVVC | | (SEQ ID |
| | | | GSGTDFTLTISSLQAEDVAVYYC | | | LLNNFYPRE | | NO: 94) |
| | | | QQIIEDPWTFGQGTKVEIKRTVA | | | AKVQWKVDN | | |
| | | | APSVFIFPPSDEQLKSGTASVVC | | | ALQSGNSQE | | |
| | | | LLNNFYPREAKVQWKVDNALQSG | | | SVTEQDSKD | | |
| | | | NSQESVTEQDSKDSTYSLSSTLT | | | STYSLSSTL | | |
| | | | LSKADYEKHKVYACEVTHQGLSS | | | TLSKADYEK | | |
| | | | PVTKSFNRGECGWSHPQFEK | | | HKVYACEVT | | |
| | | | (SEQ ID NO: 110) | | | HQGLSSPVT | | |
| | | | | | | KSFNRGEC | | |
| | | | | | | (SEQ ID | | |
| | | | | | | NO: 99) | | |

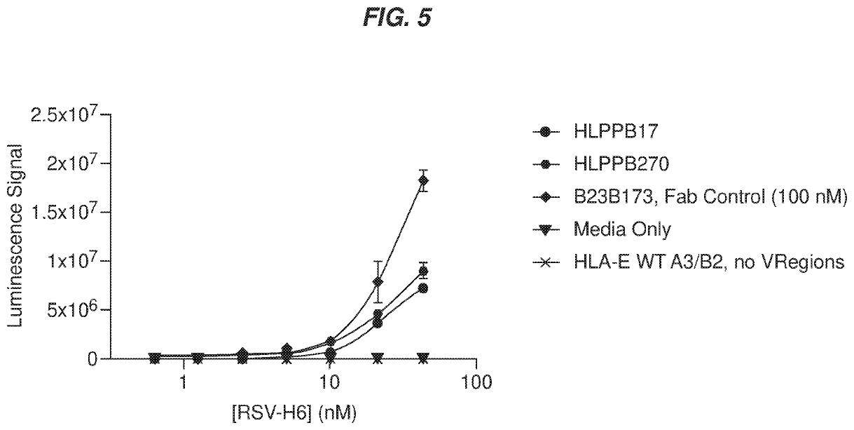

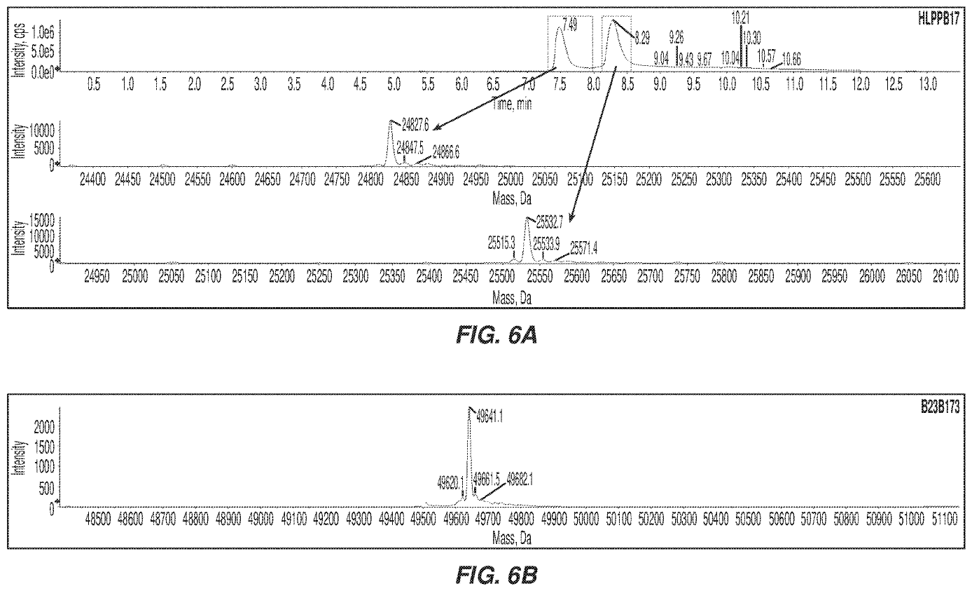

The tested pseudo-Fab molecules HLPPB17 and HLPPB270 expressed at similar levels to a normal Fab control (B323B3173) having the same variable region, but displayed weaker ligand binding (FIG. 5). It was reasoned that although the heterodimeric complex expressed, HLPPB17 and HLPPB270 pseudo-Fabs dissociated during purification and/or analysis, leading to weaker binding due to partial dissociation of the LC (See FIGS. 6A-6B).

Polypeptides were designed having stabilizing mutations at the HLA-E α3-β2m interface to increase dimerization affinity. These stabilizing mutations included introduction of hydrophobic interactions and introduction of electrostatic interactions (see TABLES 10-13). Variant HLA-E α3-β2m dimers comprising these mutations were assembled according to TABLE 14.

Variant HLA-E α3 domain sequences having these dimer-stabilizing mutations were constructed, and their sequences are shown below in TABLE 10 (bolded relative to human WT). Affinity tags were appended onto the C-terminus of HLA-E α3 variants along with spacer sequences amino-terminal to the tags (TABLE 11).

TABLE 10
Variant HLA-E α3 Domains: Hydrophobic and Electrostatic Mutations

| | Sequence |
|---|---|
| Identifier | (Mutations relative to WT are bolded and underlined) |
| EA3 (R234A) | LHLEPPKTHVTHHPISDHEATLRCWALGFYPAEITLTWQQDGEGHTQDTELVETAPAGDGTF |
| | QKWAAVVVPSGEEQRYTCHVQHEGLPEPVTLRW (SEQ ID NO: 35) |
| EA3 (R234L) | LHLEPPKTHVTHHPISDHEATLRCWALGFYPAEITLTWQQDGEGHTQDTELVETLPAGDGTF |
| | QKWAAVVVPSGEEQRYTCHVQHEGLPEPVTLRW (SEQ ID NO: 36) |
| EA3 (R234A, Q242A) | LHLEPPKTHVTHHPISDHEATLRCWALGFYPAEITLTWQQDGEGHTQDTELVETAPAGDGTF |
| | AKWAAVVVPSGEEQRYTCHVQHEGLPEPVTLRW (SEQ ID NO: 37) |
| EA3 (R234L, Q242L) | LHLEPPKTHVTHHPISDHEATLRCWALGFYPAEITLTWQQDGEGHTQDTELVETLPAGDGTF |
| | LKWAAVVVPSGEEQRYTCHVQHEGLPEPVTLRW (SEQ ID NO: 38) |
| EA3 (R234A, Q242L) | LHLEPPKTHVTHHPISDHEATLRCWALGFYPAEITLTWQQDGEGHTQDTELVETAPAGDGTF |
| | LKWAAVVVPSGEEQRYTCHVQHEGLPEPVTLRW (SEQ ID NO: 39) |
| EA3 (R234L, Q242A) | LHLEPPKTHVTHHPISDHEATLRCWALGFYPAEITLTWQQDGEGHTQDTELVETLPAGDGTF |
| | AKWAAVVVPSGEEQRYTCHVQHEGLPEPVTLRW (SEQ ID NO: 40) |
| EA3 (R202A) | LHLEPPKTHVTHHPISDHEATLACWALGFYPAEITLTWQQDGEGHTQDTELVETRPAGDGTF |
| | QKWAAVVVPSGEEQRYTCHVQHEGLPEPVTLRW (SEQ ID NO: 41) |

TABLE 11
Variant HLA-E α3 Domain Polypeptides: Hydrophobic and Electrostatic
Mutations

| | Polypeptide Sequence | | | | |
|---|---|---|---|---|---|
| | (Mutations relative to | | | | |
| | WT are bolded and | | | C-terminus | |
| Name | underlined) | Elbow | Insert | Spacer | Tag |
| [EA3 (R234A)]- | LHLEPPKTHVTHHPISDHEATLRCWALGFYPAE | None | SEQ ID | EPKSS | HHHHHH |
| EPKSS-His6 | ITLTWQQDGEGHTQDTELVETAPAGDGTFQKWA | | NO: 35 | (SEQ ID | (SEQ ID |
| | AVVVPSGEEQRYTCHVQHEGLPEPVTLRWEPKS | | | NO: 87) | NO: 93) |
| | SHHHHHH (SEQ ID NO: 111) | | | | |
| [EA3 (R234L)]- | LHLEPPKTHVTHHPISDHEATLRCWALGFYPAE | None | SEQ ID | EPKSS | HHHHHH |
| EPKSS-His6 | ITLTWQQDGEGHTQDTELVETLPAGDGTFQKWA | | NO: 36 | (SEQ ID | (SEQ ID |
| | AVVVPSGEEQRYTCHVQHEGLPEPVTLRWEPKS | | | NO: 87) | NO: 93) |
| | SHHHHHH (SEQ ID NO: 112) | | | | |
| [EA3 (R234A, | LHLEPPKTHVTHHPISDHEATLRCWALGFYPAE | None | SEQ ID | EPKSS | HHHHHH |
| Q242A)]- | ITLTWQQDGEGHTQDTELVETAPAGDGTFAKWA | | NO: 37 | (SEQ ID | (SEQ ID |
| EPKSS-His6 | AVVVPSGEEQRYTCHVQHEGLPEPVTLRWEPKS | | | NO: 87) | NO: 93) |
| | SHHHHHH (SEQ ID NO: 113) | | | | |
| [EA3 (R234L, | LHLEPPKTHVTHHPISDHEATLRCWALGFYPAE | None | SEQ ID | EPKSS | HHHHHH |
| Q242L)]- | ITLTWQQDGEGHTQDTELVETLPAGDGTFLKWA | | NO: 38 | (SEQ ID | (SEQ ID |
| EPKSS-His6 | AVVVPSGEEQRYTCHVQHEGLPEPVTLRWEPKS | | | NO: 87) | NO: 93) |
| | SHHHHHH (SEQ ID NO: 114) | | | | |
| [EA3 (R234A, | LHLEPPKTHVTHHPISDHEATLRCWALGFYPAE | None | SEQ ID | EPKSS | HHHHHH |
| Q242L)]- | ITLTWQQDGEGHTODTELVETAPAGDGTFLKWA | | NO: 39 | (SEQ ID | (SEQ ID |
| EPKSS-His6 | AVVVPSGEEQRYTCHVQHEGLPEPVTLRWEPKS | | | NO: 87) | NO: 93) |
| | SHHHHHH (SEQ ID NO: 115) | | | | |
| EA3 (R234L, | LHLEPPKTHVTHHPISDHEATLRCWALGFYPAE | None | SEQ ID | EPKSS | HHHHHH |
| Q242A)]- | ITLTWQQDGEGHTODTELVETLPAGDGTFAKWA | | NO: 40 | (SEQ ID | (SEQ ID |
| EPKSS-His6 | AVVVPSGEEQRYTCHVQHEGLPEPVTLRWEPKS | | | NO:87) | NO: 93) |
| | SHHHHHH (SEQ ID NO: 116) | | | | |
| [EA3 (R202A)]- | LHLEPPKTHVTHHPISDHEATLACWALGFYPAE | None | SEQ ID | EPKSS | HHHHHH |
| EPKSS-His6 | ITLTWQQDGEGHTQDTELVETRPAGDGTFQKWA | | NO: 41 | (SEQ ID | (SEQ ID |
| | AVVVPSGEEQRYTCHVQHEGLPEPVTLRWEPKS | | | NO: 87) | NO: 93) |
| | SHHHHHH (SEQ ID NO: 117) | | | | |

Variant β2m domain sequences having these dimer-stabilizing mutations were constructed, and their sequences are shown below in TABLE 12 (bolded relative to human WT). The three amino acids Arg-Thr-Val were optionally appended to the N-terminus of the β2m domain in insert sequences, and thus form part of the elbow sequence in those insert sequences. Affinity tags were appended onto the C-terminus of β2m variants along with spacer sequences amino-terminal to the tags (TABLE 13).

TABLE 12
Variant β2m Domains: Hydrophobic and Electrostatic Mutations

| | Sequence |
|---|---|
| Identifier | (Mutations relative to WT are bolded and underlined) |
| B2M (SST, Y10F) | RTPKIQVFSRHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 7) |
| RTV-[B2M (SST, Y10F)] | RTVRTPKIQVFSRHPAENGKSNFLNCYVSGFHPSDIEVDLLKNGERIEKVEHSDLS |
| | SSKDSSTYLLYYTEFTPTEKDEYACRVNHVTLSQPKIVKWDRDM (SEQ ID NO: 8) |
| B2M (SST, Y10W) | RTPKIQVWSRHPAENGKSNFLNCYVSGFHPSDIEVDLLKNGERIEKVEHSDLS |
| | SSKDSSTYLLYYTEFTPTEKDEYACRVNHVTLSQPKIVKWDRDM (SEQ ID NO: 9) |
| RTV-[B2M (SST, Y10W)] | RTVRTPKIQVWSRHPAENGKSNFLNCYVSGFHPSDIEVDLLKNGERIEKVEHSDLS |
| | SSKDSSTYLLYYTEFTPTEKDEYACRVNHVTLSQPKIVKWDRDM (SEQ ID NO: 10) |
| B2M (SST, D98F) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRFM (SEQ ID NO: 11) |
| RTV-[B2M (SST, D98F)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRFM (SEQ ID NO: 12) |
| B2M (SST, D98W) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRWM (SEQ ID NO: 13) |
| RTV-[B2M (SST, D98W)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRWM (SEQ ID NO: 14) |
| B2M (SST, M99F) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDF (SEQ ID NO: 15) |
| RTV-[B2M (SST, M99F)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDF (SEQ ID NO: 16) |
| B2M (SST, M99W) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDW (SEQ ID NO: 17) |
| RTV-[B2M (SST, M99W)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDW (SEQ ID NO: 18) |

TABLE 13
Variant β2m Domain Polypeptides: Hydrophobic and Electrostatic Mutations

| | Polypeptide Sequence | | | | |
|---|---|---|---|---|---|
| | (Mutations relative to | | | | |
| | WT are bolded and | | | C-terminus | |
| Name | underlined) | Elbow | Insert | Spacer | Tag |
| [B2M (SST, | RTPKIQVFSRHPAENGKSNFLNCYVSGFHPS | None | SEQ ID | SG | WSHPQFE |
| Y10F)]-SG- | DIEVDLLKNGERIEKVEHSDLSSSKDSSTYLLYY | | NO: 7 | | K |
| Strep | TEFTPTEKDEYACRVNHVTLSQPKIVKWDRDMSG | | | | (SEQ ID |
| | WSHPQFEK (SEQ ID NO: 118) | | | | NO: 94) |
| RTV- | RTVRTPKIQVFSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 7 | | K |
| Y10F)]-SG- | TEFTPTEKDEYACRVNHVTLSQPKIVKWDRDMSG | | | | (SEQ ID |
| Strep | WSHPQFEK (SEQ ID NO: 119) | | | | NO: 94) |
| [B2M (SST, | RTPKIQVWSRHPAENGKSNFLNCYVSGFHPS | None | SEQ ID | SG | WSHPQFE |
| Y10W)]-SG- | DIEVDLLKNGERIEKVEHSDLSSSKDSSTYLLYY | | NO: 9 | | K |
| Strep | TEFTPTEKDEYACRVNHVTLSQPKIVKWDRDMSG | | | | (SEQ ID |
| | WSHPQFEK (SEQ ID NO: 120) | | | | NO: 94) |
| RTV- | RTVRTPKIQVWSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 9 | | K |
| Y10W)]-SG- | TEFTPTEKDEYACRVNHVTLSQPKIVKWDRDMSG | | | | (SEQ ID |
| Strep | WSHPQFEK (SEQ ID NO: 121) | | | | NO: 94) |
| [B2M (SST, | RTPKIQVYSRHPAENGKSNFLNCYVSGFHPS | None | SEQ ID | SG | WSHPOFE |
| D98F)]-SG- | DIEVDLLKNGERIEKVEHSDLSSSKDSSTYLLYY | | NO: 11 | | K |
| Strep | TEFTPTEKDEYACRVNHVTLSQPKIVKWDRFMSG | | | | (SEQ ID |
| | WSHPQFEK (SEQ ID NO: 122) | | | | NO: 94) |
| RTV- | RTVRTPKIQVYSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 11 | | K |
| D98F)]-SSG- | TEFTPTEKDEYACRVNHVTLSQPKIVKWDRFMSG | | | | (SEQ ID |
| Strep | WSHPQFEK (SEQ ID NO: 123) | | | | NO: 94) |
| [B2M (SST, | RTPKIQVYSRHPAENGKSNFLNCYVSGFHPS | None | SEQ ID | SG | WSHPQFE |
| D98W)]-SG- | DIEVDLLKNGERIEKVEHSDLSSSKDSSTYLLYY | | NO: 13 | | K |
| Strep | TEFTPTEKDEYACRVNHVTLSQPKIVKWDRWMSG | | | | (SEQ ID |
| | WSHPQFEK (SEQ ID NO: 124) | | | | NO: 94) |
| RTV- | RTVRTPKIQVYSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 13 | | K |
| D98W)]-SG- | TEFTPTEKDEYACRVNHVTLSQPKIVKWDRWMSG | | | | (SEQ ID |
| Strep | WSHPQFEK (SEQ ID NO: 125) | | | | NO: 94) |
| [B2M (SST, | RTPKIQVYSRHPAENGKSNFLNCYVSGFHPS | None | SEQ ID | SG | WSHPQFE |
| M99F)]-SG- | DIEVDLLKNGERIEKVEHSDLSSSKDSSTYLLYY | | NO: 15 | | (SEQ ID |
| Strep | TEFTPTEKDEYACRVNHVTLSQPKIVKWDRDFSG | | | | NO: 94) |
| | WSHPQFEK (SEQ ID NO: 126) | | | | |
| RTV- | RTVRTPKIQVYSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 15 | | K |
| M99F)]-SG- | TEFTPTEKDEYACRVNHVTLSQPKIVKWDRDFSG | | | | (SEQ ID |
| Strep | WSHPQFEK (SEQ ID NO: 127) | | | | NO: 94) |
| [B2M (SST, | RTPKIQVYSRHPAENGKSNFLNCYVSGFHPS | None | SEQ ID | SG | WSHPQFE |
| M99W)]-SG- | DIEVDLLKNGERIEKVEHSDLSSSKDSSTYLLYY | | NO: 17 | | K |
| Strep | TEFTPTEKDEYACRVNHVTLSQPKIVKWDRDWSG | | | | (SEQ ID |
| | WSHPQFEK (SEQ ID NO: 128) | | | | NO: 94) |
| RTV- | RTVRTPKIQVYSRHPAENGKSNFLNCYVSGFHPS | RTV | SEQ ID | SG | WSHPQFE |
| [B2M (SST, | DIEVDLLKNGERIEKVEHSDLSSSKDSSTYLLYY | | NO: 17 | | K |
| M99W)]-SG- | TEFTPTEKDEYACRVNHVTLSQPKIVKWDRDWSG | | | | (SEQ ID |
| Strep | WSHPQFEK (SEQ ID NO: 129) | | | | NO: 94) |

TABLE 14
Dimers of Stabilizing Mutations

| Dimer | Chain | Chain | Chain Polypeptide | | | C-terminus | |
|---|---|---|---|---|---|---|---|
| Identifier | | Polypeptide | Description | Elbow | Insert | Spacer | Tag |
| HLPPW9 | A | SEQ ID NO: 100 | [EA3]-EPKSS-His6 | None | SEQ ID | EPKSS | HHHHHH |
| | | | | | NO: 33 | (SEQ ID | (SEQ ID |
| | | | | | | NO: 87) | NO: 93) |
| | B | SEQ ID NO: 103 | RTV-[B2M]-SG-Strep | RTV | SEQ ID | SG | WSHPQFEK |
| | | | | | NO: 5 | | (SEQ ID |
| | | | | | | | NO: 94) |
| HLPPW10 | A | SEQ ID NO: 100 | [EA3]-EPKSS-His6 | None | SEQ ID | EPKSS | HHHHHH |
| | | | | | NO:33 | (SEQ ID | (SEQ ID |
| | | | | | | NO: 87) | NO: 93) |
| | B | SEQ ID NO: 102 | RTV-[B2M (SST)]-SG- | RTV | SEQ ID | SG | WSHPQFEK |
| | | | Strep | | NO: 4 | | (SEQ ID |
| | | | | | | | NO: 94) |
| HLPPW13 | A | SEQ ID NO: 100 | [EA3]-EPKSS-His6 | None | SEQ ID | EPKSS | HHHHHH |
| | | | | | NO:33 | (SEQ ID | (SEQ ID |
| | | | | | | NO: 87) | NO: 93) |
| | B | SEQ ID NO: 119 | RTV-[B2M (SST, | RTV | SEQ ID | SG | WSHPQFEK |
| | | | Y10F)]-SG-Strep | | NO: 7 | | (SEQ ID |
| | | | | | | | NO: 94) |
| HLPPW14 | A | SEQ ID NO: 100 | [EA3]-EPKSS-His6 | None | SEQ ID | EPKSS | HHHHHH |
| | | | | | NO: 33 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW15 | A | SEQ ID NO: 111 | [EA3 (R234A)]-EPKSS- | None | SEQ ID | EPKSS | HHHHHH |
| | | | His6 | | NO: 35 | (SEQ ID | (SEQ ID |
| | | | | | | NO: 87) | NO: 93) |
| | B | SEQ ID NO: 104) | RTV-[B2M (SST)]-SG- | RTV | SEQ ID | SG | WSHPQFEK |
| | | | Strep | | NO: 6 | | (SEQ ID |
| | | | | | | | NO: 94) |
| HLPPW16 | A | SEQ ID NO: 111 | [EA3 (R234A)]-EPKSS- | None | SEQ ID | EPKSS | HHHHHH |
| | | | His6 | | NO: 35 | (SEQ ID | (SEQ ID |
| | | | | | | NO: 87) | NO: 93) |
| | B | SEQ ID NO: 119 | RTV-[B2M (SST, | RTV | SEQ ID | SG | WSHPQFEK |
| | | | Y10F)]-SG-Strep | | NO: 7 | | (SEQ ID |
| | | | | | | | NO: 94) |
| HLPPW17 | A | SEQ ID NO: 111 | [EA3 (R234A)]-EPKSS- | None | SEQ ID | EPKSS | HHHHHH |
| | | | His6 | | NO: 35 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW18 | A | SEQ ID NO: 112 | [EA3 (R234L)]-EPKSS- | None | SEQ ID | EPKSS | HHHHHH |
| | | | His6 | | NO: 36 | (SEQ ID | (SEQ ID |
| | | | | | | NO: 87) | NO: 93) |
| | B | SEQ ID NO: 104) | RTV-[B2M(SST)]-SG- | RTV | SEQ ID | SG | WSHPQFEK |
| | | | Strep | | NO: 6 | | (SEQ ID |
| | | | | | | | NO: 94) |
| HLPPW19 | A | SEQ ID NO: 112 | [EA3 (R234L)]-EPKSS- | None | SEQ ID | EPKSS | HHHHHH |
| | | | His6 | | NO: 36 | (SEQ ID | (SEQ ID |
| | | | | | | NO: 87) | NO: 93) |
| | B | SEQ ID NO: 119 | RTV-[B2M (SST, | RTV | SEQ ED | SG | WSHPQFEK |
| | | | Y10F)]-SG-Strep | | NO: 7 | | (SEQ ID |
| | | | | | | | NO: 94) |
| HLPPW20 | A | SEQ ID NO: 112 | [EA3 (R234L)]-EPKSS- | None | SEQ ID | EPKSS | HHHHHH |
| | | | His6 | | NO: 36 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW21 | A | SEQ ID NO: 113 | [EA3 (R234A, Q242A)]- | None | SEQ ID | EPKSS | HHHHHH |
| | | | EPKSS-His6 | | NO: 37 | (SEQ ID | (SEQ ID |
| | | | | | | NO: 87) | NO: 93) |
| | B | SEQ ID NO: 104) | RTV-[B2M(SST)]-SG- | RTV | SEQ ID | SG | WSHPQFEK |
| | | | Strep | | NO: 6 | | (SEQ ID |
| | | | | | | | NO: 94) |
| HLPPW22 | A | SEQ ID NO: 113 | [EA3 (R234A, Q242A)]- | None | SEQ ID | EPKSS | HHHHHH |
| | | | EPKSS-His6 | | NO: 37 | (SEQ ID | (SEQ ID |
| | | | | | | NO: 87) | NO: 93) |
| | B | SEQ ID NO: 119 | RTV-[B2M (SST, | RTV | SEQ ED | SG | WSHPQFEK |
| | | | Y10F)]-SG-Strep | | NO: 7 | | (SEQ ID |
| | | | | | | | NO: 94) |
| HLPPW23 | A | SEQ ID NO: 113 | [EA3 (R234A, Q242A)]- | None | SEQ ID | EPKSS | HHHHHH |
| | | | EPKSS-His6 | | NO: 37 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW24 | A | SEQ ID NO: 114 | [EA3 (R234L, Q242L)]- | None | SEQ ID | EPKSS | HHHHHH |
| | | | EPKSS-His6 | | NO: 38 | (SEQ ID | (SEQ ID |
| | | | | | | NO: 87) | NO: 93) |
| | B | SEQ ID NO: 104) | RTV-[B2M(SST)]-SG- | RTV | SEQ ID | SG | WSHPQFEK |
| | | | Strep | | NO: 6 | | (SEQ ID |
| | | | | | | | NO: 94) |
| HLPPW25 | A | SEQ ID NO: 114 | [EA3 (R234L, Q242L)]- | None | SEQ ID | EPKSS | HHHHHH |
| | | | EPKSS-His6 | | NO: 38 | (SEQ ID | (SEQ ID |
| | | | | | | NO: 87) | NO: 93) |
| | B | SEQ ID NO: 119 | RTV-[B2M (SST, | RTV | SEQ ID | SG | WSHPQFEK |
| | | | Y10F)]-SG-Strep | | NO: 7 | | (SEQ ID |
| | | | | | | | NO: 94) |
| HLPPW26 | A | SEQ ID NO: 114 | [EA3 (R234L, Q242L)]- | None | SEQ ID | EPKSS | HHHHHH |
| | | | EPKSS-His6 | | NO: 38 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW27 | A | SEQ ID NO: 115 | [EA3 (R234A, Q242L)]- | None | SEQ ID | EPKSS | HHHHHH |
| | | | EPKSS-His6 | | NO: 39 | (SEQ ID | (SEQ ID |
| | | | | | | NO: 87) | NO: 93) |
| | B | SEQ ID NO: 104) | RTV-[B2M (SST)]-SG- | RTV | SEQ ID | SG | WSHPQFEK |
| | | | Strep | | NO: 6 | | (SEQ ID |
| | | | | | | | NO: 94) |
| HLPPW28 | A | SEQ ID NO: 115 | [EA3 (R234A, Q242L)]- | None | SEQ ID | EPKSS | HHHHHH |
| | | | EPKSS-His6 | | NO: 39 | (SEQ ID | (SEQ ID |
| | | | | | | NO: 87) | NO: 93) |
| | B | SEQ ID NO: 119 | RTV-[B2M (SST, | RTV | SEQ ID | SG | WSHPQFEK |
| | | | Y10F)]-SG-Strep | | NO: 7 | | (SEQ ID |
| | | | | | | | NO: 94) |
| HLPPW29 | A | SEQ ID NO: 115 | [EA3 (R234A, Q242L)]- | None | SEQ ID | EPKSS | HHHHHH |
| | | | EPKSS-His6 | | NO: 39 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW30 | A | SEQ ID NO: 116 | [EA3 (R234L, Q242A)]- | None | SEQ ID | EPKSS | HHHHHH |
| | | | EPKSS-His6 | | NO: 40 | (SEQ ID | (SEQ ID |
| | | | | | | NO: 87) | NO: 93) |
| | B | SEQ ID NO: 104) | RTV-[B2M(SST)]-SG- | RTV | SEQ ID | SG | WSHPQFEK |
| | | | Strep | | NO: 6 | | (SEQ ID |
| | | | | | | | NO: 94) |
| HLPPW31 | A | SEQ ID NO: 116 | [EA3 (R234L, Q242A)]- | None | SEQ ID | EPKSS | HHHHHH |
| | | | EPKSS-His6 | | NO: 40 | (SEQ ID | (SEQ ID |
| | | | | | | NO: 87) | NO: 93) |
| | B | SEQ ID NO: 119 | RTV-[B2M (SST, | RTV | SEQ ID | SG | WSHPQFEK |
| | | | Y10F)]-SG-Strep | | NO: 7 | | (SEQ ID |
| | | | | | | | NO: 94) |
| HLPPW32 | A | SEQ ID NO: 116 | [EA3 (R234L, Q242A)]- | None | SEQ ID | EPKSS | HHHHHH |
| | | | EPKSS-His6 | | NO: 40 | (SEQ ID | (SEQ ID |
| | | | | | | NO: 87) | NO: 93) |
| | B | SEQ ID NO: 121 | RTV-[B2M (SST, | RTV | SEQ ID | SG | WSHPQFEK |
| | | | Y10W)]-SG-Strep | | NO: 9 | | (SEQ ID |
| | | | | | | | NO: 94) |
| HLPPW33 | A | SEQ ID NO: 100 | [EA3]-EPKSS-His6 | None | SEQ ID | EPKSS | HHHHHH |
| | | | | | NO: 33 | (SEQ ID | (SEQ ID |
| | | | | | | NO: 87) | NO: 93) |
| | B | SEQ ID NO: 123 | RTV-[B2M (SST, | RTV | SEQ ID | SG | WSHPQFEK |
| | | | D98F)]-SG-Strep | | NO: 11 | | (SEQ ID |
| | | | | | | | NO: 94) |
| HLPPW34 | A | SEQ ID NO: 100 | [EA3]-EPKSS-His6 | None | SEQ ID | EPKSS | HHHHHH |
| | | | | | NO: 33 | (SEQ ID | (SEQ ID |
| | | | | | | NO: 87) | NO: 93) |
| | B | SEQ ID NO: 125 | RTV-[B2M (SST, | RTV | SEQ ID | SG | WSHPQFEK |
| | | | D98W)]-SG-Strep | | NO: 13 | | (SEQ ID |
| | | | | | | | NO: 94) |
| HLPPW35 | A | SEQ ID NO: 100 | [EA3]-EPKSS-His6 | None | SEQ ID | EPKSS | HHHHHH |
| | | | | | NO:33 | (SEQ ID | (SEQ ID |
| | | | | | | NO: 87) | NO: 93) |
| | B | SEQ ID NO: 127 | RTV-[B2M (SST, | RTV | SEQ ID | SG | WSHPQFEK |
| | | | M99F)]-SG-Strep | | NO: 15 | | (SEQ ID |
| | | | | | | | NO: 94) |
| HLPPW36 | A | SEQ ID NO: 100 | [EA3]-EPKSS-His6 | None | SEQ ID | EPKSS | HHHHHH |
| | | | | | NO: 33 | (SEQ ID | (SEQ ID |
| | | | | | | NO: 87) | NO: 93) |
| | B | SEQ ID NO: 129 | RTV-[B2M (SST, | RTV | SEQ ID | SG | WSHPQFEK |
| | | | M99W)]-SG-Strep | | NO: 17 | | (SEQ ID |
| | | | | | | | NO: 94) |
| HLPPW37 | A | SEQ ID NO: 117 | [EA3 (R202A)]-EPKSS- | None | SEQ ID | EPKSS | HHHHHH |
| | | | His6 | | NO: 41 | (SEQ ID | (SEQ ID |
| | | | | | | NO: 87) | NO: 93) |
| | B | SEQ ID NO: 104) | RTV-[B2M(SST)]-SG- | RTV | SEQ ID | SG | WSHPQFEK |
| | | | Strep | | NO: 6 | | (SEQ ID |
| | | | | | | | NO: 94) |
| HLPPW38 | A | SEQ ID NO: 117 | [EA3 (R202A)]-EPKSS- | None | SEQ ID | EPKSS | HHHHHH |
| | | | His6 | | NO: 41 | (SEQ ID | (SEQ ID |
| | | | | | | NO: 87) | NO: 93) |
| | B | SEQ ID NO: 123 | RTV-[B2M (SST, | RTV | SEQ ID | SG | WSHPQFEK |
| | | | D98F)]-SG-Strep | | NO: 11 | | (SEQ ID |
| | | | | | | | NO: 94) |
| HLPPW39 | A | SEQ ID NO: 117 | [EA3 (R202A)]-EPKSS- | None | SEQ ID | EPKSS | HHHHHH |
| | | | His6 | | NO: 41 | (SEQ ID | (SEQ ID |
| | | | | | | NO: 87) | NO: 93) |
| | B | SEQ ID NO: 125 | RTV-[B2M (SST, | RTV | SEQ ID | SG | WSHPQFEK |
| | | | D98W)]-SG-Strep | | NO: 13 | | (SEQ ID |
| | | | | | | | NO: 94) |
| HLPPW40 | A | SEQ ID NO: 117 | [EA3 (R202A)]-EPKSS- | None | SEQ ID | EPKSS | HHHHHH |
| | | | His6 | | NO: 41 | (SEQ ID | (SEQ ID |
| | | | | | | NO: 87) | NO: 93) |
| | B | SEQ ID NO: 127 | RTV-[B2M (SST, | RTV | SEQ ID | SG | WSHPQFEK |
| | | | M99F)]-SG-Strep | | NO: 15 | | (SEQ ID |
| | | | | | | | NO: 94) |
| HLPPW41 | A | SEQ ID NO: 117 | [EA3 (R202A)]-EPKSS- | None | SEQ ID | EPKSS | HHHHHH |
| | | | His6 | | NO: 41 | (SEQ ID | (SEQ ID |
| | | | | | | NO: 87) | NO: 93) |
| | B | SEQ ID NO: 129 | RTV-[B2M (SST, | RTV | SEQ ID | SG | WSHPQFEK |
| | | | M99W)]-SG-Strep | | NO: 17 | | (SEQ ID |
| | | | | | | | NO: 94) |

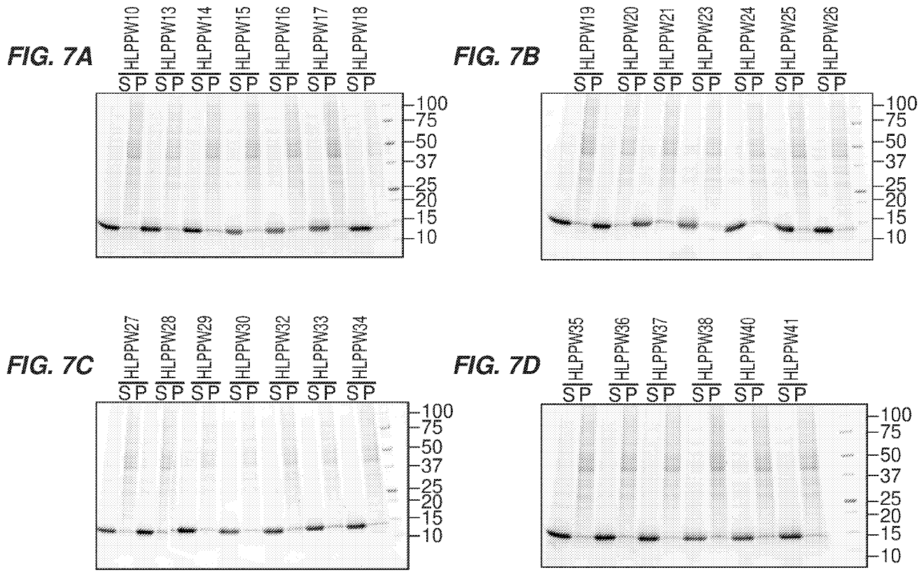

Expression of pseudo-Fab molecules HLPPW10, HLPPW13, HLPPW14, HLPPW15, HLPPW16, HLPPW17, HLPPW18, HLPPW19, HLPPW20, HLPPW21, HLPPW23, HLPPW24, HLPPW25, HLPPW26, HLPPW27, HLPPW28, HLPPW29, HLPPW30, HLPPW32, HLPPW33, HLPPW34, HLPPW35, HLPPW36, HLPPW37, HLPPW38, HLPPW40, and HLPPW41 was assayed, and is shown in FIGS. 7A-7D.

The HLA-E α3/β2m interface was alternatively further stabilized to increase dimerization affinity by introducing cysteines whose side chains were oriented to favor disulfide bond formation (See TABLES 15-18). One additional design featured additional sequences on the C-termini of HLA-E α3 and β2m to form a disulfide bond analogous to the intermolecular disulfide bond between C214 of IgG light chains and C220 of IgG heavy chains. Pseudo-Fabs with the sequences shown in TABLE 19 were assembled from variant HLA-E α3-β2m dimers comprising these mutations. HLA-E α3 dimer-stabilizing disulfide mutations that were introduced are shown below in TABLE 15 (bolded relative to human WT). Affinity tags were appended onto the C-terminus of HLA-E α3 disulfide variants along with spacers to facilitate purification (TABLE 11).

TABLE 15
Variant HLA-E α3 Domains: Disulfide Mutations

| | Sequence |
|---|---|
| Identifier | (Mutations relative to WT are bolded and underlined) |
| EA3 (R202C) | LHLEPPKTHVTHHPISDHEATLCCWALGFYPAEITLTWQQDGEGHTQDTELVETRPAGDGTFQKWAAV |
| | VVPSGEEQRYTCHVQHEGLPEPVTLRW (SEQ ID NO: 42) |
| EA3 (H192C) | LHLEPPKTHVTHCPISDHEATLRCWALGFYPAEITLTWQQDGEGHTODTELVETRPAGDGTFQKWAAV |
| | VVPSGEEQRYTCHVQHEGLPEPVTLRW (SEQ ID NO: 43) |
| EA3 (E232C) | LHLEPPKTHVTHHPISDHEATLRCWALGFYPAEITLTWQQDGEGHTQDTELVCTRPAGDGTFQKWAAV |
| | VVPSGEEQRYTCHVQHEGLPEPVTLRW (SEQ ID NO: 44) |
| EA3 (R234C) | LHLEPPKTHVTHHPISDHEATLRCWALGFYPAEITLTWQQDGEGHTQDTELVETCPAGDGTFQKWAAV |
| | VVPSGEEQRYTCHVOHEGLPEPVTLRW (SEQ ID NO: 45) |
| EA3 (D238C) | LHLEPPKTHVTHHPISDHEATLRCWALGFYPAEITLTWQQDGEGHTQDTELVETRPAGCGTFQKWAAV |
| | VVPSGEEQRYTCHVQHEGLPEPVTLRW (SEQ ID NO: 46) |

TABLE 16
Variant HLA-E α3 Domain Polypeptides: Disulfide Mutations

| | Polypeptide | Elbow | Insert | | |
|---|---|---|---|---|---|
| | (Mutations relative to WT are | | | C-terminus | |
| Identifier | bolded and underlined) | | | Spacer | Tag |
| [EA3]-EPKSC-His6 | LHLEPPKTHVTHHPISDHEATLRCWALGFYPAEITLT | None | SEQ ID | EPKSC | HHHHHH |
| | WQQDGEGHTQDTELVETRPAGDGTFQKWAAVVVPSGE | | NO: 33 | (SEQ ID | (SEQ ID |
| | EQRYTCHVQHEGLPEPVTLRWEPKSCHHHHHH | | | NO: 88) | NO: 93) |
| | (SEQ ID NO: 130) | | | | |
| [EA3 (R202C)]- | LHLEPPKTHVTHHPISDHEATLCCWALGFYPAEITLT | None | SEQ ID | EPKSS | HHHHHH |
| EPKSS-His6 | WQQDGEGHTQDTELVETRPAGDGTFQKWAAVVVPSGE | | NO: 42 | (SEQ ID | (SEQ ID |
| | EQRYTCHVQHEGLPEPVTLRWEPKSSHHHHHH | | | NO: 87) | NO: 93) |
| | (SEQ ID NO: 131) | | | | |
| [EA3 (H192C)]- | LHLEPPKTHVTHCPISDHEATLRCWALGFYPAEITLT | None | SEQ ID | EPKSS | HHHHHH |
| EPKSS-His6 | WQQDGEGHTQDTELVETRPAGDGTFQKWAAVVVPSGE | | NO: 43 | (SEQ ID | (SEQ ID |
| | EQRYTCHVQHEGLPEPVTLRWEPKSSHHHHHH | | | NO: 87) | NO: 93) |
| | (SEQ ID NO: 132) | | | | |
| [EA3 (E232C)]- | LHLEPPKTHVTHHPISDHEATLRCWALGFYPAEITLT | None | SEQ ID | EPKSS | HHHHHH |
| EPKSS-His6 | WQQDGEGHTQDTELVCTRPAGDGTFQKWAAVVVPSGE | | NO: 44 | (SEQ ID | (SEQ ID |
| | EQRYTCHVQHEGLPEPVTLRWEPKSSHHHHHH | | | NO: 87) | NO: 93) |
| | (SEQ ID NO: 133) | | | | |
| [EA3 (R234C)]- | LHLEPPKTHVTHHPISDHEATLRCWALGFYPAEITLT | None | SEQ ID | EPKSS | HHHHHH |
| EPKSS-His6 | WQQDGEGHTQDTELVETCPAGDGTFQKWAAVVVPSGE | | NO: 45 | (SEQ ID | (SEQ ID |
| | EQRYTCHVQHEGLPEPVTLRWEPKSSHHHHHH | | | NO: 87) | NO: 93) |
| | (SEQ ID NO: 134) | | | | |
| [EA3 (D238C)]- | LHLEPPKTHVTHHPISDHEATLRCWALGFYPAEITLT | None | SEQ ID | EPKSS | HHHHHH |
| EPKSS-His6 | WQQDGEGHTQDTELVETRPAGCGTFQKWAAVVVPSGE | | NO: 46 | (SEQ ID | (SEQ ID |
| | EQRYTCHVQHEGLPEPVTLRWEPKSSHHHHHH | | | NO: 87) | NO: 93) |
| | (SEQ ID NO: 135) | | | | |

TABLE 17
Variant β2m Domains: Disulfide Mutations

| | Sequence |
|---|---|
| Identifier | (Mutations relative to WT are bolded and underlined) |
| B2M (SST, M99del) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRD (SEQ ID NO: 19) |
| RTV-[B2M (SST, | RTVRTPKIQVYSRHPAENGKSNFLNCYVSGFHPSDIEVDLLKNGERIEKVEHSDLSSSKDSS |
| M99del)] | TYLLYYTEFTPTEKDEYACRVNHVTLSQPKIVKWDRD (SEQ ID NO: 20) |
| B2M (SST, M99C) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDC (SEQ ID NO: 21) |
| RTV-[B2M (SST, M99C)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDC (SEQ ID NO: 22) |
| B2M (SST, D98C) | RTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRCM (SEQ ID NO: 23) |
| RTV-[B2M (SST, D98C)] | RTVRTPKIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRCM (SEQ ID NO: 24) |
| B2M (SST, K6C) | RTPCIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 25) |
| RTV-[B2M (SST, K6C)] | RTVRTPCIQVYSRHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 26) |
| B2M (SST, Y10C) | RTPKIQVCSRHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 27) |
| RTV-[B2M (SST, Y10C)] | RTVRTPKIQVCSRHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 28) |
| B2M (SST, R12C) | RTPKIQVYSCHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 29) |
| RTV-[B2M (SST, R12C)] | RTVRTPKIQVYSCHPAENGKSNFLNCYVSGFHPSDIEVDLLKNGERIEKVEHSDLSSSKDSS |
| | TYLLYYTEFTPTEKDEYACRVNHVTLSQPKIVKWDRDM (SEQ ID NO: 30) |

TABLE 18
Variant β2m Domain Polypeptides: Disulfide Mutations

| | Polypeptide | | | | |
|---|---|---|---|---|---|
| | (Mutations relative to WT | | | | |
| | are bolded and | | | | |
| Identifier | underlined) | Elbow | Insert | Spacer | C-term Tag |
| RTV- | RTVRTPKIQVYSRHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 19 | G | A |
| M99del)]- | FTPTEKDEYACRVNHVTLSQPKIVKWDRDGGSGEC | | | (SEQ ID | (SEQ ID |
| GGSGECS | SGYPYDVPDYA (SEQ ID NO: 136) | | | NO: 89) | NO: 95) |
| G-HA | | | | | |
| RTV- | RTVRTPKIQVYSRHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 19 | SG | A |
| M99del)]- | FTPTEKDEYACRVNHVTLSQPKIVKWDRDGGGSGE | | | (SEQ ID | (SEQ ID |
| GGGSGEC | CSGYPYDVPDYA (SEQ ID NO: 137) | | | NO: 90) | NO: 95) |
| SG-HA | | | | | |
| RTV- | RTVRTPKIQVYSRHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 21 | G | A |
| M99C)]- | FTPTEKDEYACRVNHVTLSQPKIVKWDRDCGGSGE | | | (SEQ ID | (SEQ ID |
| GGSGECS | SSGYPYDVPDYA (SEQ ID NO: 138) | | | NO: 89) | NO: 95) |
| G-HA | | | | | |
| RTV- | RTVRTPKIQVYSRHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 21 | SG | A |
| M99C]- | FTPTEKDEYACRVNHVTLSQPKIVKWDRDCGGGSG | | | (SEQ ID | (SEQ ID |
| GGGSGEC | ESSGYPYDVPDYA (SEQ ID NO: 139) | | | NO: 90) | NO: 95) |
| SG-HA | | | | | |
| RTV- | RTVRTPKIQVYSRHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 23 | G | A |
| D98C]- | FTPTEKDEYACRVNHVTLSQPKIVKWDRCMGGSGE | | | (SEQ ID | (SEQ ID |
| GGSGECS | SSGYPYDVPDYA (SEQ ID NO: 140) | | | NO: 89) | NO: 95) |
| G-HA | | | | | |
| RTV- | RTVRTPKIQVYSRHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 23 | SG | A |
| D98C]- | FTPTEKDEYACRVNHVTLSQPKIVKWDRCMGGGSG | | | (SEQ ID | (SEQ ID |
| GGGSGEC | ESSGYPYDVPDYA (SEQ ID NO: 141) | | | NO: 90) | NO: 95) |
| SG-HA | | | | | |
| RTV- | RTVRTPCIQVYSRHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 25 | G | A |
| K6C]- | FTPTEKDEYACRVNHVTLSQPKIVKWDRDMGGSGE | | | (SEQ ID | (SEQ ID |
| GGSGECS | SSGYPYDVPDYA (SEQ ID NO: 142) | | | NO: 89) | NO: 95) |
| G-HA | | | | | |
| RTV- | RTVRTPCIQVYSRHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 25 | SG | A |
| K6C]- | FTPTEKDEYACRVNHVTLSQPKIVKWDRDMGGGSG | | | (SEQ ID | (SEQ ID |
| GGGSGEC | ESSGYPYDVPDYA (SEQ ID NO: 143) | | | NO: 90) | NO: 95) |
| SG-HA | | | | | |
| RTV- | RTVRTPKIQVCSRHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 27 | G | A |
| Y10C]- | FTPTEKDEYACRVNHVTLSQPKIVKWDRDMGGSGE | | | (SEQ ID | (SEQ ID |
| GGSGECS | SSGYPYDVPDYA (SEQ ID NO: 144) | | | NO: 89) | NO: 95) |
| G-HA | | | | | |
| RTV- | RTVRTPKIQVCSRHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 27 | SG | A |
| Y10C]- | FTPTEKDEYACRVNHVTLSQPKIVKWDRDMGGGSG | | | (SEQ ID | (SEQ ID |
| GGGSGEC | ESSGYPYDVPDYA (SEQ ID NO: 145) | | | NO: 90) | NO: 95) |
| SG-HA | | | | | |
| RTV- | RTVRTPKIQVYSCHPAENGKSNFLNCYVSGFHPSD | RTV | SEQ ID | GGSGECS | YPYDVPDY |
| [B2M (SST, | IEVDLLKNGERIEKVEHSDLSSSKDSSTYLLYYTE | | NO: 29 | G | A |
| R12C]- | FTPTEKDEYACRVNHVTLSQPKIVKWDRDMGGSGE | | | (SEQ ID | (SEQ ID |
| GGSGECS | SSGYPYDVPDYA (SEQ ID NO: 146) | | | NO: 89) | NO: 95) |
| G-HA | | | | | |
| RTV- | RTVRTPKIQVYSCHPAENGKSNFLNCYVSGFHPSD | RTV | SEQ ID | GGGSGEC | YPYDVPDY |
| [B2M (SST, | IEVDLLKNGERIEKVEHSDLSSSKDSSTYLLYYTE | | NO: 29 | SG | A |
| R12C]- | FTPTEKDEYACRVNHVTLSQPKIVKWDRDMGGGSG | | | (SEQ ID | (SEQ ID |
| GGGSGEC | ESSGYPYDVPDYA (SEQ ID NO: 147) | | | NO: 90) | NO: 95) |
| SG-HA | | | | | |

TABLE 19
Pseudo-Fabs Having HLA-E α3 and β2m Dimerization Domains

| | | | Polypeptide | | | | | |
|---|---|---|---|---|---|---|---|---|
| | | | (Mutations relative | | | | | |
| | | | to WT are | Variable | | | | C-term |
| pFab ID | Chain | Chain ID | bolded and underlined) | Region | Elbow | Insert | Spacer | tag |
| HLPPW42 | HC | [VH]-GGS- | QITLKESGPTLVKPTQTLTLTC | SEQ ID | GGS | SEQ ID | EPKSC | SEQ ID |
| (Native | frag | [EA3]- | TFSGFSLSTSGMGVSWIRQPPG | NO: 96 | | NO: 33 | (SEQ ID | NO: 93 |
| Hinge) | | EPKSC- | KALEWLAHIYWDDDKRYNPSLK | | | | NO: 88) | |
| | | His6 | SRLTITKDTSKNQVVLTMTNMD | | | | | |
| | | | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSCHHHHHH | | | | | |
| | | | (SEQ ID NO: 148) | | | | | |
| | LC | [VL]- | DIVMTQSPDSLAVSLGERATIN | SEQ ID | RTVG | SEQ ID | GGSGEC | YPYDVP |
| | frag | RTVGGSR | CRASQSVDYNGISYMHWYQQKP | NO: 97 | GSRT | NO: 19 | SG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 89) | NO: 95) |
| | | M99del)]- | VYYCQQIIEDPWTFGQGTKVEI | | ID | | | |
| | | GGSGECS | KRTVGGSRTVRTPKIQVYSRHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDGGSG | | | | | |
| | | | ECSGYPYDVPDYA | | | | | |
| | | | (SEQ ID NO: 149) | | | | | |
| HLPPW43 | HC | [VH]-GGS- | QITLKESGPTLVKPTQTLTLTC | SEQ ID | GGS | SEQ ID | EPKSC | SEQ ID |
| (Native | frag | [EA3]- | TFSGFSLSTSGMGVSWIRQPPG | NO: 96 | | NO: 33 | (SEQ ID | NO: 93 |
| Hinge) | | EPKSC- | KALEWLAHIYWDDDKRYNPSLK | | | | NO: 88) | |
| | | His6 | SRLTITKDTSKNQVVLTMTNMD | | | | | |
| | | | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSCHHHHHH | | | | | |
| | | | (SEQ ID NO: 150) | | | | | |
| | LC | [VL]- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 19 | CSG | DYA |
| | | TV- | GQPPKLLIYAASNPESGVPDRF | | V | | (SEQ ID | (SEQ ID |
| | | [B2M | SGSGSGTDFTLTISSLQAEDVA | | (SEQ | | NO: 90) | NO: 95) |
| | | (SST)]- | VYYCQQIIEDPWTFGQGTKVEI | | ID | | | |
| | | GGGSGEC | KRTVGGSRTVRTPKIQVYSRHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDGGGS | | | | | |
| | | | GECSGYPYDVPDYA | | | | | |
| | | | (SEQ ID NO: 151) | | | | | |
| HLPPW54 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 42 | (SEQ ID | (SEQ ID |
| | | (R202C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLCCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 152) | | | | | |
| | LC | [VL]- | DIVMTQSPDSLAVSLGERATIN | SEQ ID | RTVG | SEQ ID | GGSGES | YPYDVP |
| | frag | RTVGGSR | CRASQSVDYNGISYMHWYQQKP | NO: 97 | GSRT | NO: 21 | SG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 91) | NO: 95) |
| | | M99C)]- | VYYCQQIIEDPWTFGQGTKVEI | | ID | | | |
| | | GGSGESS | KRTVGGSRTVRTPKIQVYSRHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDCGGS | | | | | |
| | | | GESSGYPYDVPDYA | | | | | |
| | | | (SEQ ID NO: 153) | | | | | |
| HLPPW56 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 43 | (SEQ ID | (SEQ ID |
| | | (H192C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHCPISDHEATLRCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 154) | | | | | |
| | LC | [VL]- | DIVMTQSPDSLAVSLGERATIN | SEQ ID | RTVG | SEQ ID | GGSGES | YPYDVP |
| | frag | RTVGGSR | CRASQSVDYNGISYMHWYQQKP | NO: 97 | GSRT | NO: 24 | SG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 91) | NO: 95) |
| | | D98C)]- | VYYCQQIIEDPWTFGQGTKVEI | | ID | | | |
| | | GGSGESS | KRTVGGSRTVRTPKIQVYSRHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRCMGGS | | | | | |
| | | | GESSGYPYDVPDYA | | | | | |
| | | | (SEQ ID NO: 155) | | | | | |
| HLPPW48 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 44 | (SEQ ID | (SEQ ID |
| | | (E232C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVC | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 156) | | | | | |
| | LC | [VL]- | DIVMTQSPDSLAVSLGERATIN | SEQ ID | RTVG | SEQ ID | GGSGES | YPYDVP |
| | frag | RTVGGSR | CRASQSVDYNGISYMHWYQQKP | NO: 97 | GSRT | NO: 26 | SG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 91) | NO: 95) |
| | | K6C)]- | VYYCQQIIEDPWTFGQGTKVEI | | ID | | | |
| | | GGSGESS | KRTVGGSRTVRTPCIQVYSRHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGS | | | | | |
| | | | GESSGYPYDVPDYA | | | | | |
| | | | (SEQ ID NO: 157) | | | | | |
| HLPPW50 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 45 | (SEQ ID | (SEQ ID |
| | | (R234C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TCPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 158) | | | | | |
| | LC | [VL]- | DIVMTQSPDSLAVSLGERATIN | SEQ ID | RTVG | SEQ ID | GGSGES | YPYDVP |
| | frag | RTVGGSR | CRASQSVDYNGISYMHWYQQKP | NO: 97 | GSRT | NO: 28 | SG | DYA |
| | | TV-[B2M | GQPPKLLIYAASNPESGVPDRF | | V | | (SEQ ID | (SEQ ID |
| | | (SST, | SGSGSGTDFTLTISSLQAEDVA | | (SEQ | | NO: 91) | NO: 95) |
| | | Y10C)]- | VYYCQQIIEDPWTFGQGTKVEI | | ID | | | |
| | | GGSGESS | KRTVGGSRTVRTPKIQVCSRHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGS | | | | | |
| | | | GESSGYPYDVPDYA | | | | | |
| | | | (SEQ ID NO: 159) | | | | | |
| HLPPW52 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 46 | (SEQ ID | (SEQ ID |
| | | (D238C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TRPAGCGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 160) | | | | | |
| | LC | VL1- | DIVMTQSPDSLAVSLGERATIN | SEQ ID | RTVG | SEQ ID | GGSGES | YPYDVP |
| | frag | RTVGGSR | CRASQSVDYNGISYMHWYQQKP | NO: 97 | GSRT | NO: 30 | SG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 91) | NO: 95) |
| | | R12C)]- | VYYCQQIIEDPWTFGQGTKVEI | | ID | | | |
| | | GGSGESS | KRTVGGSRTVRTPKIQVYSCHP | | NO: | | | |
| | | G-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGS | | | | | |
| | | | GESSGYPYDVPDYA | | | | | |
| | | | (SEQ ID NO: 161) | | | | | |
| HLPPW55 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 42 | (SEQ ID | (SEQ ID |
| | | (R202C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLCCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 162) | | | | | |
| | LC | [VL]- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 22 | SSG | DYA |
| | | TV- | GQPPKLLIYAASNPESGVPDRF | | V | | (SEQ ID | (SEQ ID |
| | | [B2M (SST | SGSGSGTDFTLTISSLQAEDVA | | (SEQ | | NO: 92) | NO: 95) |
| | | M99C)]- | VYYCQQIIEDPWTFGQGTKVEI | | ID | | | |
| | | GGGSGES | KRTVGGSRTVRTPKIQVYSRHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDCGGG | | | | | |
| | | | SGESSGYPYDVPDYA | | | | | |
| | | | (SEQ ID NO: 163) | | | | | |
| HLPPW57 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 43 | (SEQ ID | (SEQ ID |
| | | (H192C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHCPISDHEATLRCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 164) | | | | | |
| | LC | VL1- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 24 | SSG | DYA |
| | | TV- | GQPPKLLIYAASNPESGVPDRF | | V | | (SEQ ID | (SEQ ID |
| | | B2M (SST, | SGSGSGTDFTLTISSLQAEDVA | | (SEQ | | NO: 9) | NO: 95) |
| | | D98C)- | VYYCQQIIEDPWTFGQGTKVEI | | ID | | | |
| | | GGGSGES | KRTVGGSRTVRTPKIQVYSRHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRCMGGG | | | | | |
| | | | SGESSGYPYDVPDYA | | | | | |
| | | | (SEQ ID NO: 165) | | | | | |
| HLPPW49 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 44 | (SEQ ID | (SEQ ID |
| | | (E232C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVC | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 166) | | | | | |
| | LC | [VL]- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 26 | SSG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 9) | NO: 95) |
| | | K6C)]- | VYYCQQIIEDPWTFGQGTKVEI | | ID | | | |
| | | GGGSGES | KRTVGGSRTVRTPCIQVYSRHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGG | | | | | |
| | | | SGESSGYPYDVPDYA | | | | | |
| | | | (SEQ ID NO: 167) | | | | | |
| HLPPW51 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 45 | (SEQ ID | (SEQ ID |
| | | (R234C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TCPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 168) | | | | | |
| | LC | [VL]- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 28 | SSG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 9) | NO: 95) |
| | | Y10C)]- | VYYCQQIIEDPWTFGQGTKVEI | | ID | | | |
| | | GGGSGES | KRTVGGSRTVRTPKIQVCSRHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGG | | | | | |
| | | | SGESSGYPYDVPDYA | | | | | |
| | | | (SEQ ID NO: 169) | | | | | |
| HLPPW53 | HC | [VH]-GGS- | QITLKESGPTLVKPTQTLTLTC | SEQ ID | GGS | SEQ ID | EPKSS | HHHHHH |
| | frag | [EA3 | TFSGFSLSTSGMGVSWIRQPPG | NO: 96 | | NO: 46 | (SEQ ID | (SEQ ID |
| | | (D238C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSGGSLHLEPPKTH | | | | | |
| | | | VTHHPISDHEATLRCWALGFYP | | | | | |
| | | | AEITLTWQQDGEGHTQDTELVE | | | | | |
| | | | TRPAGCGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 170) | | | | | |
| | LC | [VL]- | DIVMTQSPDSLAVSLGERATIN | SEQ ID | RTVG | SEQ ID | GGGSGE | YPYDVP |
| | frag | RTVGGSR | CRASQSVDYNGISYMHWYQQKP | NO: 97 | GSRT | NO: 30 | SSG | DYA |
| | | TV- | GQPPKLLIYAASNPESGVPDRF | | V | | (SEQ ID | (SEQ ID |
| | | [B2M (SST, | SGSGSGTDFTLTISSLQAEDVA | | (SEQ | | NO: 9) | NO: 95) |
| | | R12C)]- | VYYCQQIIEDPWTFGQGTKVEI | | ID | | | |
| | | GGGSGES | KRTVGGSRTVRTPKIQVYSCHP | | NO: | | | |
| | | SG-HA | AENGKSNFLNCYVSGFHPSDIE | | 60) | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRDMGGG | | | | | |
| | | | SGESSGYPYDVPDYA | | | | | |
| | | | (SEQ ID NO: 171) | | | | | |
| HLPPW58 | HC | [VH]- | QITLKESGPTLVKPTQTLTLTC | SEQ ID | None | ASTKGP | EPKSC | HHHHHH |
| | frag | [hIgG1HC] | TFSGFSLSTSGMGVSWIRQPPG | NO: 96 | | SVFPLA | (SEQ ID | (SEQ ID |
| | | -EPKSC- | KALEWLAHIYWDDDKRYNPSLK | | | PSSKST | NO: 88) | NO: 93) |
| | | His6 | SRLTITKDTSKNQVVLTMTNMD | | | SGGTAA | | |
| | | | PVDTATYYCARLYGFTYGFAYW | | | LGCLVK | | |
| | | | GQGTLVTVSSASTKGPSVFPLA | | | DYFPEP | | |
| | | | PSSKSTSGGTAALGCLVKDYFP | | | VTVSWN | | |
| | | | EPVTVSWNSGALTSGVHTFPAV | | | SGALTS | | |
| | | | LQSSGLYSLSSVVTVPSSSLGT | | | GVHTFP | | |
| | | | QTYICNVNHKPSNTKVDKKVEP | | | AVLQSS | | |
| | | | KSCHHHHHH | | | GLYSLS | | |
| | | | (SEQ ID NO: 172) | | | SVVTVP | | |
| | | | | | | SSSLGT | | |
| | | | | | | QTYICN | | |
| | | | | | | VNHKPS | | |
| | | | | | | NTKVDK | | |
| | | | | | | KV | | |
| | | | | | | (SEQ ID | | |
| | | | | | | NO: 98) | | |
| | LC | [VL]-[hIg | DIVMTQSPDSLAVSLGERATIN | SEQ ID | RTV | RTVAAP | GS | YPYDVP |
| | frag | Kappa]- | CRASQSVDYNGISYMHWYQQKP | NO: 97 | | SVFIFP | | DYA |
| | | GS-HA | GQPPKLLIYAASNPESGVPDRF | | | PSDEQL | | (SEQ ID |
| | | | SGSGSGTDFTLTISSLQAEDVA | | | KSGTAS | | NO: 95) |
| | | | VYYCQQIIEDPWTFGQGTKVEI | | | VVCLLN | | |
| | | | KRTVRTVAAPSVFIFPPSDEQL | | | NFYPRE | | |
| | | | KSGTASVVCLLNNFYPREAKVQ | | | AKVQWK | | |
| | | | WKVDNALQSGNSQESVTEQDSK | | | VDNALQ | | |
| | | | DSTYSLSSTLTLSKADYEKHKV | | | SGNSQE | | |
| | | | YACEVTHQGLSSPVTKSENRGE | | | SVTEQD | | |
| | | | CGSYPYDVPDYA | | | SKDSTY | | |
| | | | (SEQ ID NO: 173) | | | SLSSTL | | |
| | | | | | | TLSKAD | | |
| | | | | | | YEKHKV | | |
| | | | | | | YACEVT | | |
| | | | | | | HQGLSS | | |
| | | | | | | PVTKSF | | |
| | | | | | | NRGEC | | |
| | | | | | | (SEQ ID | | |
| | | | | | | NO: 99) | | |

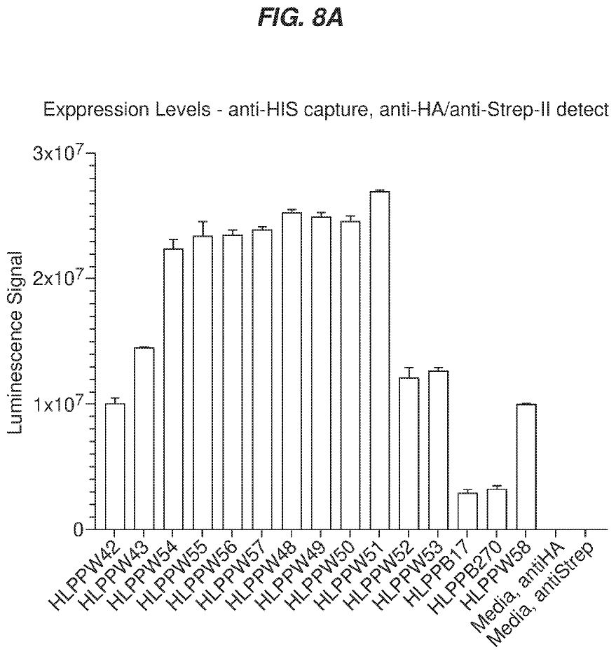

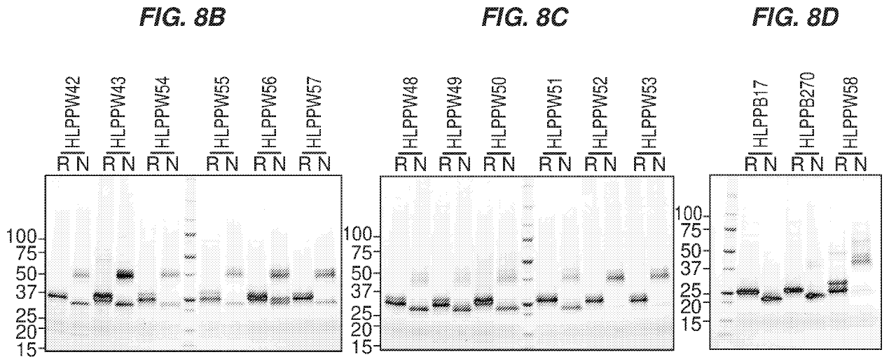

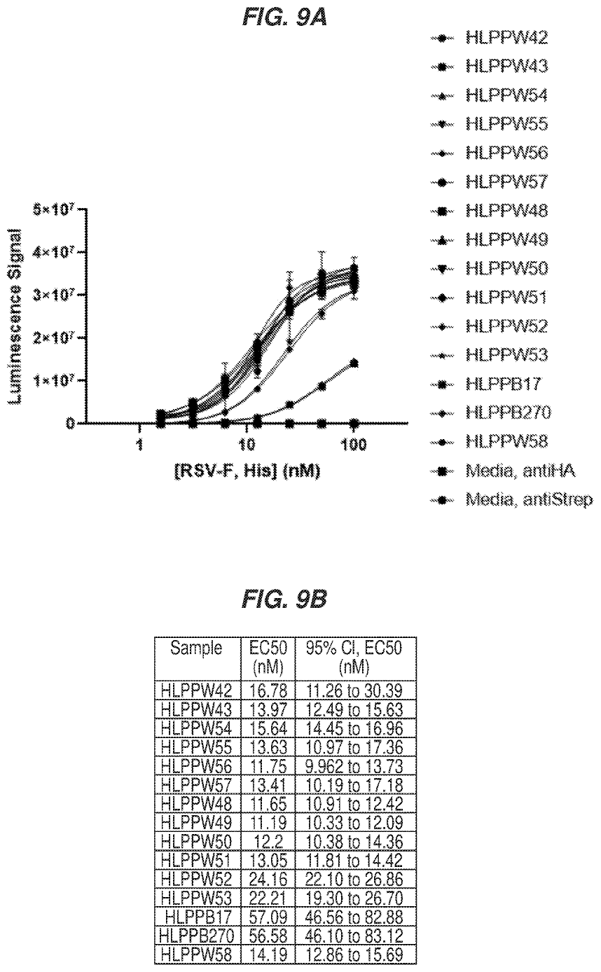

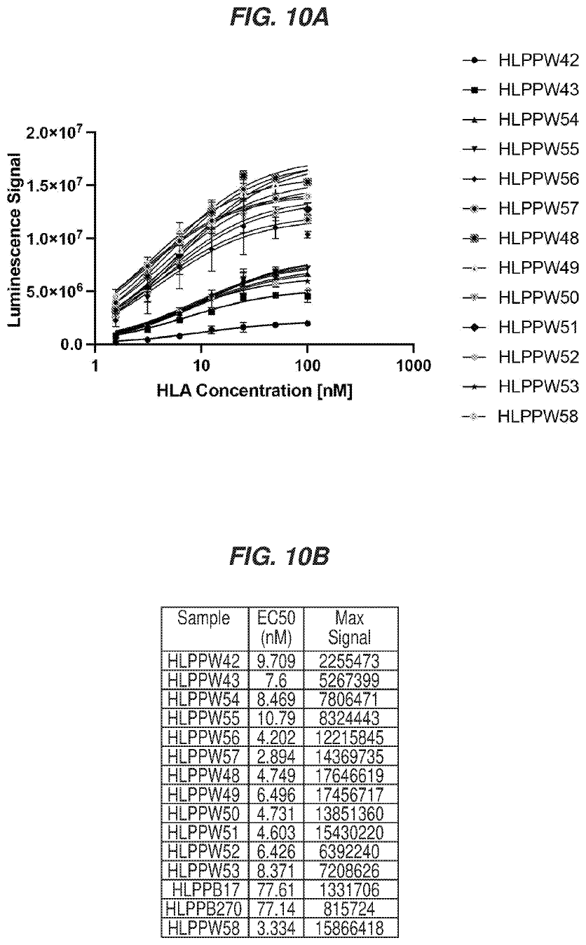

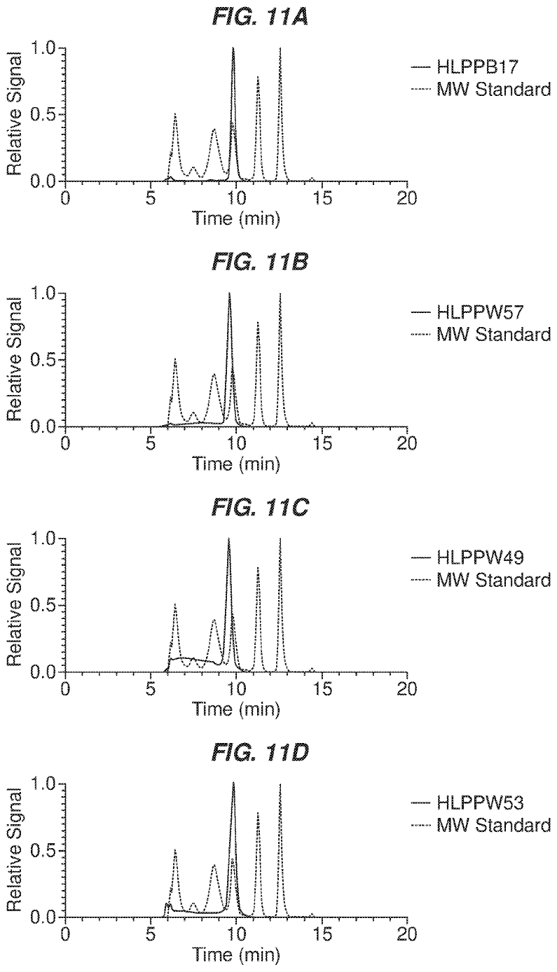

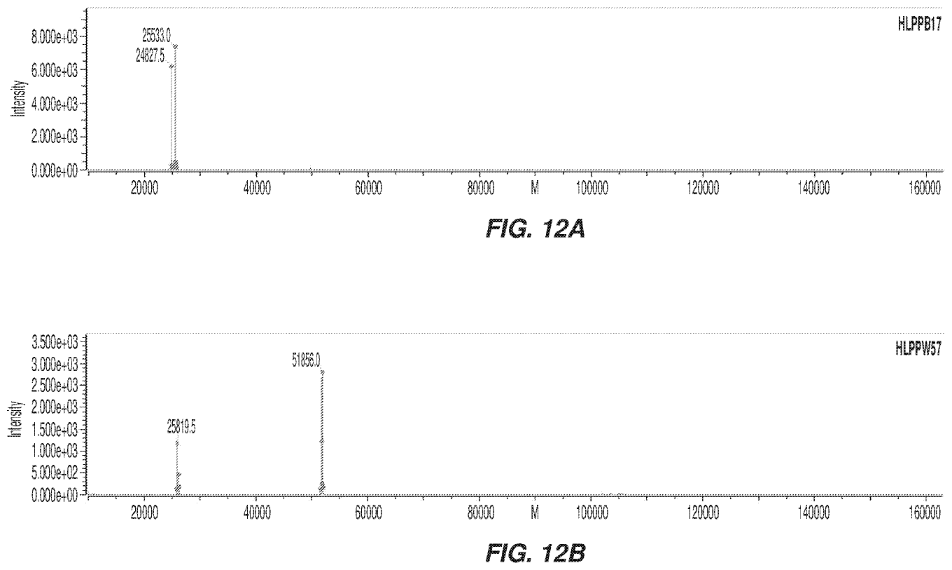

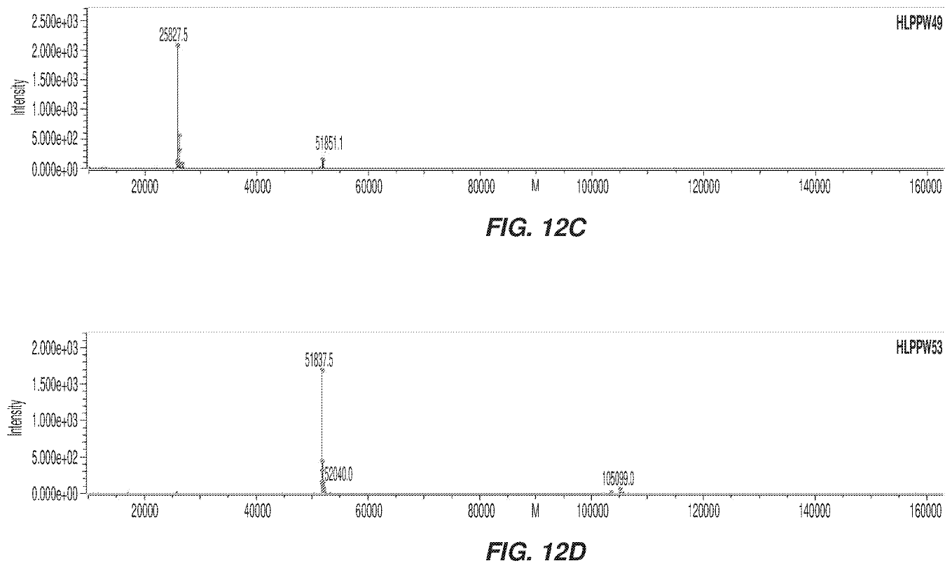

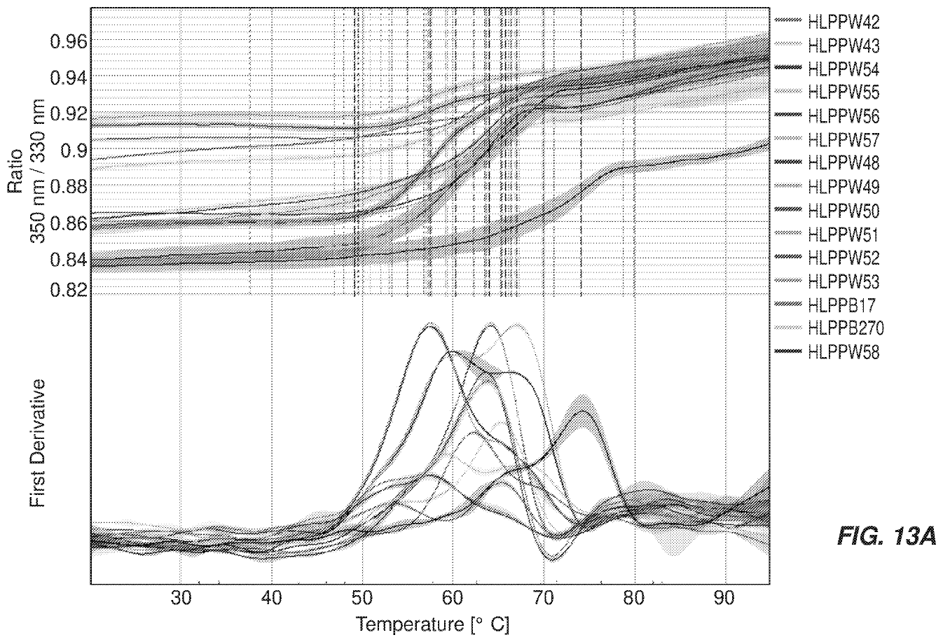

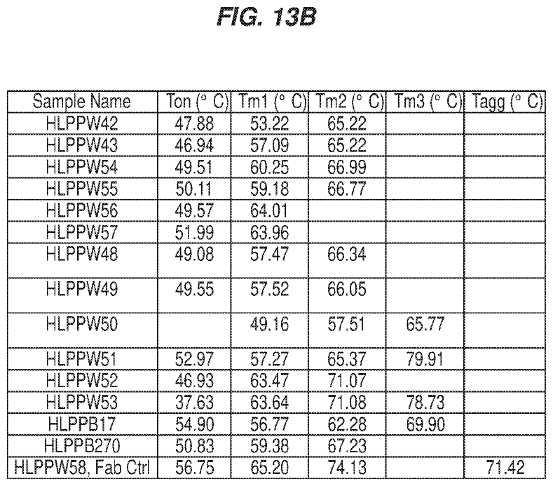

Disulfide-engineered pseudo-Fabs were tested as to: A) dimerization efficiency; B) whether expression titers were similar to a (native) Fab (FIGS. 8A-8D); C) target binding (FIGS. 9A-9B and 10A-10B); D) high monodispersity by SEC (FIG. 11); E) efficient disulfide bond formation by mass spectrometry (FIG. 12), and F) having melting temperatures amenable to therapeutic development as measured by differential scanning fluorimetry (FIGS. 13A-13B).

The results for the pseudo-Fab designs featuring the HLA-E α3 domain/β2m are summarized below. Expression of pseudo-Fab molecules HLPPW42, HLPPW43, HLPPW54, HLPPW55, HLPPW56, HLPPW57, HLPPW48, HLPPW49, HLPPW50, HLPPW51, HLPPW52, HLPPW53, and HLPPW58 (normal Fab reference) is shown in FIGS. 8A-8D along with pseudo-Fabs HLPPB17 and HLPPB270. Most designs expressed at levels similar to a normal Fab (HLPPW58), except for HLPPW42 and HLPPW43, which contained HLA-E α3 native hinge cysteines. Several disulfide-bonded HLA-E α3/β2m D1 heterodimers produced pseudo-Fabs that displayed binding identical to the normal Fab (FIGS. 9A-9B). Most designs, except for HLPPW48, HLPPW49, and HLPPB50 showed efficient disulfide-induced dimerization. HLPPW57 comprising HLA-E A3 (H192C) and B2M (SST, D98C) was selected for further analysis since it displayed favorable dimerization, titer, target (antigen) binding, % monomer (of heterodimer), and thermal stability similar to the native Fab having an identical variable region. A summary of the results for HLA-E α3 and β2m Disulfide Variant pFabs is shown in TABLE 20.

TABLE 20
Summary of Results for HLA-E α3 and β2m Disulfide Variant pFabs

| | | | Target | | | | |
|---|---|---|---|---|---|---|---|
| | | Titer | Binding EC50 | % Monomer | Ton | Tm1 | Tm2 |
| Name | Dimerization | (mg/mL) | (nM) | (SEC) | (° C.) | (° C.) | (° C.) |
| HLPPW42 | + | 0.76 | 9.71 | 45.7 | 47.88 | 53.22 | 65.22 |
| HLPPW43 | + | 0.74 | 7.6 | 90.4 | 46.94 | 57.09 | 65.22 |
| HLPPW54 | + | 1.46 | 8.47 | 76.8 | 49.51 | 60.25 | 66.99 |
| HLPPW55 | + | 2.14 | 10.79 | 66.6 | 50.11 | 59.18 | 66.77 |
| HLPPW56 | ++ | 2.28 | 4.2 | 84.6 | 49.57 | 64.01 | |
| HLPPW57 | ++ | 3.42 | 2.89 | 86 | 51.99 | 63.96 | |
| HLPPW48 | − | 2.17 | 4.75 | 59.5 | 49.08 | 57.47 | 66.34 |
| HLPPW49 | − | 2.07 | 6.5 | 58.9 | 49.55 | 57.52 | 66.05 |
| HLPPW50 | − | 3.68 | 4.73 | 24.9 | | 49.16 | 57.51 |
| HLPPW51 | + | 4.58 | 4.6 | 27.1 | 52.97 | 57.27 | 65.37 |
| HLPPW52 | ++ | 0.64 | 6.43 | 86.4 | 46.93 | 63.47 | 71.07 |
| HLPPW53 | ++ | 0.69 | 8.37 | 91.7 | 37.63 | 63.64 | 71.08 |
| HLPPW58 | ++ | 4.27 | 3.33 | 66.8 | 56.75 | 65.20 | 74.13 |

The disulfide bonded variants which displayed favorable properties were then used as a starting point to optimize the “elbow” sequence that connected the variable domain to the “pseudo constant domains”. Using HLPPW57 as a template, a set of variants was designed to determine the optimal elbow sequence between the variable domain and constant domain of the pseudo-Fabs. Affinity tags were appended onto the C-terminus of the variant polypeptides along with spacers N-terminal to the affinity tags (TABLE 21). Pseudo-Fabs were assembled from HLA-E α3 (H192C) and B2M (SST, D98C) elbow variants with the sequences shown in TABLE 23.

TABLE 21
Elbow HLA-E α3 Variant Polypeptides

| | Polypeptide | | | | |
|---|---|---|---|---|---|
| | (Mutations relative to | | | | |
| Identifier | WT are bolded and underlined) | Elbow | Insert | Spacer | Tag |
| AST-[EA3 | ASTLHLEPPKTHVTHCPISDHEATLRCWALGFYPAEI | AST | SEQ ID | EPKSS | HHHHHH |
| (H192C)]-EPKSS- | TLTWQQDGEGHTQDTELVETRPAGDGTFQKWAAVVVP | | NO: 43 | (SEQ ID | (SEQ ID |
| His6 | SGEEQRYTCHVQHEGLPEPVTLRWEPKSSHHHHHH | | | NO: 87) | NO: 93) |
| | (SEQ ID NO: 174) | | | | |
| ASTK-[EA3 | ASTKLHLEPPKTHVTHCPISDHEATLRCWALGFYPAE | ASTK | SEQ ID | EPKSS | HHHHHH |
| (H192C)]-EPKSS- | ITLTWQQDGEGHTQDTELVETRPAGDGTFQKWAAVVV | (SEQ ID NO: | NO: 43 | (SEQ ID | (SEQ ID |
| His6 | PSGEEQRYTCHVQHEGLPEPVTLRWEPKSSHHHHHH | 61) | | NO: 87) | NO: 93) |
| | (SEQ ID NO: 175) | | | | |
| ASTKG-[EA3 | ASTKGLHLEPPKTHVTHCPISDHEATLRCWALGFYPA | ASTKG | SEQ ID | EPKSS | HHHHHH |
| (H192C)]-EPKSS- | EITLTWQQDGEGHTQDTELVETRPAGDGTFQKWAAVV | (SEQ ID NO: | NO: 43 | (SEQ ID | (SEQ ID |
| His6 | VPSGEEQRYTCHVQHEGLPEPVTLRWEPKSSHHHHHH | 62) | | NO: 87) | NO: 93) |
| | (SEQ ID NO: 176) | | | | |
| ASTKGG-[EA3 | ASTKGGLHLEPPKTHVTHCPISDHEATLRCWALGFYP | ASTKGG | SEQ ID | EPKSS | HHHHHH |
| (H192C)]-EPKSS- | AEITLTWQQDGEGHTQDTELVETRPAGDGTFQKWAAV | (SEQ ID NO: | NO: 43 | (SEQ ID | (SEQ ID |
| His6 | VVPSGEEQRYTCHVQHEGLPEPVTLRWEPKSSHHHHH | 63) | | NO: 87) | NO: 93) |
| | H (SEQ ID NO: 177) | | | | |
| ASTKGGS-[EA3 | ASTKGGSLHLEPPKTHVTHCPISDHEATLRCWALGFY | ASTKGGS | SEQ ID | EPKSS | HHHHHH |
| (H192C)]-EPKSS- | PAEITLTWQQDGEGHTQDTELVETRPAGDGTFQKWAA | (SEQ ID NO: | NO: 43 | (SEQ ID | (SEQ ID |
| His6 | VVVPSGEEQRYTCHVQHEGLPEPVTLRWEPKSSHHHH | 64) | | NO: 87) | NO: 93) |
| | HH (SEQ ID NO: 178) | | | | |
| ASTKGGGS-[EA3 | ASTKGGGSLHLEPPKTHVTHCPISDHEATLRCWALGF | ASTKGGGS | SEQ ID | EPKSS | HHHHHH |
| (H192C)]-EPKSS- | YPAEITLTWQQDGEGHTQDTELVETRPAGDGTFQKWA | (SEQ ID NO: | NO: 43 | (SEQ ID | (SEQ ID |
| His6 | AVVVPSGEEQRYTCHVQHEGLPEPVTLRWEPKSSHHH | 65) | | NO: 87) | NO: 93) |
| | HHH (SEQ ID NO: 179) | | | | |
| ASTKG₄S-[EA3 | ASTKGGGGSLHLEPPKTHVTHCPISDHEATLRCWALG | ASTKGGGG | SEQ ID | EPKSS | HHHHHH |
| (H192C)]-EPKSS- | FYPAEITLTWQQDGEGHTQDTELVETRPAGDGTFQKW | S | NO: 43 | (SEQ ID | (SEQ ID |
| His6 | AAVVVPSGEEQRYTCHVQHEGLPEPVTLRWEPKSSHH | (SEQ ID NO: | | NO: 87) | NO: 93) |
| | HHHH (SEQ ID NO: 180) | 66) | | | |
| ASTKG₄SG-[EA3 | ASTKGGGGSGLHLEPPKTHVTHCPISDHEATLRCWAL | ASTKGGGG | SEQ ID | EPKSS | HHHHHH |
| (H192C)]-EPKSS- | GFYPAEITLTWQQDGEGHTQDTELVETRPAGDGTFQK | SG | NO: 43 | (SEQ ID | (SEQ ID |
| His6 | WAAVVVPSGEEQRYTCHVQHEGLPEPVTLRWEPKSSH | (SEQ ID NO: | | NO: 87) | NO: 93) |
| | HHHHH (SEQ ID NO: 181) | 67) | | | |
| ASTKG₄SGG-[EA3 | ASTKGGGGSGGLHLEPPKTHVTHCPISDHEATLRCWA | ASTKGGGG | SEQ ID | EPKSS | HHHHHH |
| (H192C)]-EPKSS- | LGFYPAEITLTWQQDGEGHTQDTELVETRPAGDGTFQ | SGG | NO: 43 | (SEQ ID | (SEQ ID |
| His6 | KWAAVVVPSGEEQRYTCHVQHEGLPEPVTLRWEPKSS | (SEQ ID NO: | | NO: 87) | NO: 93) |
| | HHHHHH (SEQ ID NO: 182) | 68) | | | |
| ASTKG₄SGGS- | ASTKGGGGSGGSLHLEPPKTHVTHCPISDHEATLRCW | ASTKGGGG | SEQ ID | EPKSS | HHHHHH |
| [EA3 (H192C)]- | ALGFYPAEITLTWQQDGEGHTQDTELVETRPAGDGTF | SGGS | NO: 43 | (SEQ ID | (SEQ ID |
| EPKSS-His6 | QKWAAVVVPSGEEQRYTCHVQHEGLPEPVTLRWEPKS | (SEQ ID NO: | | NO: 87) | NO: 93) |
| | SHHHHHH (SEQ ID NO: 183) | 69) | | | |
| ASTKG₄SGGGS- | ASTKGGGGSGGGSLHLEPPKTHVTHCPISDHEATLRC | ASTKGGGG | SEQ ID | EPKSS | HHHHHH |
| [EA3 (H192C)]- | WALGFYPAEITLTWQQDGEGHTQDTELVETRPAGDGT | SGGGS | NO: 43 | (SEQ ID | (SEQ ID |
| EPKSS-His6 | FQKWAAVVVPSGEEQRYTCHVQHEGLPEPVTLRWEPK | (SEQ ID NO: | | NO: 87) | NO: 93) |
| | SSHHHHHH (SEQ ID NO: 184) | 70) | | | |
| ASTK(G₄S),-[EA3 | ASTKGGGGSGGGGSLHLEPPKTHVTHCPISDHEATLR | ASTKGGGG | SEQ ID | EPKSS | HHHHHH |
| (H192C)]-EPKSS- | CWALGFYPAEITLTWQQDGEGHTQDTELVETRPAGDG | SGGGGS | NO: 43 | (SEQ ID | (SEQ ID |
| His6 | TFQKWAAVVVPSGEEQRYTCHVQHEGLPEPVTLRWEP | (SEQ ID NO: | | NO: 87) | NO: 93) |
| | KSSHHHHHH (SEQ ID NO: 185) | 71 | | | |

TABLE 22
Elbow-β2m Variant Polypeptides

| | Polypeptide | | | | |
|---|---|---|---|---|---|
| | (Mutations relative to WT | | | | |
| Identifier | are bolded and underlined) | Elbow | Insert | Spacer | Tag |
| RTV- | RTVRTPKIQVYSRHPAENGKSNFLNCYVSGFHPSDIEVDL | RTV | SEQ ID | GS | YPYDVP |
| [B2M (SST, | LKNGERIEKVEHSDLSSSKDSSTYLLYYTEFTPTEKDEYA | | NO: 24 | | DYA |
| D98C)]-GS-HA | CRVNHVTLSQPKIVKWDRCMGSYPYDVPDYA | | | | (SEQ ID |
| | (SEQ ID NO: 186) | | | | NO: 95) |
| RTVA- | RTVARTPKIQVYSRHPAENGKSNFLNCYVSGFHPSDIEVD | RTVA | SEQ ID | GS | YPYDVP |
| [B2M (SST, | LLKNGERIEKVEHSDLSSSKDSSTYLLYYTEFTPTEKDEY | (SEQ ID | NO: 24 | | DYA |
| D98C)]-GS-HA | ACRVNHVTLSQPKIVKWDRCMGSYPYDVPDYA | NO: 72) | | | (SEQ ID |
| | (SEQ ID NO: 187) | | | | NO: 95) |
| RTVAG- | RTVAGRTPKIQVYSRHPAENGKSNFLNCYVSGFHPSDIEV | RTVAG | SEQ ID | GS | YPYDVP |
| [B2M (SST, | DLLKNGERIEKVEHSDLSSSKDSSTYLLYYTEFTPTEKDE | (SEQ ID | NO: 24 | | DYA |
| D98C)]-GS-HA | YACRVNHVTLSQPKIVKWDRCMGSGYPYDVPDYA | NO: 73) | | | (SEQ ID |
| -HA | (SEQ ID NO: 188) | | | | NO: 95) |
| RTVAGG- | RTVAGGRTPKIQVYSRHPAENGKSNFLNCYVSGFHPSDIE | RTVAGG | SEQ ID | GS | YPYDVP |
| [B2M (SST, | VDLLKNGERIEKVEHSDLSSSKDSSTYLLYYTEFTPTEKD | (SEQ ID | NO: 24 | | DYA |
| D98C)]-GS-HA | EYACRVNHVTLSQPKIVKWDRCMGSYPYDVPDYA | NO: 74) | | | (SEQ ID |
| | (SEQ ID NO: 189) | | | | NO: 95) |
| RTVAGGS- | RTVAGGSRTPKIQVYSRHPAENGKSNFLNCYVSGFHPSDI | RTVAGGS | SEQ ID | GS | YPYDVP |
| [B2M (SST, | EVDLLKNGERIEKVEHSDLSSSKDSSTYLLYYTEFTPTEK | (SEQ ID | NO: 24 | | DYA |
| D98C)]-GS-HA | DEYACRVNHVTLSQPKIVKWDRCMGSYPYDVPDYA | NO: 75) | | | (SEQ ID |
| | (SEQ ID NO: 190) | | | | NO: 95) |
| RTVAGGGS- | RTVAGGGSRTPKIQVYSRHPAENGKSNFLNCYVSGFHPSD | RTVAGGGS | SEQ ID | GS | YPYDVP |
| [B2M (SST, | IEVDLLKNGERIEKVEHSDLSSSKDSSTYLLYYTEFTPTE | (SEQ ID | NO: 24 | | DYA |
| D98C)]-GS-HA | KDEYACRVNHVTLSQPKIVKWDRCMGSYPYDVPDYA | NO: 76) | | | (SEQ ID |
| | (SEQ ID NO: 191) | | | | NO: 95) |
| RTVAG₄S- | RTVAGGGGSRTPKIQVYSRHPAENGKSNFLNCYVSGFHPS | RTVAGGGG | SEQ ID | GS | YPYDVP |
| [B2M (SST, | DIEVDLLKNGERIEKVEHSDLSSSKDSSTYLLYYTEFTPT | S | NO: 24 | | DYA |
| D98C)]-GS-HA | EKDEYACRVNHVTLSQPKIVKWDRCMGSYPYDVPDYA | (SEQ ID | | | (SEQ ID |
| | (SEQ ID NO: 192) | NO: 77) | | | NO: 95) |
| RTVAG SG- | RTVAGGGGSGRTPKIQVYSRHPAENGKSNFLNCYVSGFHP | RTVAGGGG | SEQ ID | GS | YPYDVP |
| [B2M (SST, | SDIEVDLLKNGERIEKVEHSDLSSSKDSSTYLLYYTEFTP | SG | NO: 24 | | DYA |
| D98C)]-GS-HA | TEKDEYACRVNHVTLSQPKIVKWDRCMGSYPYDVPDYA | (SEQ ID | | | (SEQ ID |
| | (SEQ ID NO: 193) | NO: 78) | | | NO: 95) |
| RTVAG₄SGG- | RTVAGGGGSGGRTPKIQVYSRHPAENGKSNFLNCYVSGFH | RTVAGGGG | SEQ ID | GS | YPYDVP |
| [B2M (SST, | PSDIEVDLLKNGERIEKVEHSDLSSSKDSSTYLLYYTEFT | SGG | NO: 24 | | DYA |
| D98C)]-GS-HA | PTEKDEYACRVNHVTLSQPKIVKWDRCMGSYPYDVPDYA | (SEQ ID | | | (SEQ ID |
| | (SEQ ID NO: 194) | NO: 79) | | | NO: 95) |
| RTVAG₄SGGS- | RTVAGGGGSGGSRTPKIQVYSRHPAENGKSNFLNCYVSGF | RTVAGGGG | SEQ ID | GS | YPYDVP |
| [B2M (SST, | HPSDIEVDLLKNGERIEKVEHSDLSSSKDSSTYLLYYTEF | SGGS | NO: 24 | | DYA |
| D98C)]-GS-HA | TPTEKDEYACRVNHVTLSQPKIVKWDRCMGSYPYDVPDYA | (SEQ ID | | | (SEQ ID |
| | (SEQ ID NO: 195) | NO: 80) | | | NO: 95) |
| RTVAG₄SGGG | RTVAGGGGSGGGSRTPKIQVYSRHPAENGKSNFLNCYVSG | RTVAGGGG | SEQ ID | GS | YPYDVP |
| S-[B2M (SST, | FHPSDIEVDLLKNGERIEKVEHSDLSSSKDSSTYLLYYTE | SGGGS | NO: 24 | | DYA |
| D98C)]-GS-HA | FTPTEKDEYACRVNHVTLSQPKIVKWDRCMGSYPYDVPDY | (SEQ ID | | | |
| | A (SEQ ID NO: 196) | NO: 81) | | | (SEQ ID |
| | | | | | NO: 95) |
| RTVAAG₄SG₄S | RTVAGGGGSGGGGSRTPKIQVYSRHPAENGKSNFLNCYVS | RTVAGGGG | SEQ ID | GS | YPYDVP |
| -[B2M (SST, | GFHPSDIEVDLLKNGERIEKVEHSDLSSSKDSSTYLLYYT | SGGGGS | NO: 24 | | DYA |
| D98C)]-GS-HA | EFTPTEKDEYACRVNHVTLSQPKIVKWDRCMGSYPYDVPD | (SEQ ID | | | (SEQ ID |
| | YA (SEQ ID NO: 197) | NO: 82) | | | NO: 95) |

TABLE 23
Pseudo-Fabs Having HLA-E α3 and β2m Domains

| | | | Polypeptide | | | | | |
|---|---|---|---|---|---|---|---|---|
| | | | (Mutations relative | | | | | |
| | | | to WT are | Variable | | | | C-term |
| pFab ID | Chain | Chain ID | bolded and underlined) | Region | Elbow | Insert | Spacer | tag |
| HLPPW59 | HC | [VH]-AST- | QITLKESGPTLVKPTQTLTLTC | SEQ ID | AST | SEQ ID | EPKSS | HHHHHH |
| | frag | [EA3 | TFSGFSLSTSGMGVSWIRQPPG | NO: 96 | | NO: 43 | (SEQ ID | (SEQ ID |
| | | (H192C)]- | KALEWLAHIYWDDDKRYNPSLK | | | | NO: 87) | NO: 93) |
| | | EPKSS- | SRLTITKDTSKNQVVLTMTNMD | | | | | |
| | | His6 | PVDTATYYCARLYGFTYGFAYW | | | | | |
| | | | GQGTLVTVSSASTLHLEPPKTH | | | | | |
| | | | VTHCPISDHEATLRCWALGFYP | | | | | |
| | | | AEITLTWQQDGEGHTQDTELVE | | | | | |
| | | | TRPAGDGTFQKWAAVVVPSGEE | | | | | |
| | | | QRYTCHVQHEGLPEPVTLRWEP | | | | | |
| | | | KSSHHHHHH | | | | | |
| | | | (SEQ ID NO: 198) | | | | | |
| | LC | [VL]-RTV- | DIVMTQSPDSLAVSLGERATIN | SEQ ID | RTV | SEQ ID | GS | YPYDVP |
| | frag | [B2M (SST | CRASQSVDYNGISYMHWYQQKP | NO: 97 | | NO: 24 | | DYA |
| | | D98C)]- | GQPPKLLIYAASNPESGVPDRF | | | | | (SEQ ID |
| | | GGGSGES | SGSGSGTDFTLTISSLQAEDVA | | | | | NO: 95) |
| | | SG-HA | VYYCQQIIEDPWTFGQGTKVEI | | | | | |
| | | | KRTVRTPKIQVYSRHPAENGKS | | | | | |
| | | | NFLNCYVSGFHPSDIEVDLLKN | | | | | |
| | | | GERIEKVEHSDLSSSKDSSTYL | | | | | |
| | | | LYYTEFTPTEKDEYACRVNHVT | | | | | |
| | | | LSQPKIVKWDRCMGSYPYDVPD | | | | | |
| | | | YA (SEQ ID NO: 199) | | | | | |
| HLPPW60 | HC | [VH]- | QITLKESGPTLVKPTQTLTLTC | SEQ ID | ASTK | SEQ ID | EPKSS | HHHHHH |
| | frag | ASTK- | TFSGFSLSTSGMGVSWIRQPPG | NO: 96 | (SEQ | NO: 43 | (SEQ ID | (SEQ ID |
| | | [EA3 | KALEWLAHIYWDDDKRYNPSLK | | ID NO: | | NO: 87) | NO: 93) |
| | | (H192C)]- | SRLTITKDTSKNQVVLTMTNMD | | 61) | | | |
| | | EPKSS- | PVDTATYYCARLYGFTYGFAYW | | | | | |
| | | His6 | GQGTLVTVSSASTKLHLEPPKT | | | | | |
| | | | HVTHCPISDHEATLRCWALGFY | | | | | |
| | | | PAEITLTWQQDGEGHTQDTELV | | | | | |
| | | | ETRPAGDGTFQKWAAVVVPSGE | | | | | |
| | | | EQRYTCHVQHEGLPEPVTLRWE | | | | | |
| | | | PKSSHHHHHH | | | | | |
| | | | (SEQ ID NO: 200) | | | | | |
| | LC | [VL]- | DIVMTQSPDSLAVSLGERATIN | SEQ ID | RTVA | SEQ ID | GS | YPYDVP |
| | frag | RTVA- | CRASQSVDYNGISYMHWYQQKP | NO: 97 | (SEQ | NO: 24 | | DYA |
| | | [B2M (SST, | GQPPKLLIYAASNPESGVPDRF | | ID NO: | | | (SEQ ID |
| | | D98C)]- | SGSGSGTDFTLTISSLQAEDVA | | 72) | | | NO: 95) |
| | | GGGSGES | VYYCQQIIEDPWTFGQGTKVEI | | | | | |
| | | SG-HA | KRTVARTPKIQVYSRHPAENGK | | | | | |
| | | | SNFLNCYVSGFHPSDIEVDLLK | | | | | |
| | | | NGERIEKVEHSDLSSSKDSSTY | | | | | |
| | | | LLYYTEFTPTEKDEYACRVNHV | | | | | |
| | | | TLSQPKIVKWDRCMGSYPYDVP | | | | | |
| | | | DYA (SEQ ID NO: 201) | | | | | |
| HLPPW61 | HC | [VH]- | QITLKESGPTLVKPTQTLTLTC | SEQ ID | ASTK | SEQ ID | EPKSS | HHHHHH |
| | frag | ASTKG- | TFSGFSLSTSGMGVSWIRQPPG | NO: 96 | G | NO: 43 | (SEQ ID | (SEQ ID |
| | | [EA3 | KALEWLAHIYWDDDKRYNPSLK | | (SEQ | | NO: 87) | NO: 93) |
| | | (H192C)]- | SRLTITKDTSKNQVVLTMTNMD | | ID NO: | | | |
| | | EPKSS- | PVDTATYYCARLYGFTYGFAYW | | 62) | | | |
| | | His6 | GQGTLVTVSSASTKGLHLEPPK | | | | | |
| | | | THVTHCPISDHEATLRCWALGF | | | | | |
| | | | YPAEITLTWQQDGEGHTQDTEL | | | | | |
| | | | VETRPAGDGTFQKWAAVVVPSG | | | | | |
| | | | EEQRYTCHVQHEGLPEPVTLRW | | | | | |
| | | | EPKSSHHHHHH | | | | | |
| | | | (SEQ ID NO: 202) | | | | | |
| | LC | [VL]- | DIVMTQSPDSLAVSLGERATIN | SEQ ID | RTVA | SEQ ID | GS | YPYDVP |
| | frag | RTVAG- | CRASQSVDYNGISYMHWYQQKP | NO: 97 | G | NO: 24 | | DYA |
| | | [B2M (SST, | GQPPKLLIYAASNPESGVPDRF | | (SEQ | | | (SEQ ID |
| | | D98C)]- | SGSGSGTDFTLTISSLQAEDVA | | ID NO: | | | NO: 95) |
| | | GGGSGES | VYYCQQIIEDPWTFGQGTKVEI | | 73) | | | |
| | | SG-HA | KRTVAGRTPKIQVYSRHPAENG | | | | | |
| | | | KSNFLNCYVSGFHPSDIEVDLL | | | | | |
| | | | KNGERIEKVEHSDLSSSKDSST | | | | | |
| | | | YLLYYTEFTPTEKDEYACRVNH | | | | | |
| | | | VTLSQPKIVKWDRCMGSYPYDV | | | | | |
| | | | PDYA (SEQ ID NO: 203) | | | | | |
| HLPPW62 | HC | [VH]- | QITLKESGPTLVKPTQTLTLTC | SEQ ID | ASTK | SEQ ID | EPKSS | HHHHHH |
| | frag | ASTKGG- | TFSGFSLSTSGMGVSWIRQPPG | NO: 96 | GG | NO: 43 | (SEQ ID | (SEQ ID |
| | | [EA3 | KALEWLAHIYWDDDKRYNPSLK | | (SEQ | | NO: 87) | NO: 93) |
| | | (H192C)]- | SRLTITKDTSKNQVVLTMTNMD | | ID NO: | | | |
| | | EPKSS- | PVDTATYYCARLYGFTYGFAYW | | 63) | | | |
| | | His6 | GQGTLVTVSSASTKGGLHLEPP | | | | | |
| | | | KTHVTHCPISDHEATLRCWALG | | | | | |
| | | | FYPAEITLTWQQDGEGHTQDTE | | | | | |
| | | | LVETRPAGDGTFQKWAAVVVPS | | | | | |
| | | | GEEQRYTCHVQHEGLPEPVTLR | | | | | |
| | | | WEPKSSHHHHHH | | | | | |
| | | | (SEQ ID NO: 204) | | | | | |
| | LC | [VL]- | DIVMTQSPDSLAVSLGERATIN | SEQ ID | RTVA | SEQ ID | GS | YPYDVP |
| | frag | RTVAGG- | CRASQSVDYNGISYMHWYQQKP | NO: 97 | GG | NO: 24 | | DYA |
| | | [B2M (SST, | GQPPKLLIYAASNPESGVPDRF | | (SEQ | | | (SEQ ID |
| | | D98C)]- | SGSGSGTDFTLTISSLQAEDVA | | ID NO: | | | NO: 95) |
| | | GGGSGES | VYYCQQIIEDPWTFGQGTKVEI | | 74) | | | |
| | | SG-HA | KRTVAGGRTPKIQVYSRHPAEN | | | | | |
| | | | GKSNFLNCYVSGFHPSDIEVDL | | | | | |
| | | | LKNGERIEKVEHSDLSSSKDSS | | | | | |
| | | | TYLLYYTEFTPTEKDEYACRVN | | | | | |
| | | | HVTLSQPKIVKWDRCMGSYPYD | | | | | |
| | | | VPDYA (SEQ ID NO: 205) | | | | | |
| HLPPW63 | HC | [VH]- | QITLKESGPTLVKPTQTLTLTC | SEQ ID | ASTK | SEQ ID | EPKSS | HHHHHH |
| | frag | ASTKGGS | TFSGFSLSTSGMGVSWIRQPPG | NO: 96 | GGS | NO: 43 | (SEQ ID | (SEQ ID |
| | | -[EA3 | KALEWLAHIYWDDDKRYNPSLK | | (SEQ | | NO: 87) | NO: 93) |
| | | (H192C)]- | SRLTITKDTSKNQVVLTMTNMD | | ID NO: | | | |
| | | EPKSS- | PVDTATYYCARLYGFTYGFAYW | | 64) | | | |
| | | His6 | GQGTLVTVSSASTKGGSLHLEP | | | | | |
| | | | PKTHVTHCPISDHEATLRCWAL | | | | | |
| | | | GFYPAEITLTWQQDGEGHTQDT | | | | | |
| | | | ELVETRPAGDGTFQKWAAVVVP | | | | | |
| | | | SGEEQRYTCHVQHEGLPEPVTL | | | | | |
| | | | RWEPKSSHHHHHH | | | | | |
| | | | (SEQ ID NO: 206) | | | | | |
| | LC | [VL]- | DIVMTQSPDSLAVSLGERATIN | SEQ ID | RTVA | SEQ ID | GS | YPYDVP |
| | frag | RTVAGGS | CRASQSVDYNGISYMHWYQQKP | NO: 97 | GGS | NO: 24 | | DYA |
| | | [B2M (SST, | GQPPKLLIYAASNPESGVPDRF | | (SEQ | | | (SEQ ID |
| | | D98C)]- | SGSGSGTDFTLTISSLQAEDVA | | ID NO: | | | NO: 95) |
| | | GGGSGES | VYYCQQIIEDPWTFGQGTKVEI | | 75) | | | |
| | | SG-HA | KRTVAGGSRTPKIQVYSRHPAE | | | | | |
| | | | NGKSNFLNCYVSGFHPSDIEVD | | | | | |
| | | | LLKNGERIEKVEHSDLSSSKDS | | | | | |
| | | | STYLLYYTEFTPTEKDEYACRV | | | | | |
| | | | NHVTLSQPKIVKWDRCMGSYPY | | | | | |
| | | | DVPDYA (SEQ ID NO: 207) | | | | | |
| HLPPW64 | HC | [VH]- | QITLKESGPTLVKPTQTLTLTC | SEQ ID | ASTK | SEQ ID | EPKSS | HHHHHH |
| | frag | ASTKGGG | TFSGFSLSTSGMGVSWIRQPPG | NO: 96 | GGGS | NO: 43 | (SEQ ID | (SEQ ID |
| | | S-[EA3 | KALEWLAHIYWDDDKRYNPSLK | | (SEQ | | NO: 87) | NO: 93) |
| | | (H192C)]- | SRLTITKDTSKNQVVLTMTNMD | | ID NO: | | | |
| | | EPKSS- | PVDTATYYCARLYGFTYGFAYW | | 65) | | | |
| | | His6 | GQGTLVTVSSASTKGGGSLHLE | | | | | |
| | | | PPKTHVTHCPISDHEATLRCWA | | | | | |
| | | | LGFYPAEITLTWQQDGEGHTQD | | | | | |
| | | | TELVETRPAGDGTFQKWAAVVV | | | | | |
| | | | PSGEEQRYTCHVQHEGLPEPVT | | | | | |
| | | | LRWEPKSSHHHHHH | | | | | |
| | | | (SEQ ID NO: 208) | | | | | |
| | LC | [VL]- | DIVMTQSPDSLAVSLGERATIN | SEQ ID | RTVA | SEQ ID | GS | YPYDVP |
| | frag | RTVAGG | CRASQSVDYNGISYMHWYQQKP | NO: 97 | GGGS | NO: 24 | | DYA |
| | | GS- | GQPPKLLIYAASNPESGVPDRF | | (SEQ | | | (SEQ ID |
| | | [B2M (SST, | SGSGSGTDFTLTISSLQAEDVA | | ID NO: | | | NO: 95) |
| | | D98C)]- | VYYCQQIIEDPWTFGQGTKVEI | | 76) | | | |
| | | GGGSGES | KRTVAGGGSRTPKIQVYSRHPA | | | | | |
| | | SG-HA | ENGKSNFLNCYVSGFHPSDIEV | | | | | |
| | | | DLLKNGERIEKVEHSDLSSSKD | | | | | |
| | | | SSTYLLYYTEFTPTEKDEYACR | | | | | |
| | | | VNHVTLSQPKIVKWDRCMGSYP | | | | | |
| | | | YDVPDYA (SEQ ID NO: 209) | | | | | |
| HLPPW65 | HC | [VH]- | QITLKESGPTLVKPTQTLTLTC | SEQ ID | ASTK | SEQ ID | EPKSS | HHHHHH |
| | frag | ASTKG4S- | TFSGFSLSTSGMGVSWIRQPPG | NO: 96 | GGGG | NO: 43 | (SEQ ID | (SEQ ID |
| | | [EA3 | KALEWLAHIYWDDDKRYNPSLK | | S | | NO: 87) | NO: 93) |
| | | (H192C)]- | SRLTITKDTSKNQVVLTMTNMD | | (SEQ | | | |
| | | EPKSS- | PVDTATYYCARLYGFTYGFAYW | | ID NO: | | | |
| | | His6 | GQGTLVTVSSASTKGGGGSLHL | | 66) | | | |
| | | | EPPKTHVTHCPISDHEATLRCW | | | | | |
| | | | ALGFYPAEITLTWQQDGEGHTQ | | | | | |
| | | | DTELVETRPAGDGTFQKWAAVV | | | | | |
| | | | VPSGEEQRYTCHVQHEGLPEPV | | | | | |
| | | | TLRWEPKSSHHHHHH | | | | | |
| | | | (SEQ ID NO: 210) | | | | | |
| | LC | [VL]- | DIVMTQSPDSLAVSLGERATIN | SEQ ID | RTVA | SEQ ID | GS | YPYDVP |
| | frag | RTVAGG | CRASQSVDYNGISYMHWYQQKP | NO: 97 | GGGG | NO: 24 | | DYA |
| | | GGS- | GQPPKLLIYAASNPESGVPDRF | | S | | | (SEQ ID |
| | | [B2M (SST, | SGSGSGTDFTLTISSLQAEDVA | | (SEQ | | | NO: 95) |
| | | D98C)]- | VYYCQQIIEDPWTFGQGTKVEI | | ID NO: | | | |
| | | GGGSGES | KRTVAGGGGSRTPKIQVYSRHP | | 77) | | | |
| | | SG-HA | AENGKSNFLNCYVSGFHPSDIE | | | | | |
| | | | VDLLKNGERIEKVEHSDLSSSK | | | | | |
| | | | DSSTYLLYYTEFTPTEKDEYAC | | | | | |
| | | | RVNHVTLSQPKIVKWDRCMGSY | | | | | |
| | | | PYDVPDYA (SEQ ID NO: 211) | | | | | |
| HLPPW66 | HC | [VH]- | QITLKESGPTLVKPTQTLTLTC | SEQ ID | ASTK | SEQ ID | EPKSS | HHHHHH |
| | frag | ASTKG4S | TFSGFSLSTSGMGVSWIRQPPG | NO: 96 | GGGG | NO: 43 | (SEQ ID | (SEQ ID |
| | | G-[EA3 | KALEWLAHIYWDDDKRYNPSLK | | SG | | NO: 87) | NO: 93) |
| | | (H192C)]- | SRLTITKDTSKNQVVLTMTNMD | | (SEQ | | | |
| | | EPKSS- | PVDTATYYCARLYGFTYGFAYW | | | | | |
| | | His6 | GQGTLVTVSSASTKGGGGSGLH | | ID NO: | | | |
| | | | LEPPKTHVTHCPISDHEATLRC | | 67) | | | |
| | | | WALGFYPAEITLTWQQDGEGHT | | | | | |
| | | | QDTELVETRPAGDGTFQKWAAV | | | | | |
| | | | VVPSGEEQRYTCHVQHEGLPEP | | | | | |
| | | | VTLRWEPKSSHHHHHH | | | | | |
| | | | (SEQ ID NO: 212) | | | | | |
| | LC | [VL]- | DIVMTQSPDSLAVSLGERATIN | SEQ ID | RTVA | SEQ ID | GS | YPYDVP |
| | frag | RTVAG4S | CRASQSVDYNGISYMHWYQQKP | NO: 97 | GGGG | NO: 24 | | DYA |
| | | G-[B2M | GQPPKLLIYAASNPESGVPDRF | | SG | | | (SEQ ID |
| | | (SST, | SGSGSGTDFTLTISSLQAEDVA | | (SEQ | | | NO: 95) |
| | | D98C)]- | VYYCQQIIEDPWTFGQGTKVEI | | ID NO: | | | |
| | | GGGSGES | KRTVAGGGGSGRTPKIQVYSRH | | 78) | | | |
| | | SG-HA | PAENGKSNFLNCYVSGFHPSDI | | | | | |
| | | | EVDLLKNGERIEKVEHSDLSSS | | | | | |
| | | | KDSSTYLLYYTEFTPTEKDEYA | | | | | |
| | | | CRVNHVTLSQPKIVKWDRCMGS | | | | | |
| | | | YPYDVPDYA | | | | | |
| | | | (SEQ ID NO: 213) | | | | | |
| HLPPW67 | HC | [VH]- | QITLKESGPTLVKPTQTLTLTC | SEQ ID | ASTK | SEQ ID | EPKSS | HHHHHH |
| | frag | ASTKG4S | TFSGFSLSTSGMGVSWIRQPPG | NO: 96 | GGGG | NO: 43 | (SEQ ID | (SEQ ID |
| | | GG-[EA3 | KALEWLAHIYWDDDKRYNPSLK | | SGG | | NO: 87) | NO: 93) |
| | | (H192C)]- | SRLTITKDTSKNQVVLTMTNMD | | (SEQ | | | |
| | | EPKSS- | PVDTATYYCARLYGFTYGFAYW | | ID NO: | | | |
| | | His6 | GQGTLVTVSSASTKGGGGSGGL | | 68) | | | |
| | | | HLEPPKTHVTHCPISDHEATLR | | | | | |
| | | | CWALGFYPAEITLTWQQDGEGH | | | | | |
| | | | TQDTELVETRPAGDGTFQKWAA | | | | | |
| | | | VVVPSGEEQRYTCHVQHEGLPE | | | | | |
| | | | PVTLRWEPKSSHHHHHH | | | | | |
| | | | (SEQ ID NO: 214) | | | | | |
| | LC | [VL]- | DIVMTQSPDSLAVSLGERATIN | SEQ ID | RTVA | SEQ ID | GS | YPYDVP |
| | frag | RTVAG4S | CRASQSVDYNGISYMHWYQQKP | NO: 97 | GGGG | NO: 24 | | DYA |
| | | GG- | GQPPKLLIYAASNPESGVPDRF | | SGG | | | (SEQ ID |
| | | [B2M (SST, | SGSGSGTDFTLTISSLQAEDVA | | (SEQ | | | NO: 95) |
| | | D98C)]- | VYYCQQIIEDPWTFGQGTKVEI | | ID NO: | | | |
| | | GGGSGES | KRTVAGGGGSGGRTPKIQVYSR | | 79) | | | |
| | | SG-HA | HPAENGKSNFLNCYVSGFHPSD | | | | | |
| | | | IEVDLLKNGERIEKVEHSDLSS | | | | | |
| | | | SKDSSTYLLYYTEFTPTEKDEY | | | | | |
| | | | ACRVNHVTLSQPKIVKWDRCMG | | | | | |
| | | | SYPYDVPDYA | | | | | |
| | | | (SEQ ID NO: 215) | | | | | |
| HLPPW68 | HC | [VH]- | QITLKESGPTLVKPTQTLTLTC | SEQ ID | ASTK | SEQ ID | EPKSS | HHHHHH |
| | frag | ASTKG4S | TFSGFSLSTSGMGVSWIRQPPG | NO: 96 | GGGG | NO: 43 | (SEQ ID | (SEQ ID |
| | | GGS-[EA3 | KALEWLAHIYWDDDKRYNPSLK | | SGGS | | NO: 87) | NO: 93) |
| | | (H192C)]- | SRLTITKDTSKNQVVLTMTNMD | | (SEQ | | | |
| | | EPKSS- | PVDTATYYCARLYGFTYGFAYW | | ID NO: | | | |
| | | His6 | GQGTLVTVSSASTKGGGGSGGS | | 69) | | | |
| | | | LHLEPPKTHVTHCPISDHEATL | | | | | |
| | | | RCWALGFYPAEITLTWQQDGEG | | | | | |
| | | | HTQDTELVETRPAGDGTFQKWA | | | | | |
| | | | AVVVPSGEEQRYTCHVQHEGLP | | | | | |
| | | | EPVTLRWEPKSSHHHHHH | | | | | |
| | | | (SEQ ID NO: 216) | | | | | |
| | LC | [VL]- | DIVMTQSPDSLAVSLGERATIN | SEQ ID | RTVA | SEQ ID | GS | YPYDVP |
| | frag | RTVAG4S | CRASQSVDYNGISYMHWYQQKP | NO: 97 | GGGG | NO: 24 | | DYA |
| | | GGS- | GQPPKLLIYAASNPESGVPDRF | | SGGS | | | (SEQ ID |
| | | [B2M (SST, | SGSGSGTDFTLTISSLQAEDVA | | (SEQ | | | NO: 95) |
| | | D98C)]- | VYYCQQIIEDPWTFGQGTKVEI | | ID NO: | | | |
| | | GGGSGES | KRTVAGGGGSGGSRTPKIQVYS | | 80) | | | |
| | | SG-HA | RHPAENGKSNFLNCYVSGFHPS | | | | | |
| | | | DIEVDLLKNGERIEKVEHSDLS | | | | | |
| | | | SSKDSSTYLLYYTEFTPTEKDE | | | | | |
| | | | YACRVNHVTLSQPKIVKWDRCM | | | | | |
| | | | GSYPYDVPDYA | | | | | |
| | | | (SEQ ID NO: 217) | | | | | |
| HLPPW69 | HC | [VH]- | QITLKESGPTLVKPTQTLTLTC | SEQ ID | ASTK | SEQ ID | EPKSS | HHHHHH |
| | frag | ASTKG4S | TFSGFSLSTSGMGVSWIRQPPG | NO: 96 | GGGG | NO: 43 | (SEQ ID | (SEQ ID |
| | | GGGS- | KALEWLAHIYWDDDKRYNPSLK | | SGGG | | NO: 87) | NO: 93) |
| | | [EA3 | SRLTITKDTSKNQVVLTMTNMD | | S | | | |
| | | (H192C)]- | PVDTATYYCARLYGFTYGFAYW | | (SEQ | | | |
| | | EPKSS- | GQGTLVTVSSASTKGGGGSGGG | | ID NO: | | | |
| | | His6 | SLHLEPPKTHVTHCPISDHEAT | | 70) | | | |
| | | | LRCWALGFYPAEITLTWQQDGE | | | | | |
| | | | GHTQDTELVETRPAGDGTFQKW | | | | | |
| | | | AAVVVPSGEEQRYTCHVQHEGL | | | | | |
| | | | PEPVTLRWEPKSSHHHHHH | | | | | |
| | | | (SEQ ID NO: 218) | | | | | |
| | LC | [VL]- | DIVMTQSPDSLAVSLGERATIN | SEQ ID | RTVA | SEQ ID | GS | YPYDVP |
| | frag | RTVAG4S | CRASQSVDYNGISYMHWYQQKP | NO: 97 | GGGG | NO: 24 | | DYA |
| | | GGGS- | GQPPKLLIYAASNPESGVPDRF | | SGGG | | | (SEQ ID |
| | | [B2M (SST, | SGSGSGTDFTLTISSLQAEDVA | | S | | | NO: 95) |
| | | D98C)]- | VYYCQQIIEDPWTFGQGTKVEI | | (SEQ | | | |
| | | GGGSGES | KRTVAGGGGSGGGSRTPKIQVY | | ID NO: | | | |
| | | SG-HA | SRHPAENGKSNFLNCYVSGFHP | | 81) | | | |
| | | | SDIEVDLLKNGERIEKVEHSDL | | | | | |
| | | | SSSKDSSTYLLYYTEFTPTEKD | | | | | |
| | | | EYACRVNHVTLSQPKIVKWDRC | | | | | |
| | | | MGSYPYDVPDYA | | | | | |
| | | | (SEQ ID NO: 219) | | | | | |
| HLPPW70 | HC | [VH]- | QITLKESGPTLVKPTQTLTLTC | SEQ ID | ASTK | SEQ ID | EPKSS | HHHHHH |
| | frag | ASTK(G4S | TFSGFSLSTSGMGVSWIRQPPG | NO: 96 | GGGG | NO: 43 | (SEQ ID | (SEQ ID |
| | | )2-[EA3 | KALEWLAHIYWDDDKRYNPSLK | | SGGG | | NO: 87) | NO: 93) |
| | | (H192C)]- | SRLTITKDTSKNQVVLTMTNMD | | GS | | | |
| | | EPKSS- | PVDTATYYCARLYGFTYGFAYW | | (SEQ | | | |
| | | His6 | GQGTLVTVSSASTKGGGGSGGG | | ID NO: | | | |
| | | | SLHLEPPKTHVTHCPISDHEAT | | 71) | | | |
| | | | LRCWALGFYPAEITLTWQQDGE | | | | | |
| | | | GHTQDTELVETRPAGDGTFQKW | | | | | |
| | | | AAVVVPSGEEQRYTCHVQHEGL | | | | | |
| | | | PEPVTLRWEPKSSHHHHHH | | | | | |
| | | | (SEQ ID NO: 220) | | | | | |
| | LC | [VL]- | DIVMTQSPDSLAVSLGERATIN | SEQ ID | RTVA | SEQ ID | GS | YPYDVP |
| | frag | RTVAG4S | CRASQSVDYNGISYMHWYQQKP | NO: 97 | GGGG | NO: 24 | | DYA |
| | | G4S- | GQPPKLLIYAASNPESGVPDRF | | SGGG | | | (SEQ ID |
| | | [B2M (SST, | SGSGSGTDFTLTISSLQAEDVA | | GS | | | NO: 95) |
| | | D98C)]- | VYYCQQIIEDPWTFGQGTKVEI | | (SEQ | | | |
| | | GGGSGES | KRTVAGGGGSGGGSRTPKIQVY | | ID NO: | | | |
| | | SG-HA | SRHPAENGKSNFLNCYVSGFHP | | 82) | | | |
| | | | SDIEVDLLKNGERIEKVEHSDL | | | | | |
| | | | SSSKDSSTYLLYYTEFTPTEKD | | | | | |
| | | | EYACRVNHVTLSQPKIVKWDRC | | | | | |
| | | | MGSYPYDVPDYA | | | | | |
| | | | (SEQ ID NO: 221) | | | | | |

Pseudo-Fabs comprising HLA-E α3 (H192C) and B2M (SST, D98C) elbow variants were expressed, purified and then assayed for: expression titers, purity; monodispersity by SEC; target binding; and thermal stability using methods as described above.

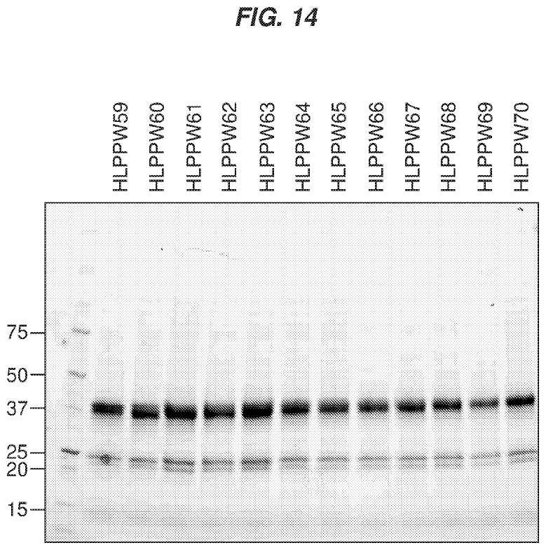

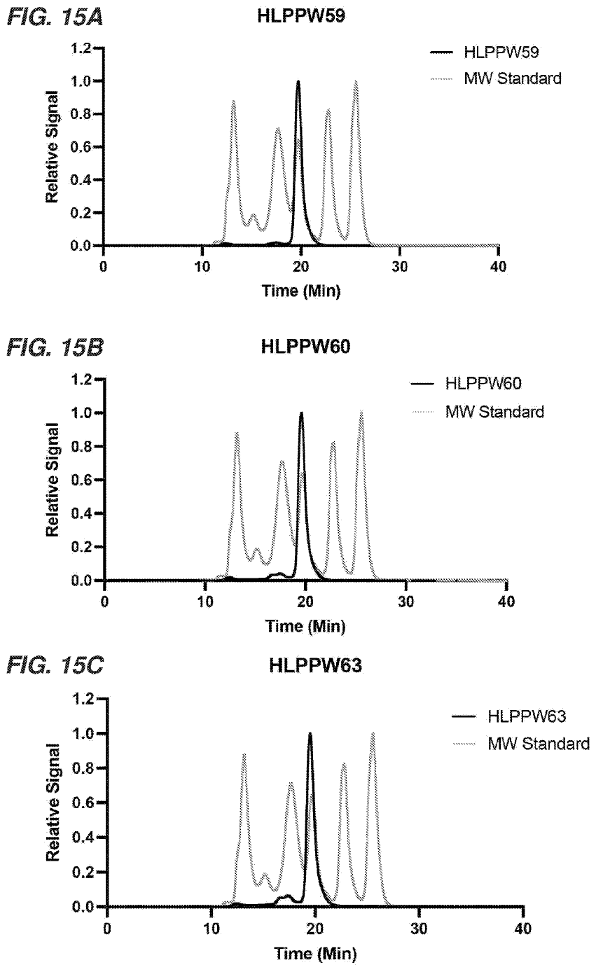

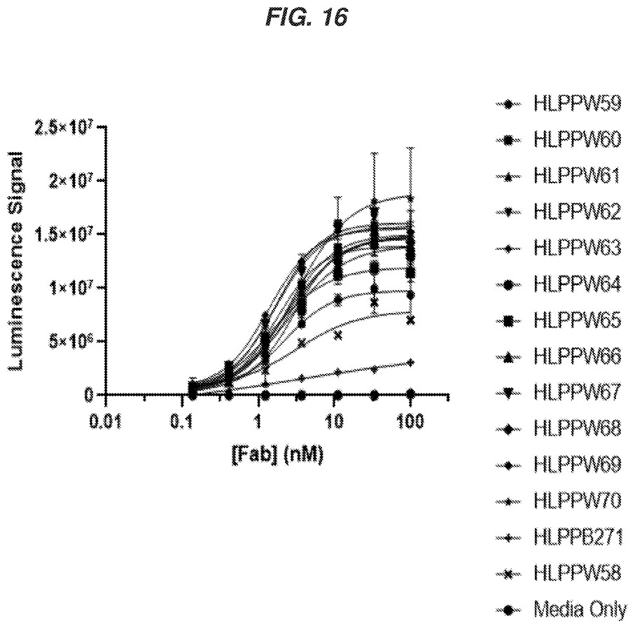

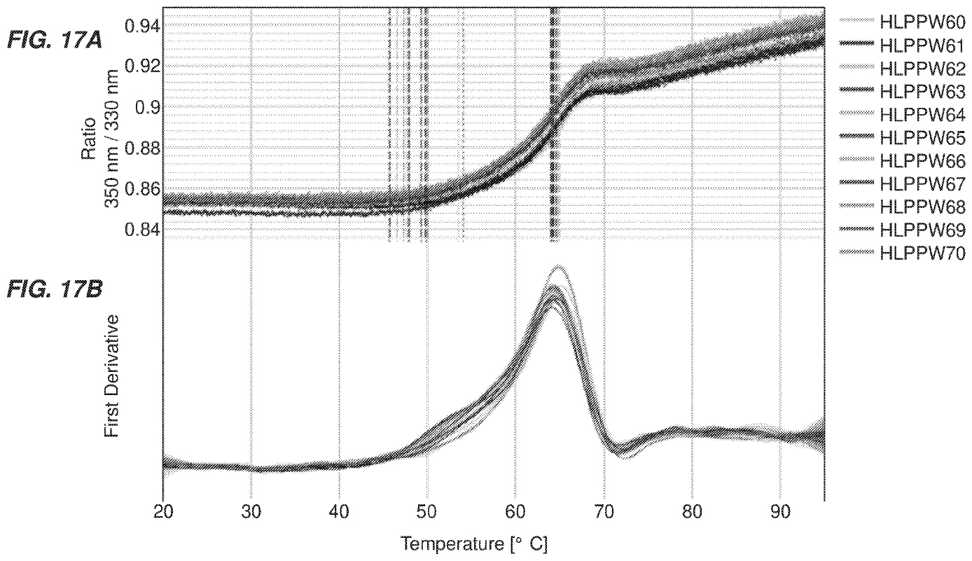

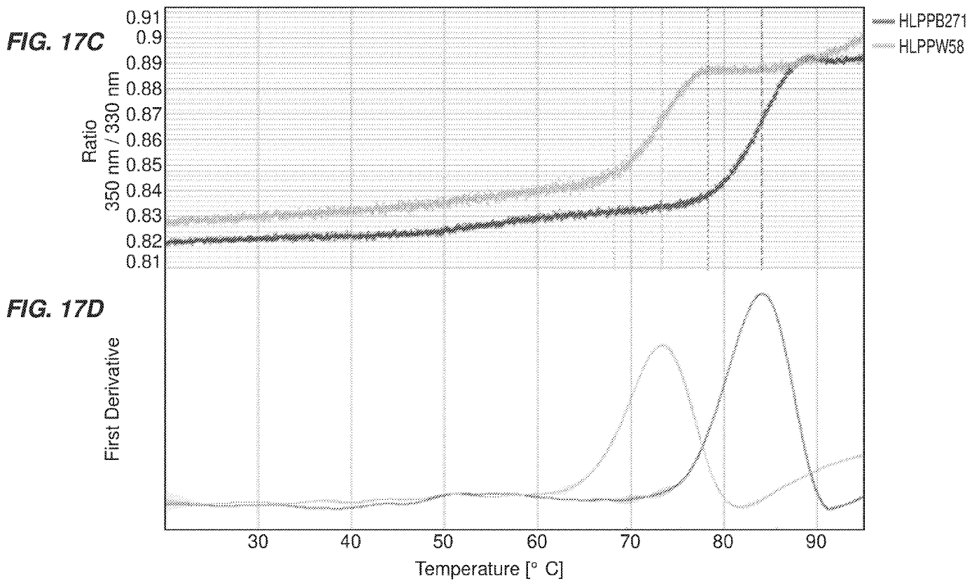

The results for the pseudo-Fab designs comprising the HLA-E α3 (H192C) and E2M (SST, D98C) elbow variants are summarized below (TABLE 24). Expression of pseudo-Fabs HLPPW59-HLPPW70 was verified (FIG. 14). Yield and purity values are reported in TABLE 24. The tested HLPPW59, HLPPW60, and HLPPW63 elbow variant pFabs appeared as a monodisperse species in size exclusion chromatography (FIG. 15). The elbow variant pseudo-Fabs HLPPW59-HLPPW70 showed improved target binding compared to the native Fab HLPPW58 EC50 (FIG. 16, See TABLE 24 for EC50 values and 9500 confidence intervals). In thermal stability assays, each of the pFabs HLPPW59-HLPPW70 had melting temperatures of between 64.04-65.03° C., with HLPPW59 and HLPPW60 having the highest onset of melting at 54.10° C. and 53.48° C., respectively. See TABLE 24.

TABLE 24
Summary of Results for Elbow Variant Pseudo-Fabs

| | Elbow sequence N- | Elbow sequence N- | | | | | | |
|---|---|---|---|---|---|---|---|---|
| | terminal to EA3 | terminal to | Titer | Purity | EC50 | 95% Cl, | Tonset | Tml |
| Name | (H192C) | BSM (SST, D98C) | (mg/mL) | (%) | (nM) | EC50 (nM) | (C) | (C) |
| HLPPW59 | AST | RTV | 1.10 | 94.6 | 2.138 | - to 3.750 | 54.10 | 64.81 |
| HLPPW60 | ASTK | RTVA | 1.00 | 87.4 | 1.737 | 1.124 to 2.482 | 53.48 | 65.03 |
| | (SEQ ID NO: 61) | (SEQ ID NO: 72) | | | | | | |
| HLPPW61 | ASTKG | RTVAG | 0.95 | 86.8 | 2.316 | | 49.83 | 64.62 |
| | (SEQ ID NO: 62) | (SEQ ID NO: 73) | | | | | | |
| HLPPW62 | ASTKGG | RTVAGG | 1.10 | 82.9 | 1.669 | - to 2.162 | 49.38 | 64.36 |
| | (SEQ ID NO: 63) | (SEQ ID NO: 74) | | | | | | |
| HLPPW63 | ASTKGGS | RTVAGGS | 1.13 | 87.7 | 1.381 | 1.125 to 1.654 | 49.99 | 64.11 |
| | (SEQ ID NO: 64) | (SEQ ID NO: 75) | | | | | | |
| HLPPW64 | ASTKGGGS | RTVAGGGS | 0.83 | 90.1 | 2.502 | 1.433 to 3.806 | 46.50 | 64.36 |
| | (SEQ ID NO: 65) | (SEQ ID NO: 76) | | | | | | |
| HLPPW65 | ASTKGGGGS | RTVAGGGGS | 0.55 | 85.6 | 1.466 | - to 2.104 | 47.99 | 64.04 |
| | (SEQ ID NO: 66) | (SEQ ID NO: 77) | | | | | | |
| HLPPW66 | ASTKGGGGSG | RTVAGGGGSG | 1.28 | 83.2 | 3.028 | 2.152 to 4.116 | 47.34 | 64.29 |
| | (SEQ ID NO: 67) | (SEQ ID NO: 78) | | | | | | |
| HLPPW67 | ASTKGGGGSGG | RTVAGGGGSGG | 1.38 | 82.6 | 1.673 | 1.079 to 2.907 | 49.28 | 64.23 |
| | (SEQ ID NO: 68) | (SEQ ID NO: 79) | | | | | | |
| HLPPW68 | ASTKGGGGSGGS | RTVAGGGGSGGS | 1.32 | 82.0 | 2.575 | 1.533 to 3.950 | 47.80 | 64.12 |
| | (SEQ ID NO: 69) | (SEQ ID NO: 80) | | | | | | |
| HLPPW69 | ASTKGGGGSGGGS | RTVAGGGGSGGGS | 0.81 | 82.0 | 2.224 | 1.218 to 3.503 | 45.79 | 64.32 |
| | (SEQ ID NO: 70) | (SEQ ID NO: 81) | | | | | | |
| HLPPW70 | ASTKGGGGSGGGGS | RTVAGGGGSGGGGS | 0.85 | 85.2 | 3.06 | 0.2445 to | 47.34 | 64.20 |
| | (SEQ ID NO: 71) | (SEQ ID NO: 82) | | | | 7.986 | | |
| HLPPB271 | None | None | | | 3.535 | | 78.29 | 84.03 |
| HLPPW58 | None | RTV | | | 3.206 | | 68.24 | 73.33 |

### 9.2 Example 2: ICAM-1-Based CL-CH1-Replacement

### 9.2.1 Introduction

ICAM family molecules are non-antibody immunoglobulin superfamily members that resemble IgG CH1 domains in terms of sequence based properties (see, e.g., Hebditch et al., 2017, Sci Rep; 7:12404). Domain 1 (D1) of ICAM-1 (ICAM-1 D1) was selected as a candidate to replace IgG Fb because of its structural similarity with CH1, and availability of information on its dimerization and function. ICAM-1 D1 cannot be expressed or homodimerized by itself, but can be engineered to do so. The E34 residue can be mutated (E34K) to knock out LFA-1 binding (see Jun et al., 2001, Proc Natl Acad Sci USA; 98:6830-5). Also, structural studies have shown that the introduction of a cysteine at the homo-dimerization interface of ICAM-1 D1 make the dimer stable (see Jun et al., 2001, J Biol Chem; 276:29019-27). Also, engineering of D1 has led to expression of D1 alone and its dimerization (Owens et al. 2010, J Biol Chem; 285:15906-15).

It was envisaged that the interface of D1 could be engineered to create a hetero-dimerization module, which could be substituted in the Fb domain to facilitate “Fab” heterodimerization. The ICAM-1 D1 heterodimer structurally aligns with the CH1-CL heterodimer with r.m.s.d. of ~5.4° Å (FIG. 2C). The combined distance between the N-termini of the CH1-CL domains and the C-term of the VH-VL domains is ~21 Å, whereas the analogous distance when the CH1-CL dimer is replaced by the ICAM-1 D1 dimer is ~40° Å. This suggests that ICAM-1 D1 heterodimers can be used for CH1 replacement. However, the “elbow” sequences linking the variable domains to the ICAM-1 D1 domains may be modified as described below, to accommodate appropriate VH-VL pairing.

### 9.2.2 Experimental Development (ICAM-1 D1)

Molecules were constructed having mutations at the ICAM-1 D1 interface to generate pairs that would selectively interact in a hetero-dimeric manner to allow for specific pairing between a Fab light chain variable domain and heavy chain variable domain (See TABLES 25-28). Generation of heterodimeric mutations were incorporated in the background of established mutation sets (see Jun et al., 2001, Proc Natl Acad Sci USA; 98:6830-5; see Jun et al., 2001, J Biol Chem; 276:29019-27; Owens et al. 2010, J Biol Chem; 285:15906-15). Mutations included knockout of LFA-1 binding (e.g., E34K, such E34K mutant termed hereinafter “LFA-1 Binding Knockout”) (see Jun et al., 2001, J Biol Chem; 276:29019-27), as well as mutations at ICAM-1 D1 positions 2, 10, 23, 38, 63, 57, or 78 (e.g., T2V, I10T, T23A, E34K, P38T, P63V, S67A, T78A). Cysteines for disulfide bond formation were introduced at position 13 by substitution (R13C), and at position 84 by appending onto the C-terminus of the insert (e.g., 84C), with positions numbered according to the ICAM-1 D1 amino acid numbering in TABLE 3. In specific constructs, salt-bridges were incorporated for electrostatic steering at positions 49 and 53 (e.g., R49E, E53R). Multiple versions of “knob-into-hole” type mutations were generated, where a large, hydrophobic residue was inserted into one ICAM-1 domain, and an accommodating binding pocket was mutated into the second ICAM-1 domain. See, e.g., L18F or L18W into L18A, T20A, T23A, E34K, P38T, L42A, V51A. Pseudo-Fabs with the sequences shown in TABLE 28 were assembled from variant HLA-E α3-β2m dimers comprising these mutations. ICAM-1 D1 variant sequences that were constructed are shown below in TABLES 25 (bolded relative to human WT). Affinity tags were appended onto the C-terminus of ICAM-1 D1 variant with spacers (TABLE 26). Dimer pairs were assembled from variant ICAM-1 D1 sequences as shown in TABLE 27.

TABLE 25
ICAM-1 D1 Variant Sequences

| | Insert Sequence |
|---|---|
| Name | (Mutations relative to WT are bolded and underlined) |
| ICAM1 D1 | QTSVSPSKVILPRGGSVLVTCSTSCDQPKLLGIETPLPKKELLLPGNNRKVY |
| (wild-type) | ELSNVQEDSQPMCYSNCPDGQSTAKTFLTVY (SEQ ID NO: 49) |
| ICAM1 D1 (E34K) | QTSVSPSKVILPRGGSVLVTCSTSCDQPKLLGIKTPLPKKELLLPGNNRKVY |
| LFA-1 Binding Knockout | ELSNVQEDSQPMCYSNCPDGQSTAKTFLTVY (SEQ ID NO: 51) |
| ICAM1 D1 (T2V, 110T, T23A, E34K, P38T, | QVSVSPSKVTLPRGGSVLVTCSASCDQPKLLGIKTPLTKKELLLPGNNRKVY |
| P63V, S67A, T78A) | ELSNVQEDSQVMCYANCPDGQSTAKAFLTVY (SEQ ID NO: 52) |
| Stabilized | |
| ICAM1 D1 (T2V, 110T, R13C, T23A, E34K, | QVSVSPSKVTLPCGGSVLVTCSASCDQPKLLGIKTPLTKKELLLPGNNRKVY |
| P38T, P63V, S67A, T78A, 84C) | ELSNVQEDSQVMCYANCPDGQSTAKAFLTVYC (SEQ ID NO: 53) |
| Disulfide | |
| ICAM1 D1 (T2V, 110T, R13C, T23A, E34K, | QVSVSPSKVTLPCGGSVLVTCSASCDQPKLLGIKTPLTKKELLLPGNNEKVY |
| P38T, R49E, P63V, S67A, T78A, 84C) | ELSNVQEDSQVMCYANCPDGQSTAKAFLTVYC (SEQ ID NO: 54) |
| Salt Bridge | |
| ICAM1 D1 (T2V, 110T, R13C, T23A, E34K, | QVSVSPSKVTLPCGGSVLVTCSASCDQPKLLGIKTPLTKKELLLPGNNRKVY |
| P38T, E53R, P63V, S67A, T78A, 84C) | RLSNVQEDSQVMCYANCPDGQSTAKAFLTVYC (SEQ ID NO: 55) |
| Salt Bridge | |
| ICAM1 D1 (T2V, 110T, R13C, L18F, T23A, | QVSVSPSKVTLPCGGSVFVTCSASCDQPKLLGIKTPLTKKELLLPGNNRKVY |
| E34K, P38T, P63V, S67A, T78A, 84C) | ELSNVQEDSQVMCYANCPDGQSTAKAFLTVYC (SEQ ID NO: 56) |
| Knob-Into-Hole | |
| ICAM1 D1 (T2V, 110T, R13C, L18A, T20A, | QVSVSPSKVTLPCGGSVAVACSASCDQPKLLGIKTPLTKKEALLPGNNRKAY |
| T23A, E34K, P38T, L42A, V51A, P63V, | ELSNVQEDSQVMCYANCPDGQSTAKAFLTVYC (SEQ ID NO: 57) |
| S67A, T78A, 84C) | |
| Knob-Into-Hole | |
| ICAM1 D1 (T2V, 110T, R13C, L18W, | QVSVSPSKVTLPCGGSVWVTCSASCDQPKLLGIKTPLTKKELLLPGNNRKVY |
| T23A, E34K, P38T, P63V, S67A, T78A, | ELSNVQEDSQVMCYANCPDGQSTAKAFLTVYC (SEQ ID NO: 58) |
| 84C) | |
| Knob-Into-Hole | |

TABLE 26
ICAM-1 Variant Polypeptides

| | Polypeptide Sequence | | | | |
|---|---|---|---|---|---|
| | (Mutations relative to | | | | |
| | WT are bolded and | | | C-terminus | |
| Name | underlined) | Elbow | Insert | Spacer | Tag |
| [ICAM1 D1]-EPKSS- | QTSVSPSKVILPRGGSVLVTCSTSCDQP | None | SEQ ID | EPKSS | HHHHHH |
| His6 | KLLGIETPLPKKELLLPGNNRKVYELSN | | NO: 49 | (SEQ ID | (SEQ ID |
| | VQEDSQPMCYSNCPDGQSTAKTFLTVYE | | | NO: 87) | NO: 93) |
| | PKSSHHHHHH (SEQ ID NO: 222) | | | | |
| [ICAM1 D1 (E34K)]- | QTSVSPSKVILPRGGSVLVTCSTSCDQP | None | SEQ ID | EPKSS | HHHHHH |
| EPKSS-His6 | KLLGIKTPLPKKELLLPGNNRKVYELSN | | NO: 51 | (SEQ ID | (SEQ ID |
| | VQEDSQPMCYSNCPDGQSTAKTFLTVYE | | | NO: 87) | NO: 93) |
| | PKSSHHHHHH (SEQ ID NO: 223) | | | | |
| [ICAM1 D1 (T2V, | QVSVSPSKVTLPRGGSVLVTCSASCDQP | None | SEQ ID | EPKSS | HHHHHH |
| I10T, T23A, E34K, | KLLGIKTPLTKKELLLPGNNRKVYELSN | | NO: 52 | (SEQ ID | (SEQ ID |
| P38T, P63V, S67A, | VQEDSQVMCYANCPDGQSTAKAFLTVYE | | | NO: 87) | NO: 93) |
| T78A)]-EPKSS-His6 | PKSSHHHHHH (SEQ ID NO: 224) | | | | |
| [ICAM1 D1 (T2V, | QVSVSPSKVTLPCGGSVLVTCSASCDQP | None | SEQ ID | EPKSS | HHHHHH |
| I10T, R13C, T23A, | KLLGIKTPLTKKELLLPGNNRKVYELSN | | NO: 53 | (SEQ ID | (SEQ ID |
| E34K, P38T, P63V, | VQEDSQVMCYANCPDGQSTAKAFLTVYC | | | NO: 87) | NO: 93) |
| S67A, T78A, 84C)]- | EPKSSHHHHHH (SEQ ID NO: 225) | | | | |
| EPKSS-His6 | | | | | |
| [ICAM1 D1 (T2V, | QVSVSPSKVTLPCGGSVLVTCSASCDQP | None | SEQ ID | EPKSS | HHHHHH |
| I10T, R13C, T23A, | KLLGIKTPLTKKELLLPGNNEKVYELSN | | NO: 54 | (SEQ ID | (SEQ ID |
| E34K, P38T, R49E, | VQEDSQVMCYANCPDGQSTAKAFLTVYC | | | NO: 87) | NO: 93) |
| P63V, S67A, T78A, | EPKSSHHHHHH (SEQ ID NO: 226) | | | | |
| 84C)]-EPKSS-His6 | | | | | |
| [ICAM1 D1 (T2V, | QVSVSPSKVTLPCGGSVLVTCSASCDQP | None | SEQ ID | SG | WSHPQFE |
| I10T, R13C, T23A, | KLLGIKTPLTKKELLLPGNNRKVYRLSN | | NO: 55 | | K |
| E34K, P38T, E53R, | VQEDSQVMCYANCPDGQSTAKAFLTVYC | | | | (SEQ ID |
| P63V, S67A, T78A, | SGWSHPQFEK (SEQ ID NO: 227) | | | | NO: 94) |
| 84C)]-SG-Strep | | | | | |
| [ICAM1 D1 (T2V, | QVSVSPSKVTLPCGGSVEVTCSASCDQP | None | SEQ ID | EPKSS | HHHHHH |
| I10T, R13C, L18F, | KLLGIKTPLTKKELLLPGNNRKVYELSN | | NO: 56 | (SEQ ID | (SEQ ID |
| T23A, E34K, P38T, | VQEDSQVMCYANCPDGQSTAKAFLTVYC | | | NO: 87) | NO: 93) |
| P63V, S67A, T78A, | EPKSSHHHHHH (SEQ ID NO: 228) | | | | |
| 84C)]-EPKSS-His6 | | | | | |
| [ICAM1 D1 (T2V, | QVSVSPSKVTLPCGGSVAVACSASCDQP | None | SEQ ID | SG | WSHPQFE |
| I10T, R13C, L18A, | KLLGIKTPLTKKEALLPGNNRKAYELSN | | NO: 57 | | K |
| T20A, T23A, E34K, | VQEDSQVMCYANCPDGQSTAKAFLTVYC | | | | (SEQ ID |
| P38T, L42A, V51A, | SGWSHPQFEK (SEQ ID NO: 229) | | | | NO: 94) |
| P63V, S67A, T78A, | | | | | |
| 84C)]-SG-Strep | | | | | |
| [ICAM1 D1 (T2V, | QVSVSPSKVTLPCGGSVWVTCSASCDQP | None | SEQ ID | EPKSS | HHHHHH |
| I10T, R13C, L18W, | KLLGIKTPLTKKELLLPGNNRKVYELSN | | NO: 58 | (SEQ ID | (SEQ ID |
| T23A, E34K, P38T, | VQEDSQVMCYANCPDGQSTAKAFLTVYC | | | NO: 87) | NO: 93) |
| P63V, S67A, T78A, | EPKSSHHHHHH (SEQ ID NO: 230) | | | | |
| 84C)]-EPKSS-His6 | | | | | |

Pseudo-Fabs having the sequences shown in TABLES 27 and TABLE 28 were assembled from variant HLA-E α3-β2m dimers comprising these mutations.

TABLE 27
ICAM-1 Variant Polypeptide Homodimers and Heterodimers

| Dimer | Chain | Chain | Chain Polypeptide | | | C-terminus | |
|---|---|---|---|---|---|---|---|
| Identifier | | Polypeptide | Description | Elbow | Insert | Spacer | Tag |
| HLPPW2 | A | SEQ ID NO: 222 | ICAM1 D1-EPKSS-His6 | None | SEQ ID | EPKSS | HHHHHH |
| | | | | | NO: 49 | (SEQ ID | (SEQ ID |
| | B | | | | | NO: 87) | NO: 93) |
| HLPPW3 | A | SEQ ID NO: 223 | [ICAM1 D1 (E34K)]- | None | SEQ ID | EPKSS | HHHHHH |
| | | | EPKSS-His6 | | NO: 51 | (SEQ ID | (SEQ ID |
| | B | | | | | NO: 87) | NO: 93) |
| HLPPW4 | A | SEQ ID NO: 224 | [ICAM1 D1 (T2V, I10T, | None | SEQ ID | EPKSS | HHHHHH |
| | | | T23A, E34K, P38T, | | NO: 52 | (SEQ ID | (SEQ ID |
| | B | | P63V, S67A, T78A)]- | | | NO: 87) | NO: 93) |
| | | | EPKSS-His6 | | | | |
| HLPPW5 | A | SEQ ID NO: 225 | [ICAM1 D1 (T2V, I10T, | None | SEQ ID | EPKSS | HHHHHH |
| | | | R13C, T23A, E34K, | | NO: 53 | (SEQ ID | (SEQ ID |
| | | | P38T, P63V, S67A, | | | | |
| | B | | T78A, 84C)]-EPKSS- | | | NO: 87) | NO: 93) |
| | | | His6 | | | | |
| HLPPW6 | A | SEQ ID NO: 226 | [ICAM1 D1 (T2V, I10T, | None | SEQ ID | EPKSS | HHHHHH |
| | | | R13C, T23A, E34K, | | NO: 54 | (SEQ ID | (SEQ ID |
| | | | P38T, R49E, P63V, | | | NO: 87) | NO: 93) |
| | | | S67A, T78A, 84C)]- | | | | |
| | | | EPKSS-His6 | | | | |
| | B | SEQ ID NO: 227 | [ICAM1 D1 (T2V, I10T, | None | SEQ ID | SG | WSHPQF |
| | | | R13C, T23A, E34K, | | NO: 55 | | EK |
| | | | P38T, E53R, P63V, | | | | (SEQ ID |
| | | | S67A, T78A, 84C)]-SG- | | | | NO: 94) |
| | | | Strep | | | | |
| HLPPW7 | A | SEQ ID NO: 228 | [ICAM1 D1 (T2V, I10T, | None | SEQ ID | EPKSS | HHHHHH |
| | | | R13C, L18F, T23A, | | NO: 56 | (SEQ ID | (SEQ ID |
| | | | E34K, P38T, P63V, | | | NO: 87) | NO: 93) |
| | | | S67A, T78A, 84C)]- | | | | |
| | | | EPKSS-His6 | | | | |
| | B | SEQ ID NO: 229 | [ICAM1 D1 (T2V, I10T, | None | SEQ ID | SG | WSHPQF |
| | | | R13C, L18A, T20A, | | NO: 57 | | EK |
| | | | T23A, E34K, P38T, | | | | (SEQ ID |
| | | | L42A, V51A, P63V, | | | | NO: 94) |
| | | | S67A, T78A, 84C)]-SG- | | | | |
| | | | Strep | | | | |
| HLPPW8 | A | SEQ ID NO: 230 | [ICAM1 D1 (T2V, I10T, | None | SEQ ID | EPKSS | HHHHHH |
| | | | R13C, L18W, T23A, | | NO: 58 | (SEQ ID | (SEQ ID |
| | | | E34K, P38T, P63V, | | | NO: 87) | NO: 93) |
| | | | S67A, T78A, 84C)]- | | | | |
| | | | EPKSS-His6 | | | | |
| | B | SEQ ID NO: 229 | [ICAM1 D1 (T2V, I10T, | None | SEQ ID | SG | WSHPQF |
| | | | R13C, L18A, T20A, | | NO: 57 | | EK |
| | | | T23A, E34K, P38T, | | | | (SEQ ID |
| | | | L42A, V51A, P63V, | | | | NO: 94) |
| | | | S67A, T78A, 84C)]-SG- | | | | |
| | | | Strep | | | | |

TABLE 28
Pseudo-Fabs Comprising Variant ICAM-1 D1 Homo- and Hetero-dimerization

| | | | Polypeptide | | | | | |
|---|---|---|---|---|---|---|---|---|
| | | | (Mutations relative | | | | | |
| | | | to WT are | Variable | | | | C-term |
| pFab ID | Chain | Chain ID | bolded and underlined) | Region | Elbow | Insert | Spacer | tag |
| HLPPB1 | HC | [VH]-(G₄S)₂- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 53 | (SEQ ID | (SEQ ID |
| | | (T2V, I10T, | KALEWLAHIYWDDDKRYNPSLK | | (SEQ | | NO: 87) | NO: 93) |
| | | R13C, T23A, | SRLTITKDTSKNQVVLTMTNMD | | ID NO: | | | |
| | | E34K, P38T, | PVDTATYYCARLYGFTYGFAYW | | 83) | | | |
| | | P63V, S67A, | GQGTLVTVSSGGGGSGGGGSQV | | | | | |
| | | T78A, 84C)]- | SVSPSKVTLPCGGSVLVTCSAS | | | | | |
| | | EPKSS-His6 | CDQPKLLGIKTPLTKKELLLPG | | | | | |
| | | | NNRKVYELSNVQEDSQVMCYAN | | | | | |
| | | | CPDGQSTAKAFLTVYCEPKSSH | | | | | |
| | | | HHHHH (SEQ ID NO: 231) | | | | | |
| | LC | [VL]-(G₄S)₂- | DIVMTQSPDSLAVSLGERATIN | SEQ ID | GGGGS | SEQ ID | SG | WSHPOF |
| | frag | [ICAM1 D1 | CRASQSVDYNGISYMHWYQQKP | NO: 97 | GGGGS | NO: 53 | | EK |
| | | (T2V, I10T, | GQPPKLLIYAASNPESGVPDRF | | (SEQ | | | (SEQ ID |
| | | R13C, T23A, | SGSGSGTDFTLTISSLQAEDVA | | ID NO: | | | NO: 94) |
| | | E34K, P38T, | VYYCQQIIEDPWTFGQGTKVEI | | 83) | | | |
| | | P63V, S67A, | KGGGGSGGGGSQVSVSPSKVTL | | | | | |
| | | T78A, 84C)]- | PCGGSVLVTCSASCDQPKLLGI | | | | | |
| | | SG-Strep | KTPLTKKELLLPGNNRKVYELS | | | | | |
| | | | NVQEDSQVMCYANCPDGQSTAK | | | | | |
| | | | AFLTVYCSGWSHPQFEK | | | | | |
| | | | (SEQ ID NO: 232) | | | | | |
| HLPPB2 | HC | [VH]-(G₄S)₃- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 53 | (SEQ DD | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | (SEQ | | | |
| | | E34K, P38T, | PVDTATYYCARLYGFTYGFAYW | | ID NO: | | | |
| | | P63V, S67A, | GQGTLVTVSSGGGGSGGGGSGG | | 84) | | | |
| | | T78A, 84C)]- | GGSQVSVSPSKVTLPCGGSVLV | | | | | |
| | | EPKSS-His6 | TCSASCDQPKLLGIKTPLTKKE | | | | | |
| | | | LLLPGNNRKVYELSNVQEDSQV | | | | | |
| | | | MCYANCPDGQSTAKAFLTVYCE | | | | | |
| | | | PKSSHHHHHH | | | | | |
| | | | (SEQ ID NO: 233) | | | | | |
| | LC | [VL]-(G₄S)₃- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 53 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | (SEQ | | | NO: 94) |
| | | E34K, P38T, | VYYCQQIIEDPWTFGQGTKVEI | | ID NO: | | | |
| | | P63V, S67A, | KGGGGSGGGGSGGGGSQVSVSP | | 84) | | | |
| | | T78A, 84C)]- | SKVTLPCGGSVLVTCSASCDQP | | | | | |
| | | SG-Strep | KLLGIKTPLTKKELLLPGNNRK | | | | | |
| | | | VYELSNVQEDSQVMCYANCPDG | | | | | |
| | | | QSTAKAFLTVYCSGWSHPQFEK | | | | | |
| | | | (SEQ ID NO: 234) | | | | | |
| HLPPB3 | HC | [VH]-(G₄S)₄- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 53 | (SEQ ID | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | GGGGS | | | |
| | | E34K, P38T, | PVDTATYYCARLYGFTYGFAYW | | (SEQ | | | |
| | | P63V, S67A, | GQGTLVTVSSGGGGSGGGGSGG | | ID NO: | | | |
| | | T78A, 84C)]- | GGSGGGGSQVSVSPSKVTLPCG | | 85) | | | |
| | | EPKSS-His6 | GSVLVTCSASCDQPKLLGIKTP | | | | | |
| | | | LTKKELLLPGNNRKVYELSNVQ | | | | | |
| | | | EDSQVMCYANCPDGQSTAKAFL | | | | | |
| | | | TVYCEPKSSHHHHHH | | | | | |
| | | | (SEQ ID NO: 235) | | | | | |
| | LC | [VL]-(G₄S)₄- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 53 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | GGGGS | | | NO: 94) |
| | | E34K, P38T, | VYYCQQIIEDPWTFGQGTKVEI | | (SEQ | | | |
| | | P63V, S67A, | KGGGGSGGGGSGGGGSGGGGSQ | | ID NO: | | | |
| | | T78A, 84C)]- | VSVSPSKVTLPCGGSVLVTCSA | | 85) | | | |
| | | SG-Strep | SCDQPKLLGIKTPLTKKELLLP | | | | | |
| | | | GNNRKVYELSNVQEDSQVMCYA | | | | | |
| | | | NCPDGQSTAKAFLTVYCSGWSH | | | | | |
| | | | PQFEK(SEQ ID NO: 236) | | | | | |
| HLPPB4 | HC | [VH]-(G₄S)₅- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 53 | (SEQ ID | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | GGGGS | | | |
| | | E34K, P38T, | PVDTATYYCARLYGFTYGFAYW | | GGGGS | | | |
| | | P63V, S67A, | GQGTLVTVSSGGGGSGGGGSGG | | (SEQ | | | |
| | | T78A, 84C)]- | GGSGGGGSGGGGSQVSVSPSKV | | ID NO: | | | |
| | | EPKSS-His6 | TLPCGGSVLVTCSASCDQPKLL | | 86) | | | |
| | | | GIKTPLTKKELLLPGNNRKVYE | | | | | |
| | | | LSNVQEDSQVMCYANCPDGQST | | | | | |
| | | | AKAFLTVYCEPKSSHHHHHH | | | | | |
| | | | (SEQ ID NO: 237) | | | | | |
| | LC | [VL]-(G₄S)₅- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 53 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | GGGGS | | | NO: 94) |
| | | E34K, P38T, | VYYCQQIIEDPWTFGQGTKVEI | | GGGGS | | | |
| | | P63V, S67A, | KGGGGSGGGGSGGGGSGGGGSG | | (SEQ ID | | | |
| | | T78A, 84C)]- | GGGSQVSVSPSKVTLPCGGSVL | | NO: 8 | | | |
| | | SG-Strep | VTCSASCDQPKLLGIKTPLTKK | | 6) | | | |
| | | | ELLLPGNNRKVYELSNVQEDSQ | | | | | |
| | | | VMCYANCPDGQSTAKAFLTVYC | | | | | |
| | | | SGWSHPQFEK | | | | | |
| | | | (SEQ ID NO: 238) | | | | | |
| HLPPB5 | HC | [VH]-(G₄S)₂- | DIVMTQSPDSLAVSLGERATIN | | GGGGS | SEQ ID | EPKSS | HHHHHH |
| | frag | [ICAM1 D1 | CRASQSVDYNGISYMHWYQQKP | | GGGGS | NO: 54 | (SEQ ID | (SEQ ID |
| | | (T2V, I10T, | GQPPKLLIYAASNPESGVPDRF | | (SEQ ID | | NO: 87) | NO: 93) |
| | | R13C, T23A, | SGSGSGTDFTLTISSLQAEDVA | | NO: 8 | | | |
| | | E34K, P38T, | VYYCQQIIEDPWTFGQGTKVEI | | 3) | | | |
| | | R49E, P63V, | KGGGGSGGGGSQVSVSPSKVTL | | | | | |
| | | S67A, T78A, | PCGGSVLVTCSASCDQPKLLGI | | | | | |
| | | 84C)]- | KTPLTKKELLLPGNNEKVYELS | | | | | |
| | | EPKSS-His6 | NVQEDSQVMCYANCPDGQSTAK | | | | | |
| | | | AFLTVYCEPKSSHHHHHH | | | | | |
| | | | (SEQ ID NO: 239) | | | | | |
| | LC | [VL]-(G₄S)₂- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 55 | | EK |
| | | (T2V, I10T, | GQPPKLLIYAASNPESGVPDRF | | (SEQ ID | | | (SEQ ID |
| | | R13C, T23A, | SGSGSGTDFTLTISSLQAEDVA | | NO: 8 | | | NO: 94) |
| | | E34K, P38T, | VYYCQQIIEDPWTFGQGTKVEI | | 3) | | | |
| | | E53R, P63V, | KGGGGSGGGGSQVSVSPSKVTL | | | | | |
| | | S67A, T78A, | PCGGSVLVTCSASCDQPKLLGI | | | | | |
| | | 84C)]-SG- | KTPLTKKELLLPGNNRKVYRLS | | | | | |
| | | Strep | NVQEDSQVMCYANCPDGQSTAK | | | | | |
| | | | AFLTVYCSGWSHPQFEK | | | | | |
| | | | (SEQ ID NO: 240) | | | | | |
| HLPPB6 | HC | [VH]-(G₄S)₃- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 54 | (SEQ ID | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | (SEQ ID | | | |
| | | E34K, P38T, | PVDTATYYCARLYGFTYGFAYW | | NO: 8 | | | |
| | | R49E, P63V, | GQGTLVTVSSGGGGSGGGGSGG | | 4) | | | |
| | | S67A, T78A, | GGSQVSVSPSKVTLPCGGSVLV | | | | | |
| | | 84C)]- | TCSASCDQPKLLGIKTPLTKKE | | | | | |
| | | EPKSS-His6 | LLLPGNNEKVYELSNVQEDSQV | | | | | |
| | | | MCYANCPDGQSTAKAFLTVYCE | | | | | |
| | | | PKSSHHHHHH | | | | | |
| | | | (SEQ ID NO: 241) | | | | | |
| | LC | [VL]-(G₄S)₃- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 55 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | (SEQ ID | | | NO: 94) |
| | | E34K, P38T, | VYYCQQIIEDPWTFGQGTKVEI | | NO: 84) | | | |
| | | E53R, P63V, | KGGGGSGGGGSGGGGSQVSVSP | | | | | |
| | | S67A, T78A, | SKVTLPCGGSVLVTCSASCDQP | | | | | |
| | | 84C)]-SG- | KLLGIKTPLTKKELLLPGNNRK | | | | | |
| | | Strep | VYRLSNVQEDSQVMCYANCPDG | | | | | |
| | | | QSTAKAFLTVYCSGWSHPQFEK | | | | | |
| | | | (SEQ ID NO: 242) | | | | | |
| HLPPB7 | HC | [VH]-(G₄S)₄- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 54 | (SEQ ID | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | GGGGS | | | |
| | | E34K, P38T, | PVDTATYYCARLYGFTYGFAYW | | (SEQ ID | | | |
| | | R49E, P63V, | GQGTLVTVSSGGGGSGGGGSGG | | NO: 85) | | | |
| | | S67A, T78A, | GGSGGGGSQVSVSPSKVTLPCG | | | | | |
| | | 84C)]- | GSVLVTCSASCDQPKLLGIKTP | | | | | |
| | | EPKSS-His6 | LTKKELLLPGNNEKVYELSNVQ | | | | | |
| | | | EDSQVMCYANCPDGQSTAKAFL | | | | | |
| | | | TVYCEPKSSHHHHHH | | | | | |
| | | | (SEQ ID NO: 243) | | | | | |
| | LC | [VL]-(G₄S)₄- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 55 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | GGGGS | | | NO: 94) |
| | | E34K, P38T, | VYYCQQIIEDPWTFGQGTKVEI | | (SEQ ID | | | |
| | | E53R, P63V, | KGGGGSGGGGSGGGGSGGGGSQ | | NO: 85) | | | |
| | | S67A, T78A | VSVSPSKVTLPCGGSVLVTCSA | | | | | |
| | | 84C)]-SG- | SCDQPKLLGIKTPLTKKELLLP | | | | | |
| | | Strep | GNNRKVYRLSNVQEDSQVMCYA | | | | | |
| | | | NCPDGQSTAKAFLTVYCSGWSH | | | | | |
| | | | PQFEK(SEQ ID NO: 244) | | | | | |
| HLPPB8 | HC | [VH]-(G₄S)₅- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 54 | (SEQ ID | (SEQ ID |
| | | (T2V, I10T, | KALEWLAHIYWDDDKRYNPSLK | | GGGGS | | NO: 87) | NO: 93) |
| | | R13C, T23A, | SRLTITKDTSKNQVVLTMTNMD | | GGGGS | | | |
| | | E34K, P38T, | PVDTATYYCARLYGFTYGFAYW | | GGGGS | | | |
| | | R49E, P63V, | GQGTLVTVSSGGGGSGGGGSGG | | (SEQ ID | | | |
| | | S67A, T78A, | GGSGGGGSGGGGSQVSVSPSKV | | NO: 86) | | | |
| | | 84C)]- | TLPCGGSVLVTCSASCDQPKLL | | | | | |
| | | EPKSS-His6 | GIKTPLTKKELLLPGNNEKVYE | | | | | |
| | | | LSNVQEDSQVMCYANCPDGQST | | | | | |
| | | | AKAFLTVYCEPKSSHHHHHH | | | | | |
| | | | (SEQ ID NO: 245) | | | | | |
| | LC | [VL]-(G₄S)₅- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 55 | | EK |
| | | (T2V, I10T, | GQPPKLLIYAASNPESGVPDRF | | GGGGS | | | (SEQ ID |
| | | R13C, T23A, | SGSGSGTDFTLTISSLQAEDVA | | GGGGS | | | NO: 94) |
| | | E34K, P38T, | VYYCQQIIEDPWTFGQGTKVEI | | GGGGS | | | |
| | | E53R, P63V, | KGGGGSGGGGSGGGGSGGGGSG | | (SEQ ID | | | |
| | | S67A, T78A, | GGGSQVSVSPSKVTLPCGGSVL | | NO: 86) | | | |
| | | 84C)]-SG- | VTCSASCDQPKLLGIKTPLTKK | | | | | |
| | | Strep | ELLLPGNNRKVYRLSNVQEDSQ | | | | | |
| | | | VMCYANCPDGQSTAKAFLTVYC | | | | | |
| | | | SGWSHPQFEK | | | | | |
| | | | (SEQ ID NO: 246) | | | | | |
| HLPPB9 | HC | [VH]-(G₄S)₂- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 56 | (SEQ ID | (SEQ ID |
| | | (T2V, I10T, | KALEWLAHIYWDDDKRYNPSLK | | (SEQ ID | | NO: 87) | NO: 93) |
| | | R13C, L18F, | SRLTITKDTSKNQVVLTMTNMD | | NO: 83) | | | |
| | | T23A, E34K, | PVDTATYYCARLYGFTYGFAYW | | | | | |
| | | P38T, P63V, | GQGTLVTVSSGGGGSGGGGSQV | | | | | |
| | | S67A, T78A, | SVSPSKVTLPCGGSVFVTCSAS | | | | | |
| | | 84C)]- | CDQPKLLGIKTPLTKKELLLPG | | | | | |
| | | EPKSS-His6 | NNRKVYELSNVQEDSQVMCYAN | | | | | |
| | | | CPDGQSTAKAFLTVYCEPKSSH | | | | | |
| | | | HHHHH(SEQ ID NO: 247) | | | | | |
| | LC | [VL]-(G₄S)₂- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | (SEQ ID | | | (SEQ ID |
| | | R13C, L18A, | SGSGSGTDFTLTISSLQAEDVA | | NO: 83) | | | NO: 94) |
| | | T20A, T23A, | VYYCQQIIEDPWTFGQGTKVEI | | | | | |
| | | E34K, P38T, | KGGGGSGGGGSQVSVSPSKVIL | | | | | |
| | | L42A, V51A, | PCGGSVAVACSASCDQPKLLGI | | | | | |
| | | P63V, S67A, | KTPLTKKEALLPGNNRKAYELS | | | | | |
| | | T78A, 84C)]- | NVQEDSQVMCYANCPDGQSTAK | | | | | |
| | | SG-Strep | AFLTVYCSGWSHPQFEK | | | | | |
| | | | (SEQ ID NO: 248) | | | | | |
| HLPPB10 | HC | [VH]-(G₄S)₃- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 56 | (SEQ ID | (SEQ ID |
| | | (T2V, I10T, | KALEWLAHIYWDDDKRYNPSLK | | GGGGS | | NO: 87) | NO: 93) |
| | | R13C, L18F, | SRLTITKDTSKNQVVLTMTNMD | | (SEQ ID | | | |
| | | T23A, E34K, | PVDTATYYCARLYGFTYGFAYW | | NO: 84) | | | |
| | | P38T, P63V, | GQGTLVTVSSGGGGGGGGSGG | | | | | |
| | | S67A, T78A, | GGSQVSVSPSKVTLPCGGSVEV | | | | | |
| | | 84C)]- | TCSASCDQPKLLGIKTPLTKKE | | | | | |
| | | EPKSS-His6 | LLLPGNNRKVYELSNVQEDSQV | | | | | |
| | | | MCYANCPDGQSTAKAFLTVYCE | | | | | |
| | | | PKSSHHHHHH | | | | | |
| | | | (SEQ ID NO: 249) | | | | | |
| | LC | [VL]-(G₄S)₃- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | (SEQ ID | | | NO: 94) |
| | | T20A, T23A, | VYYCQQIIEDPWTFGQGTKVEI | | NO: 84) | | | |
| | | E34K, P38T, | KGGGGSGGGGSGGGGSQVSVSP | | | | | |
| | | L42A, V51A, | SKVTLPCGGSVAVACSASCDQP | | | | | |
| | | P63V, S67A, | KLLGIKTPLTKKEALLPGNNRK | | | | | |
| | | T78A, 84C)]- | AYELSNVQEDSQVMCYANCPDG | | | | | |
| | | SG-Strep | QSTAKAFLTVYCSGWSHPQFEK | | | | | |
| | | | (SEQ ID NO: 250) | | | | | |
| HLPPB11 | HC | [VH]-(G₄S)₄- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 56 | (SEQ ID | (SEQ ID |
| | | (T2V, I10T, | KALEWLAHIYWDDDKRYNPSLK | | GGGGS | | NO: 87) | NO: 93) |
| | | R13C, L18F, | SRLTITKDTSKNQVVLTMTNMD | | GGGGS | | | |
| | | T23A, E34K, | PVDTATYYCARLYGFTYGFAYW | | (SEQ ID | | | |
| | | P38T, P63V, | GQGTLVTVSSGGGGSGGGGSGG | | NO: 85) | | | |
| | | S67A, T78A, | GGSGGGGSQVSVSPSKVTLPCG | | | | | |
| | | 84C)]- | GSVFVTCSASCDQPKLLGIKTP | | | | | |
| | | EPKSS-His6 | LTKKELLLPGNNRKVYELSNVQ | | | | | |
| | | | EDSQVMCYANCPDGQSTAKAFL | | | | | |
| | | | TVYCEPKSSHHHHHH | | | | | |
| | | | (SEQ ID NO: 251) | | | | | |
| | LC | [VL]-(G₄S)₄- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | GGGGS | | | NO: 94) |
| | | T20A, T23A, | VYYCQQIIEDPWTFGQGTKVEI | | (SEQ ID | | | |
| | | E34K, P38T, | KGGGGSGGGGSGGGGSGGGGSQ | | NO: 85) | | | |
| | | L42A, V51A, | VSVSPSKVTLPCGGSVAVACSA | | | | | |
| | | P63V, S67A, | SCDQPKLLGIKTPLTKKEALLP | | | | | |
| | | T78A, 84C)]- | GNNRKAYELSNVQEDSQVMCYA | | | | | |
| | | SG-Strep | NCPDGQSTAKAFLTVYCSGWSH | | | | | |
| | | | PQFEK(SEQ ID NO: 252) | | | | | |
| HLPPB12 | HC | [VH]-(G₄S)₅- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 56 | (SEQ ID | (SEQ ID |
| | | (T2V, I10T, | KALEWLAHIYWDDDKRYNPSLK | | GGGGS | | NO: 87) | NO: 93) |
| | | R13C, L18F, | SRLTITKDTSKNQVVLTMTNMD | | GGGGS | | | |
| | | T23A, E34K, | PVDTATYYCARLYGFTYGFAYW | | GGGGS | | | |
| | | P38T, P63V, | GQGTLVTVSSGGGGSGGGGSGG | | (SEQ ID | | | |
| | | S67A, T78A, | GGSGGGGSGGGGSQVSVSPSKV | | NO: 86) | | | |
| | | 84C)]- | TLPCGGSVFVTCSASCDQPKLL | | | | | |
| | | EPKSS-His6 | GIKTPLTKKELLLPGNNRKVYE | | | | | |
| | | | LSNVQEDSQVMCYANCPDGQST | | | | | |
| | | | AKAFLTVYCEPKSSHHHHHH | | | | | |
| | | | (SEQ ID NO: 253) | | | | | |
| | LC | [VL]-(G₄S)₅- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | GGGGS | | | NO: 94) |
| | | T20A, T23A, | VYYCQQIIEDPWTFGQGTKVEI | | GGGGS | | | |
| | | E34K, P38T, | KGGGGSGGGGSGGGGSGGGGSG | | (SEQ ID | | | |
| | | L42A, V51A, | GGGSQVSVSPSKVTLPCGGSVA | | NO: 86) | | | |
| | | P63V, S67A, | VACSASCDQPKLLGIKTPLTKK | | | | | |
| | | T78A, 84C)]- | EALLPGNNRKAYELSNVQEDSQ | | | | | |
| | | SG-Strep | VMCYANCPDGQSTAKAFLTVYC | | | | | |
| | | | SGWSHPQFEK | | | | | |
| | | | (SEQ ID NO: 254) | | | | | |
| HLPPB13 | HC | [VH]-(G₄S)₂- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 58 | (SEQ ID | (SEQ ID |
| | | (T2V, I10T, | KALEWLAHIYWDDDKRYNPSLK | | (SEQ ID | | NO: 87) | NO: 93) |
| | | R13C, L18W, | SRLTITKDTSKNQVVLTMTNMD | | NO: 83) | | | |
| | | T23A, E34K, | PVDTATYYCARLYGFTYGFAYW | | | | | |
| | | P38T, P63V, | GQGTLVTVSSGGGGSGGGGSQV | | | | | |
| | | S67A, T78A, | SVSPSKVTLPCGGSVWVTCSAS | | | | | |
| | | 84C)]- | CDQPKLLGIKTPLTKKELLLPG | | | | | |
| | | EPKSS-His6 | NNRKVYELSNVQEDSQVMCYAN | | | | | |
| | | | CPDGQSTAKAFLTVYCEPKSSH | | | | | |
| | | | HHHHH(SEQ ID NO: 255) | | | | | |
| | LC | [VL]-(G₄S)₂- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | (SEQ ID | | | (SEQ ID |
| | | R13C, L18A, | SGSGSGTDFTLTISSLQAEDVA | | NO: 83) | | | NO: 94) |
| | | T20A, T23A, | VYYCQQIIEDPWTFGQGTKVEI | | | | | |
| | | E34K, P38T, | KGGGGSGGGGSQVSVSPSKVTL | | | | | |
| | | L42A, V51A, | PCGGSVAVACSASCDQPKLLGI | | | | | |
| | | P63V, S67A, | KTPLTKKEALLPGNNRKAYELS | | | | | |
| | | T78A, 84C)]- | NVQEDSQVMCYANCPDGQSTAK | | | | | |
| | | SG-Strep | AFLTVYCSGWSHPQFEK | | | | | |
| | | | (SEQ ID NO: 256) | | | | | |
| HLPPB14 | HC | [VH]-(G₄S)₃- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 58 | (SEQ ID | (SEQ ID |
| | | (T2V, I10T, | KALEWLAHIYWDDDKRYNPSLK | | GGGGS | | NO: 87) | NO: 93) |
| | | R13C, L18W, | SRLTITKDTSKNQVVLTMTNMD | | (SEQ ID | | | |
| | | T23A, E34K, | PVDTATYYCARLYGFTYGFAYW | | NO: 84) | | | |
| | | P38T, P63V, | GQGTLVTVSSGGGGSGGGGSGG | | | | | |
| | | S67A, T78A, | GGSQVSVSPSKVTLPCGGSVWV | | | | | |
| | | 84C)]- | TCSASCDQPKLLGIKTPLTKKE | | | | | |
| | | EPKSS-His6 | LLLPGNNRKVYELSNVQEDSQV | | | | | |
| | | | MCYANCPDGQSTAKAFLTVYCE | | | | | |
| | | | PKSSHHHHHH | | | | | |
| | | | (SEQ ID NO: 257) | | | | | |
| | LC | [VL]-(G₄S)₃- | DIVMTQSPDSLAVSLGERATIN | SEQ ID | GGGGS | SEQ ID | SG | WSHPOF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | (SEQ ID | | | NO: 94) |
| | | T20A, T23A, | VYYCQQIIEDPWTFGQGTKVEI | | NO: 84) | | | |
| | | E34K, P38T, | KGGGGSGGGGSGGGGSQVSVSP | | | | | |
| | | L42A, V51A, | SKVTLPCGGSVAVACSASCDQP | | | | | |
| | | P63V, S67A, | KLLGIKTPLTKKEALLPGNNRK | | | | | |
| | | T78A, 84C)]- | AYELSNVQEDSQVMCYANCPDG | | | | | |
| | | SG-Strep | QSTAKAFLTVYCSGWSHPQFEK | | | | | |
| | | | (SEQ ID NO: 258) | | | | | |
| HLPPB15 | HC | [VH]-(G₄S)₄- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 58 | (SEQ ID | (SEQ ID |
| | | (T2V, I10T, | KALEWLAHIYWDDDKRYNPSLK | | GGGGS | | NO: 87) | NO: 93) |
| | | R13C, L18W, | SRLTITKDTSKNQVVLTMTNMD | | GGGGS | | | |
| | | T23A, E34K, | PVDTATYYCARLYGFTYGFAYW | | (SEQ ID | | | |
| | | P38T, P63V, | GQGTLVTVSSGGGGSGGGGSGG | | NO: 85) | | | |
| | | S67A, T78A, | GGSGGGGSQVSVSPSKVTLPCG | | | | | |
| | | 84C)]- | GSVWVTCSASCDQPKLLGIKTP | | | | | |
| | | EPKSS-His6 | LTKKELLLPGNNRKVYELSNVQ | | | | | |
| | | | EDSQVMCYANCPDGQSTAKAFL | | | | | |
| | | | TVYCEPKSSHHHHHH | | | | | |
| | | | (SEQ ID NO: 259) | | | | | |
| | LC | [VL]-(G₄S)₄- | DIVMTQSPDSLAVSLGERATIN | SEQ ID | GGGGS | SEQ ID | SG | WSHPOF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | GGGGS | | | NO: 94) |
| | | T20A, T23A, | VYYCQQIIEDPWTFGQGTKVEI | | (SEQ ID | | | |
| | | E34K, P38T, | KGGGGSGGGGSGGGGSGGGGSQ | | NO: 85) | | | |
| | | L42A, V51A, | VSVSPSKVTLPCGGSVAVACSA | | | | | |
| | | P63V, S67A, | SCDQPKLLGIKTPLTKKEALLP | | | | | |
| | | T78A, 84C)]- | GNNRKAYELSNVQEDSQVMCYA | | | | | |
| | | SG-Strep | NCPDGQSTAKAFLTVYCSGWSH | | | | | |
| | | | PQFEK(SEQ ID NO: 260) | | | | | |
| HLPPB16 | HC | [VH]-(G₄S)₅- | QITLKESGPTLVKPTQTLTLTC | SEQ ID | GGGGS | SEQ ID | EPKSS | HHHHHH |
| | frag | [ICAM1 D1 | TFSGFSLSTSGMGVSWIRQPPG | NO: 96 | GGGGS | NO: 58 | (SEQ ID | (SEQ ID |
| | | (T2V, I10T, | KALEWLAHIYWDDDKRYNPSLK | | GGGGS | | NO: 87) | NO: 93) |
| | | R13C, L18W, | SRLTITKDTSKNQVVLTMTNMD | | GGGGS | | | |
| | | T23A, E34K, | PVDTATYYCARLYGFTYGFAYW | | GGGGS | | | |
| | | P38T, P63V, | GQGTLVTVSSGGGGSGGGGSGG | | (SEQ ID | | | |
| | | S67A, T78A, | GGSGGGGSGGGGSQVSVSPSKV | | NO: 86) | | | |
| | | 84C)]- | TLPCGGSVWVTCSASCDQPKLL | | | | | |
| | | EPKSS-His6 | GIKTPLTKKELLLPGNNRKVYE | | | | | |
| | | | LSNVQEDSQVMCYANCPDGQST | | | | | |
| | | | AKAFLTVYCEPKSSHHHHHH | | | | | |
| | | | (SEQ ID NO: 261) | | | | | |
| | LC | [VL]-(G₄S)₅- | DIVMTQSPDSLAVSLGERATIN | SEQ ID | GGGGS | SEQ ID | SG | WSHPQF |
| | frag | [ICAM1 D1 | CRASQSVDYNGISYMHWYQQKP | NO: 97 | GGGGS | NO: 57 | | EK |
| | | (T2V, I10T, | GQPPKLLIYAASNPESGVPDRF | | GGGGS | | | (SEQ ID |
| | | R13C, L18A, | SGSGSGTDFTLTISSLQAEDVA | | GGGGS | | | NO: 94) |
| | | T20A, T23A, | VYYCQQIIEDPWTFGQGTKVEI | | GGGGS | | | |
| | | E34K, P38T, | KGGGGSGGGGSGGGGSGGGGSG | | (SEQ ID | | | |
| | | L42A, V51A, | GGGSQVSVSPSKVTLPCGGSVA | | NO: 86) | | | |
| | | P63V, S67A, | VACSASCDQPKLLGIKTPLTKK | | | | | |
| | | T78A, 84C)]- | EALLPGNNRKAYELSNVQEDSQ | | | | | |
| | | SG-Strep | VMCYANCPDGQSTAKAFLTVYC | | | | | |
| | | | SGWSHPQFEK | | | | | |
| | | | (SEQ ID NO: 262) | | | | | |

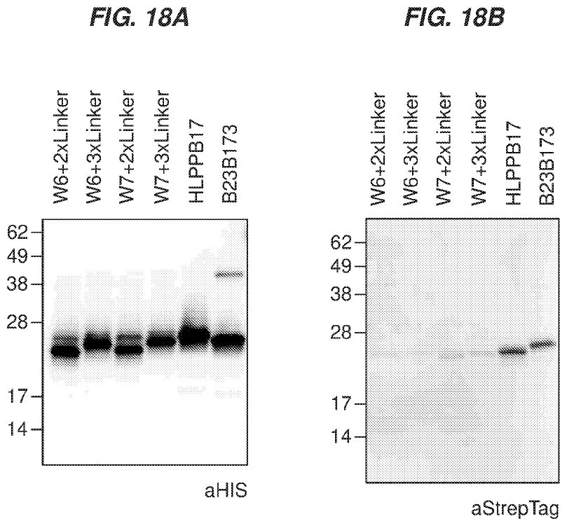

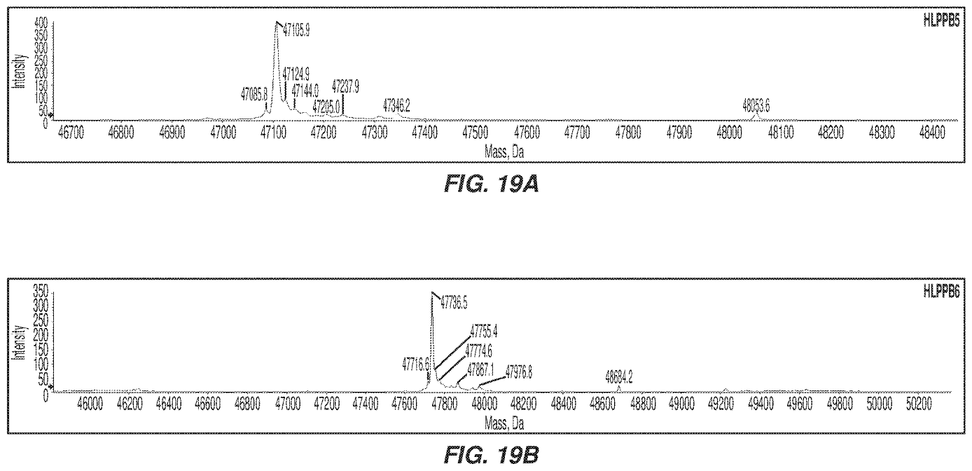

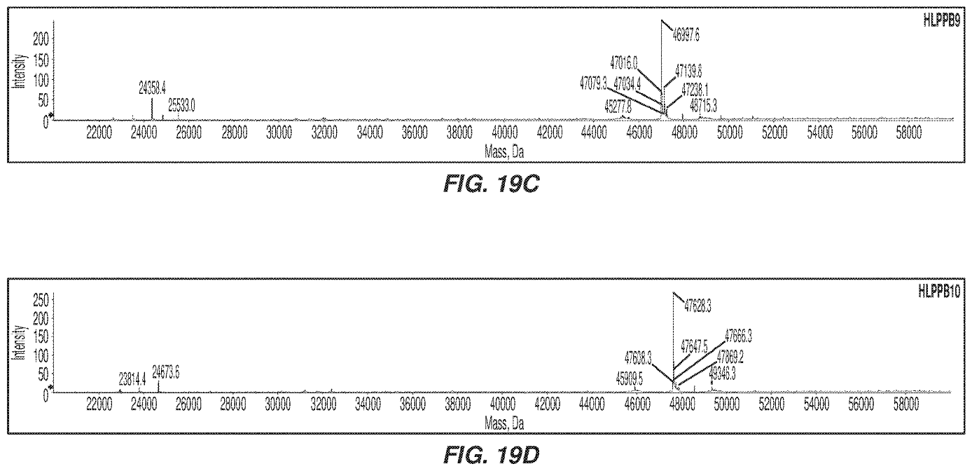

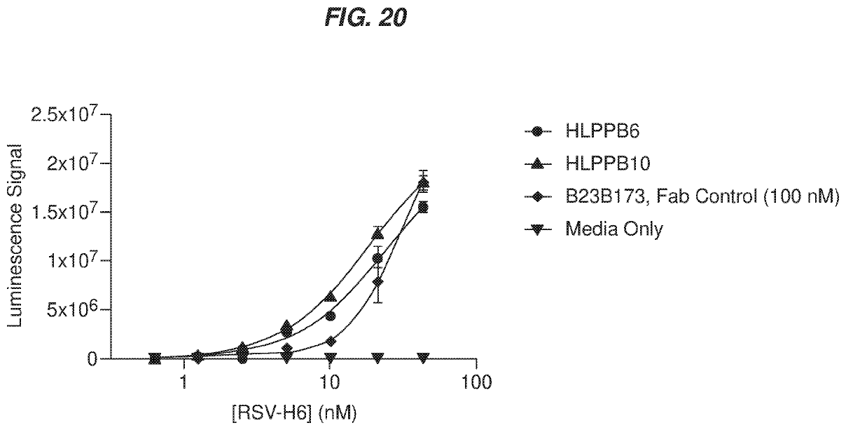

The same VH and VL regions used to construct the HLA-E pseudo-Fabs were fused to the paired ICAM D1 heterodimer domains, joined with elbows of lengths of 10, 15, 20, or 25 amino acids (e.g., (G₄S)₂ (SEQ ID NO: 83), (G₄S)₃ (SEQ ID NO: 84), (G₄S)₄ (SEQ ID NO: 85), (G₄S)₅ (SEQ ID NO: 86), respectively). Pseudo-Fabs comprising variant ICAM1 D1 dimer sequences of varying elbow sequence lengths were evaluated for expression (FIGS. 18A-18B) and for the presence of fully formed heterodimers (FIGS. 19A-19D). ICAM derived pseudo-Fabs also exhibited binding to RSV glycoprotein at levels comparable to their Fab counterpart (FIG. 20).

Polynucleotide sequences utilized throughout the Examples to encode the antigen-binding molecules, or variable regions, elbow regions, dimerization domains, spacer regions, or C-terminal tags thereof were CHO optimized and are presented in TABLE 29. In specific embodiments, a polynucleotide as described herein comprises or consists of a sequence selected from TABLE 29. In specific embodiments, a polynucleotide as described herein comprises or consists a fragment or portion of a sequence selected from TABLE 29 (e.g., wherein the fragment or portion encodes a variable domain, elbow region, dimerization domain, spacer region, or C-terminal tag in the polynucleotide sequence of TABLE 29).

TABLE 29
Polynucleotides Encoding Antigen-Binding Molecules and Fragments Thereof

| Descriptors | Polynucleotide Sequence |
|---|---|
| B21 VH | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCT (SEQ ID NO: 263) |
| B21 VL | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAG |
| | (SEQ ID NO: 264) |
| EA3 (C-terminal H6 | CTGCATCTGGAACCCCCCAAAACTCATGTCACCCATCATCCCATATCTGACCACGAAGCAACCCTGC |
| tag) | GTTGCTGGGCACTTGGGTTTTACCCCGCAGAGATCACTCTCACTTGGCAACAAGATGGGGAGGGTCA |
| | TACCCAGGATACCGAGCTTGTCGAAACAAGGCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 265) |
| B2M (C-terminal Strep- | CGCACAGTGAGAACCCCTAAGATACAAGTTTATAGTCGGCACCCCGCAGAGAACGGGAAAAGTAACT |
| tag II) | TCCTTAATTGCTATGTCTCTGGCTTCCATCCTTCTGACATCGAGGTGGACTTGTTGAAAAACGGGGA |
| | GAGAATTGAAAAAGTGGAACACAGCGATCTGAGCTTCTCCAAAGATTGGTCTTTTTATCTCCTCTAC |
| | TACACCGAATTTACCCCAACTGAGAAAGATGAATATGCCTGTCGGGTTAATCACGTAACTTTGTCTC |
| | AACCAAAAATTGTCAAATGGGACAGGGACATGTCAGGGTGGAGTCACCCTCAGTTCGAAAAG |
| | (SEQ ID NO: 266) |
| B2M (SST) ‘RTV’ (C- | AGAACCGTTAGAACTCCTAAGATTCAGGTCTATTCCCGACATCCTGCTGAGAATGGAAAGTCTAATT |
| terminal Strep-tag II) | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 267) |
| EA3 R234A-His | CTGCATCTGGAACCCCCCAAAACTCATGTCACCCATCATCCCATATCTGACCACGAAGCAACCCTGC |
| | GTTGCTGGGCACTTGGGTTTTACCCCGCAGAGATCACTCTCACTTGGCAACAAGATGGGGAGGGTCA |
| | TACCCAGGATACCGAGCTTGTCGAAACAgccCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 268) |
| EA3 R234L-His | CTGCATCTGGAACCCCCCAAAACTCATGTCACCCATCATCCCATATCTGACCACGAAGCAACCCTGC |
| | GTTGCTGGGCACTTGGGTTTTACCCCGCAGAGATCACTCTCACTTGGCAACAAGATGGGGAGGGTCA |
| | TACCCAGGATACCGAGCTTGTCGAAACActgCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 269) |
| EA3 R234A, Q242A- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACAGCCCCCGCCGGGGATGGAACCTTTgctAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 270) |
| EA3 R234L, Q242L- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACACTGCCCGCCGGGGATGGAACCTTTctgAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 271) |
| EA3 R234A, Q242L- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACAGCCCCCGCCGGGGATGGAACCTTTctgAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 272) |
| EA3 R234L, Q242A- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACACTGCCCGCCGGGGATGGAACCTTTgccAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 273) |
| EA3 R202A-His | CTGCATCTGGAACCCCCCAAAACTCATGTCACCCATCATCCCATATCTGACCACGAAGCAACCCTGg |
| | CCTGCTGGGCACTTGGGTTTTACCCCGCAGAGATCACTCTCACTTGGCAACAAGATGGGGAGGGTCA |
| | TACCCAGGATACCGAGCTTGTCGAAACAAGGCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 274) |
| EA3 Native Hinge | CTTCATCTTGAACCACCTAAGACCCACGTTACTCATCATCCAATAAGCGACCACGAAGCCACCCTGA |
| GGGS | GATGCTGGGCACTTGGTTTTTATCCAGCAGAGATCACTCTGACTTGGCAGCAGGACGGCGAAGGTCA |
| | CACTCAAGACACAGAACTCGTGGAAACTAGACCAGCCGGGGATGGCACTTTCCAAAAATGGGCAGCC |
| | GTCGTAGTACCTTCTGGGGAAGAGCAGAGATACACCTGCCACGTCCAGCATGAAGGGCTGCCTGAGC |
| | CCGTAACCCTGAGGTGGGAACCTAAGTCCTGCCACCACCATCATCATCAT (SEQ ID NO: 275) |
| EA3 R202C | CTCCATTTGGAACCTCCTAAAACACATGTCACTCACCATCCCATCTCCGACCACGAAGCTACCTTGT |
| | GCTGTTGGGCTCTTGGATTCTACCCAGCCGAAATAACTCTGACATGGCAACAGGATGGAGAAGGTCA |
| | TACCCAGGATACAGAGCTGGTAGAAACTCGGCCAGCCGGGGATGGTACATTCCAGAAGTGGGCAGCA |
| | GTAGTTGTGCCATCCGGGGAGGAGCAAAGATATACCTGTCATGTCCAACACGAAGGTTTGCCTGAAC |
| | CAGTGACTCTTCGTTGGGAGCCCAAGAGTAGCcaccatcaccaccatcat (SEQ ID NO: 276) |
| EA3 H192C | TTGCATCTTGAACCCCCTAAGACACACGTTACACATTGCCCTATCTCCGATCACGAGGCAACTCTCC |
| | GATGTTGGGCACTGGGTTTCTATCCCGCAGAGATAACATTGACTTGGCAACAGGATGGGGAAGGGCA |
| | TACACAAGATACAGAACTGGTCGAAACAAGGCCCGCCGGAGACGGAACATTTCAAAAGTGGGCAGCA |
| | GTTGTAGTTCCCTCTGGGGAGGAACAGCGCTACACTTGCCATGTGCAGCACGAAGGTCTGCCAGAGC |
| | CTGTCACTCTCAGATGGGAGCCCAAGAGTAGCcaccatcaccaccatcat (SEQ ID NO: 277) |
| EA3 E232C | TTGCACCTCGAACCACCCAAAACCCACGTGACTCATCATCCCATCTCTGACCACGAGGCCACACTTC |
| | GGTGTTGGGCTCTGGGCTTCTACCCAGCAGAGATAACCCTGACCTGGCAACAGGATGGGGAGGGTCA |
| | CACACAGGACACAGAATTGGTCTGCACTAGACCTGCCGGTGACGGCACCTTTCAAAAGTGGGCAGCA |
| | GTGGTTGTGCCAAGCGGAGAGGAGCAACGATACACCTGCCACGTACAACACGAAGGGCTCCCTGAAC |
| | CAGTAACACTGCGCTGGGAGCCCAAGAGTAGCcaccatcaccaccatcat (SEQ ID NO: 278) |
| EA3 R234C | TTGCATCTTGAACCTCCTAAAACTCATGTAACTCACCACCCAATTTCAGACCATGAAGCTACTCTCC |
| | GATGCTGGGCTCTTGGCTTTTATCCTGCCGAAATCACATTGACATGGCAACAGGATGGAGAGGGGCA |
| | TACACAGGACACCGAGTTGGTGGAAACTTGTCCCGCAGGGGATGGCACATTTCAAAAATGGGCTGCT |
| | GTTGTTGTGCCCAGCGGCGAAGAACAGCGGTACACATGTCACGTCCAACATGAAGGCTTGCCAGAGC |
| | CCGTAACTCTTCGCTGGGAGCCCAAGAGTAGCcaccatcaccaccatcat (SEQ ID NO: 279) |
| EA3 D238C | TTGCATCTCGAGCCCCCCAAGACCCACGTTACTCACCACCCTATAAGCGATCACGAGGCAACATTGA |
| | GGTGCTGGGCCTTGGGTTTCTACCCCGCTGAGATCACACTCACTTGGCAGCAAGATGGAGAAGGGCA |
| | TACACAGGACACCGAACTCGTGGAAACCCGACCAGCCGGTTGTGGTACATTCCAGAAATGGGCTGCC |
| | GTAGTAGTCCCTAGCGGTGAGGAGCAGCGATATACCTGTCATGTGCAACACGAGGGCTTGCCCGAAC |
| | CAGTCACTTTGAGATGGGAGCCCAAGAGTAGCcaccatcaccaccatcat (SEQ ID NO: 280) |
| B2M (SST) Y10F | AGAACCGTTAGAACTCCTAAGATTCAGGTCttcTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 281) |
| B2M (SST) Y10W | AGAACCGTTAGAACTCCTAAGATTCAGGTCtggTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 282) |
| B2M (SST) D98F | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGttcATGTCAGGGTGGAGTCACCCTCAGTTCGAAAAG |
| | (SEQ ID NO: 283) |
| B2M (SST) D98W | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGtggATG-TCAGGGTGGAGTCACCCTCAGTTCGAAAAG |
| | (SEQ ID NO: 284) |
| B2M (SST) M99F | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATttcTCAGGGTGGAGTCACCCTCAGTTCGAAAAG |
| | (SEQ ID NO: 285) |
| B2M (SST) M99W | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATtggTCAGGGTGGAGTCACCCTCAGTTCGAAAAG |
| | (SEQ ID NO: 286) |
| B2M (SST) Native | AGAACTGTACGCACACCAAAGATACAAGTATATTCCAGACACCCCGCAGAAAATGGTAAGAGCAATT |
| Hinge GGS | TCTTGAACTGTTATGTATCCGGTTTTCACCCTTCTGATATAGAGGTAGATTTGCTGAAAAATGGTGA |
| | GCGGATAGAGAAAGTTGAGCACTCCGATCTTAGCTCATCTAAGGATTCAAGCACTTATCTCTTGTAT |
| | TACACAGAGTTTACTCCCACCGAGAAAGACGAGTATGCCTGCCGTGTCAACCATGTCACTTTGTCCC |
| | AGCCCAAGATAGTGAAATGGGACCGAGATGGAGGCAGTGGAGAGTGCTCTGGGTACCCTTACGATGT |
| | CCCTGATTACGCA (SEQ ID NO: 287) |
| B2M (SST) Native | CGGACAGTGCGGACTCCAAAGATACAAGTTTATAGCAGGCATCCCGCTGAGAATGGGAAGTCCAACT |
| Hinge GGGS | TTCTTAATTGCTATGTCTCCGGGTTCCACCCCTCTGACATCGAGGTGGACTTGCTGAAGAATGGGGA |
| | AAGAATAGAGAAAGTTGAGCACTCTGATCTCAGTTCATCCAAGGATTCCTCAACATACCTCTTGTAC |
| | TACACTGAGTTCACCCCAACCGAAAAAGATGAATATGCTTGCAGAGTCAATCACGTGACCCTGTCAC |
| | AGCCTAAAATAGTCAAGTGGGATAGGGACGGAGGTGGGAGCGGTGAGTGC-TCTGGGTACCCTTACG |
| | ATGTCCCTGATTACGCA (SEQ ID NO: 288) |
| B2M (SST) M99C GGS | CGGACTGTTCGCACACCCAAAATTCAGGTTTACTCCCGACATCCAGCTGAGAACGGTAAGTCTAATT |
| | TCCTGAACTGTTACGTGTCTGGTTTTCATCCTTCAGATATAGAAGTTGACCTCCTCAAAAACGGCGA |
| | AAGGATAGAAAAGGTAGAGCACAGCGACTTGAGTAGCAGCAAAGATTCTAGCACATATCTGCTGTAT |
| | TATACCGAATTTACACCCACAGAGAAGGACGAGTACGCATGTCGGGTAAACCATGTGACACTCTCTC |
| | AGCCTAAGATCGTCAAGTGGGACCGAGACTGTGGAGGTTCTGGTGAGAGTTCCGGCTATCCTTATGA |
| | TGTGCCCGACTACGCC (SEQ ID NO: 289) |
| B2M (SST) D98C GGS | CGCACAGTACGTACACCAAAGATCCAGGTTTACTCACGTCACCCCGCCGAGAATGGCAAGAGCAACT |
| | TCCTCAACTGCTACGTGAGTGGATTCCACCCTAGTGACATCGAGGTTGACCTGCTGAAAAATGGTGA |
| | GCGCATTGAAAAAGTTGAACATTCTGACTTGAGCAGCAGTAAAGACAGTTCAACATATCTCCTCTAC |
| | TACACCGAGTTTACACCAACCGAAAAAGACGAGTACGCATGTAGAGTTAATCATGTGACTCTGTCAC |
| | AACCTAAAATCGTAAAGTGGGACCGATGCATGGGAGGTTCTGGTGAGAGTTCCGGCTATCCTTATGA |
| | TGTGCCCGACTACGCC (SEQ ID NO: 290) |
| B2M (SST) K6C GGS | CGAACCGTCCGAACACCCTGCATTCAAGTTTACAGCCGGCATCCAGCCGAGAATGGGAAGTCTAATT |
| | TTCTGAATTGTTATGTAAGCGGATTCCACCCAAGCGACATTGAGGTGGACCTTTTGAAGAATGGCGA |
| | ACGCATAGAGAAAGTCGAACATAGCGACCTTTCAAGCTCTAAAGATTCCAGTACCTACCTCCTTTAT |
| | TATACTGAGTTTACCCCCACTGAAAAGGACGAATATGCTTGTAGGGTGAACCACGTCACTCTCAGCC |
| | AGCCAAAGATTGTTAAATGGGATAGGGATATGGGAGGTTCTGGTGAGAGTTCCGGCTATCCTTATGA |
| | TGTGCCCGACTACGCC (SEQ ID NO: 291) |
| B2M (SST) Y10C GGS | CGCACTGTGAGAACACCTAAGATCCAGGTCTGTTCTAGGCATCCCGCTGAAAACGGCAAATCAAACT |
| | TTTTGAACTGTTATGTGAGTGGTTTCCATCCCTCCGATATAGAAGTTGACTTGCTTAAAAATGGAGA |
| | ACGCATCGAAAAAGTGGAACATTCTGATCTTTCTTCCAGCAAGGATAGCTCTACATACCTCCTTTAC |
| | TACACCGAGTTTACTCCAACAGAGAAGGATGAGTACGCTTGTAGAGTTAACCATGTCACTCTCAGCC |
| | AACCTAAGATTGTTAAATGGGACAGAGATATGGGAGGTTCTGGTGAGAGTTCCGGCTATCCTTATGA |
| | TGTGCCCGACTACGCC (SEQ ID NO: 292) |
| B2M (SST) R12C GGS | CGCACTGTAAGGACCCCCAAAATACAGGTCTACTCATGTCACCCCGCCGAGAATGGAAAGTCCAATT |
| | TCCTGAACTGCTACGTAAGTGGGTTCCATCCTTCTGATATAGAAGTTGATCTGCTCAAGAACGGAGA |
| | GCGTATAGAAAAAGTTGAACATAGTGATCTTTCAAGCAGCAAAGACTCTTCAACCTATCTGCTTTAC |
| | TATACTGAGTTCACTCCAACAGAAAAGGACGAATACGCTTGCCGGGTGAACCACGTCACTCTTTCTC |
| | AACCTAAGATTGTAAAATGGGACCGAGACATGGGAGGTTCTGGTGAGAGTTCCGGCTATCCTTATGA |
| | TGTGCCCGACTACGCC (SEQ ID NO: 293) |
| B2M (SST) M99C | CGGACTGTTCGCACACCCAAAATTCAGGTTTACTCCCGACATCCAGCTGAGAACGGTAAGTCTAATT |
| GGGS | TCCTGAACTGTTACGTGTCTGGTTTTCATCCTTCAGATATAGAAGTTGACCTCCTCAAAAACGGCGA |
| | AAGGATAGAAAAGGTAGAGCACAGCGACTTGAGTAGCAGCAAAGATTCTAGCACATATCTGCTGTAT |
| | TATACCGAATTTACACCCACAGAGAAGGACGAGTACGCATGTCGGGTAAACCATGTGACACTCTCTC |
| | AGCCTAAGATCGTCAAGTGGGACCGAGACTGTGGAGGAGGTAGCGGAGAGAGCAGTGGATATCCCTA |
| | CGATGTACCTGATTACGCA (SEQ ID NO: 294) |
| B2M (SST) D98C | CGCACAGTACGTACACCAAAGATCCAGGTTTACTCACGTCACCCCGCCGAGAATGGCAAGAGCAACT |
| GGGS | TCCTCAACTGCTACGTGAGTGGATTCCACCCTAGTGACATCGAGGTTGACCTGCTGAAAAATGGTGA |
| | GCGCATTGAAAAAGTTGAACATTCTGACTTGAGCAGCAGTAAAGACAGTTCAACATATCTCCTCTAC |
| | TACACCGAGTTTACACCAACCGAAAAAGACGAGTACGCATGTAGAGTTAATCATGTGACTCTGTCAC |
| | AACCTAAAATCGTAAAGTGGGACCGATGCATGGGAGGAGGTAGCGGAGAGAGCAGTGGATATCCCTA |
| | CGATGTACCTGATTACGCA (SEQ ID NO: 295) |
| B2M (SST) K6C | CGAACCGTCCGAACACCCTGCATTCAAGTTTACAGCCGGCATCCAGCCGAGAATGGGAAGTCTAATT |
| GGGS | TTCTGAATTGTTATGTAAGCGGATTCCACCCAAGCGACATTGAGGTGGACCTTTTGAAGAATGGCGA |
| | ACGCATAGAGAAAGTCGAACATAGCGACCTTTCAAGCTCTAAAGATTCCAGTACCTACCTCCTTTAT |
| | TATACTGAGTTTACCCCCACTGAAAAGGACGAATATGCTTGTAGGGTGAACCACGTCACTCTCAGCC |
| | AGCCAAAGATTGTTAAATGGGATAGGGATATGGGAGGAGGTAGCGGAGAGAGCAGTGGATATCCCTA |
| | CGATGTACCTGATTACGCA (SEQ ID NO: 296) |
| B2M (SST) Y10C | CGCACTGTGAGAACACCTAAGATCCAGGTCTGTTCTAGGCATCCCGCTGAAAACGGCAAATCAAACT |
| GGGS | TTTTGAACTGTTATGTGAGTGGTTTCCATCCCTCCGATATAGAAGTTGACTTGCTTAAAAATGGAGA |
| | ACGCATCGAAAAAGTGGAACATTCTGATCTTTCTTCCAGCAAGGATAGCTCTACATACCTCCTTTAC |
| | TACACCGAGTTTACTCCAACAGAGAAGGATGAGTACGCTTGTAGAGTTAACCATGTCACTCTCAGCC |
| | AACCTAAGATTGTTAAATGGGACAGAGATATGGGAGGAGGTAGCGGAGAGAGCAGTGGATATCCCTA |
| | CGATGTACCTGATTACGCA (SEQ ID NO: 297) |
| B2M (SST) R12C | CGCACTGTAAGGACCCCCAAAATACAGGTCTACTCATGTCACCCCGCCGAGAATGGAAAGTCCAATT |
| GGGS | TCCTGAACTGCTACGTAAGTGGGTTCCATCCTTCTGATATAGAAGTTGATCTGCTCAAGAACGGAGA |
| | GCGTATAGAAAAAGTTGAACATAGTGATCTTTCAAGCAGCAAAGACTCTTCAACCTATCTGCTTTAC |
| | TATACTGAGTTCACTCCAACAGAAAAGGACGAATACGCTTGCCGGGTGAACCACGTCACTCTTTCTC |
| | AACCTAAGATTGTAAAATGGGACCGAGACATGGGAGGAGGTAGCGGAGAGAGCAGTGGATATCCCTA |
| | CGATGTACCTGATTACGCA (SEQ ID NO: 298) |
| GGS | GGGGGATCA |
| RTVGGS (SEQ ID NO: | AGAACCGTTGGGGGATCA (SEQ ID NO: 300) |
| 59) | |
| GGGGSGGGGS (SEQ | GGCGGAGGCGGCTCTGGTGGCGGCGGGTCT (SEQ ID NO: 301) |
| ID NO: 83) | |
| GGGGSGGGGSGGG | GGGGGCGGCGGAAGTGGAGGCGGGGGTTCAGGTGGGGGAGGGAGC (SEQ ID NO: 302) |
| GS (SEQ ID NO: 84) | |
| GGGGSGGGGSGGG | GGGGGCGGAGGATCTGGCGGGGGAGGTTCTGGAGGAGGTGGAAGTGGCGGCGGTGGATCT |
| GSGGGGS (SEQ ID | (SEQ ID NO: 303) |
| NO: 85) | |
| GGGGSGGGGSGGG | GGCGGCGGGGGGAGCGGCGGTGGCGGCTCCGGCGGAGGGGGTTCAGGCGGAGGTGGGTCCGGCGGAG |
| GSGGGGSGGGGS | GAGGTTCT (SEQ ID NO: 304) |
| (SEQ ID NO: 86) | |
| EA3 6xHis | CTGCATCTGGAACCCCCCAAAACTCATGTCACCCATCATCCCATATCTGACCACGAAGCAACCCTGC |
| | GTTGCTGGGCACTTGGGTTTTACCCCGCAGAGATCACTCTCACTTGGCAACAAGATGGGGAGGGTCA |
| | TACCCAGGATACCGAGCTTGTCGAAACAAGGCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCcaccatcaccaccatcat (SEQ ID NO: 305) |
| B2M STREP | CGCACAGTGAGAACCCCTAAGATACAAGTTTATAGTCGGCACCCCGCAGAGAACGGGAAAAGTAACT |
| | TCCTTAATTGCTATGTCTCTGGCTTCCATCCTTCTGACATCGAGGTGGACTTGTTGAAAAACGGGGA |
| | GAGAATTGAAAAAGTGGAACACAGCGATCTGAGCTTCTCCAAAGATTGGTCTTTTTATCTCCTCTAC |
| | TACACCGAATTTACCCCAACTGAGAAAGATGAATATGCCTGTCGGGTTAATCACGTAACTTTGTCTC |
| | AACCAAAAATTGTCAAATGGGACAGGGACATGTCAGGGTGGAGTCACCCTCAGTTCGAAAAG |
| | (SEQ ID NO: 3) |
| B2M SER MUT | AGAACCGTTAGAACTCCTAAGATTCAGGTCTATTCCCGACATCCTGCTGAGAATGGAAAGTCTAATT |
| STREP | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 306) |
| EA3 HC H6 | CAGATAACCCTCAAGGAGTCAGGGCCAACCCTGGTGAAGCCAACTCAAACACTTACTCTTACCTGCA |
| (HLPPB17) | CTTTTTCTGGATTTAGCCTTAGTACAAGTGGGATGGGCGTAAGTTGGATACGCCAACCTCCTGGCAA |
| | GGCCCTGGAATGGCTGGCACACATATACTGGGACGATGACAAGCGCTATAACCCATCTTTGAAGAGC |
| | CGGTTGACTATCACTAAGGACACCAGTAAAAATCAAGTAGTGCTTACAATGACTAACATGGACCCTG |
| | TGGATACCGCCACCTATTACTGTGCTAGGTTGTATGGCTTTACCTATGGCTTCGCATACTGGGGACA |
| | GGGGACACTCGTAACTGTATCCTCTCTGCATCTGGAACCCCCCAAAACTCATGTCACCCATCATCCC |
| | ATATCTGACCACGAAGCAACCCTGCGTTGCTGGGCACTTGGGTTTTACCCCGCAGAGATCACTCTCA |
| | CTTGGCAACAAGATGGGGAGGGTCATACCCAGGATACCGAGCTTGTCGAAACAAGGCCCGCCGGGGA |
| | TGGAACCTTTCAGAAATGGGCTGCTGTAGTGGTCCCTAGCGGAGAAGAACAACGGTACACTTGTCAT |
| | GTCCAGCATGAGGGTCTTCCAGAACCAGTCACCCTTCGGTGGGAGCCCAAGAGTAGCcaccatcacc |
| | accatcat (SEQ ID NO: 307) |
| B2M LC STREP | GATATCGTGATGACACAGAGTCCTGATTCCTTGGCCGTCTCATTGGGTGAAAGGGCAACAATTAACT |
| (HLPPB17) | GTCGTGCCAGTCAGTCTGTCGACTACAATGGCATCTCTTACATGCACTGGTATCAACAAAAGCCAGG |
| | ACAACCTCCAAAACTCCTGATTTATGCCGCCTCCAACCCCGAGAGTGGCGTTCCCGATAGATTCTCA |
| | GGATCAGGGTCTGGGACTGACTTTACTCTCACCATTAGTTCCCTCCAGGCAGAAGATGTTGCAGTAT |
| | ATTATTGTCAACAAATTATCGAAGACCCTTGGACTTTCGGTCAGGGAACAAAAGTAGAAATCAAAAG |
| | AACCGTTAGAACTCCTAAGATTCAGGTCTATTCCCGACATCCTGCTGAGAATGGAAAGTCTAATTTT |
| | CTCAACTGTTATGTATCAGGGTTCCATCCAAGTGATATAGAAGTTGATCTTCTCAAGAACGGTGAGC |
| | GTATCGAAAAAGTCGAGCATTCTGACCTCTCCAGTTCCAAGGATAGTTCCACATACCTGCTGTATTA |
| | TACCGAGTTTACTCCAACAGAAAAAGATGAATACGCCTGTCGAGTAAATCACGTCACTCTTTCCCAG |
| | CCTAAGATTGTGAAGTGGGATCGGGATATGTCAGGGTGGAGTCACCCTCAGTTCGAAAAG |
| | (SEQ ID NO: 308) |
| EA3 R234A-His | CTGCATCTGGAACCCCCCAAAACTCATGTCACCCATCATCCCATATCTGACCACGAAGCAACCCTGC |
| | GTTGCTGGGCACTTGGGTTTTACCCCGCAGAGATCACTCTCACTTGGCAACAAGATGGGGAGGGTCA |
| | TACCCAGGATACCGAGCTTGTCGAAACAgccCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 309) |
| EA3 R234L-His | CTGCATCTGGAACCCCCCAAAACTCATGTCACCCATCATCCCATATCTGACCACGAAGCAACCCTGC |
| | GTTGCTGGGCACTTGGGTTTTACCCCGCAGAGATCACTCTCACTTGGCAACAAGATGGGGAGGGTCA |
| | TACCCAGGATACCGAGCTTGTCGAAACActgCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 310) |
| EA3 R234A, Q242A- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACAGCCCCCGCCGGGGATGGAACCTTTgctAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 311) |
| EA3 R234L, Q242L- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACACTGCCCGCCGGGGATGGAACCTTTctgAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 312) |
| EA3 R234A, Q242L- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACAGCCCCCGCCGGGGATGGAACCTTTctgAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 313) |
| EA3 R234L, Q242A- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACACTGCCCGCCGGGGATGGAACCTTTgccAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 314) |
| EA3 R202A-His | CTGCATCTGGAACCCCCCAAAACTCATGTCACCCATCATCCCATATCTGACCACGAAGCAACCCTGg |
| | ccTGCTGGGCACTTGGGTTTTACCCCGCAGAGATCACTCTCACTTGGCAACAAGATGGGGAGGGTCA |
| | TACCCAGGATACCGAGCTTGTCGAAACAAGGCCCGCCGGGGATGGAACCTTTCAGAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 315) |
| B2M (SST) Y10F-strep | AGAACCGTTAGAACTCCTAAGATTCAGGTCttcTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 316) |
| B2M (SST) Y10W- | AGAACCGTTAGAACTCCTAAGATTCAGGTCtggTCCCGACATCCTGCTGAGAATGGAAAGTCTAATT |
| strep | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATATGTCAGGGTGGAGTCACCCTCAGTTCGAAAAG |
| | (SEQ ID NO: 317) |
| B2M (SST) D98F-strep | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGttcATGTCAGGGTGGAGTCACCCTCAGTTCGAAAAG |
| | (SEQ ID NO: 318) |
| EA3 R234L, Q242L- | CTGCATCTGGAACCCCCCAAAACTCATGTCACCCATCATCCCATATCTGACCACGAAGCAACCCTGC |
| His | GTTGCTGGGCACTTGGGTTTTACCCCGCAGAGATCACTCTCACTTGGCAACAAGATGGGGAGGGTCA |
| | TACCCAGGATACCGAGCTTGTCGAAACACTGCCCGCCGGGGATGGAACCTTTctgAAATGGGCTGCT |
| | GTAGTGGTCCCTAGCGGAGAAGAACAACGGTACACTTGTCATGTCCAGCATGAGGGTCTTCCAGAAC |
| | CAGTCACCCTTCGGTGGGAGCCCAAGAGTAGCCACCACCATCATCATCAT (SEQ ID NO: 319) |
| B2M (SST) D98W- | AGAACCGTTAGAACTCCTAAGATTCAGGTCTATTCCCGACATCCTGCTGAGAATGGAAAGTCTAATT |
| strep | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGtggATGTCAGGGTGGAGTCACCCTCAGTTCGAAAAG |
| | (SEQ ID NO: 320) |
| B2M (SST) M99F-strep | AGAACCGTTAGAACTCCTAAGATTCAGGTCTATTCCCGACATCCTGCTGAGAATGGAAAGTCTAATT |
| | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATttcTCAGGGTGGAGTCACCCTCAGTTCGAAAAG |
| | (SEQ ID NO: 321) |
| B2M (SST) M99W- | AGAACCGTTAGAACTCCTAAGATTCAGGTCTATTCCCGACATCCTGCTGAGAATGGAAAGTCTAATT |
| strep | TTCTCAACTGTTATGTATCAGGGTTCCATCCAAGTGATATAGAAGTTGATCTTCTCAAGAACGGTGA |
| | GCGTATCGAAAAAGTCGAGCATTCTGACCTCTCCAGTTCCAAGGATAGTTCCACATACCTGCTGTAT |
| | TATACCGAGTTTACTCCAACAGAAAAAGATGAATACGCCTGTCGAGTAAATCACGTCACTCTTTCCC |
| | AGCCTAAGATTGTGAAGTGGGATCGGGATtggTCAGGGTGGAGTCACCCTCAGTTCGAAAAG |
| | (SEQ ID NO: 322) |
| HLPPB270 HC | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCACTGCATCTGGAACCCCCCAAAACTCATGTCACC |
| | CATCATCCCATATCTGACCACGAAGCAACCCTGCGTTGCTGGGCACTTGGGTTTTACCCCGCAGAGA |
| | TCACTCTCACTTGGCAACAAGATGGGGAGGGTCATACCCAGGATACCGAGCTTGTCGAAACAAGGCC |
| | CGCCGGGGATGGAACCTTTCAGAAATGGGCTGCTGTAGTGGTCCCTAGCGGAGAAGAACAACGGTAC |
| | ACTTGTCATGTCCAGCATGAGGGTCTTCCAGAACCAGTCACCCTTCGGTGGGAGCCCAAGAGTAGCC |
| | ACCACCATCATCATCAT (SEQ ID NO: 323) |
| HLPPB270 LC | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCAAGAACTCCTAAGATTCAGGTCTATTCCCGACATCCTGCTGAGAATGGAAAG |
| | TCTAATTTTCTCAACTGTTATGTATCAGGGTTCCATCCAAGTGATATAGAAGTTGATCTTCTCAAGA |
| | ACGGTGAGCGTATCGAAAAAGTCGAGCATTCTGACCTCTCCAGTTCCAAGGATAGTTCCACATACCT |
| | GCTGTATTATACCGAGTTTACTCCAACAGAAAAAGATGAATACGCCTGTCGAGTAAATCACGTCACT |
| | CTTTCCCAGCCTAAGATTGTGAAGTGGGATCGGGATATGTCAGGGTGGAGTCACCCTCAGTTCGAAA |
| | AG (SEQ ID NO: 324) |
| EA3 Native Hinge GGS | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCACTCCATTTGGAGCCACCAAAAACCCATGTAACC |
| | CACCACCCTATAAGTGACCACGAAGCTACTCTGCGGTGTTGGGCACTGGGCTTTTACCCAGCAGAAA |
| | TAACACTCACTTGGCAACAAGATGGAGAAGGTCATACTCAGGATACAGAACTCGTGGAGACACGTCC |
| | CGCAGGTGATGGTACTTTTCAGAAGTGGGCTGCTGTTGTGGTCCCCTCCGGGGAGGAACAGAGGTAC |
| | ACCTGTCATGTCCAACACGAGGGTTTGCCCGAACCCGTTACTCTCCGATGGGAGCCCAAGTCATGCC |
| | ACCACCATCATCATCAT (SEQ ID NO: 325) |
| EA3 Native Hinge | CAGATCACCCTGAAAGAGTCTGGCCCCACACTGGTCAAGCCCACACAGACCCTGACACTGACCTGCA |
| GGGS | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCACTTCATCTTGAACCACCTAAGACCCACGTTACT |
| | CATCATCCAATAAGCGACCACGAAGCCACCCTGAGATGCTGGGCACTTGGTTTTTATCCAGCAGAGA |
| | TCACTCTGACTTGGCAGCAGGACGGCGAAGGTCACACTCAAGACACAGAACTCGTGGAAACTAGACC |
| | AGCCGGGGATGGCACTTTCCAAAAATGGGCAGCCGTCGTAGTACCTTCTGGGGAAGAGCAGAGATAC |
| | ACCTGCCACGTCCAGCATGAAGGGCTGCCTGAGCCCGTAACCCTGAGGTGGGAACCTAAGTCCTGCC |
| | ACCACCATCATCATCAT (SEQ ID NO: 326) |
| EA3 R202C | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCACTCCATTTGGAACCTCCTAAAACACATGTCACT |
| | CACCATCCCATCTCCGACCACGAAGCTACCTTGTGCTGTTGGGCTCTTGGATTCTACCCAGCCGAAA |
| | TAACTCTGACATGGCAACAGGATGGAGAAGGTCATACCCAGGATACAGAGCTGGTAGAAACTCGGCC |
| | AGCCGGGGATGGTACATTCCAGAAGTGGGCAGCAGTAGTTGTGCCATCCGGGGAGGAGCAAAGATAT |
| | ACCTGTCATGTCCAACACGAAGGTTTGCCTGAACCAGTGACTCTTCGTTGGGAGCCCAAGAGTAGCC |
| | ACCACCATCATCATCAT (SEQ ID NO: 327) |
| EA3 H192C | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCATTGCATCTTGAACCCCCTAAGACACACGTTACA |
| | CATTGCCCTATCTCCGATCACGAGGCAACTCTCCGATGTTGGGCACTGGGTTTCTATCCCGCAGAGA |
| | TAACATTGACTTGGCAACAGGATGGGGAAGGGCATACACAAGATACAGAACTGGTCGAAACAAGGCC |
| | CGCCGGAGACGGAACATTTCAAAAGTGGGCAGCAGTTGTAGTTCCCTCTGGGGAGGAACAGCGCTAC |
| | ACTTGCCATGTGCAGCACGAAGGTCTGCCAGAGCCTGTCACTCTCAGATGGGAGCCCAAGAGTAGCC |
| | ACCACCATCATCATCAT (SEQ ID NO: 328) |
| EA3 E232C | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCATTGCACCTCGAACCACCCAAAACCCACGTGACT |
| | CATCATCCCATCTCTGACCACGAGGCCACACTTCGGTGTTGGGCTCTGGGCTTCTACCCAGCAGAGA |
| | TAACCCTGACCTGGCAACAGGATGGGGAGGGTCACACACAGGACACAGAATTGGTCTGCACTAGACC |
| | TGCCGGTGACGGCACCTTTCAAAAGTGGGCAGCAGTGGTTGTGCCAAGCGGAGAGGAGCAACGATAC |
| | ACCTGCCACGTACAACACGAAGGGCTCCCTGAACCAGTAACACTGCGCTGGGAGCCCAAGAGTAGCC |
| | ACCACCATCATCATCAT (SEQ ID NO: 329) |
| EA3 R234C | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCATTGCATCTTGAACCTCCTAAAACTCATGTAACT |
| | CACCACCCAATTTCAGACCATGAAGCTACTCTCCGATGCTGGGCTCTTGGCTTTTATCCTGCCGAAA |
| | TCACATTGACATGGCAACAGGATGGAGAGGGGCATACACAGGACACCGAGTTGGTGGAAACTTGTCC |
| | CGCAGGGGATGGCACATTTCAAAAATGGGCTGCTGTTGTTGTGCCCAGCGGCGAAGAACAGCGGTAC |
| | ACATGTCACGTCCAACATGAAGGCTTGCCAGAGCCCGTAACTCTTCGCTGGGAGCCCAAGAGTAGCC |
| | ACCACCATCATCATCAT (SEQ ID NO: 330) |
| EA3 D238C | CAGATCACCCTGAAAGAGTCTGGCCCCACACTGGTCAAGCCCACACAGACCCTGACACTGACCTGCA |
| | CCTTCTCCGGCTTCTCCCTGTCTACCTCTGGCATGGGCGTGTCCTGGATCAGACAGCCTCCTGGCAA |
| | AGCCCTGGAATGGCTGGCTCACATCTACTGGGACGACGACAAGCGGTACAACCCCAGCCTGAAGTCT |
| | CGGCTGACCATCACCAAGGACACCTCCAAGAACCAGGTGGTGCTGACCATGACAAACATGGACCCTG |
| | TGGACACCGCCACCTACTACTGCGCTAGACTGTACGGCTTCACCTACGGCTTTGCCTATTGGGGCCA |
| | GGGCACCCTGGTTACCGTTTCTTCTGGGGGATCATTGCATCTCGAGCCCCCCAAGACCCACGTTACT |
| | CACCACCCTATAAGCGATCACGAGGCAACATTGAGGTGCTGGGCCTTGGGTTTCTACCCCGCTGAGA |
| | TCACACTCACTTGGCAGCAAGATGGAGAAGGGCATACACAGGACACCGAACTCGTGGAAACCCGACC |
| | AGCCGGTTGTGGTACATTCCAGAAATGGGCTGCCGTAGTAGTCCCTAGCGGTGAGGAGCAGCGATAT |
| | ACCTGTCATGTGCAACACGAGGGCTTGCCCGAACCAGTCACTTTGAGATGGGAGCCCAAGAGTAGCC |
| | ACCACCATCATCATCAT (SEQ ID NO: 331) |
| B2M Native Hinge | GACATCGTGATGACCCAGTCTCCAGACAGCCTGGCTGTGTCTCTGGGCGAGAGAGCCACCATCAACT |
| GGS | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCAAGAACTGTACGCACACCAAAGATACAAGTATATTCCAGACACCCCGCAGAA |
| | AATGGTAAGAGCAATTTCTTGAACTGTTATGTATCCGGTTTTCACCCTTCTGATATAGAGGTAGATT |
| | TGCTGAAAAATGGTGAGCGGATAGAGAAAGTTGAGCACTCCGATCTTAGCTCATCTAAGGATTCAAG |
| | CACTTATCTCTTGTATTACACAGAGTTTACTCCCACCGAGAAAGACGAGTATGCCTGCCGTGTCAAC |
| | CATGTCACTTTGTCCCAGCCCAAGATAGTGAAATGGGACCGAGATGGAGGCAGTGGAGAGTGCTCTG |
| | GGTACCCTTACGATGTCCCTGATTACGCA (SEQ ID NO: 332) |
| B2M Native Hinge | GACATCGTGATGACCCAGTCTCCAGACAGCCTGGCTGTGTCTCTGGGCGAGAGAGCCACCATCAACT |
| GGGS | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGGACAGTGCGGACTCCAAAGATACAAGTTTATAGCAGGCATCCCGCTGAG |
| | AATGGGAAGTCCAACTTTCTTAATTGCTATGTCTCCGGGTTCCACCCCTCTGACATCGAGGTGGACT |
| | TGCTGAAGAATGGGGAAAGAATAGAGAAAGTTGAGCACTCTGATCTCAGTTCATCCAAGGATTCCTC |
| | AACATACCTCTTGTACTACACTGAGTTCACCCCAACCGAAAAAGATGAATATGCTTGCAGAGTCAAT |
| | CACGTGACCCTGTCACAGCCTAAAATAGTCAAGTGGGATAGGGACGGAGGTGGGAGCGGTGAGTGCT |
| | CTGGGTACCCTTACGATGTCCCTGATTACGCA (SEQ ID NO: 333) |
| B2M M99C GGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGGACTGTTCGCACACCCAAAATTCAGGTTTACTCCCGACATCCAGCTGAG |
| | AACGGTAAGTCTAATTTCCTGAACTGTTACGTGTCTGGTTTTCATCCTTCAGATATAGAAGTTGACC |
| | TCCTCAAAAACGGCGAAAGGATAGAAAAGGTAGAGCACAGCGACTTGAGTAGCAGCAAAGATTCTAG |
| | CACATATCTGCTGTATTATACCGAATTTACACCCACAGAGAAGGACGAGTACGCATGTCGGGTAAAC |
| | CATGTGACACTCTCTCAGCCTAAGATCGTCAAGTGGGACCGAGACTGTGGAGGTTCTGGTGAGAGTT |
| | CCGGCTATCCTTATGATGTGCCCGACTACGCC (SEQ ID NO: 334) |
| B2M D98C GGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACAGTACGTACACCAAAGATCCAGGTTTACTCACGTCACCCCGCCGAG |
| | AATGGCAAGAGCAACTTCCTCAACTGCTACGTGAGTGGATTCCACCCTAGTGACATCGAGGTTGACC |
| | TGCTGAAAAATGGTGAGCGCATTGAAAAAGTTGAACATTCTGACTTGAGCAGCAGTAAAGACAGTTC |
| | AACATATCTCCTCTACTACACCGAGTTTACACCAACCGAAAAAGACGAGTACGCATGTAGAGTTAAT |
| | CATGTGACTCTGTCACAACCTAAAATCGTAAAGTGGGACCGATGCATGGGAGGTTCTGGTGAGAGTT |
| | CCGGCTATCCTTATGATGTGCCCGACTACGCC (SEQ ID NO: 335) |
| B2M K6C GGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGAACCGTCCGAACACCCTGCATTCAAGTTTACAGCCGGCATCCAGCCGAG |
| | AATGGGAAGTCTAATTTTCTGAATTGTTATGTAAGCGGATTCCACCCAAGCGACATTGAGGTGGACC |
| | TTTTGAAGAATGGCGAACGCATAGAGAAAGTCGAACATAGCGACCTTTCAAGCTCTAAAGATTCCAG |
| | TACCTACCTCCTTTATTATACTGAGTTTACCCCCACTGAAAAGGACGAATATGCTTGTAGGGTGAAC |
| | CACGTCACTCTCAGCCAGCCAAAGATTGTTAAATGGGATAGGGATATGGGAGGTTCTGGTGAGAGTT |
| | CCGGCTATCCTTATGATGTGCCCGACTACGCC (SEQ ID NO: 336) |
| B2M Y10C GGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACTGTGAGAACACCTAAGATCCAGGTCTGTTCTAGGCATCCCGCTGAA |
| | AACGGCAAATCAAACTTTTTGAACTGTTATGTGAGTGGTTTCCATCCCTCCGATATAGAAGTTGACT |
| | TGCTTAAAAATGGAGAACGCATCGAAAAAGTGGAACATTCTGATCTTTCTTCCAGCAAGGATAGCTC |
| | TACATACCTCCTTTACTACACCGAGTTTACTCCAACAGAGAAGGATGAGTACGCTTGTAGAGTTAAC |
| | CATGTCACTCTCAGCCAACCTAAGATTGTTAAATGGGACAGAGATATGGGAGGTTCTGGTGAGAGTT |
| | CCGGCTATCCTTATGATGTGCCCGACTACGCC (SEQ ID NO: 337) |
| B2M R12C GGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACTGTAAGGACCCCCAAAATACAGGTCTACTCATGTCACCCCGCCGAG |
| | AATGGAAAGTCCAATTTCCTGAACTGCTACGTAAGTGGGTTCCATCCTTCTGATATAGAAGTTGATC |
| | TGCTCAAGAACGGAGAGCGTATAGAAAAAGTTGAACATAGTGATCTTTCAAGCAGCAAAGACTCTTC |
| | AACCTATCTGCTTTACTATACTGAGTTCACTCCAACAGAAAAGGACGAATACGCTTGCCGGGTGAAC |
| | CACGTCACTCTTTCTCAACCTAAGATTGTAAAATGGGACCGAGACATGGGAGGTTCTGGTGAGAGTT |
| | CCGGCTATCCTTATGATGTGCCCGACTACGCC (SEQ ID NO: 338) |
| B2M M99C GGGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGGACTGTTCGCACACCCAAAATTCAGGTTTACTCCCGACATCCAGCTGAG |
| | AACGGTAAGTCTAATTTCCTGAACTGTTACGTGTCTGGTTTTCATCCTTCAGATATAGAAGTTGACC |
| | TCCTCAAAAACGGCGAAAGGATAGAAAAGGTAGAGCACAGCGACTTGAGTAGCAGCAAAGATTCTAG |
| | CACATATCTGCTGTATTATACCGAATTTACACCCACAGAGAAGGACGAGTACGCATGTCGGGTAAAC |
| | CATGTGACACTCTCTCAGCCTAAGATCGTCAAGTGGGACCGAGACTGTGGAGGAGGTAGCGGAGAGA |
| | GCAGTGGATATCCCTACGATGTACCTGATTACGCA (SEQ ID NO: 339) |
| B2M D98C GGGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACAGTACGTACACCAAAGATCCAGGTTTACTCACGTCACCCCGCCGAG |
| | AATGGCAAGAGCAACTTCCTCAACTGCTACGTGAGTGGATTCCACCCTAGTGACATCGAGGTTGACC |
| | TGCTGAAAAATGGTGAGCGCATTGAAAAAGTTGAACATTCTGACTTGAGCAGCAGTAAAGACAGTTC |
| | AACATATCTCCTCTACTACACCGAGTTTACACCAACCGAAAAAGACGAGTACGCATGTAGAGTTAAT |
| | CATGTGACTCTGTCACAACCTAAAATCGTAAAGTGGGACCGATGCATGGGAGGAGGTAGCGGAGAGA |
| | GCAGTGGATATCCCTACGATGTACCTGATTACGCA (SEQ ID NO: 340) |
| B2M K6C GGGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGAACCGTCCGAACACCCTGCATTCAAGTTTACAGCCGGCATCCAGCCGAG |
| | AATGGGAAGTCTAATTTTCTGAATTGTTATGTAAGCGGATTCCACCCAAGCGACATTGAGGTGGACC |
| | TTTTGAAGAATGGCGAACGCATAGAGAAAGTCGAACATAGCGACCTTTCAAGCTCTAAAGATTCCAG |
| | TACCTACCTCCTTTATTATACTGAGTTTACCCCCACTGAAAAGGACGAATATGCTTGTAGGGTGAAC |
| | CACGTCACTCTCAGCCAGCCAAAGATTGTTAAATGGGATAGGGATATGGGAGGAGGTAGCGGAGAGA |
| | GCAGTGGATATCCCTACGATGTACCTGATTACGCA (SEQ ID NO: 341) |
| B2M Y10C GGGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACTGTGAGAACACCTAAGATCCAGGTCTGTTCTAGGCATCCCGCTGAA |
| | AACGGCAAATCAAACTTTTTGAACTGTTATGTGAGTGGTTTCCATCCCTCCGATATAGAAGTTGACT |
| | TGCTTAAAAATGGAGAACGCATCGAAAAAGTGGAACATTCTGATCTTTCTTCCAGCAAGGATAGCTC |
| | TACATACCTCCTTTACTACACCGAGTTTACTCCAACAGAGAAGGATGAGTACGCTTGTAGAGTTAAC |
| | CATGTCACTCTCAGCCAACCTAAGATTGTTAAATGGGACAGAGATATGGGAGGAGGTAGCGGAGAGA |
| | GCAGTGGATATCCCTACGATGTACCTGATTACGCA (SEQ ID NO: 342) |
| B2M R12C GGGS | GACATCGTGATGACCCAGTCTCCAGACAGCCTGGCTGTGTCTCTGGGCGAGAGAGCCACCATCAACT |
| | GCAGAGCCTCTCAGTCCGTGGACTACAACGGCATCTCCTACATGCACTGGTATCAGCAGAAGCCCGG |
| | CCAGCCTCCTAAGCTGCTGATCTACGCCGCCTCCAATCCTGAGTCTGGCGTGCCCGATAGATTCTCC |
| | GGCTCTGGCTCTGGCACCGACTTTACCCTGACAATCAGCTCCCTGCAGGCCGAGGATGTGGCCGTGT |
| | ACTACTGCCAGCAGATCATCGAGGACCCCTGGACCTTTGGCCAGGGCACCAAGGTGGAAATCAAGAG |
| | AACCGTTGGGGGATCACGCACTGTAAGGACCCCCAAAATACAGGTCTACTCATGTCACCCCGCCGAG |
| | AATGGAAAGTCCAATTTCCTGAACTGCTACGTAAGTGGGTTCCATCCTTCTGATATAGAAGTTGATC |
| | TGCTCAAGAACGGAGAGCGTATAGAAAAAGTTGAACATAGTGATCTTTCAAGCAGCAAAGACTCTTC |
| | AACCTATCTGCTTTACTATACTGAGTTCACTCCAACAGAAAAGGACGAATACGCTTGCCGGGTGAAC |
| | CACGTCACTCTTTCTCAACCTAAGATTGTAAAATGGGACCGAGACATGGGAGGAGGTAGCGGAGAGA |
| | GCAGTGGATATCCCTACGATGTACCTGATTACGCA (SEQ ID NO: 343) |

### 9.3 Example 3. HLA-E Pseudo-Fab Bispecific Antibodies

HER2×CD3 and HER2×MET bispecific antibodies were developed in the FabxHLA-E format. The bispecific antibodies also included the knob-into-hole mutations (T366S, L368A and Y407V (Hole) and T366W (Knob)) and H435R and Y436F (“RF mutations”) in the CH3 domain of the Hole arm. The bispecific antibodies maintained a standard IgG Fab on the Hole arm comprising the RF mutations (“Hole-RF arm”), and an HLA-E pseudo-Fab on the Knob arm. The HLA-E pseudo-Fab arm was based on HLPPW57 which comprises HLA-E A3 (H192C) and B2M (SST, D98C) with AST and RTV elbow sequences N-terminal to HLA-E A3 and B2M, respectively. The design of the HLA-E pseudo-Fab bispecific antibodies is described in Table 30. The corresponding amino acid sequences for the bispecific antibodies are provided in Table 31.

TABLE 30
Design of HLA-E Pseudo-Fab Bispecific Antibodies

| Antibody | Target for Fab | Target for HLA-E |
|---|---|---|
| Identifier | (Hole-RF) arm | pseudo-Fab (Knob) arm |
| HLPPB421 | MET | HER2 |
| HLPPB423 | CD3 | HER2 |
| HLPPB425 | HER2 | CD3 |
| HLPPB426 | HER2 | MET |

TABLE 31
Amino Acid Sequences for Bispecific Antibodies:

| HLPPB421 | HER2 Knob H192C/D98C_RTV/AST x MET hole-RF |
|---|---|
| HC1 | EVQLVESGGGLVQPGGSLRLSCAASGFNIKDTYIHWVRQAPGKGLEWVARIYPTNGYTRYADSVKGR |
| (VH1-AST- | FTISADTSKNTAYLQMNSLRAEDTAVYYCSRWGGDGFYAMDYWGQGTLVTVSSASTLHLEPPKTHVT |
| EA3(H192C)- | HCPISDHEATLRCWALGFYPAEITLTWQQDGEGHTQDTELVETRPAGDGTFQKWAAVVVPSGEEQRY |
| Fc region | TCHVQHEGLPEPVTLRWEPKSSDKTHTCPPCPAPELLGGPSVFLFPPKPKDTLMISRTPEVTCVVVD |
| (knob)) | VSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: 344) |
| LC1 | DIQMTQSPSSLSASVGDRVTITCRASQDVNTAVAWYQQKPGKAPKLLIYSASFLYSGVPSRFSGSRS |
| (VL1-RTV- | GTDFTLTISSLQPEDFATYYCQQHYTTPPTFGQGTKVEIKRTVRTPKIQVYSRHPAENGKSNFLNCY |
| B2M) | VSGFHPSDIEVDLLKNGERIEKVEHSDLSSSKDSSTYLLYYTEFTPTEKDEYACRVNHVTLSQPKIV |
| | KWDRCM (SEQ ID NO: 345) |
| HC2 | EVQLVESGGGLVQPGGSLRLSCAASGYTFTSYWLHWVRQAPGKGLEWVGMIDPSNSDTRFNPNFKDR |
| (VH2-CH1- | FTISADTSKNTAYLQMNSLRAEDTAVYYCATYRSYVTPLDYWGQGTLVTVSSASTKGPSVFPLAPSS |
| Fc region | KSTSGGTAALGCLVKDYFPEPVTVSWNSGALTSGVHTFPAVLQSSGLYSLSSVVTVPSSSLGTQTYI |
| (hole-RF)) | CNVNHKPSNTKVDKKVEPKSCDKTHTCPPCPAPELLGGPSVFLFPPKPKDTLMISRTPEVTCVVVDV |
| | SHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLSCAVKGFYPSDIAVEWESNGQPENNYKTTPPVLDS |
| | DGSFFLVSKLTVDKSRWQQGNVFSCSVMHEALHNRFTQKSLSLSPGK (SEQ ID NO: 346) |
| LC2 | DIQMTQSPSSLSASVGDRVTITCKSSQSLLYTSSQKNYLAWYQQKPGKAPKLLIYWASTRESGVPSR |
| (VL2-CL) | FSGSGSGTDFTLTISSLQPEDFATYYCQQYYAYPWTFGQGTKVEIKRTVAAPSVFIFPPSDEQLKSG |
| | TASVVCLLNNFYPREAKVQWKVDNALQSGNSQESVTEQDSKDSTYSLSSTLTLSKADYEKHKVYACE |
| | VTHQGLSSPVTKSFNRGEC (SEQ ID NO: 347) |
| HLPPB423 | HER2 Knob H192C/D98C_RTV/AST x CD3 hole-RF |
| HC1 | EVQLVESGGGLVQPGGSLRLSCAASGFNIKDTYIHWVRQAPGKGLEWVARIYPTNGYTRYADSVKGR |
| (VH1-AST- | FTISADTSKNTAYLQMNSLRAEDTAVYYCSRWGGDGFYAMDYWGQGTLVTVSSASTLHLEPPKTHVT |
| EA3(H192C)- | HCPISDHEATLRCWALGFYPAEITLTWQQDGEGHTQDTELVETRPAGDGTFQKWAAVVVPSGEEQRY |
| Fc region | TCHVQHEGLPEPVTLRWEPKSSDKTHTCPPCPAPELLGGPSVFLFPPKPKDTLMISRTPEVTCVVVD |
| (knob)) | VSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLWCLVKGFYPSDIAVEWESNGQPENNYKTTPPVLD |
| | SDGSFFLYSKLTVDKSRWQQGNVFSCSVMHEALHNHYTQKSLSLSPGK (SEQ ID NO: 348) |
| LC1 | DIQMTQSPSSLSASVGDRVTITCRASQDVNTAVAWYQQKPGKAPKLLIYSASFLYSGVPSRFSGSRS |
| (VL1-RTV- | GTDFTLTISSLQPEDFATYYCQQHYTTPPTFGQGTKVEIKRTVRTPKIQVYSRHPAENGKSNFLNCY |
| B2M) | VSGFHPSDIEVDLLKNGERIEKVEHSDLSSSKDSSTYLLYYTEFTPTEKDEYACRVNHVTLSQPKIV |
| | KWDRCM (SEQ ID NO: 349) |
| HC2 | EVQLVESGGGLVQPGGSLRLSCAASGYSFTGYTMNWVRQAPGKGLEWVALINPYKGVSTYNQKFKDR |
| (VH2-CH1- | FTISVDKSKNTAYLQMNSLRAEDTAVYYCARSGYYGDSDWYFDVWGQGTLVTVSSASTKGPSVFPLA |
| Fc region | PSSKSTSGGTAALGCLVKDYFPEPVTVSWNSGALTSGVHTFPAVLQSSGLYSLSSVVTVPSSSLGTQ |
| (hole-RF)) | TYICNVNHKPSNTKVDKKVEPKSCDKTHTCPPCPAPELLGGPSVFLFPPKPKDTLMISRTPEVTCVV |
| | VDVSHEDPEVKFNWYVDGVEVHNAKTKPREEQYNSTYRVVSVLTVLHQDWLNGKEYKCKVSNKALPA |
| | PIEKTISKAKGQPREPQVYTLPPSREEMTKNQVSLSCAVKGFYPSDIAVEWESNGQPENNYKTTPPV |
| | LDSDGSFFLVSKLTVDKSRWQQGNVFSCSVMHEALHNRFTQKSLSLSPGK (SEQ ID NO: 350) |
| LC2 | DIQMTQSPSSLSASVGDRVTITCRASQDIRNYLNWYQQKPGKAPKLLIYYTSRLESGVPSRFSGSGS |
| (VL2-CL) | GTDYTLTISSLQPEDFATYYCQQGNTLPWTFGQGTKVEIKRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC (SEQ ID NO: 351) |
| HLPPB425 | CD3 Knob H192C/D98C_RVT/AST x HER2 Hole-RF |
| HC1 | EVQLVESGGGLVQPGGSLRLSCAASGYSFTGYTMNWVRQAPGKGLEWVALINPYKGVSTYNQKFKDR |
| (VH1-AST- | FTISVDKSKNTAYLQMNSLRAEDTAVYYCARSGYYGDSDWYFDVWGQGTLVTVSSASTLHLEPPKTH |
| EA3(H192C)- | VTHCPISDHEATLRCWALGFYPAEITLTWQQDGEGHTQDTELVETRPAGDGTFQKWAAVVVPSGEEQ |
| Fc region | RYTCHVQHEGLPEPVTLRWEPKSSDKTHTCPPCPAPELLGGPSVFLFPPKPKDTLMISRTPEVTCVV |
| (knob)) | VDVSHEDPEVKFNWYVDGVEVHNAKTKPREEQYNSTYRVVSVLTVLHQDWLNGKEYKCKVSNKALPA |
| | PIEKTISKAKGQPREPQVYTLPPSREEMTKNQVSLWCLVKGFYPSDIAVEWESNGQPENNYKTTPPV |
| | LDSDGSFFLYSKLTVDKSRWQQGNVFSCSVMHEALHNHYTQKSLSLSPGK (SEQ ID NO: 352) |
| LC1 | DIQMTQSPSSLSASVGDRVTITCRASQDIRNYLNWYQQKPGKAPKLLIYYTSRLESGVPSRFSGSGS |
| (VL1-RTV- | GTDYTLTISSLQPEDFATYYCQQGNTLPWTFGQGTKVEIKRTVRTPKIQVYSRHPAENGKSNFLNCY |
| B2M) | VSGFHPSDIEVDLLKNGERIEKVEHSDLSSSKDSSTYLLYYTEFTPTEKDEYACRVNHVTLSQPKIV |
| | KWDRCM (SEQ ID NO: 353) |
| HC2 | EVQLVESGGGLVQPGGSLRLSCAASGFNIKDTYIHWVRQAPGKGLEWVARIYPTNGYTRYADSVKGR |
| (VH2-CH1- | FTISADTSKNTAYLQMNSLRAEDTAVYYCSRWGGDGFYAMDYWGQGTLVTVSSASTKGPSVFPLAPS |
| Fc region | SKSTSGGTAALGCLVKDYFPEPVTVSWNSGALTSGVHTFPAVLQSSGLYSLSSVVTVPSSSLGTQTY |
| (hole-RF)) | ICNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNRFTQKSLSLSPGK (SEQ ID NO: 354) |
| LC2 | DIQMTQSPSSLSASVGDRVTITCRASQDVNTAVAWYQQKPGKAPKLLIYSASFLYSGVPSRFSGSRS |
| (VL2-CL) | GTDFTLTISSLQPEDFATYYCQQHYTTPPTFGQGTKVEIKRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC (SEQ ID NO: 355) |
| HLPPB426 | MET Knob H192C/D98C_RTV/AST x HER2 hole-RF |
| HC1 | EVQLVESGGGLVQPGGSLRLSCAASGYTFTSYWLHWVRQAPGKGLEWVGMIDPSNSDTRFNPNFKDR |
| (VH1-AST- | FTISADTSKNTAYLQMNSLRAEDTAVYYCATYRSYVTPLDYWGQGTLVTVSSASTLHLEPPKTHVTH |
| EA3(H192C)- | CPISDHEATLRCWALGFYPAEITLTWQQDGEGHTQDTELVETRPAGDGTFQKWAAVVVPSGEEQRYT |
| Fc region | CHVQHEGLPEPVTLRWEPKSSDKTHTCPPCPAPELLGGPSVFLFPPKPKDTLMISRTPEVTCVVVDV |
| (knob)) | SHEDPEVKFNWYVDGVEVHNAKTKPREEQYNSTYRVVSVLTVLHQDWLNGKEYKCKVSNKALPAPIE |
| | KTISKAKGQPREPQVYTLPPSREEMTKNQVSLWCLVKGFYPSDIAVEWESNGQPENNYKTTPPVLDS |
| | DGSFFLYSKLTVDKSRWQQGNVFSCSVMHEALHNHYTQKSLSLSPGK (SEQ ID NO: 356) |
| LC1 | DIQMTQSPSSLSASVGDRVTITCKSSQSLLYTSSQKNYLAWYQQKPGKAPKLLIYWASTRESGVPSR |
| (VL1-RTV- | FSGSGSGTDFTLTISSLQPEDFATYYCQQYYAYPWTFGQGTKVEIKRTVRTPKIQVYSRHPAENGKS |
| B2M) | NFLNCYVSGFHPSDIEVDLLKNGERIEKVEHSDLSSSKDSSTYLLYYTEFTPTEKDEYACRVNHVTL |
| | SQPKIVKWDRCM (SEQ ID NO: 357) |
| HC2 | EVQLVESGGGLVQPGGSLRLSCAASGFNIKDTYIHWVRQAPGKGLEWVARIYPTNGYTRYADSVKGR |
| (VH2-CH1- | FTISADTSKNTAYLQMNSLRAEDTAVYYCSRWGGDGFYAMDYWGQGTLVTVSSASTKGPSVFPLAPS |
| Fc region | SKSTSGGTAALGCLVKDYFPEPVTVSWNSGALTSGVHTFPAVLQSSGLYSLSSVVTVPSSSLGTQTY |
| (hole-RF)) | ICNVNHKPSNTKVDKKVEPKSCDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQDWLNGKEYKCKVSNKALPAPI |
| | EKTISKAKGQPREPQVYTLPPSREEMTKNQVSLSCAVKGFYPSDIAVEWESNGQPENNYKTTPPVLD |
| | SDGSFFLVSKLTVDKSRWQQGNVFSCSVMHEALHNRFTQKSLSLSPGK (SEQ ID NO: 358) |
| LC2 | DIQMTQSPSSLSASVGDRVTITCRASQDVNTAVAWYQQKPGKAPKLLIYSASFLYSGVPSRFSGSRS |
| (VL2-CL) | GTDFTLTISSLQPEDFATYYCQQHYTTPPTFGQGTKVEIKRTVAAPSVFIFPPSDEQLKSGTASVVC |
| | LLNNFYPREAKVQWKVDNALQSGNSQESVTEQDSKDSTYSLSSTLTLSKADYEKHKVYACEVTHQGL |
| | SSPVTKSFNRGEC (SEQ ID NO: 359) |

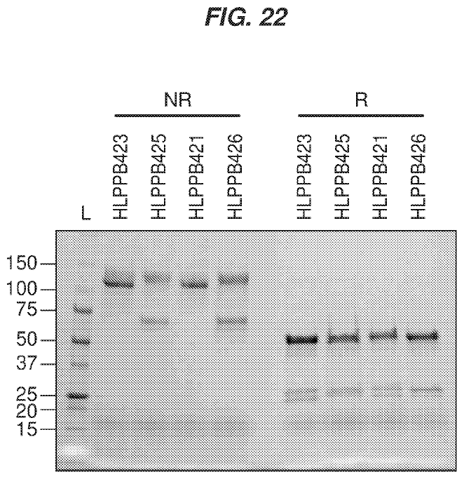

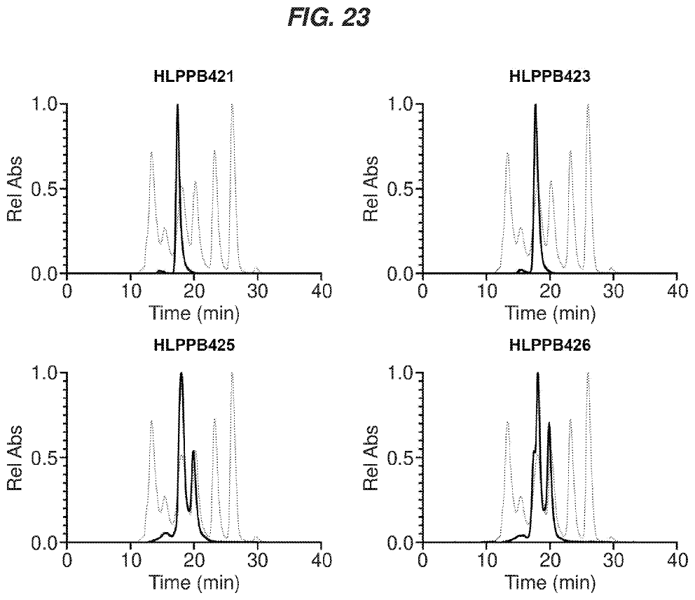

The bispecific antibodies were expressed at 40 ml scale in ExpiCHO cells. The antibodies were purified using a 2-step purification protocol using Protein A chromatography and CH1 capture, followed by dialysis in the phosphate-buffered saline (PBS) buffer. Good yields were observed from the 40 ml ExpiCHO expression. FIG. 22 depicts nonreduced (NR) and reduced (R) PAGE of purified bispecific antibodies. Size exclusion chromatography (SEC) profiles confirmed purity of the bispecific antibodies observed from the PAGE analysis (FIG. 23). Overall, pure species were observed for HLPPB421 and HLPPB423 samples. Both SEC and PAGE analyses indicated the presence of halfmer species in HLPPB425 and HLPPB426 samples. An additional peak was observed in HLPPB426 sample, which may potentially be a homodimeric species formed by the HER2 arm.

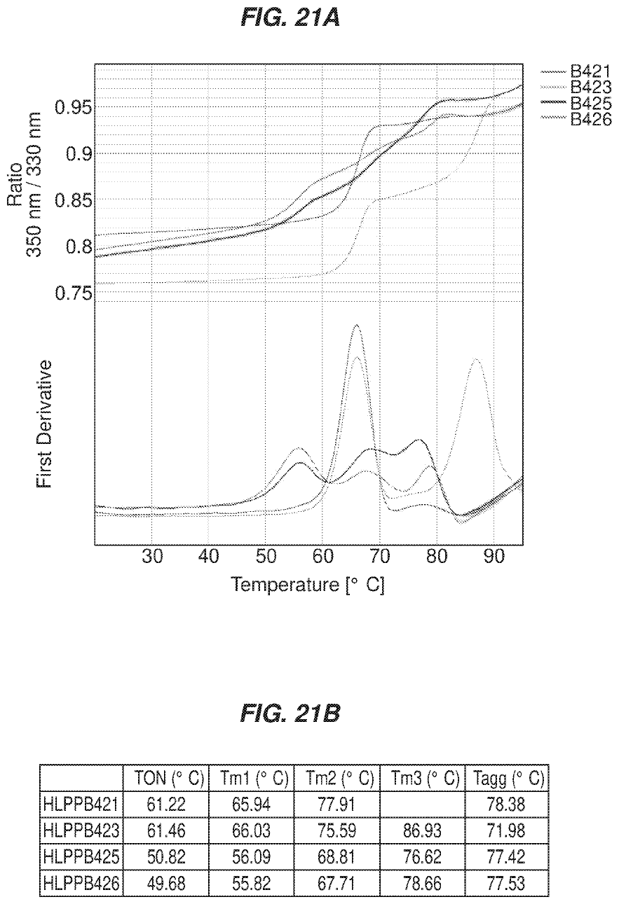

Stability analysis of the bispecific antibodies was performed by thermal melting using nanoscale differential scanning fluorimetry (NanoDSF) (FIGS. 21A-21B). Introduction of unique HLA-E containing arms shows an estimated stability near 65° C., comparable to that of an scFv domain. Of note, samples HLPPB425 and HLPPB426 have halfmer and homodimeric species which are likely melting at 55° C.

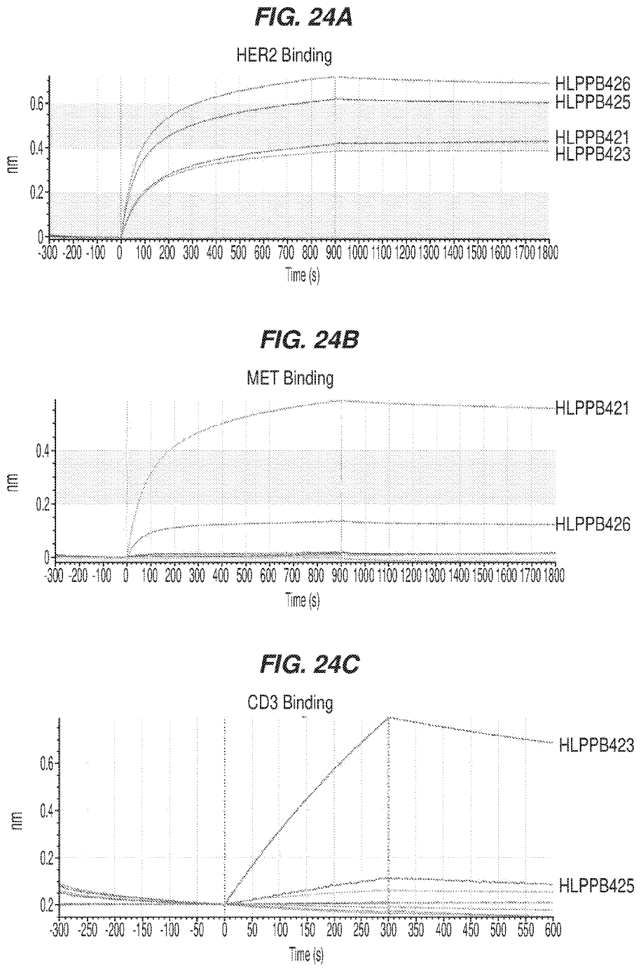

Binding of the bispecific antibodies to their respective targets was confirmed by biolayer interferometry (BLI) analysis (FIGS. 24A-24C). This suggests the correct cognate HC and LC pairing in the bispecific antibodies. In the BLI assays for HER2 and MET, the antibodies were immobilized, and the antigens were used as the soluble analyte. In the BLI assay for CD3, due to nonspecific binding from the soluble CD3 antigen (Biotinylated Human CD3 epsilon&CD3 delta Heterodimer Protein, His, Avitag™&Tag Free from AcroBiosystems), the assay was reversed, i.e., immobilizing the antigen to the tip and using the bispecific antibodies as the analyte. In the HER2 binding data (FIG. 24A), higher HER2 max signal from HLPPB426 and HLPPB425 was observed, which may be due to excess HER2 Hole-RF homodimer/halfmer compared to HLPPB421 and HLPPB423. Consistent with the HER2 binding data, lower max signal from HLPPB426 was observed in the MET binding data (FIG. 24B) and weaker binding from HLPPB425 was observed in CD3 binding data (FIG. 24C), which may also be due to presence of excess HER2-Hole RF homodimer and halfmer species.

### 9.4 Remarks

### 9.4.1 Conclusion

The results described herein indicate that variant HLA-E α3/β2m and ICAM-1 D1 domain dimers may be formatted into Fab-based molecules such as Fabs, or intact or partial antibodies (e.g., monoparatopic, biparatopic, triparatopic, tetraparatopic, etc.), bispecific antibodies, trispecific antibodies, tetraspecific antibodies, or higher multispecific antibodies by using mutation patterns, disulfide bonds, elbow sequences, and/or hinge regions as described herein.

### 10. REFERENCES CITED

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. An antigen-binding molecule, comprising
a first light chain polypeptide comprising, in amino-terminus to carboxy-terminus order: VL1-LD1, wherein VL1 is a first light chain variable domain and LD1 is a first light chain dimerization domain; and
a first heavy chain polypeptide comprising, in amino-terminus to carboxy-terminus order: VH1-HD1, wherein VH1 is a first heavy chain variable domain and HD1 is a first heavy chain dimerization domain,
wherein
a) i) LD1 comprises a beta-2 microglobulin (B2M) domain and HD1 comprises a HLA-E A3 (EA3) domain, or LD1 comprises an EA3 domain and HD1 comprises a B2M domain, wherein the B2M domain and the EA3 domain bind to each other to form a dimer; or
ii) LD1 comprises a first intracellular adhesion molecule-1 (ICAM-1) D1 domain and HD1 comprises a second ICAM-1 D1 domain, wherein the first and second ICAM-1 D1 domains bind to each other to form a dimer;
b) i) at least one of the B2M domain or EA3 domain differs from the wild-type B2M domain (SEQ ID NO:2) or wild-type EA3 domain (SEQ ID NO:33), respectively, and
ii) the first ICAM-1 D1 domain differs from the second ICAM-1 D1 domain; and
c) VL1 and VH1 form a first paratope.

2. The antigen-binding molecule of claim 1,
wherein LD1 comprises the B2M domain and HD1 comprises the EA3 domain, or LD1 comprises the EA3 domain and HD1 comprises the B2M domain, wherein the B2M domain and the EA3 domain bind to each other to form a dimer, and
wherein the B2M domain comprises
a) the amino acid sequence of
(SEQ ID NO: 2)
RTPKIQVYSRHPAENGKSNFLNCYVSGFHPSDIEVDLLKNGERIEK
VEHSDLSFSKDWSFYLLYYTEFTPTEKDEYACRVNHVTLSQPKIVK
WDRDM;
b) an amino acid sequence having at least 85% sequence identity to SEQ ID NO:2;
c) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2;
d) an amino acid sequence having one, two, three, four, five, six, seven, or eight single amino acid substitutions to the sequence of SEQ ID NO:2 at one or more amino acid positions 4, 8, 10, 54, 58, 60, 96, and 97 of SEQ ID NO:2, wherein each of the single amino acid substitutions is independently selected from the group consisting of F, W, C, S, and T;
e) an amino acid sequence of a human B2M sequence as set forth in SEQ ID NO:2 but comprising a set of substitutions of F56S, W60S, and F62T with positions numbered according to the B2M amino acid numbering in the following table, optionally further comprising one, two, three, four, or five single amino acid substitutions at positions K6, Y10, R12, D98, or M99 with positions numbered according to the B2M amino acid numbering in the following table;

| | |
|---|---|
| B2M Amino Acid Numbering | 0--------1---------2---------3---------4---------5 |
| (residues 3-50) | 12345678901234567890123456789012345678901234567890 |
| B2M WT Sequence | RTPKIQVYSRHPAENGKSNFLNCYVSGFHPSDIEVDLLKNGERIEKVE |
| (positions K6, Y10, and R12 are | (continues below) |
| bolded and underlined) | |
| B2M Amino Acid Numbering | 5--------6---------7---------8---------9--------- |
| (residues 51-99) | 1234567890123456789012345678901234567890123456789 |
| B2M WT Sequence | HSDLSFSKDWSFYLLYYTEFTPTEKDEYACRVNHVTLSQPKIVKWDRDM |
| (positions F56, W60, F62, D98, and | (SEQ ID NO: 2) |
| M99 are bolded and underlined) | |

f) an amino acid sequence of a human B2M sequence as set forth in SEQ ID NO:2 but comprising a set of substitutions selected from the following, with positions numbered according to the B2M amino acid numbering in the following table,
i) F56S, W60S, F62T, and K6C;
ii) F56S, W60S, F62T, and any one of Y10C, Y10F, and Y10W;
iii) F56S, W60S, F62T, and R12C;
iv) F56S, W60S, F62T, and any one of D98C; D98F, and D98W;
v) F56S, W60S, F62T, and any one of M99C, M99F, and M99W; or

| | |
|---|---|
| B2M Amino Acid Numbering | 0--------1---------2---------3---------4---------5 |
| (residues 3-50) | 12345678901234567890123456789012345678901234567890 |
| B2M WT Sequence | RTPKIQVYSRHPAENGKSNFLNCYVSGFHPSDIEVDLLKNGERIEKVE |
| (positions K6, Y10, and R12 are | (continues below) |
| bolded and underlined) | |
| B2M Amino Acid Numbering | 5--------6---------7---------8---------9--------- |
| (residues 51-99) | 1234567890123456789012345678901234567890123456789 |
| B2M WT Sequence | HSDLSFSKDWSFYLLYYTEFTPTEKDEYACRVNHVTLSQPKIVKWDRDM |
| (positions F56, W60, F62, D98, and | (SEQ ID NO: 2) |
| M99 are bolded and underlined) | |

g) the amino acid sequence of any one of SEQ ID NOS:4-30.

3. The antigen-binding molecule of claim 1, wherein LD1 comprises the B2M domain and HD1 comprises the EA3 domain, or LD1 comprises the EA3 domain and HD1 comprises the B2M domain, wherein the B2M domain and the EA3 domain bind to each other to form a dimer, and
wherein the EA3 domain comprises
a) the amino acid sequence of
(SEQ ID NO: 33)
LHLEPPKTHVTHHPISDHEATLRCWALGFYPAEITLTWQQDGEGHT
QDTELVETRPAGDGTFQKWAAVVVPSGEEQRYTCHVQHEGLPEPVT
LRW;
b) an amino acid sequence having at least 85% sequence identity to SEQ ID NO:33;
c) an amino acid sequence having one, two, three, four, five, or six single amino acid substitutions to the sequence of SEQ ID NO:33 at one or more amino acid positions 13, 23, 53, 55, 59, and 63 of SEQ ID NO:33;
d) an amino acid sequence having one, two, three, four, five, or six single amino acid substitutions to the sequence of SEQ ID NO:33 at one or more amino acid positions 13, 23, 53, 55, 59, and 63 of SEQ ID NO:33, wherein each of the single amino acid substitutions is independently selected from the group consisting of A, C, and L;
e) an amino acid sequence of a human EA3 sequence as set forth in SEQ ID NO:33 but comprising one, two, three, four, five, or six single amino acid substitutions at positions H192, R202, E232, R234, D238, or Q242 with positions numbered according to the EA3 amino acid numbering in the following table;

| | |
|---|---|
| EA3 Amino Acid Numbering | 18--------19--------20--------21--------22--------23 |
| (residues 180-230) | 012345678901234567890123456789012345678901234567890 |
| EA3 WT Sequence | LHLEPPKTHVTHHPISDHEATLRCWALGFYPAEITLTWQQDGEGHTQDTEL |
| (positions H192 and R202 are | (continues below) |
| bolded and-underlined) | |
| EA3 Amino Acid Numbering | 23-------24--------25--------26--------27--------- |
| (residues 231-274) | 1234567890123456789012345678901234567890123456789 |
| EA3 WT Sequence | VETRPAGDGTFQKWAAVVVPSGEEQRYTCHVQHEGLPEPVTLRW |
| (positions E232, R234, D238, | (SEQ ID NO: 33) |
| and Q242 are bolded and | |
| underlined) | |

f) an amino acid sequence of a human EA3 sequence as set forth in SEQ ID NO:33 but comprising a substitution or set of substitutions selected from the following, with positions numbered according to the EA3 amino acid numbering in the following table,
i) H192C;
ii) R202A or R202C;
iii) E232C;
iv) R234A, R234L, or R234C;
v) D238C;
vi) Q242A or Q242L;
vii) R234A and Q242A;
viii) R234A and Q242L;
ix) R234A and Q242A;
x) R234L and Q242L; or

| | |
|---|---|
| EA3 Amino Acid Numbering | 18--------19--------20--------21--------22--------23 |
| (residues 180-230) | 012345678901234567890123456789012345678901234567890 |
| EA3 WT Sequence | LHLEPPKTHVTHHPISDHEATLRCWALGFYPAEITLTWQQDGEGHTQDTEL |
| (positions H192 and R202 are | (continues below) |
| bolded and-underlined) | |
| EA3 Amino Acid Numbering | 23-------24--------25--------26--------27--------- |
| (residues 231-274) | 1234567890123456789012345678901234567890123456789 |
| EA3 WT Sequence | VETRPAGDGTFQKWAAVVVPSGEEQRYTCHVQHEGLPEPVTLRW |
| (positions E232, R234, D238, | (SEQ ID NO: 33) |
| and Q242 are bolded and | |
| underlined) | |

g) the amino acid sequence of any one of SEQ ID NOS:35-46.

4. The antigen-binding molecule of claim 1, wherein LD1 comprises the first ICAM-1 D1 domain and HD1 comprises the second ICAM-1 D1 domain, wherein the first and second ICAM-1 D1 domains bind to each other to form a dimer, wherein the first ICAM-1 D1 domain and the second ICAM-1 D1 domain each comprise a different amino acid sequence selected from the group consisting of
a) the amino acid sequence of
(SEQ ID NO: 49)
QTSVSPSKVILPRGGSVLVTCSTSCDQPKLLGIETPLPKKELL
LPGNNRKVYELSNVQEDSQPMCYSNCPDGQSTAKTFLTVY;
b) an amino acid sequence having at least 81% sequence identity to SEQ ID NO:49;
c) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, optionally further comprising a C-terminal cysteine amino acid addition;
d) an amino acid sequence having 1, 2, 3, 4, 5, 6, 7, 8, 9, 10, 11, 12, 13, 14, or 15 single amino acid substitutions to the sequence of SEQ ID NO:49 at one or more amino acid positions 2, 10, 13, 18, 20, 23, 34, 38, 42, 49, 51, 53, 63, 67, and 78 of SEQ ID NO:49, wherein each of the single amino acid substitutions is independently selected from the group consisting of V, T, F, W, A, K, E, C, and R, optionally further comprising a C-terminal cysteine amino acid addition;
e) an amino acid sequence of a human ICAM-1 D1 sequence as set forth in SEQ ID NO:49 but comprising 1, 2, 3, 4, 5, 6, 7, 8, 9, 10, 11, 12, 13, 14, or 15 single amino acid substitutions at positions T2, I10, R13, L18, T20, T23, E34, P38, L42, R49, V51, E53, P63, S67, or T78 with positions numbered according to the ICAM-1 D1 amino acid numbering in the following table, optionally further comprising a C-terminal cysteine amino acid addition (84C) with positions numbered according to the ICAM-1 D1 amino acid numbering in the following table;

| | |
|---|---|
| ICAM-1 D1 Amino Acid Numbering | 0---------1---------2---------3---------4---------5 |
| (residues 1-50) | 012345678901234567890123456789012345678901234567890 |
| ICAM-1 D1 WT Sequence | QTSVSPSKVILPRGGSVLVTCSTSCDQPKLLGIETPLPKKELLLPGNNRK |
| (positions T2, I10, R13, L18, T20, | (continues below) |
| T23, E34, P38, L42, and R49 are | |
| bolded and underlined) | |
| ICAM-1 D1 Amino Acid Numbering | 5--------6---------7---------8---- |
| (residues 51-83) | 1234567890123456789012345678901234 |
| ICAM-1 D1 WT Sequence | VYELSNVQEDSQPMCYSNCPDGQSTAKTFLTVY |
| (positions V51, E53, P63, S67, and | (SEQ ID NO: 49) |
| T78 are bolded and underlined) | |

f) an amino acid sequence of a human ICAM-1 D1 sequence as set forth in SEQ ID NO:49 but comprising a set of substitutions selected from the following, with positions numbered according to the ICAM-1 D1 amino acid numbering in the following table,
i) E34K;
ii) T2V, I10T, T23A, E34K, P38T, P63V, S67A, and T78A;
iii) T2V, I10T, R13C, T23A, E34K, P38T, R49E, P63V, S67A, T78A, and 84C;
iv) T2V, I10T, R13C, T23A, E34K, P38T, E53R, P63V, S67A, T78A, and 84C;
v) T2V, I10T, R13C, L18F, T23A, E34K, P38T, P63V, S67A, T78A, and 84C;
vi) T2V, I10T, R13C, L18A, T20A, T23A, E34K, P38T, L42A, V51A, P63V, S67A, T78A, and 84C;
vii) T2V, I10T, R13C, L18W, T23A, E34K, P38T, P63V, S67A, T78A, and 84C; or

| | |
|---|---|
| ICAM-1 D1 Amino Acid Numbering | 0---------1---------2---------3---------4---------5 |
| (residues 1-50) | 012345678901234567890123456789012345678901234567890 |
| ICAM-1 D1 WT Sequence | QTSVSPSKVILPRGGSVLVTCSTSCDQPKLLGIETPLPKKELLLPGNNRK |
| (positions T2, I10, R13, L18, T20, | (continues below) |
| T23, E34, P38, L42, and R49 are | |
| bolded and underlined) | |
| ICAM-1 D1 Amino Acid Numbering | 5--------6---------7---------8---- |
| (residues 51-83) | 1234567890123456789012345678901234 |
| ICAM-1 D1 WT Sequence | VYELSNVQEDSQPMCYSNCPDGQSTAKTFLTVY |
| (positions V51, E53, P63, S67, and | (SEQ ID NO: 49) |
| T78 are bolded and underlined) | |

g) the amino acid sequence of any one of SEQ ID NOS:51-58.

5. The antigen-binding molecule of claim 1, comprising a first light chain elbow region between VL1 and LD1.

6. The antigen-binding molecule of claim 1, comprising a first heavy chain elbow region between VH1 and HD1.

7. The antigen-binding molecule of claim 1, comprising
a first light chain polypeptide comprising, in amino-terminus to carboxy-terminus order: VL1-LE1-LD1, wherein LE1 is a first light chain elbow region; and
a first light chain polypeptide comprising, in amino-terminus to carboxy-terminus order: VH1-HE1-HD1, wherein HE1 is a first heavy chain elbow region,
wherein LE1 connects the carboxy-terminus of VL1 to the amino-terminus of LD1 and HE1 connects the carboxy-terminus of VH1 to the amino-terminus of HD1.

8. The antigen-binding molecule of claim 7, wherein LE1 or HE1 is an amino acid sequence of from 3-25 amino acids in length.

9. The antigen-binding molecule of claim 8, wherein LE1 or HE1 is an amino acid sequence selected from the group consisting of RTV; GGS; RTVGGS (SEQ ID NO:59); RTVGGSRTV (SEQ ID NO:60); AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); ASTKGGGGSGGGGS (SEQ ID NO:71); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO: 81); RTVAGGGGSGGGGS (SEQ ID NO: 82); GGGGSGGGGS (SEQ ID NO: 83); GGGGSGGGGSGGGGS (SEQ ID NO: 84); GGGGSGGGGSGGGGSGGGGS (SEQ ID NO:85); and GGGGSGGGGSGGGGSGGGGSGGGGS (SEQ ID NO:86).

10. The antigen-binding molecule of claim 7, wherein
a) LD1 is the B2M domain, and LE1 is an amino acid sequence selected from the group consisting of RTV; GGS; RTVGGS (SEQ ID NO:59); RTVGGSRTV (SEQ ID NO:60); RTVA (SEQ ID NO:72); RTVAG (SEQ ID NO:73); RTVAGG (SEQ ID NO:74); RTVAGGS (SEQ ID NO:75); RTVAGGGS (SEQ ID NO:76); RTVAGGGGS (SEQ ID NO:77); RTVAGGGGSG (SEQ ID NO:78); RTVAGGGGSGG (SEQ ID NO:79); RTVAGGGGSGGS (SEQ ID NO:80); RTVAGGGGSGGGS (SEQ ID NO:81); and RTVAGGGGSGGGGS (SEQ ID NO:82);
b) HD1 is the EA3 domain, and HE1 is an amino acid sequence selected from the group consisting of GGS; AST; ASTK (SEQ ID NO:61); ASTKG (SEQ ID NO:62); ASTKGG (SEQ ID NO:63); ASTKGGS (SEQ ID NO:64); ASTKGGGS (SEQ ID NO:65); ASTKGGGGS (SEQ ID NO:66); ASTKGGGGSG (SEQ ID NO:67); ASTKGGGGSGG (SEQ ID NO:68); ASTKGGGGSGGS (SEQ ID NO:69)l ASTKGGGGSGGGS (SEQ ID NO:70); or ASTKGGGGSGGGGS (SEQ ID NO:71); or
c) LD1 or HD1 is the first or second ICAM-1 D1 domain, and LE1 or HE1 is an amino acid sequence selected from the group consisting of GGGGSGGGGS (SEQ ID NO:83); GGGGSGGGGSGGGGS (SEQ ID NO:84); GGGGSGGGGSGGGGSGGGGS (SEQ ID NO:85); and GGGGSGGGGSGGGGSGGGGSGGGGS (SEQ ID NO:86).

11. (canceled)

12. (canceled)

13. The antigen-binding molecule of claim 1, comprising
a first light chain polypeptide comprising, in amino-terminus to carboxy-terminus order: VL1-LD1-LS1, wherein LS1 is a first light chain spacer region, and
a first heavy chain polypeptide comprising, in amino-terminus to carboxy-terminus order: VH1-HD1-HS1, wherein HS1 is a first heavy chain spacer region.

14. The antigen-binding molecule of claim 13, wherein HS1 or LS1 is an amino acid sequence of from 2-9 amino acids in length.

15. The antigen-binding molecule of claim 13, wherein HS1 or LS1 is an amino acid sequence selected from the group consisting of EPKSS (SEQ ID NO:87); SG; EPKSC (SEQ ID NO:88); GGSGECSG (SEQ ID NO:89); GGGSGECSG (SEQ ID NO:90); GGSGESSG (SEQ ID NO:91); and GGGSGESSG (SEQ ID NO:92).

16. (canceled)

17. (canceled)

18. (canceled)

19. (canceled)

20. The antigen-binding molecule of claim 1, which does not comprise a dimerization sequence of a heavy chain constant domain 1 (CH1) or light chain constant domain (CL).

21. (canceled)

22. The antigen-binding molecule of claim 1, which further comprises a second light chain polypeptide comprising a second light chain variable domain (VL2), and a second heavy chain polypeptide comprising a second heavy chain variable domain (VH2), wherein VL2 and VH2 form a second paratope.

23. The antigen-binding molecule of claim 22, wherein the first and second paratopes bind to different antigens.

24. (canceled)

25. (canceled)

26. (canceled)

27. (canceled)

28. (canceled)

29. (canceled)

30. (canceled)

31. (canceled)

32. An isolated polynucleotide encoding the antigen-binding molecule of claim 1, optionally wherein the isolated polynucleotide sequence comprises a nucleotide sequence selected from SEQ ID NOS: 3 and 263-343, or a nucleotide sequence having at least 85% sequence identity to any of SEQ ID NOS: 3 and 263-343.

33. A vector comprising the isolated polynucleotide of claim 32.

34. A host cell containing the vector of claim 33.

35. A method of producing an antigen-binding molecule, comprising:
i) culturing the host cell of claim 34 under suitable conditions such that the antigen-binding molecule is expressed by the host cell; and
ii) isolating the antigen-binding molecule.
